(19) United States
(12) Patent Application Publication
(10) Pub. No.: US 2016/0272645 A1
(43) Pub. Date: Sep. 22, 2016

(54) Heterocyclic Compounds and Methods of Use

(71) Applicant: MEDIVATION TECHNOLOGIES, INC., San Francisco, CA (US)

(72) Inventors: Roopa RAI, San Carlos, CA (US); Sarvajit CHAKRAVARTY, Mountain View, CA (US); Michael John GREEN, Half Moon Bay, CA (US); Son Minh PHAM, San Francisco, CA (US); Brahmam PUJALA, Noida (IN); Anil Kumar AGARWAL, Noida, CA (US); Anjan Kumar NAYAK, Noida (IN); Sweta KHARE, Noida (IN); Rambabu GUGULOTH, Noida (IN); Nitin Atmaram RANDIVE, Noida (IN)

(73) Assignee: Medivation Technologies, Inc., San Francisco, CA (US)

(21) Appl. No.: 15/030,045

(22) PCT Filed: Oct. 17, 2014
(86) PCT No.: PCT/US14/61170

§ 371 Date: May 9, 2016

(30) Foreign Application Priority Data
Oct. 18, 2013 (IN) 4713/CHE/2013

(51) Int. Cl. C07D 487/04

(52) U.S. Cl. CPC C07D 487/04

(57) ABSTRACT

The present application discloses compounds that are inhibitors of Btk, compounds that are inhibitors of PI3Kδ, and compounds that are dual inhibitors of both Btk and PI3Kδ. Also described are methods for synthesizing such inhibitors and methods for using such inhibitors for the treatment of diseases wherein inhibition of Btk and PI3Kδ provides a therapeutic benefit to a patient having the disease.

### BACKGROUND

Despite the many advances in treating autoimmune diseases and hematologic diseases, there remains a need for effective therapies to treat such diseases. Bruton's tyrosine kinase (Btk) is a Tec family kinase known for its role in B-cell antigen receptor (BCR) signaling. Activation of this BCR signaling pathway leads to various B-cell malignancies and autoimmune diseases, including arthritis, lupus, multiple sclerosis, B-cell lymphomas, and leukemia such as chronic lymphocytic leukemia (CLL). The development of small molecule Btk inhibitors may provide therapeutic benefits for the treatment of leukemia, lymphoma and such autoimmune diseases [Honigberg, et al. Proc. Natl. Acad. Sci. (2010), 107:29, pp 13075-13080; Barrientos, et al. Leukemia & Lymphoma (August 2013), 54:8, pp 1817-1820; Li, et al. J. Med. Chem. (2014), 57:12, pp 5112-5128]. Inhibition of the BCR signaling pathway has several intervention points: at SYK, Btk, and PI3Kδ. Phosphoinositide 3-kinases (PI3Ks) constitute a family of enzymes widely involved in cell signaling and controlling a broad number of cellular processes including cell proliferation, survival, motility and metabolism. They comprise 4 isoforms: α, β, γ, and δ, each of which has been studied extensively with potential small molecule inhibitors of pan-PI3K, or more preferentially for selective isoforms, having been developed [Ciraolo, et al. Curr. Med. Chem. (2011), 18, pp 2674-2685]. The PI3Kδ isoform has proved of particular interest as a central signaling enzyme that mediates the effects of multiple receptors on B-cells. PI3Kδ signaling is important for B-cell survival, migration and activation, and PI3Kδ kinase activation is believed to be involved in a range of cellular responses including cell growth, differentiation and apoptosis. Targeting of PI3Kδ is thus an attractive means of controlling aberrant B-cell activation [Puri, et al. Frontiers in Immunology (August 2012), 3:256, pp 1-16]. Potential therapies targeting aberrant BCR signaling, including the individual inhibition of PI3K, or SYK, or BTK, have been summarized recently [Choi, et al. Cancer J. (2012), 18, pp 404-410; Rickert, R. C. Nat. Rev. Immunol. (2013), 13:8, pp 578-591; Cushing, et al. J. Med. Chem. (2012), 55, pp 8559-8581; Xing, et al. Future Med. Chem. (2014), 6:6, pp 675-695; Shinohara, et al. Bone (2014), 60: pp 8-15]. The capability to inhibit at more than one of the intervention points with a single molecule could provide a synergistic response resulting in increased therapeutic efficacy and reduced resistance often observed with inhibition of a single kinase. As such, dual inhibitors of BTK and PI3Kδ should overcome resistance due to SYK, BTK and PI3Kδ monotherapy, and that such dual inhibitors could inhibit SYK-independent proliferation of CLL cells.

### BRIEF SUMMARY

Described herein are compounds that are dual inhibitors of both Btk and PI3Kδ. Also described are methods for synthesizing such inhibitors and methods for using such inhibitors for the treatment of diseases wherein inhibition of Btk and PI3Kδ provides a therapeutic benefit to a patient having the disease. In one aspect, the compounds are reversible inhibitors of both BTK and PI3Kδ. In another aspect, the compounds are irreversible inhibitors of both BTK and PI3Kδ.

### DETAILED DESCRIPTION

### Definitions

For use herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”.

Unless clearly indicated otherwise, “an individual” as used herein intends a mammal, including but not limited to a human, bovine, primate, equine, canine, feline, porcine, and ovine animals. Thus, the compositions and methods provided herein use in both human medicine and in the veterinary context, including use in agricultural animals and domestic pets. The individual may be a human who has been diagnosed with or is suspected of having cancer. The individual may be a human who exhibits one or more symptoms associated with cancer. The individual may be a human who has a mutated or abnormal gene associated with cancer. The individual may be a human who is genetically or otherwise predisposed to developing cancer.

As used herein, “treatment” or “treating” is an approach for obtaining beneficial or desired results including clinical results. For purposes of the compositions and methods provided herein, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, delaying or slowing the progression of the disease, ameliorating the disease state, providing a remission (whether partial or total) of the disease, decreasing the dose of one or more other medications required to treat the disease, enhancing the effect of another medication used to treat the disease, increasing the quality of life of an individual having the disease, and/or prolonging survival. A method of treating cancer encompasses a reduction of the pathological consequence of cancer. The methods described herein contemplate any one or more of these aspects of treatment.

As used herein, “delaying” the development of a disease or condition such as cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams, mammography, imaging, or biopsy. Development may also refer to disease progression that may be initially undetectable and includes occurrence, recurrence, and onset.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

As used herein, by “combination therapy” is meant a therapy that includes two or more different compounds. Thus, in one aspect, a combination therapy comprising a compound detailed herein and another compound is provided. In some embodiments, the combination therapy optionally includes one or more pharmaceutically acceptable carriers or excipients, non-pharmaceutically active compounds, and/or inert substances. In various embodiments, treatment with a combination therapy may result in an additive or even synergistic (e.g., greater than additive) result compared to administration of a single compound provided herein alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency, and/or duration of one or more side-effects associated with the compound.

As used herein, the term “effective amount” intends such amount of a compound provided herein which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In various embodiments, an effective amount of the composition or therapy may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

As is understood in the art, an “effective amount” may be in one or more doses, i.e., a single dose or multiple doses may be required to achieve the desired treatment endpoint. An effective amount may be considered in the context of administering one or more therapeutic agents, and a compound, or pharmaceutically acceptable salt thereof, may be considered to be given in an effective amount if, in conjunction with one or more other agents, a desirable or beneficial result may be or is achieved.

A “therapeutically effective amount” refers to an amount of a compound or salt thereof sufficient to produce a desired therapeutic outcome (e.g., reducing the severity or duration of, stabilizing the severity of, or eliminating one or more symptoms of cancer). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients.

A “prophylactically effective amount” refers to an amount of a compound, or pharmaceutically acceptable salt thereof, sufficient to prevent or reduce the severity of one or more future symptoms of cancer when administered to an individual who is susceptible and/or who may develop cancer. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotypes presenting during future development of the disease).

It is understood that an effective amount of a compound or pharmaceutically acceptable salt thereof, including a prophylactically effective amount, may be given to an individual in the adjuvant setting, which refers to a clinical setting in which an individual has had a history of cancer, and generally (but not necessarily) has been responsive to therapy, which includes, but is not limited to, surgery (e.g., surgical resection), radiotherapy, and chemotherapy. However, because of their history of the cancer, these individuals are considered at risk of developing cancer. Treatment or administration in the “adjuvant setting” refers to a subsequent mode of treatment.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound provided herein in its free acid or base form with a suitable organic or inorganic base or acid, respectively, and isolating the salt thus formed during subsequent purification. A pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “excipient” as used herein means an inert or inactive substance that may be used in the production of a drug or pharmaceutical, such as a tablet containing a compound provided herein as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Alkyl” refers to and includes saturated linear or branched univalent hydrocarbon structures and combinations thereof. Particular alkyl groups are those having 1 to 20 carbon atoms (a “C₁-C₂₀ alkyl”). More particular alkyl groups are those having 1 to 8 carbon atoms (a “C₁-C₈ alkyl”). When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl and iso-propyl. This term is exemplified by groups such as methyl, t-butyl, n-heptyl, octyl, and the like.

“Cycloalkyl” refers to and includes cyclic univalent hydrocarbon structures. Cycloalkyl can consist of one ring, such as cyclohexyl, or multiple rings, such as adamantyl. A cycloalkyl comprising more than one ring may be fused, spiro or bridged, or combinations thereof. A preferred cycloalkyl is a saturated cyclic hydrocarbon having from 3 to 13 annular carbon atoms. A more preferred cycloalkyl is a saturated cyclic hydrocarbon having from 3 to 8 annular carbon atoms (a “C₃-C₈ cycloalkyl”). Examples of cycloalkyl groups include adamantyl, decahydronaphthalenyl, cyclopropyl, cyclobutyl, cyclopentyl, cyclohexyl, and the like.

“Alkylene” refers to the same residues as alkyl, but having bivalency. Examples of alkylene include methylene (—CH₂—), ethylene (—CH₂CH₂—), propylene (—CH₂CH₂CH₂—), butylene (—CH₂CH₂CH₂CH₂—) and the like.

“Alkenyl” refers to an unsaturated hydrocarbon group having at least one site of olefinic unsaturation (i.e., having at least one moiety of the formula C═C) and preferably having from 2 to 10 carbon atoms and more preferably 2 to 8 carbon atoms. Examples of alkenyl include but are not limited to —CH₂—CH═CH—CH₃ and —CH═CH—CH═CH₂.

“Cycloalkenyl” refers to an unsaturated hydrocarbon group within a cycloalkyl having at least one site of olefinic unsaturation (i.e., having at least one moiety of the formula C═C). Cycloalkenyl can consist of one ring, such as cyclohexyl, or multiple rings, such as norbornenyl. A more preferred cycloalkenyl is an unsaturated cyclic hydrocarbon having from 5 to 8 annular carbon atoms (a “C₅-C₈ cycloalkenyl”). Examples of cycloalkenyl groups include cyclopentenyl, cyclohexenyl, and the like.

“Alkynyl” refers to an unsaturated hydrocarbon group having at least one site of acetylenic unsaturation (i.e., having at least one moiety of the formula CC) and preferably having from 2 to 10 carbon atoms and more preferably 2 to 8 carbon atoms and the like.

“Substituted alkyl” refers to an alkyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, substituted or unsubstituted cycloalkyl, substituted or unsubstituted cycloalkenyl, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted cycloalkyl” refers to a cycloalkyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, substituted or unsubstituted cycloalkenyl, aryl, substituted aryl, heteroaryl, substituted heteroaryl, aryloxy, substituted aryloxy, cyano, halo, hydroxyl, nitro, carboxyl, thiol, thioalkyl, substituted or unsubstituted cycloalkyl, substituted or unsubstituted cycloalkenyl, substituted or unsubstituted alkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted alkenyl” refers to alkenyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted cycloalkenyl” refers to a cycloalkenyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted alkynyl” refers to alkynyl groups having from 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted heterocyclyl, substituted or unsubstituted aralkyl, aminosulfonyl, sulfonylamino, sulfonyl, oxo, alkoxyalkylenecarbonyl and the like.

“Acyl” refers to the groups H—C(O)—, alkyl-C(O)—, substituted alkyl-C(O)—, alkenyl-C(O)—, substituted alkenyl-C(O)—, cycloalkyl-C(O)—, substituted cycloalkyl-C(O)—, cycloalkenyl-C(O)—, substituted cycloalkenyl-C(O)—, alkynyl-C(O)—, substituted alkynyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups H—C(O)O—, alkyl-C(O)O—, substituted alkyl-C(O)O—, alkenyl-C(O)O—, substituted alkenyl-C(O)O—, cycloalkenyl-C(O)O—, substituted cycloalkenyl-C(O)O—, alkynyl-C(O)O—, substituted alkynyl-C(O)O—, cycloalkyl-C(O)O—, substituted cycloalkyl-C(O)O—, aryl-C(O)O—, substituted aryl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, cycloalkenyl, substituted cycloalkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Heterocycle”, “heterocyclic”, or “heterocyclyl” refers to a saturated or an unsaturated non-aromatic group having a single ring or multiple condensed rings, and having from 1 to 10 annular carbon atoms and from 1 to 4 annular heteroatoms, such as nitrogen, sulfur or oxygen, and the like. A heterocycle comprising more than one ring may be fused, spiro or bridged, or any combination thereof. In fused ring systems, one or more of the rings can be aryl or heteroaryl. A heterocycle having more than one ring where at least one ring is aromatic may be connected to the parent structure at either a non-aromatic ring position or at an aromatic ring position. In one variation, a heterocycle having more than one ring where at least one ring is aromatic is connected to the parent structure at a non-aromatic ring position.

“Substituted heterocyclic” or “substituted heterocyclyl” refers to a heterocycle group which is substituted with from 1 to 3 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted aralkyl, aminosulfonyl, sulfonylamino, sulfonyl, oxo, alkoxyalkylenecarbonyl and the like. In one variation, a substituted heterocycle is a heterocycle substituted with an additional ring, wherein the additional ring may be aromatic or non-aromatic.

“Aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” or “HetAr” refers to an unsaturated aromatic carbocyclic group having from 1 to 10 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

“Substituted aryl” refers to an aryl group having 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted heteroaryl” refers to a heteroaryl group having 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Aralkyl” refers to a residue in which an aryl moiety is attached to an alkyl residue and wherein the aralkyl group may be attached to the parent structure at either the aryl or the alkyl residue. Preferably, an aralkyl is connected to the parent structure via the alkyl moiety. In one variation, an aralkyl is a fused ring system where at least one cycloalkyl moiety is fused with at least one aryl moiety. A “substituted aralkyl” refers to a residue in which an aryl moiety is attached to a substituted alkyl residue and wherein the aralkyl group may be attached to the parent structure at either the aryl or the alkyl residue. When an aralkyl is connected to the parent structure via the alkyl moiety, it may also be referred to as an “alkaryl”. More particular alkaryl groups are those having 1 to 3 carbon atoms in the alkyl moiety (a “C₁-C₃ alkaryl”).

“Alkoxy” refers to the group alkyl-O—, which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like. Similarly, alkenyloxy refers to the group “alkenyl-O—” and alkynyloxy refers to the group “alkynyl-O—”. “Substituted alkoxy” refers to the group substituted alkyl-O.

“Unsubstituted amino” refers to the group —NH₂.

“Substituted amino” refers to the group —NRₐR_{b}, where either (a) each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic, provided that both Rₐ and R_{b} groups are not H; or (b) Rₐ and R_{b} are joined together with the nitrogen atom to form a heterocyclic or substituted heterocyclic ring.

“Aminoacyl” refers to the group —C(O)NRₐR_{b} where Rₐ and R_{b} are independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic or Rₐ and R_{b} groups can be joined together with the nitrogen atom to form a heterocyclic or substituted heterocyclic ring.

“Acylamino” refers to the group —NRₐC(O)R_{b} where each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic or substituted heterocyclic. Preferably, Rₐ is H or alkyl.

“Sulfonylamino” refers to the groups —NRSO₂-alkyl, —NRSO₂-substituted alkyl, —NRSO₂-alkenyl, —NRSO₂-substituted alkenyl, —NRSO₂-cycloalkenyl, —NRSO₂-substituted cycloalkenyl, —NRSO₂-alkynyl, —NRSO₂-substituted alkynyl, —NRSO₂-cycloalkyl, —NRSO₂— substituted cycloalkyl, —NRSO₂-aryl, —NRSO₂-substituted aryl, —NRSO₂-heteroaryl, —NRSO₂— substituted heteroaryl, —NRSO₂-heterocyclic, and —NRSO₂-substituted heterocyclic, where R is H or alkyl.

“Aminosulfonyl” refers to the groups —SO₂NH₂, —SO₂NR-alkyl, —SO₂NR-substituted alkyl, —SO₂NR-cycloalkyl, —SO₂NR-substituted cycloalkyl, —SO₂NR-alkenyl, —SO₂NR-substituted alkenyl, —SO₂NR-cycloalkenyl, —SO₂NR-substituted cycloalkenyl, —SO₂NR-alkynyl, —SO₂NR-substituted alkynyl, —SO₂NR-aryl, —SO₂NR-substituted aryl, —SO₂NR-heteroaryl, —SO₂NR-substituted heteroaryl, —SO₂NR-heterocyclic, and —SO₂NR-substituted heterocyclic, where R is H or alkyl, or —SO₂NR₂, where the two R groups are taken together and with the nitrogen atom to which they are attached to form a heterocyclic or substituted heterocyclic ring.

“Sulfonyl” refers to the groups —SO₂-alkyl, —SO₂-substituted alkyl, —SO₂-cycloalkyl, —SO₂-substituted cycloalkyl, —SO₂-alkenyl, —SO₂-substituted alkenyl, —SO₂-cycloalkenyl, —SO₂-substituted cycloalkenyl, —SO₂-alkynyl, —SO₂-substituted alkynyl, —SO₂-aryl, —SO₂— substituted aryl, —SO₂-heteroaryl, —SO₂-substituted heteroaryl, —SO₂-heterocyclic, and —SO₂-substituted heterocyclic.

“Alkoxycarbonylamino” refers to the group —NRₐC(O)OR_{b} where each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic and substituted heterocyclyl.

“Alkoxyalkylenecarbonyl” refers to the group —C(═O)—(CH₂)ₙ—OR where R is a substituted or unsubstituted alkyl and n is an integer from 1 to 100, more preferably n is an integer from 1 to 10 or 1 to 5.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl, etc., refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each H is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Cyano” refers to the group —CN.

“Oxo” refers to the moiety ═O.

“Nitro” refers to the group —NO₂.

“Thioalkyl” refers to the groups —S-alkyl.

“Thio” refers to the group —S— that may be a —SH (a thiol or an unsubstituted thio group) or selected from the groups —S-alkyl, —S-alkenyl, —S-alkynyl, —S-cycloalkyl, —S— heterocyclyl, —S-aryl and —S-heteroaryl.

“Aminosulfonylalkylene” refers to the group —R¹SO₂NRₐR_{b} where Rₐ and R_{b} are independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic, or the Rₐ and R_{b} groups can be joined together with the nitrogen atom to form a heterocyclic or substituted heterocyclic ring and R¹ is an alkylene group.

“Alkoxycarbonyl” refers to as used herein refers to the groups —C(O)O-alkyl, —C(O)O-substituted alkyl, —C(O)O-cycloalkyl, —C(O)O-substituted cycloalkyl, —C(O)O-aryl, —C(O)O-substituted aryl, —C(O)O-alkenyl, —C(O)O-substituted alkenyl, —C(O)O-cycloalkenyl, —C(O)O-substituted cycloalkenyl, —C(O)O-alkynyl, —C(O)O-substituted alkynyl, —C(O)O— heteroaryl, —C(O)O-substituted heteroaryl, —C(O)O-heterocyclic or —C(O)O-substituted heterocyclic.

“Geminal” refers to the relationship between two moieties that are attached to the same atom. For example, in the residue —CH₂—CHR¹R², R¹ and R² are geminal and R¹ may be referred to as a geminal R group to R².

“Vicinal” refers to the relationship between two moieties that are attached to adjacent atoms. For example, in the residue —CHR¹—CH₂R², R¹ and R² are vicinal and R¹ may be referred to as a vicinal R group to R².

A composition of “substantially pure” compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

### Compounds of the Invention

Compounds according to the invention are detailed herein, including in the Brief Summary of the Invention and elsewhere. The invention includes the use of all of the compounds described herein, including any and all stereoisomers, including geometric isomers (cis/trans or E/Z isomers), tautomers, salts, N-oxides, and solvates of the compounds described herein, as well as methods of making such compounds.

In one aspect, provided are compounds of formula (I):

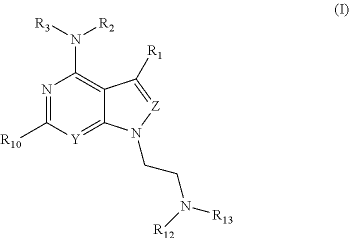

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

Y is CH or N;

Z is N or CR₉;

R₉ is H, or is selected from the group consisting of halo, CN, CF₃, —C(═O)NH₂, and substituted or unsubstituted C₁-C₆ alkyl;

R₁₀ is H, or is selected from the group consisting of halo, —NHR₂, and substituted or unsubstituted C₁-C₆ alkyl;

R₁₂ is selected from the group consisting of H, —[C₁-C₆ alkyl]-CN,

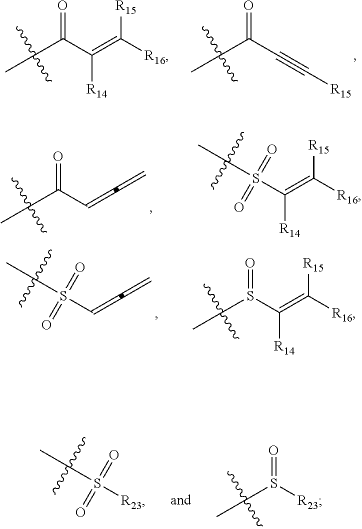

R₁₃ is selected from the group consisting of substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or are independently selected from the group consisting of substituted or unsubstituted C₁-C₆ alkyl, substituted or unsubstituted cycloalkyl, substituted or unsubstituted heterocyclyl, and substituted or unsubstituted amino; and

R₂₃ is substituted or unsubstituted C₁-C₆ alkyl or substituted or unsubstituted C₃-C₆ cycloalkyl.

In another aspect, provided are compounds of formula (II):

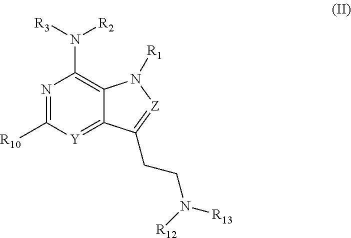

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

Y is CH or N;

Z is N or CR₉;

R₉ is H, or is selected from the group consisting of halo, CN, CF₃, —C(═O)NH₂, and substituted or unsubstituted C₁-C₆ alkyl;

R₁₀ is H, or is selected from the group consisting of halo, —NHR₂, and substituted or unsubstituted C₁-C₆ alkyl;

R₁₂ is H, or is selected from the group consisting of —[C₁-C₆ alkyl]-CN,

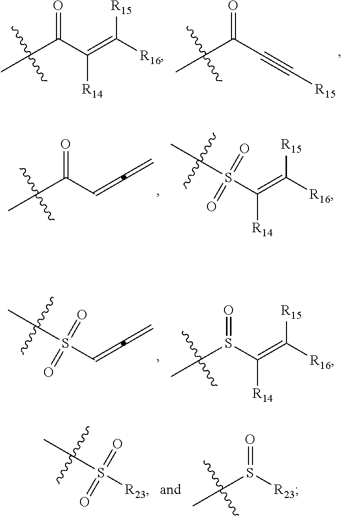

R₁₃ is selected from the group consisting of a substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino; and

R₂₃ is substituted or unsubstituted C₁-C₆ alkyl or substituted or unsubstituted C₃-C₆ cycloalkyl.

In another aspect, provided are compounds of formula (III):

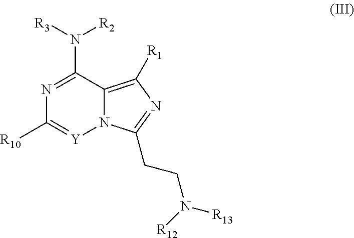

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl; Y is CH or N; R₁₀ is H, or is selected from the group consisting of halo, —NHR₂, and substituted or unsubstituted C₁-C₆ alkyl;

R₁₂ is H, or is selected from the group consisting of —[C₁-C₆ alkyl]-CN,

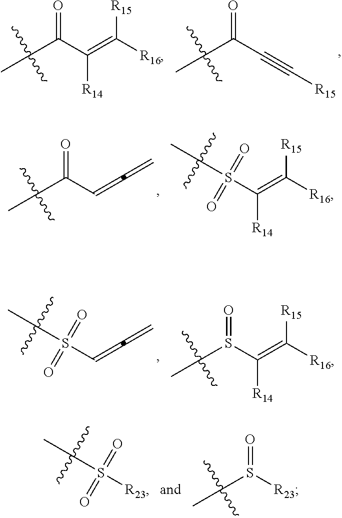

R₁₃ is selected from the group consisting of substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino; and

R₂₃ is substituted or unsubstituted C₁-C₆ alkyl or substituted or unsubstituted C₃-C₆ cycloalkyl.

In another aspect, provided are compounds of formulae (IVa) and (IVb):

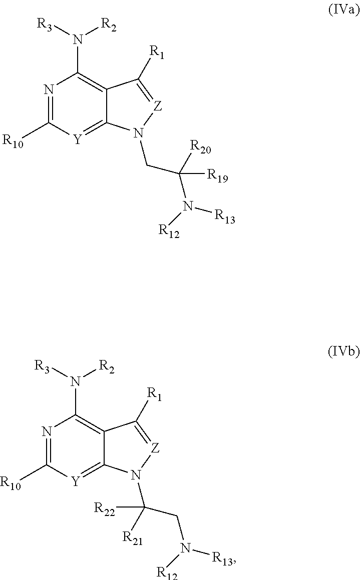

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

Y is CH or N;

Z is N or CR₉;

R₉ is H, or is selected from the group consisting of halo, CN, CF₃, —C(═O)NH₂, and substituted or unsubstituted C₁-C₆ alkyl;

R₁₀ is H, or is selected from the group consisting of halo, and substituted or unsubstituted C₁-C₆ alkyl;

R₁₂ is selected from the group consisting of H, —[C₁-C₆ alkyl]-CN,

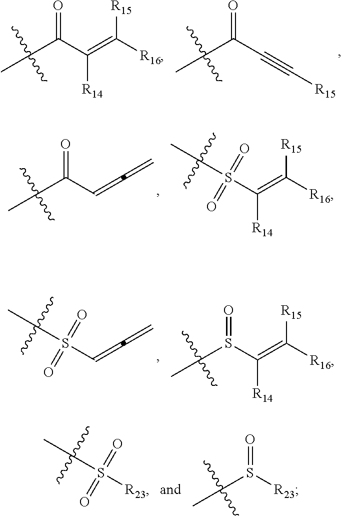

R₁₃ is selected from the group consisting of substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₁₉ and R₂₀ are both unsubstituted C₁-C₆ alkyl, or are taken together with the carbon atom to which they are attached to form an unsubstituted C₃-C₈ cycloalkyl or carbonyl;

R₂₁ and R₂₂ are both unsubstituted C₁-C₆ alkyl; or are taken together with the carbon atom to which they are attached to form an unsubstituted C₃-C₈ cycloalkyl or carbonyl; and

R₂₃ is substituted or unsubstituted C₁-C₆ alkyl or substituted or unsubstituted C₃-C₆ cycloalkyl;

with the provision that for formula (IVb), when both Y and Z are N; R₂, R₃ and R₁₀ are each H; and R₂₁ and R₂₂ are each methyl; then R₁₂ is other than H.

In another aspect, provided are compounds of formulae (Va) and (Vb):

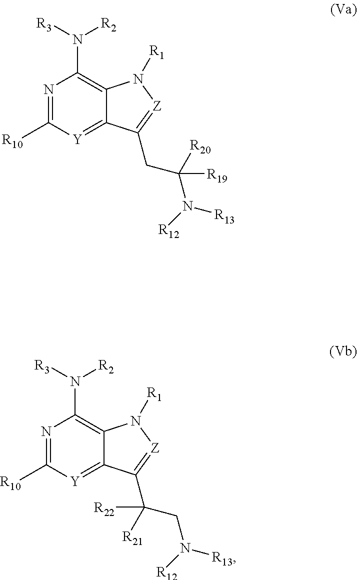

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

Y is CH or N;

Z is N or CR₉;

R₉ is H, or is selected from the group consisting of halo, CN, CF₃, —C(═O)NH₂, and substituted or unsubstituted C₁-C₆ alkyl;

R₁₀ is H, or is selected from the group consisting of halo, —NHR₂, and substituted or unsubstituted C₁-C₆ alkyl;

R₁₂ is selected from the group consisting of H, —[C₁-C₆ alkyl]-CN,

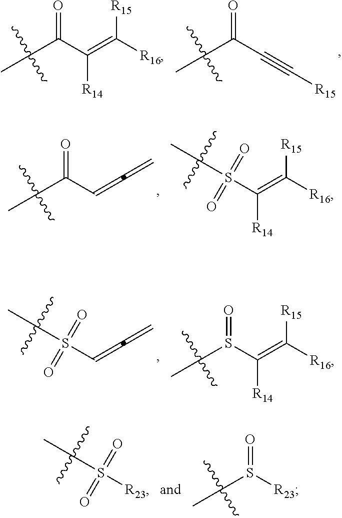

R₁₃ is selected from the group consisting of substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₁₉ and R₂₀ are both unsubstituted C₁-C₆ alkyl, or are taken together with the carbon atom to which they are attached to form an unsubstituted C₃-C₈ cycloalkyl or carbonyl;

R₂₁ and R₂₂ are both unsubstituted C₁-C₆ alkyl; or are taken together with the carbon atom to which they are attached to form an unsubstituted C₃-C₈ cycloalkyl or carbonyl; and

R₂₃ is substituted or unsubstituted C₁-C₆ alkyl or substituted or unsubstituted C₃-C₆ cycloalkyl.

In another aspect, provided are compounds of formulae (VIa) and (VIb):

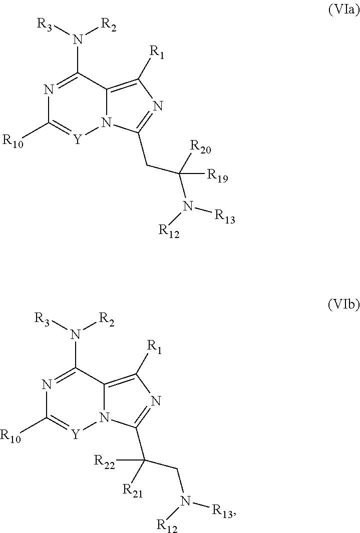

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

Y is CH or N;

R₁₀ is H, or is selected from the group consisting of halo, —NHR₂, and substituted or unsubstituted C₁-C₆ alkyl;

R₁₂ is selected from the group consisting of H, —[C₁-C₆ alkyl]-CN,

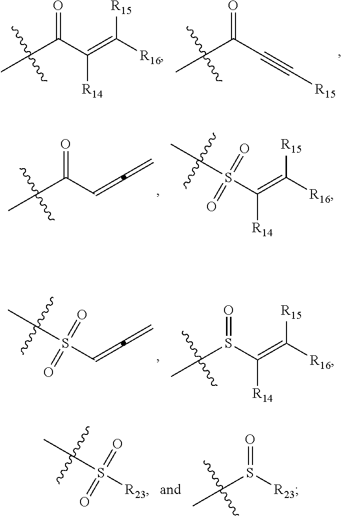

R₁₃ is substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂— substituted heteroaryl, —CH₂-unsubstituted heteroaryl, substituted or unsubstituted cycloalkyl, or a substituted or unsubstituted heterocyclyl;

R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;

R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₁₉ and R₂₀ are both unsubstituted C₁-C₆ alkyl, or are taken together with the carbon atom to which they are attached to form an unsubstituted C₃-C₈ cycloalkyl or carbonyl;

R₂₁ and R₂₂ are both unsubstituted C₁-C₆ alkyl; or are taken together with the carbon atom to which they are attached to form an unsubstituted C₃-C₈ cycloalkyl or carbonyl; and

R₂₃ is substituted or unsubstituted C₁-C₆ alkyl or substituted or unsubstituted C₃-C₆ cycloalkyl.

In some embodiments of formulae (I), (II), (III), (IVa), (IVb), (Va), (Vb), (VIa), and (VIb), R₁ is a substituted or unsubstituted C₁-C₆ alkyl. In some embodiments, R₁ is a substituted or unsubstituted C₂-C₆ alkenyl. In some embodiments, R₁ is a substituted or unsubstituted aryl. In some embodiments, R₁ is a substituted or unsubstituted cycloalkyl. In some embodiments, R₁ is a substituted or unsubstituted heterocyclyl. In some embodiments, R₁ is a substituted or unsubstituted heteroaryl. In some embodiments, R₁ is a substituted or unsubstituted alkoxy. In some embodiments, R₁ is a substituted or unsubstituted aryloxy. In some embodiments, R₁ is a substituted or unsubstituted amino. In some embodiments, R₁ is a substituted or unsubstituted thio.

In some embodiments of formulae (I), (II), (III), (IVa), (IVb), (Va), (Vb), (VIa), and (VIb), R₁ is unsubstituted phenyl. In some embodiments, R₁ is substituted phenyl. In some embodiments, R₁ is phenyl substituted with two or more substituents. In some embodiments, R₁ is phenyl substituted with two, three or more substituents selected from the group consisting of hydroxyl, halo, perhaloalkyl, C₁-C₆ alkoxy, phenoxy, aminoacyl, substituted or unsubstituted amino, substituted or unsubstituted C₁-C₆ alkyl, cyano, and allyloxy. In some embodiments, R₁ is phenyl substituted with two, three or more substituents selected from halo and hydroxyl. In some embodiments, R₁ is phenyl substituted with two, three or more substituents selected from halo and allyloxy. In some embodiments, R₁ is phenyl substituted with one halo and one hydroxyl. In some embodiments, R₁ is phenyl substituted with one halo and one allyloxy. In some embodiments, R₁ is phenyl substituted with one halo and one C₁-C₆ alkoxy.

In some embodiments of formulae (I), (II), (III), (IVa), (IVb), (Va), (Vb), (VIa), and (VIb), R₁ is a substituted phenyl moiety comprising a phenyl fused to a substituted or unsubstituted cycloalkyl or substituted or unsubstituted heterocyclyl. In some embodiments, the substituted phenyl moiety is a benzo [d][1,3]dioxolyl, a 2,3-dihydrobenzo [d][1,4]dioxinyl, a 3,4-dihydro-2H-benzo [b][1,4]dioxepinyl, a 2,3-dihydrobenzo [d]oxazolyl, a 3,4-dihydro-2H-benzo[b][1,4]oxazinyl, a 2,3,4,5-tetrahydrobenzo[b][1,4]oxazepinyl, a 2,3-dihydrobenzo [d]imidazolyl, a 1,2,3,4-tetrahydroquinoxalinyl, or a 2,3,4,5-tetrahydro-1H-benzo[b][1,4]diazepinyl. In these embodiments, the bond to R₁ can be attached at any available position on the substituted phenyl moiety.

In some embodiments, of formulae (I), (II), (III), (IVa), (IVb), (Va), (Vb), (VIa), and (VIb), R₁ is a substituted or unsubstituted heteroaryl. In some embodiments, the heteroaryl is monocyclic. In some embodiments, the heteroaryl is bicyclic. In some embodiments, the heteroaryl comprises one annular heteroatom such as nitrogen, oxygen or sulfur. In some embodiments, the heteroaryl comprises two annular heteroatoms chosen from nitrogen, oxygen and sulfur. In some embodiments, the heteroaryl comprises three annular heteroatoms chosen from nitrogen, oxygen and sulfur. In these embodiments, the bond to R₁ can be attached at any available position on the heteroaryl.

In some embodiments of formulae (I), (II), (III), (IVa), (IVb), (Va), (Vb), (VIa), and (VIb), R₁ is a substituted or unsubstituted pyridin-2-yl, substituted or unsubstituted pyridin-3-yl, or substituted or unsubstituted pyridin-4-yl. In some embodiments, the substituent is selected from the group consisting of C₁-C₆ alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted C₁-C₆ alkyl and substituted or unsubstituted cycloalkyl.

In some embodiments of formulae (I), (II), (III), (IVa), (IVb), (Va), (Vb), (VIa), and (VIb), R₁ is a substituted or unsubstituted benzimidazol-2-yl, substituted or unsubstituted benzimidazol-4-yl, or substituted or unsubstituted benzimidazol-5-yl.

In some embodiments of formulae (I), (II), (III), (IVa), (IVb), (Va), (Vb), (VIa), and (VIb), R₁ is a substituted or unsubstituted pyrazol-3-yl, or substituted or unsubstituted pyrazol-4-yl.

In some embodiments of formulae (I), (II), (III), (IVa), (IVb), (Va), (Vb), (VIa), and (VIb), R₁ is a substituted or unsubstituted indol-1-yl, indol-2-yl, indol-3-yl, indol-4-yl, indol-5-yl, indol-6-yl, or indol-7-yl. In some embodiments, R₁ is a substituted or unsubstituted indol-1-yl, indol-2-yl, or indol-6-yl. In some embodiments, R₁ is a substituted indol-1-yl, substituted indol-2-yl, or substituted indol-6-yl.

In some embodiments of formulae (I), (II), (III), (IVa), (IVb), (Va), (Vb), (VIa), and (VIb), R₁ is a substituted or unsubstituted pyrrolopyridin-2-yl, substituted or unsubstituted pyrrolopyridin-3-yl, substituted or unsubstituted pyrrolopyridin-4-yl, substituted or unsubstituted pyrrolopyridin-5-yl, or substituted or unsubstituted pyrrolopyridin-6-yl. In some embodiments, R₁ is substituted or unsubstituted pyrrolopyridin-2-yl or substituted or unsubstituted pyrrolopyridin-5-yl. In some embodiments, R₁ is substituted pyrrolopyridin-5-yl.

In some embodiments of formulae (I), (II), (III), (IVa), (IVb), (Va), (Vb), (VIa), and (VIb), R₁ is selected from the group consisting of substituted or unsubstituted indazol-3-yl, substituted or unsubstituted indazol-4-yl, substituted or unsubstituted indazol-5-yl, substituted or unsubstituted indazol-6-yl, or substituted or unsubstituted indazol-7-yl. In some embodiments, R₁ is substituted indazol-5-yl.

In some embodiments of formulae (I), (II), (III), (IVa), (IVb), (Va), (Vb), (VIa), and (VIb), R₁ is substituted or unsubstituted pyrimidin-2-yl, substituted or unsubstituted pyrimidin-4-yl or substituted or unsubstituted pyrimidin-5-yl.

In some embodiments of formulae (I), (II), (III), (IVa), (IVb), (Va), (Vb), (VIa), and (VIb), R₂ and R₃ are each H. In some embodiments, R₂ is H and R₃ is C₁-C₆ alkyl. In some embodiments, R₂ is H and R₃ is methyl. In some embodiments, each R₂ and R₃ is methyl.

In some embodiments of formulae (I), (II), (III), (IVa), (IVb), (Va), (Vb), (VIa), and (VIb). Y is CH. In some embodiments, Y is N.

In some embodiments of formulae (I), (II), (IVa), (IVb), (Va), and (Vb), Z is N. In some embodiments, Z is CR₉. In some of these embodiments, R₉ is H. In some of these embodiments, R₉ is halo. In some of these embodiments, R₉ is CN. In some of these embodiments, R₉ is CF₃. In some of these embodiments, R₉ is —C(═O)NH₂. In some of these embodiments, R₉ is substituted or unsubstituted C₁-C₆ alkyl. In some embodiments, Y is CH and Z is N. In some embodiments, Y is N and Z is N. In some embodiments, Y is CH and Z is CR₉. In some embodiments, Y is N and Z is CR₉.

In some embodiments of formulae (I), (II), (III), (IVa), (IVb), (Va), (Vb), (VIa), and (VIb), R₁₂ is

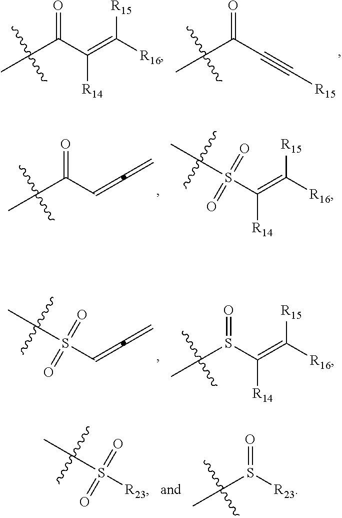

In some embodiments, R₁₂ is

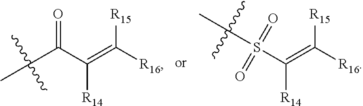

In some embodiments, R₁₂ is

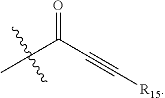

In some embodiments, R₁₂ is

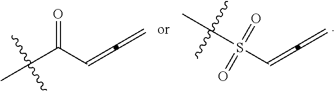

In some of these embodiments, R₁₄ is H. In some embodiments, R₁₄ is CN, halo, —NO₂, CF₃, —SO₂CH₃, —SO₂NH₂, —C(═O)R₂₃, —C(═O)OR₂₃, or —C(═O)NH₂. In some embodiments, R₁₄ is CN.

In some embodiments of formulae (I), (II) and (III), R₁₂ is

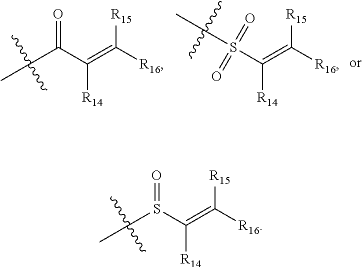

In some of these embodiments, R₁₄ is H. In some of these embodiments, R₁₄ is substituted or unsubstituted C₁-C₆ alkyl. In some of these embodiments, R₁₄ is substituted or unsubstituted cycloalkyl. In some of these embodiments, R₁₄ is substituted or unsubstituted heterocyclyl. In some of these embodiments, R₁₄ is substituted or unsubstituted amino. In some embodiments, R₁₄ is CN, halo, —NO₂, CF₃, —SO₂CH₃, —SO₂NH₂, —C(═O)R₁₂, —C(═O)OR₁₂, or —C(═O)NH₂. In some embodiments, R₁₄ is CN. In some of these embodiments, each R₁₅ and R₁₆ is H. In some of these embodiments, one of R₁₅ and R₁₆ is H, and the other of R₁₅ and R₁₆ is substituted or unsubstituted C₁-C₆ alkyl. In some of these embodiments, R₁₅ is H, and R₁₆ is substituted or unsubstituted C₁-C₆ alkyl. In some of these embodiments, R₁₅ is H, and R₁₆ is substituted or unsubstituted cycloalkyl. In some of these embodiments, R₁₅ is H, and R₁₆ is substituted or unsubstituted heterocyclyl. In some of these embodiments, R₁₅ is H, and R₁₆ is substituted or unsubstituted amino.

In some embodiments of formulae (I), (II), (III), (IVa), (IVb), (Va), (Vb), (VIa), and (VIb), R₁₃ is substituted or unsubstituted phenyl. In some embodiments, R₁₃ is substituted or unsubstituted benzoyl. In some embodiments, R₁₃ is substituted or unsubstituted benzyl. In some embodiments, R₁₃ is substituted or unsubstituted cyclopentyl. In some embodiments, R₁₃ is substituted or unsubstituted cyclohexyl. In some embodiments, R₁₃ is substituted or unsubstituted pyridinyl. In some embodiments, R₁₃ is substituted or unsubstituted pyrimidinyl. In some embodiments, R₁₃ is substituted or unsubstituted pyridinyl. In some embodiments, R₁₃ is substituted or unsubstituted benzimidazolyl.

In some embodiments of formulae (IVa), (Va), and (VIa), R₁₉ and R₂₀ are each methyl. In some embodiments, R₁₉ and R₂₀ are taken together with the carbon to which they are attached to form a cyclopropyl. In some embodiments, R₁₉ and R₂₀ are taken together with the carbon to which they are attached to form a cyclobutyl. In some embodiments, R₁₉ and R₂₀ are taken together with the carbon to which they are attached to form a cyclopentyl. In some embodiments, R₁₉ and R₂₀ are taken together with the carbon to which they are attached to form a cyclohexyl. In some embodiments, R₁₉ and R₂₀ are taken together with the carbon to which they are attached to form a carbonyl.

In some embodiments of formulae (IVb), (Vb), and (VIb), R₂₁ and R₂₂ are each methyl. In some embodiments, R₂₁ and R₂₂ are taken together with the carbon to which they are attached to form a cyclopropyl. In some embodiments, R₂₁ and R₂₂ are taken together with the carbon to which they are attached to form a cyclobutyl. In some embodiments, R₂₁ and R₂₂ are taken together with the carbon to which they are attached to form a cyclopentyl. In some embodiments, R₂₁ and R₂₂ are taken together with the carbon to which they are attached to form a cyclohexyl. In some embodiments, R₂₁ and R₂₂ are taken together with the carbon to which they are attached to form a carbonyl.

In some embodiments of formulae (I), (II) and (III), the compound is of structure B-1, B-2, B-3, B-4, B-5, B-6, B-7, B-8, B-9, or B-10:

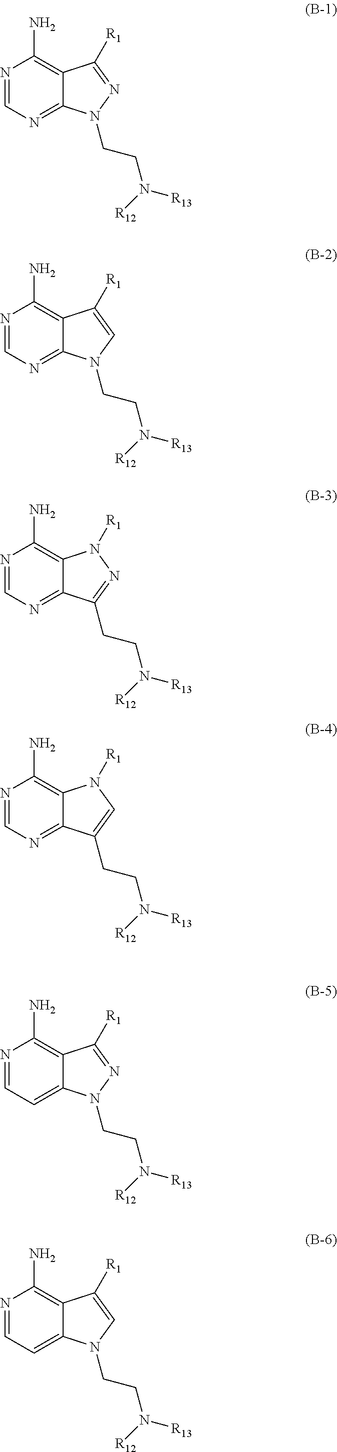

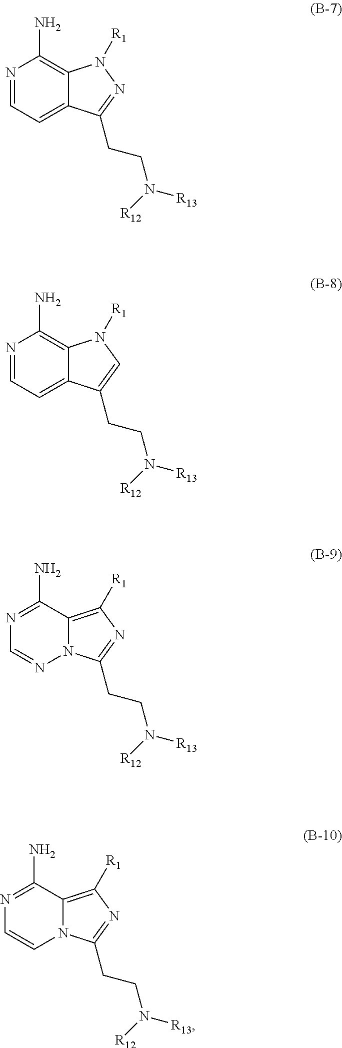

or a salt thereof; wherein R₁, R₁₂ and R₁₃ are as described for formulae (I), (II), and (III).

In some embodiments of formulae (I), (II) and (III), the compound is of structure B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, or B-10c:

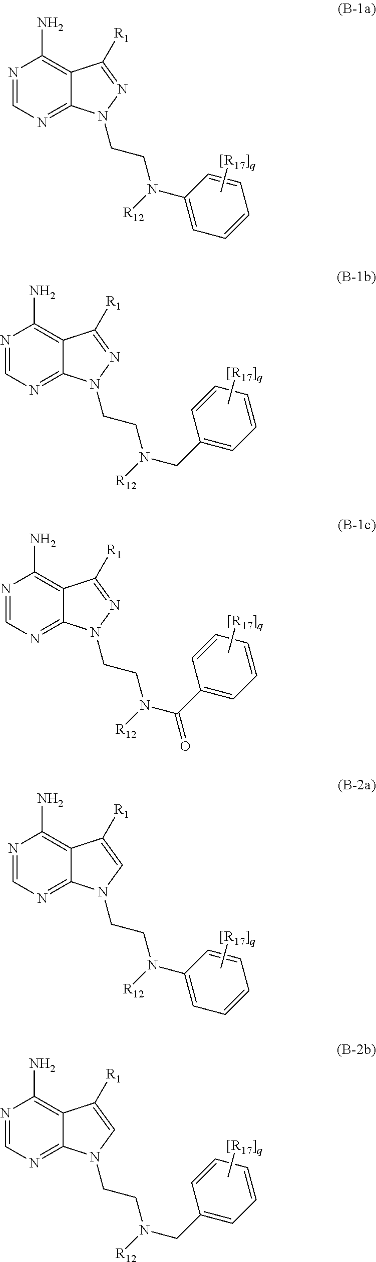

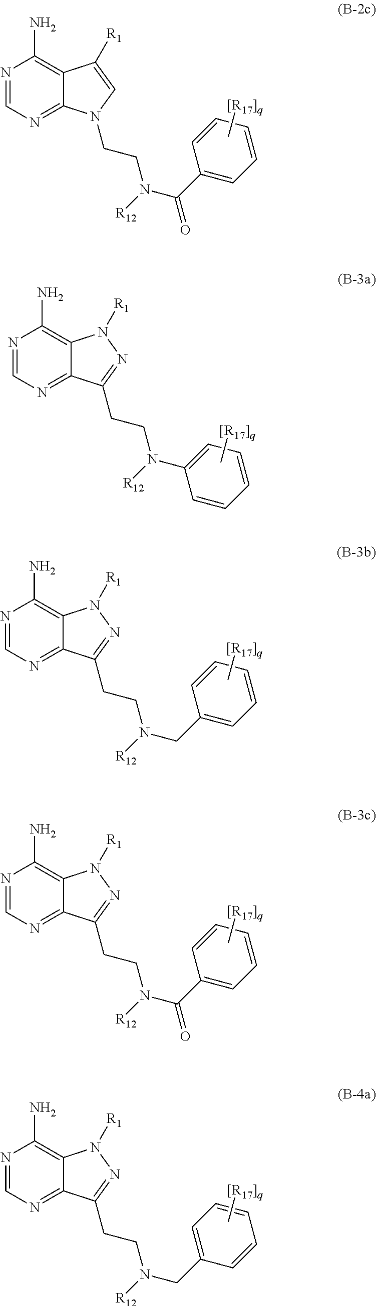

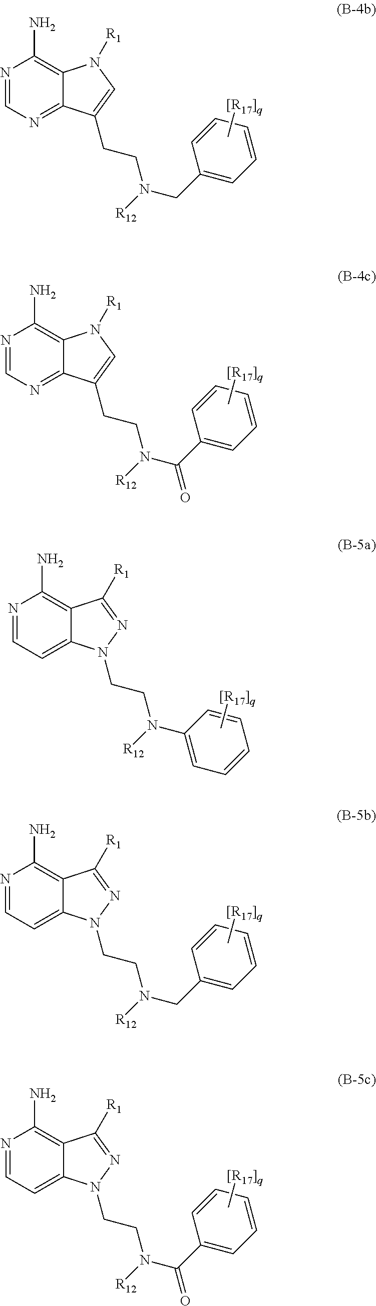

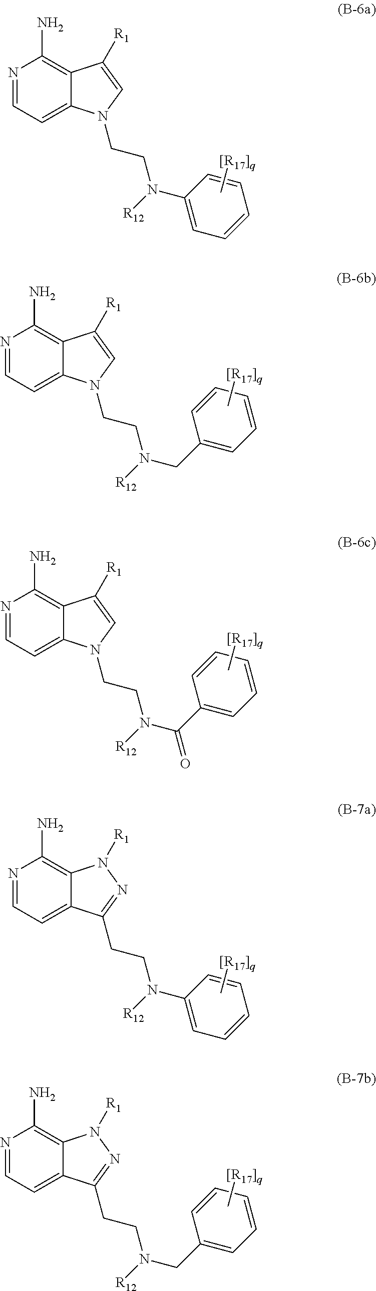

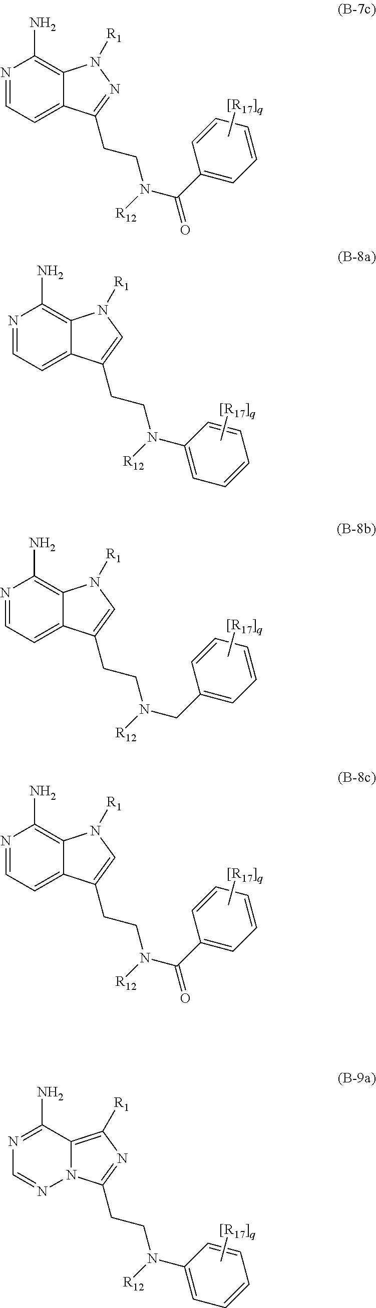

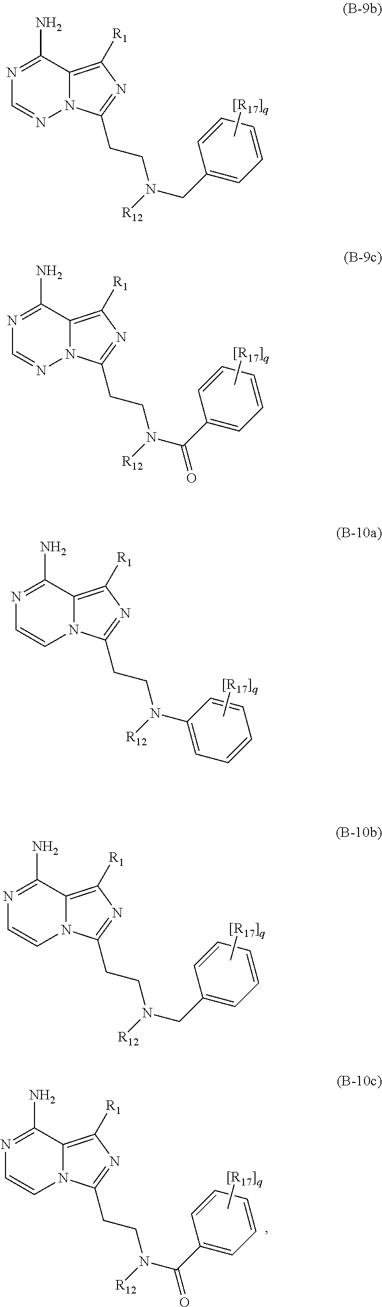

or a salt thereof; wherein R₁ and R₁₂ are as described for formulae B-1, B-2, B-3, B-4, B-5, B-6, B-7, B-8, B-9, and B-10;

each R₁₇, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₃-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl,

thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, or acyl; and

q is 0 to 3.

In some embodiments of formulae B-1a to B-10c, R₁ is substituted or unsubstituted C₁-C₆ alkyl. In some embodiments, R₁ is substituted or unsubstituted C₂-C₆ alkenyl. In some embodiments, R₁ is substituted or unsubstituted aryl. In some embodiments, R₁ is substituted or unsubstituted cycloalkyl. In some embodiments, R₁ is substituted or unsubstituted heterocyclyl. In some embodiments, R₁ is substituted or unsubstituted heteroaryl. In some embodiments, R₁ is substituted or unsubstituted phenyl. In some embodiments, R₁ is substituted or unsubstituted pyridyl. In some embodiments, R₁ is substituted or unsubstituted pyrazolyl. In some embodiments, R₁ is substituted or unsubstituted benzimidazolyl. In some embodiments, R₁ is substituted or unsubstituted pyrimidyl. In some embodiments, R₁ is substituted or unsubstituted indolyl. In some embodiments, R₁ is substituted or unsubstituted pyrrolopyridinyl. In some embodiments, R₁ is substituted or unsubstituted indazolyl.

In some embodiments of formulae B-1a to B-10c, q is O. In some embodiments, q is 1. In some embodiments, q is 2. In some embodiments, q is 3.

In some embodiments of formulae (I), (II) and (III), the compound is of structure B-1a-1, B-1a-2, B-1a-3, B-1a-4, B-1a-5, B-1a-6, B-1a-7, B-1a-8, B-1b-1, B-1b-2, B-1b-3, B-1b-4, B-1b-5, B-1b-6, B-1b-7, B-1b-8, B-1c-1, B-1c-2, B-1c-3, B-1c-4, B-1c-5, B-1c-6, B-1c-7, B-1c-8, B-2a-1, B-2a-2, B-2a-3, B-2a-4, B-2a-5, B-2a-6, B-2a-7, B-2a-8, B-2b-1, B-2b-2, B-2b-3, B-2b-4, B-2b-5, B-2b-6, B-2b-7, B-2b-8, B-2c-1, B-2c-2, B-2c-3, B-2c-4, B-2c-5, B-2c-6, B-2c-7, B-2c-8, B-3a-1, B-3a-2, B-3a-3, B-3a-4, B-3a-5, B-3a-6, B-3a-7, B-3a-8, B-3b-1, B-3b-2, B-3b-3, B-3b-4, B-3b-5, B-3b-6, B-3b-7, B-3b-8, B-3c-1, B-3c-2, B-3c-3, B-3c-4, B-3c-5, B-3c-6, B-3c-7, B-3c-8, B-4a-1, B-4a-2, B-4a-3, B-4a-4, B-4a-5, B-4a-6, B-4a-7, B-4a-8, B-4b-1, B-4b-2, B-4b-3, B-4b-4, B-4b-5, B-4b-6, B-4b-7, B-4b-8, B-4c-1, B-4c-2, B-4c-3, B-4c-4, B-4c-5, B-4c-6, B-4c-7, B-4c-8, B-5a-1, B-5a-2, B-5a-3, B-5a-4, B-5a-5, B-5a-6, B-5a-7, B-5a-8, B-5b-1, B-5b-2, B-5b-3, B-5b-4, B-5b-5, B-5b-6, B-5b-7, B-5b-8, B-5c-1, B-5c-2, B-5c-3, B-5c-4, B-5c-5, B-5c-6, B-5c-7, B-5c-8, B-6a-1, B-6a-2, B-6a-3, B-6a-4, B-6a-5, B-6a-6, B-6a-7, B-6a-8, B-6b-1, B-6b-2, B-6b-3, B-6b-4, B-6b-5, B-6b-6, B-6b-7, B-6b-8, B-6c-1, B-6c-2, B-6c-3, B-6c-4, B-6c-5, B-6c-6, B-6c-7, B-6c-8, B-7a-1, B-7a-2, B-7a-3, B-7a-4, B-7a-5, B-7a-6, B-7a-7, B-7a-8, B-7b-1, B-7b-2, B-7b-3, B-7b-4, B-7b-5, B-7b-6, B-7b-7, B-7b-8, B-7c-1, B-7c-2, B-7c-3, B-7c-4, B-7c-5, B-7c-6, B-7c-7, B-7c-8, B-8a-1, B-8a-2, B-8a-3, B-8a-4, B-8a-5, B-8a-6, B-8a-7, B-8a-8, B-8b-1, B-8b-2, B-8b-3, B-8b-4, B-8b-5, B-8b-6, B-8b-7, B-8b-8, B-8c-1, B-8c-2, B-8c-3, B-8c-4, B-8c-5, B-8c-6, B-8c-7, B-8c-8, B-9a-1, B-9a-2, B-9a-3, B-9a-4, B-9a-5, B-9a-6, B-9a-7, B-9a-8, B-9b-1, B-9b-2, B-9b-3, B-9b-4, B-9b-5, B-9b-6, B-9b-7, B-9B-9, B-9c-1, B-9c-2, B-9c-3, B-9c-4, B-9c-5, B-9c-6, B-9c-7, B-9c-8, B-10a-1, B-10a-2, B-10a-3, B-10a-4, B-10a-5, B-10a-6, B-10a-7, B-10a-8, B-10b-1, B-10b-2, B-10b-3, B-10b-4, B-10b-5, B-10b-6, B-10b-7, B-10b-8, B-10c-1, B-10c-2, B-10c-3, B-10c-4, B-10c-5, B-10c-6, B-10c-7, or B-10c-8:

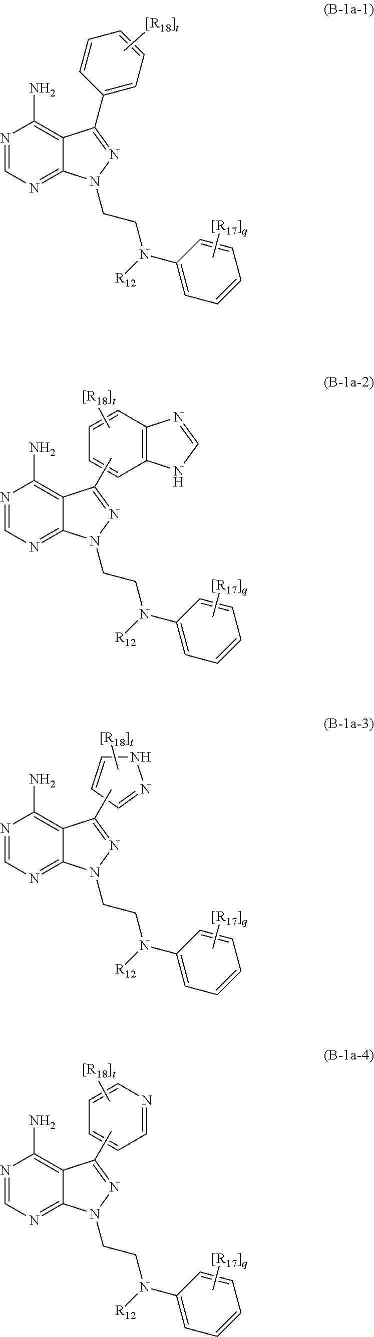

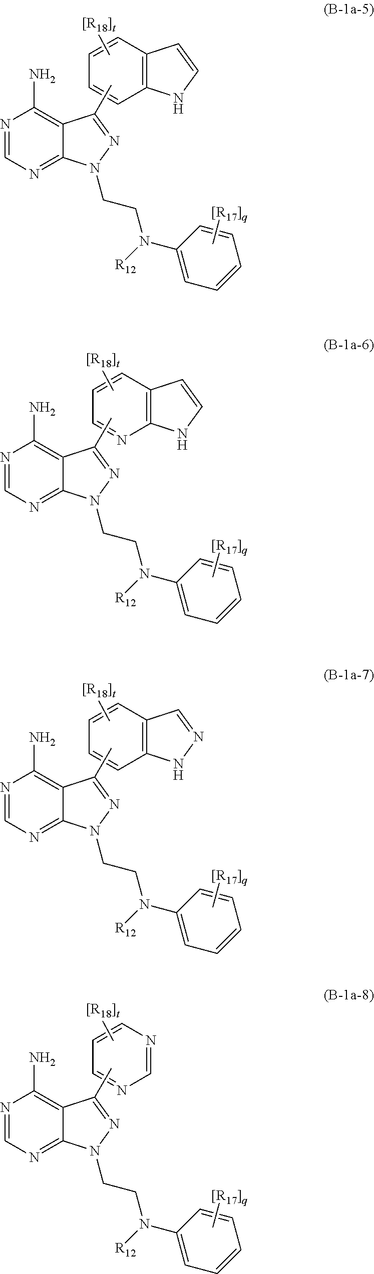

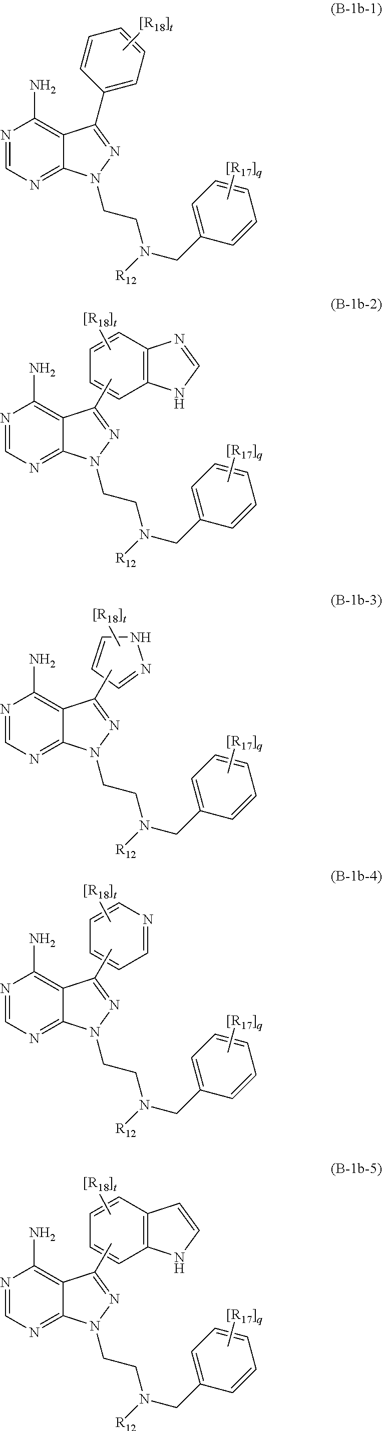

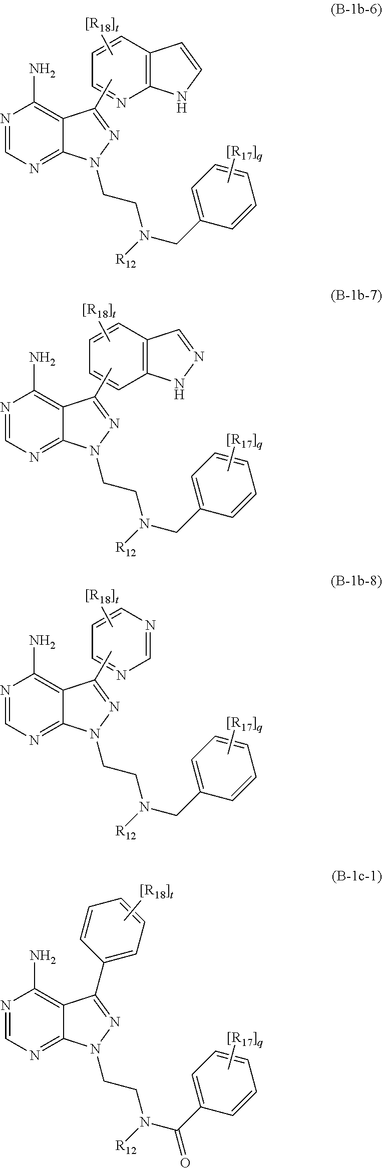

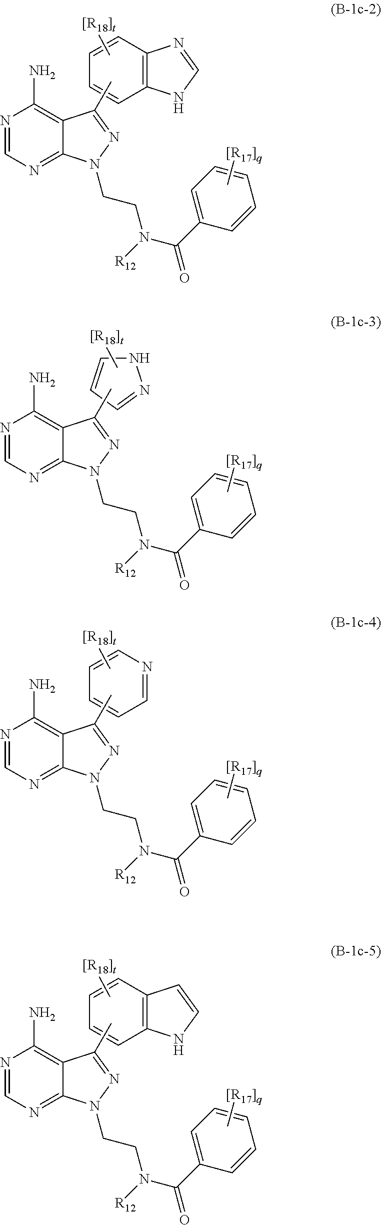

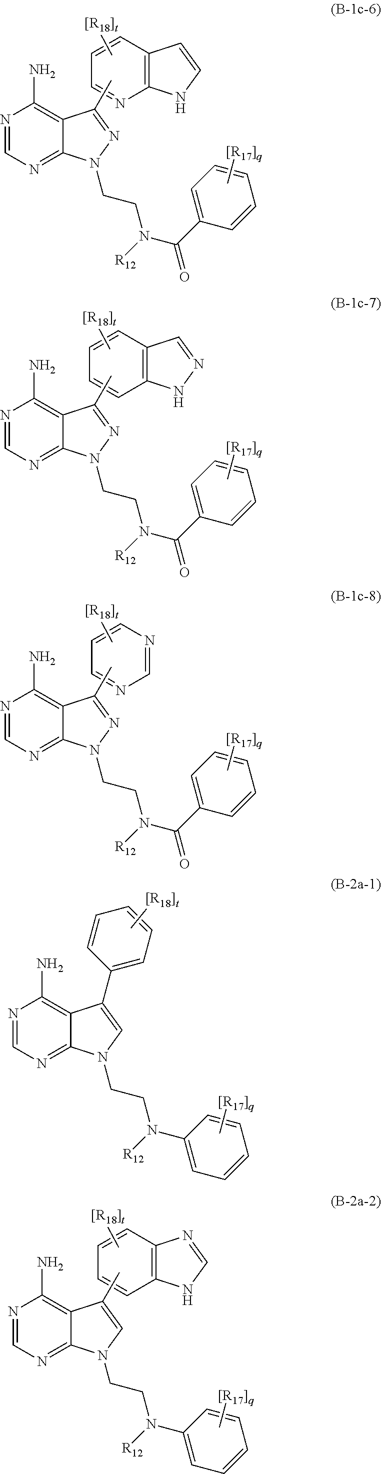

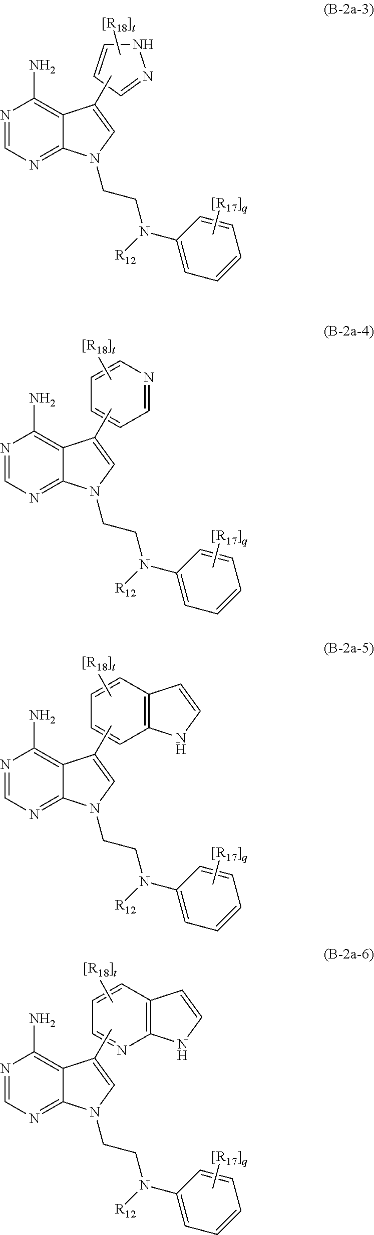

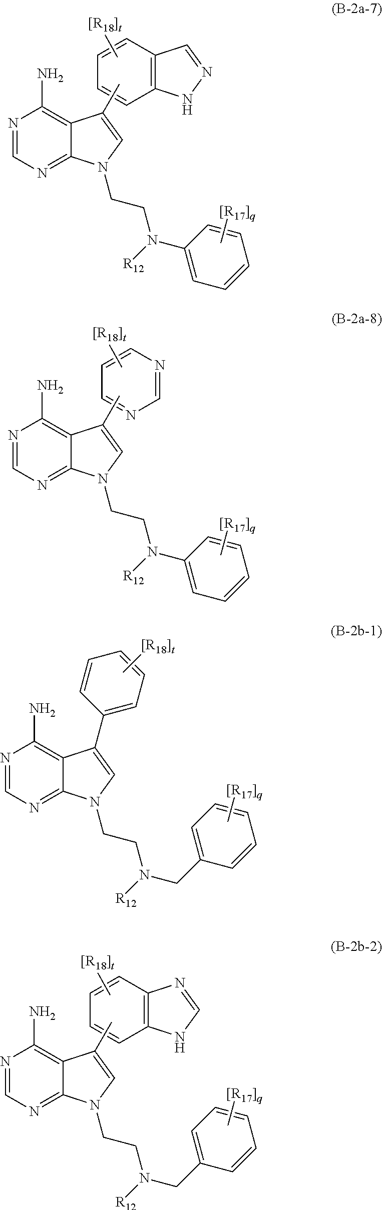

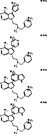

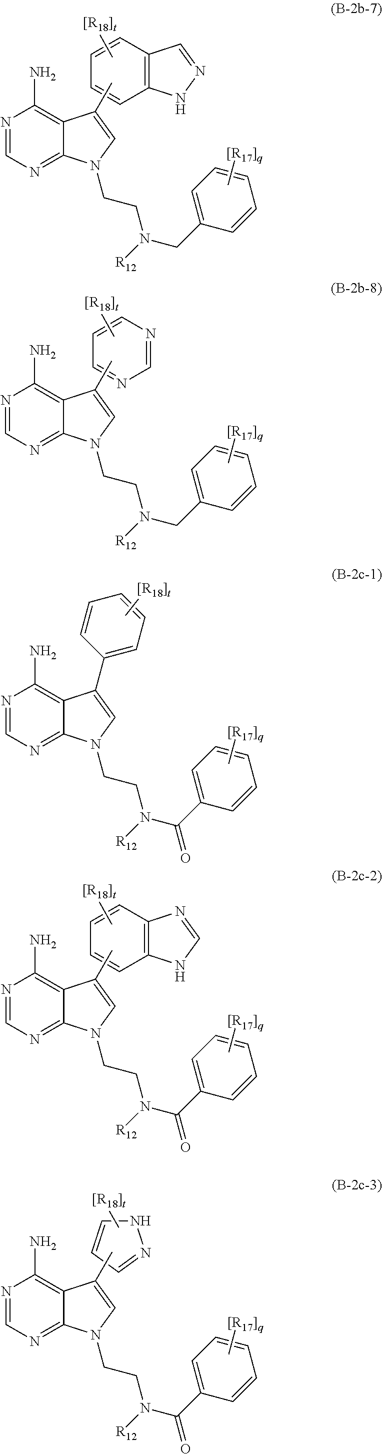

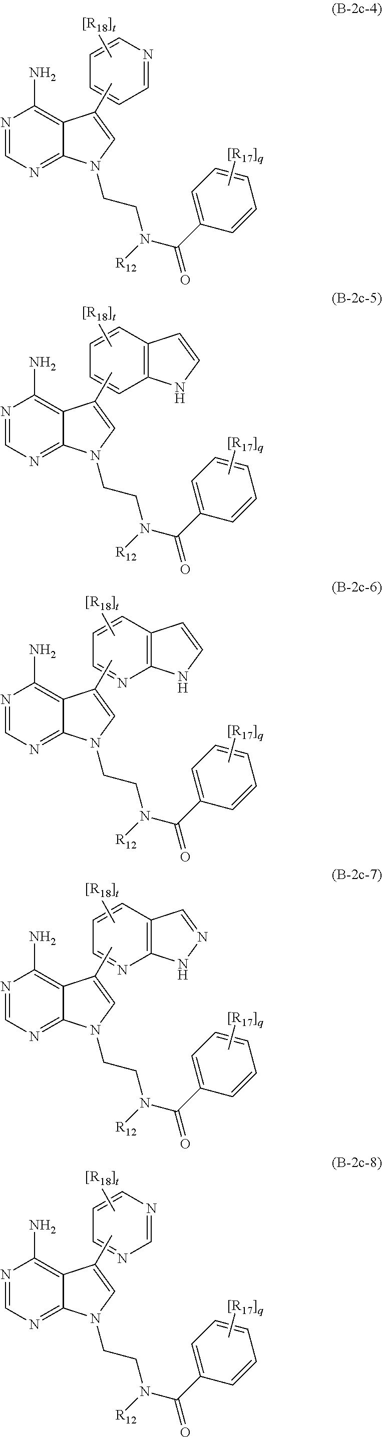

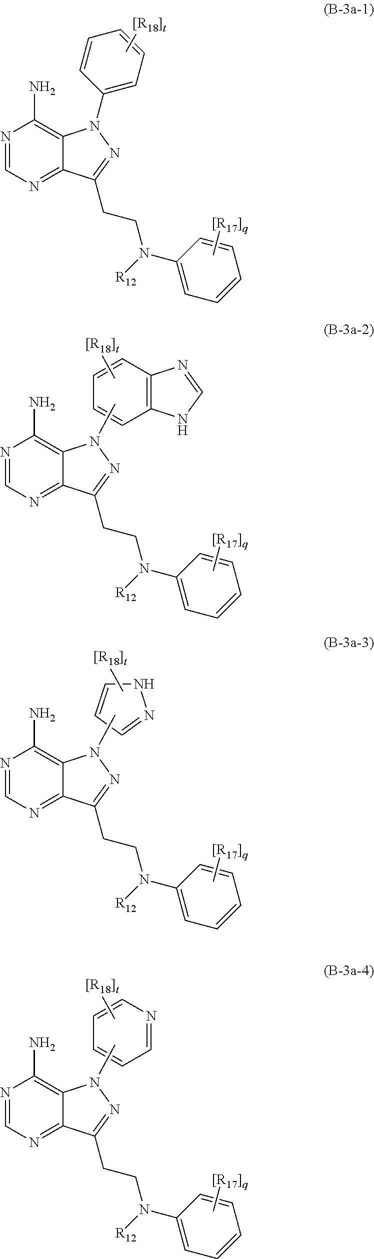

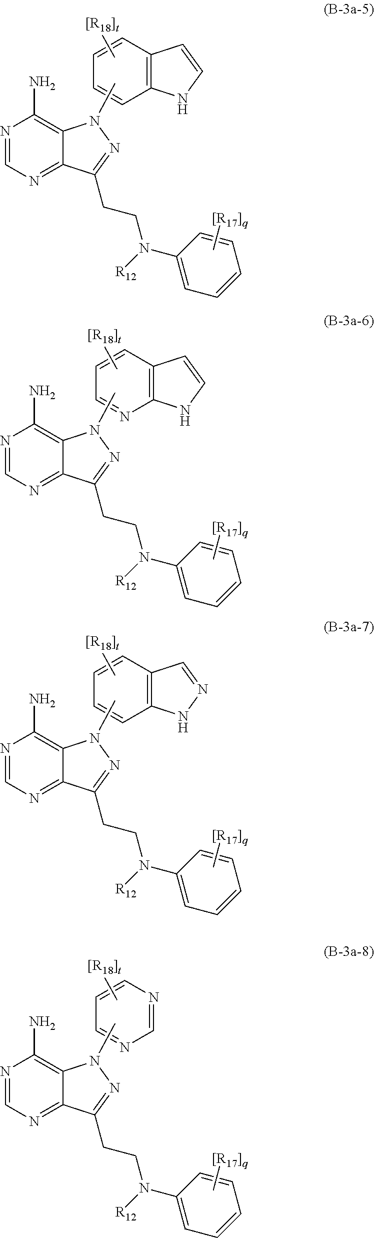

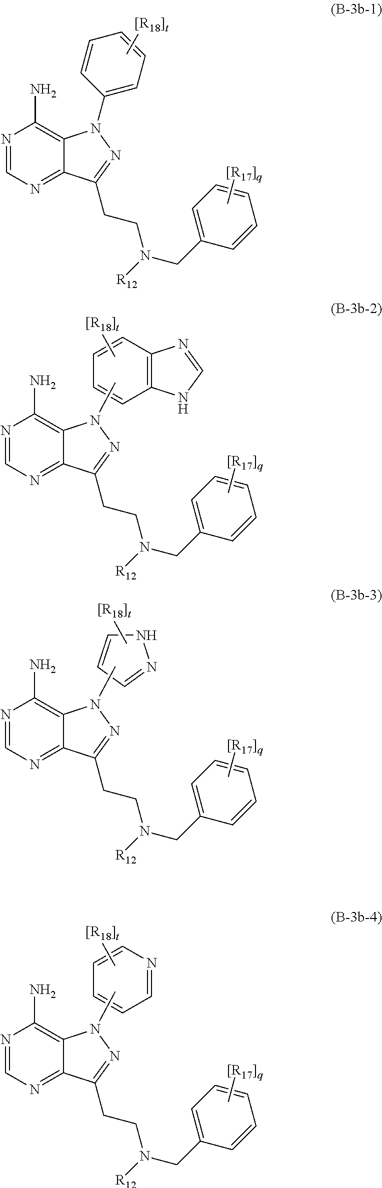

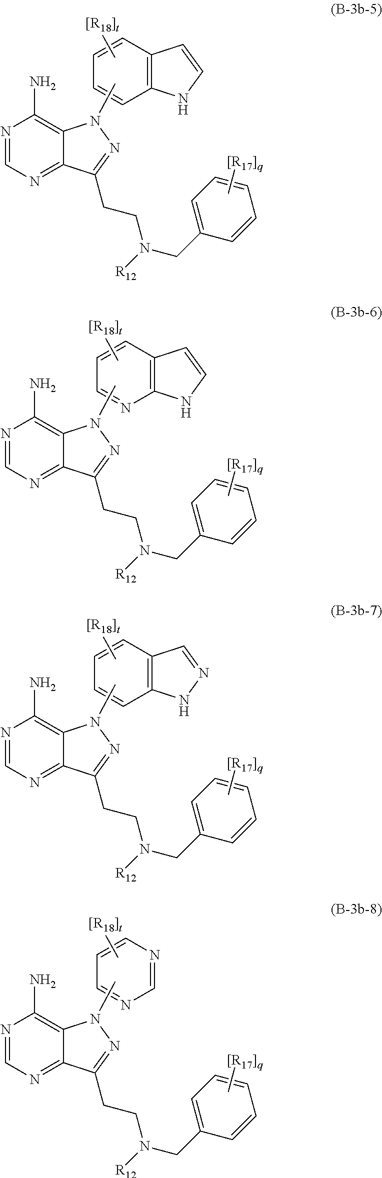

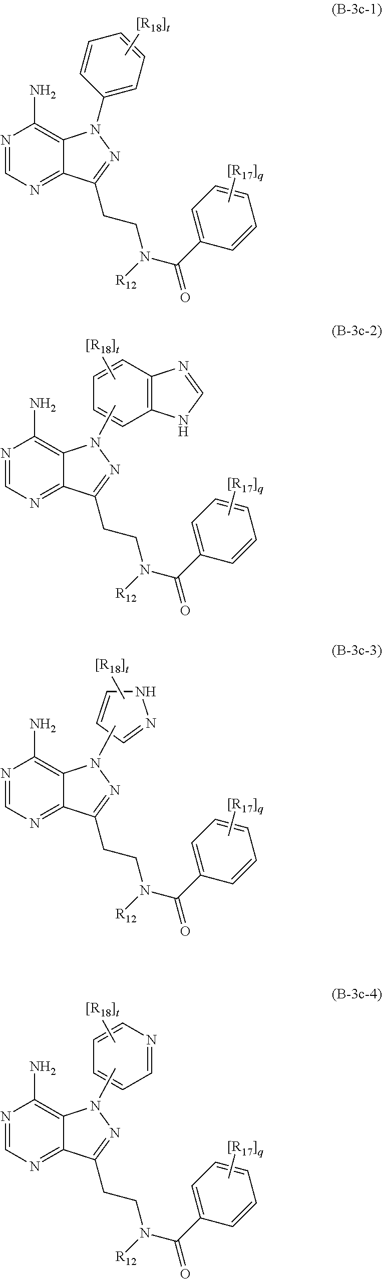

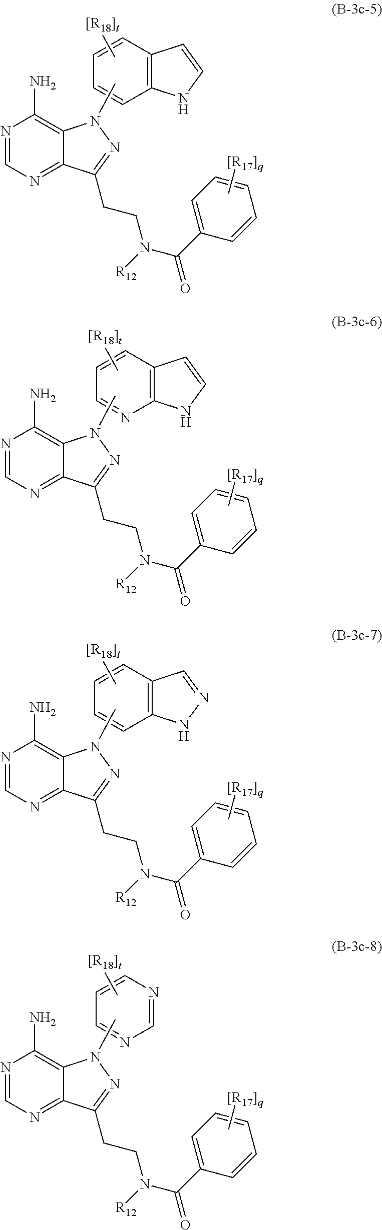

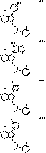

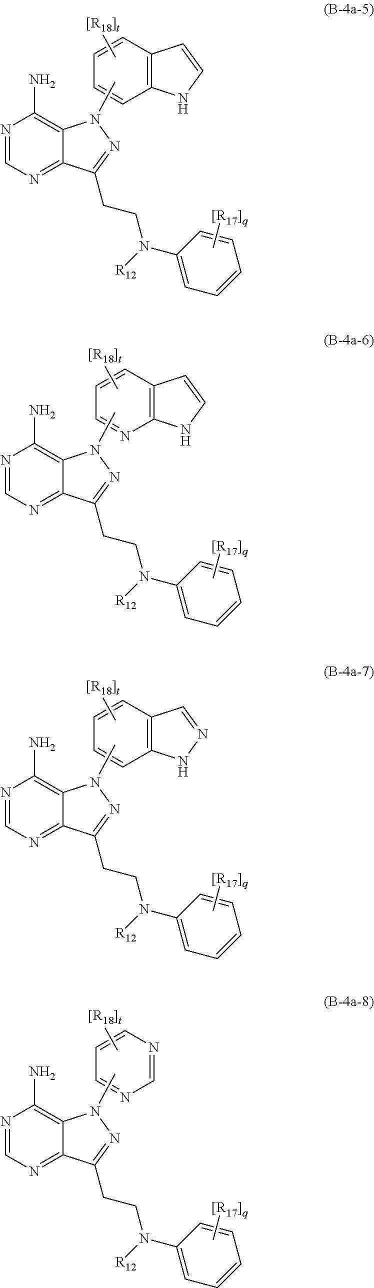

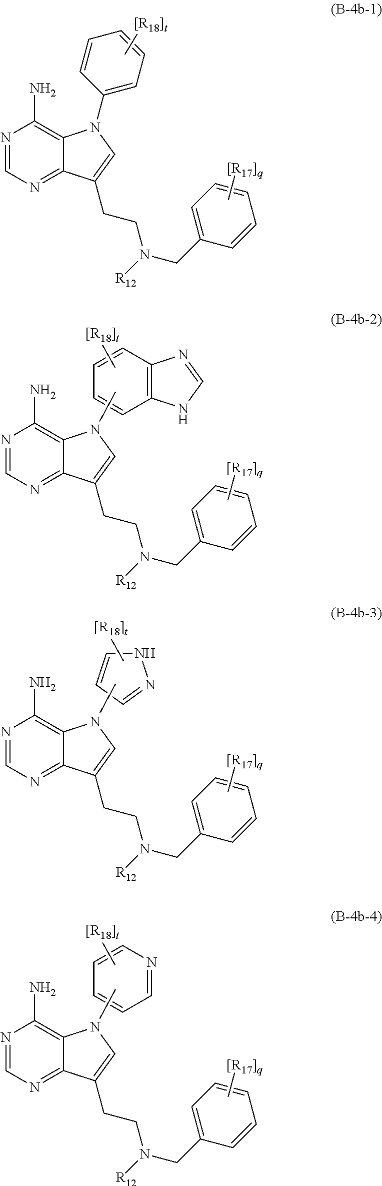

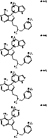

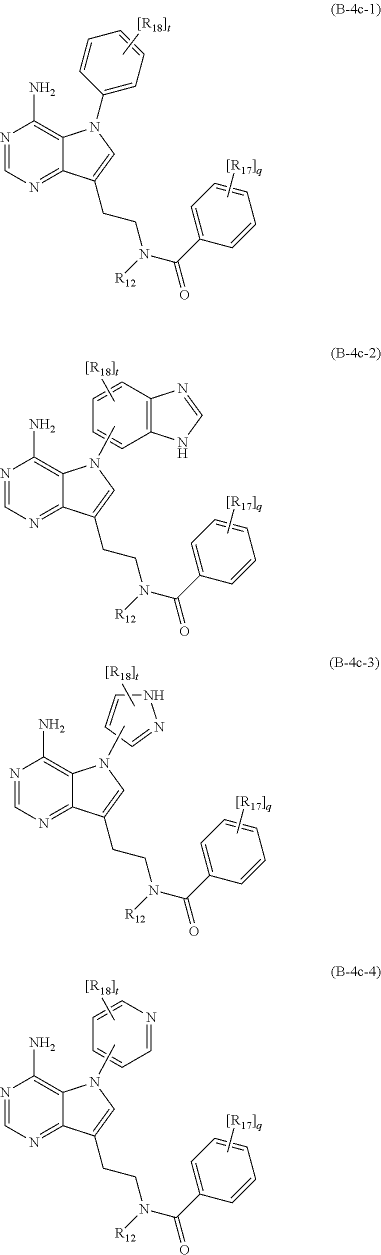

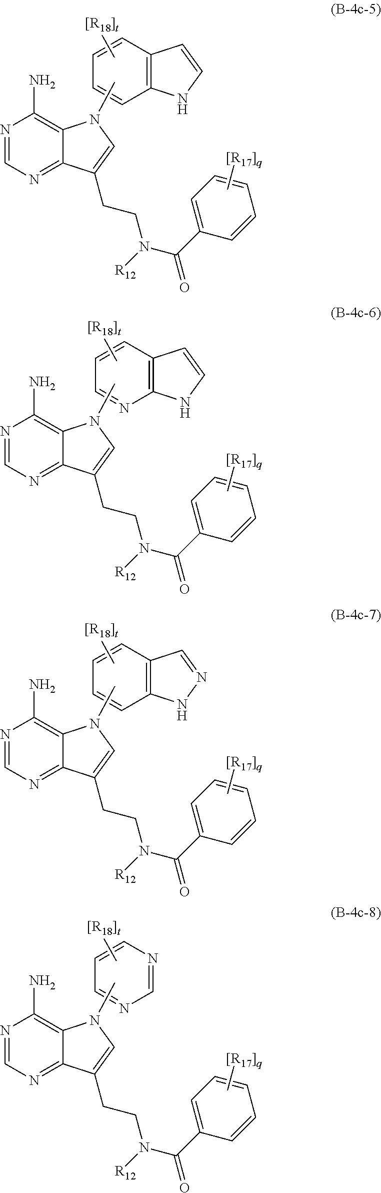

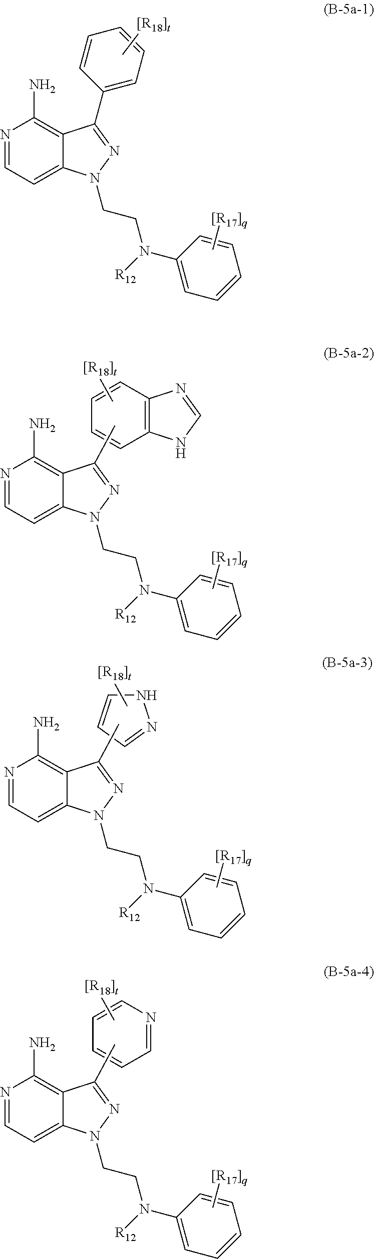

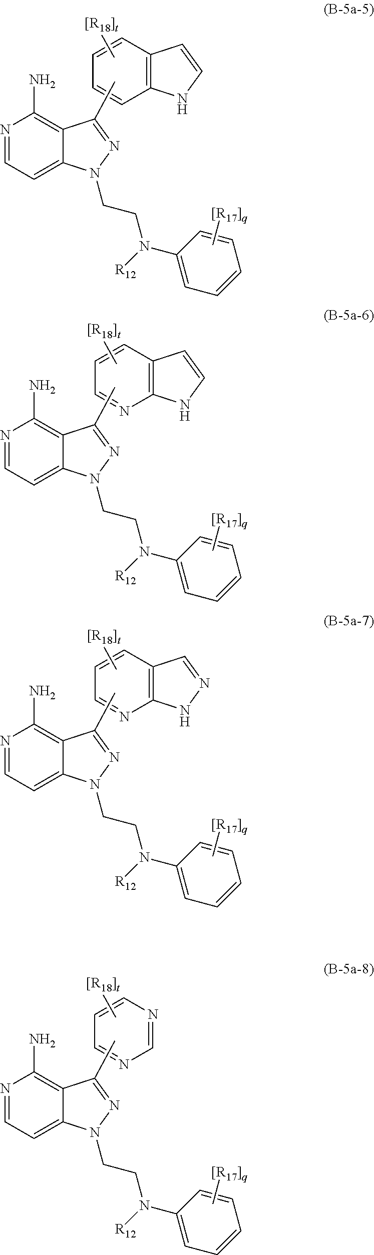

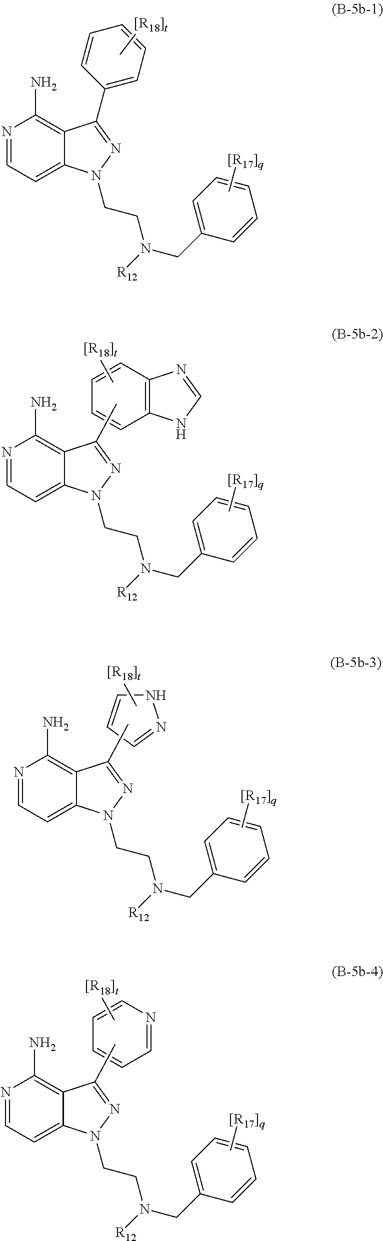

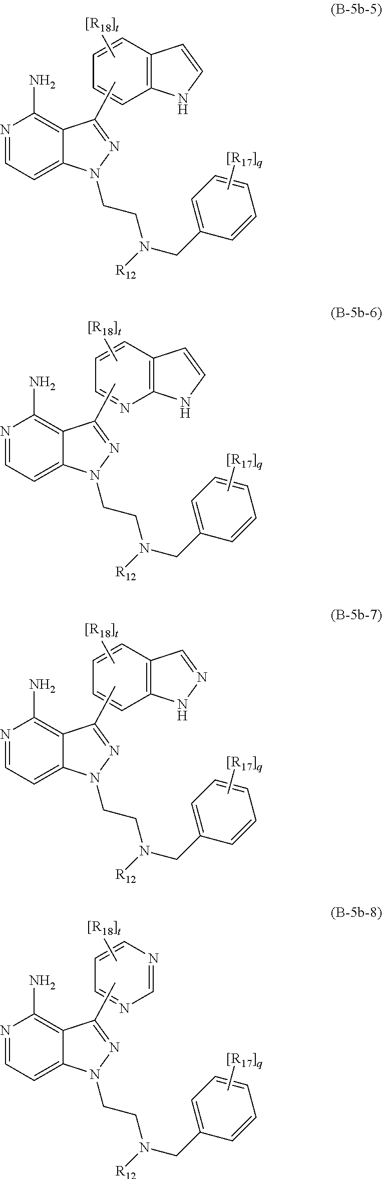

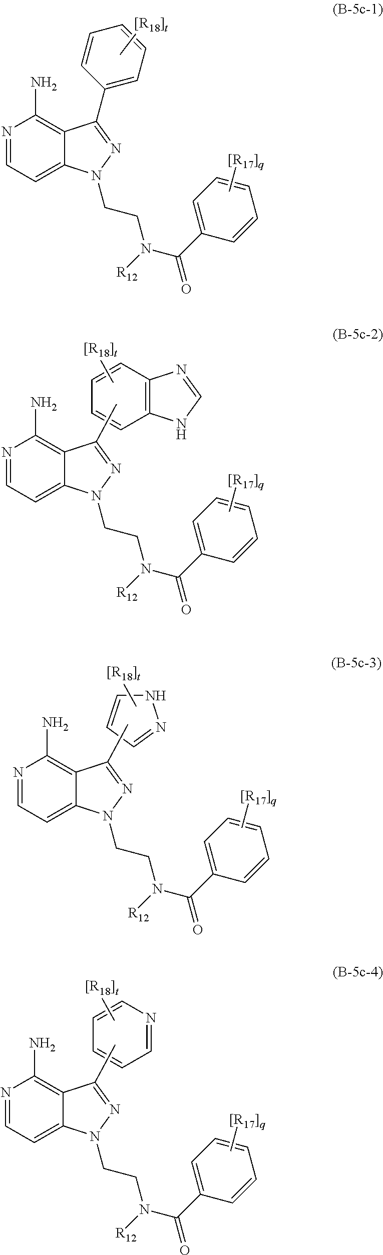

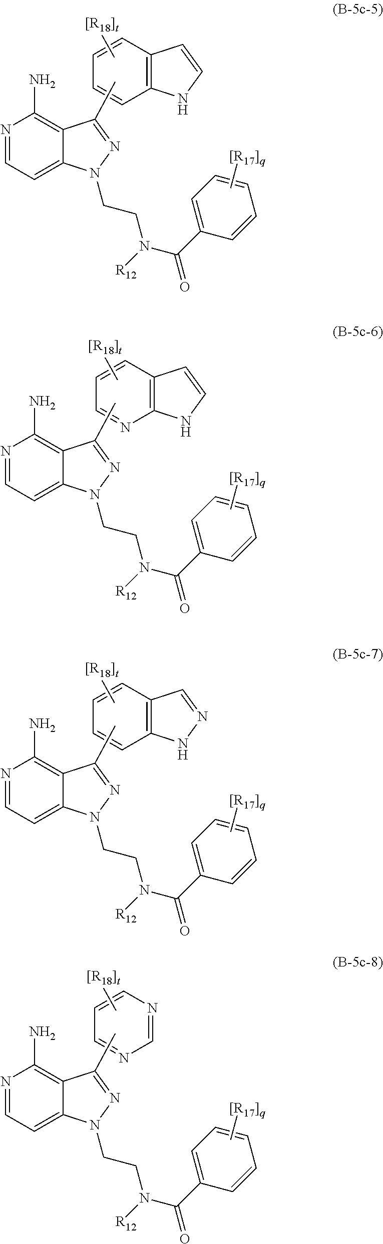

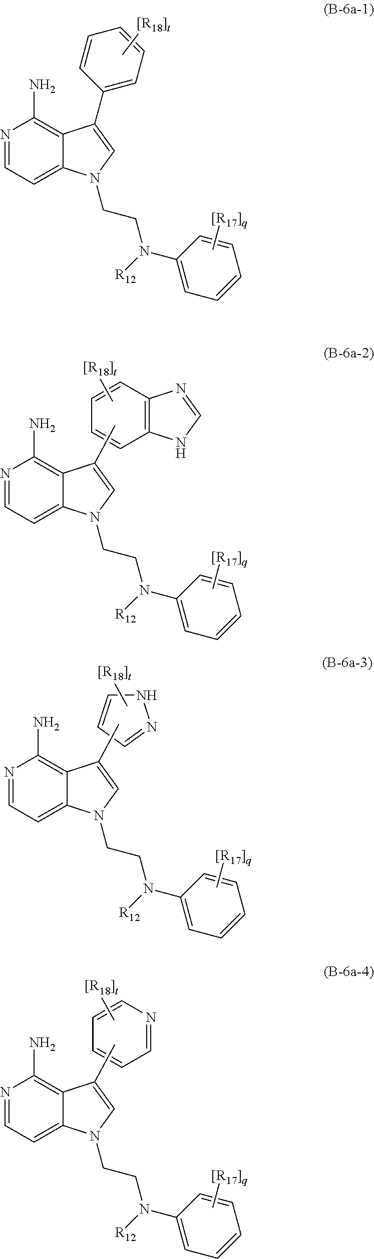

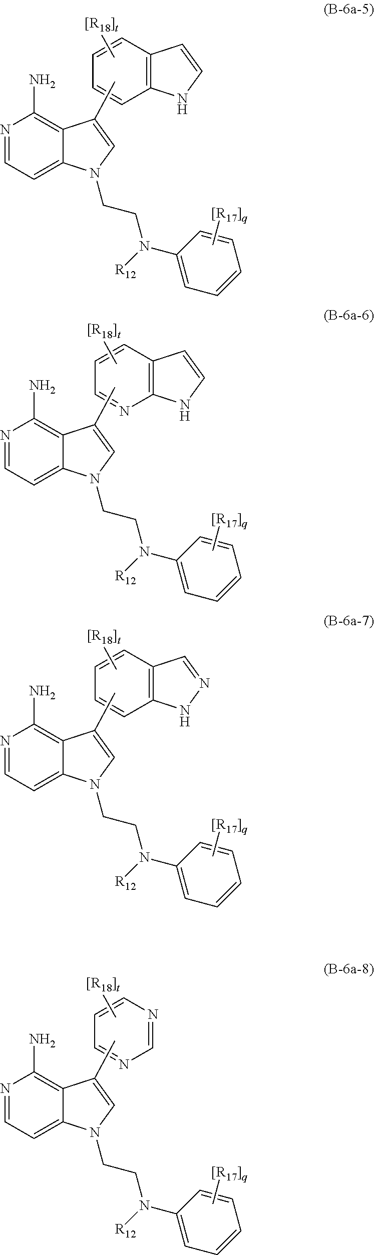

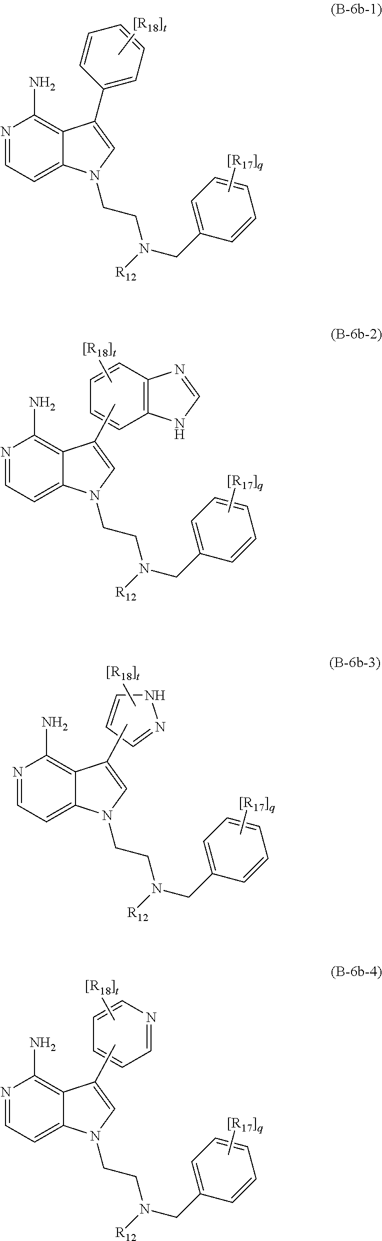

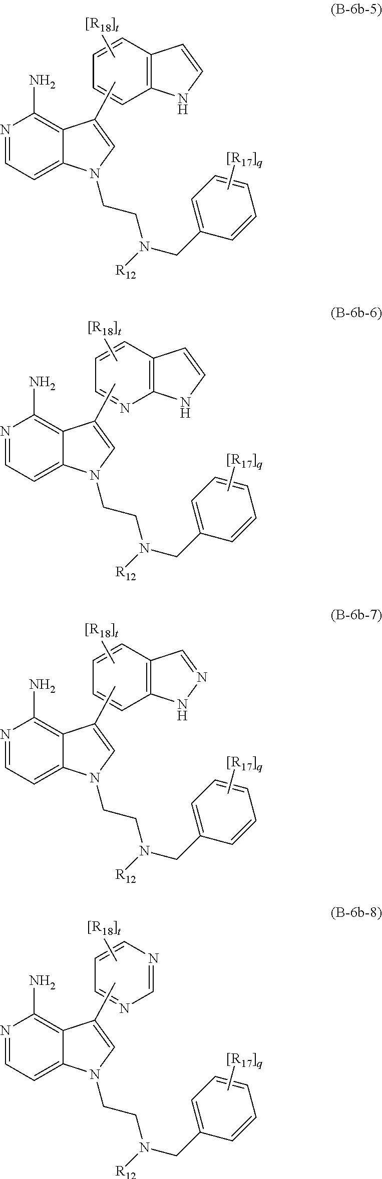

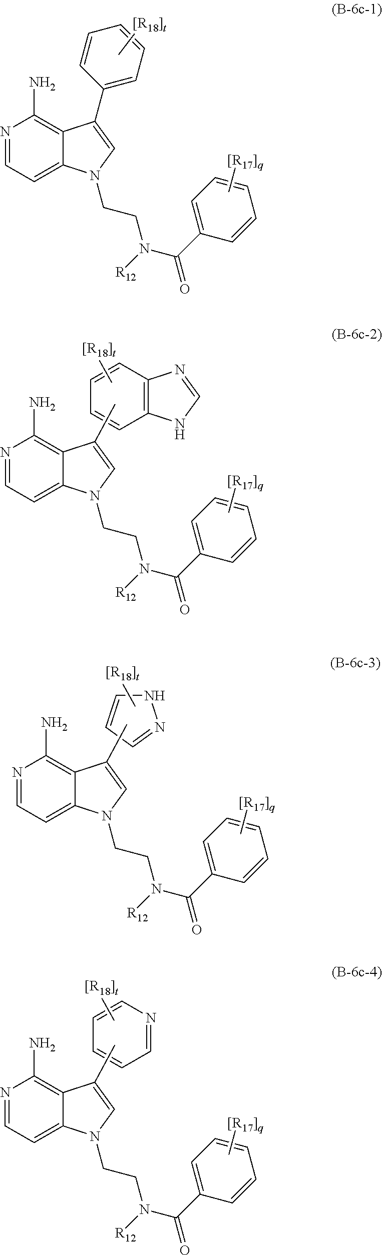

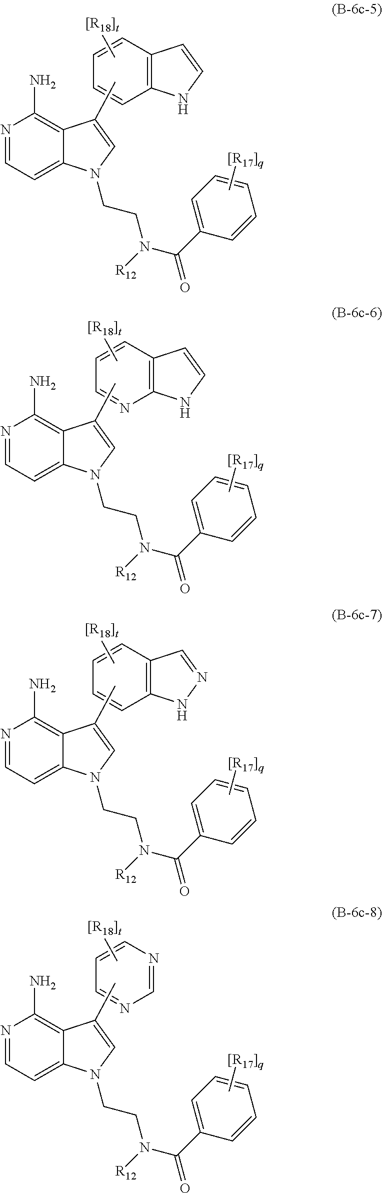

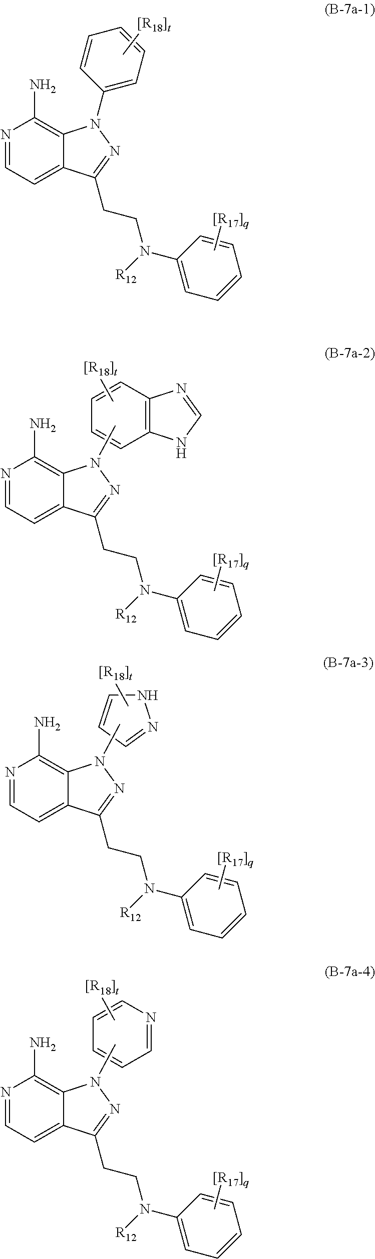

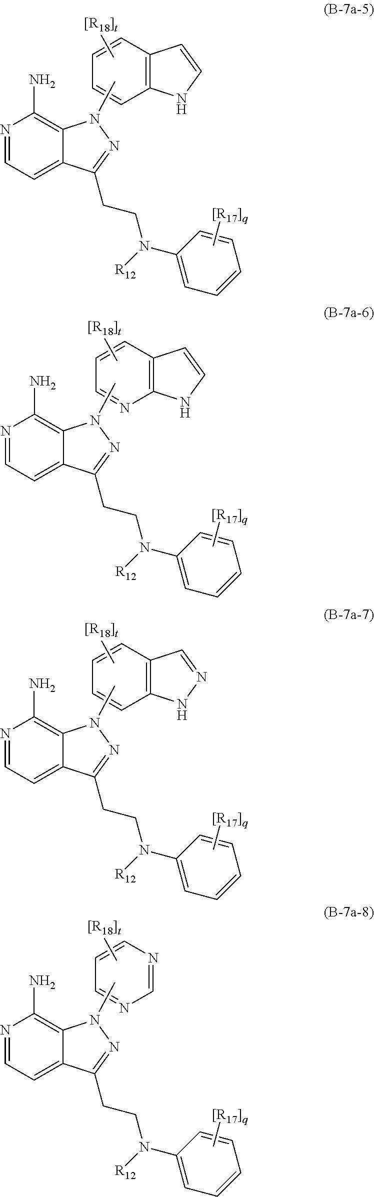

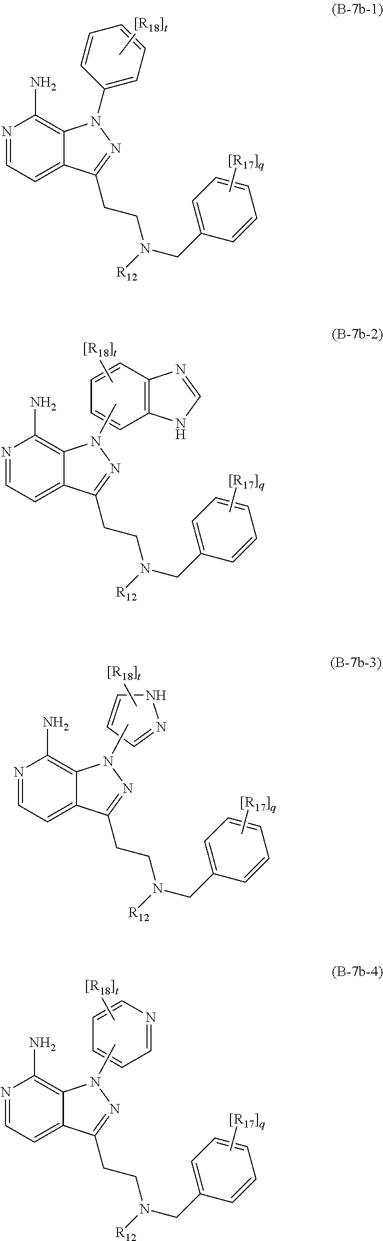

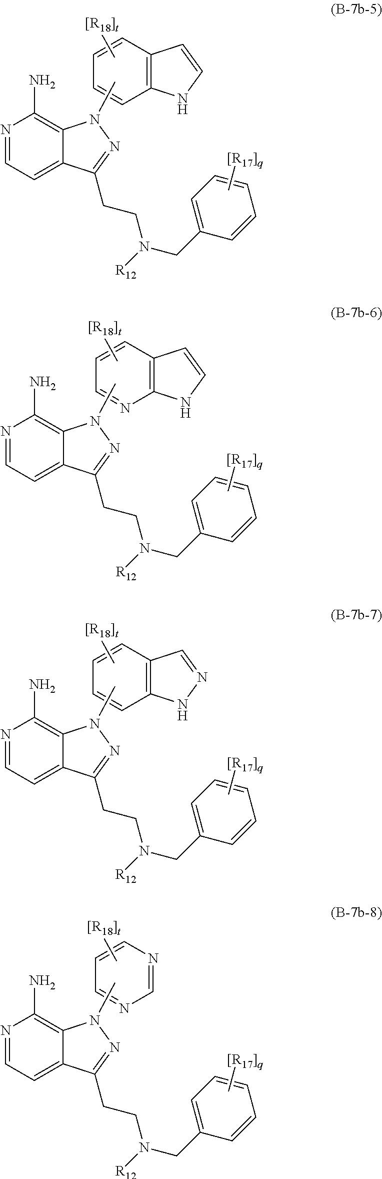

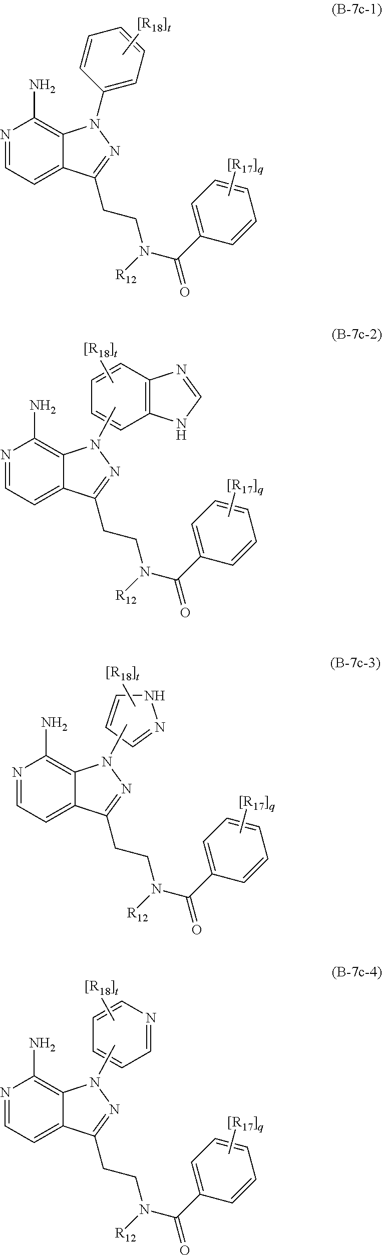

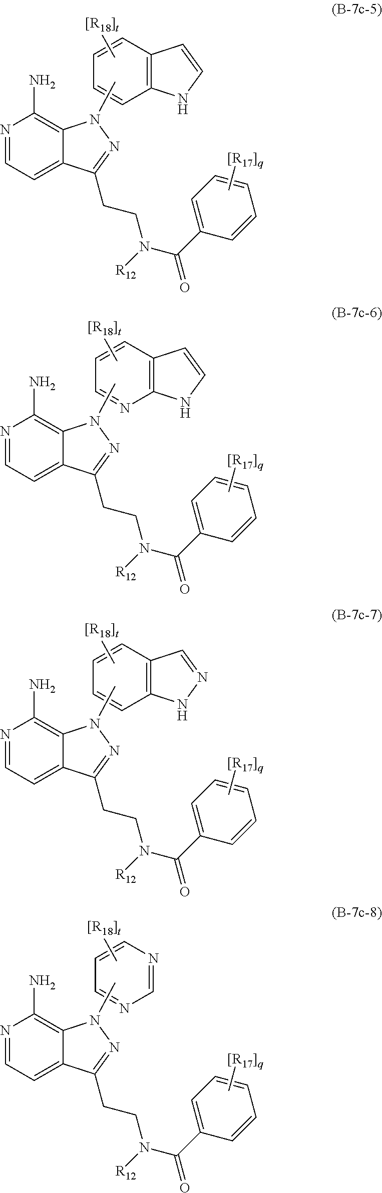

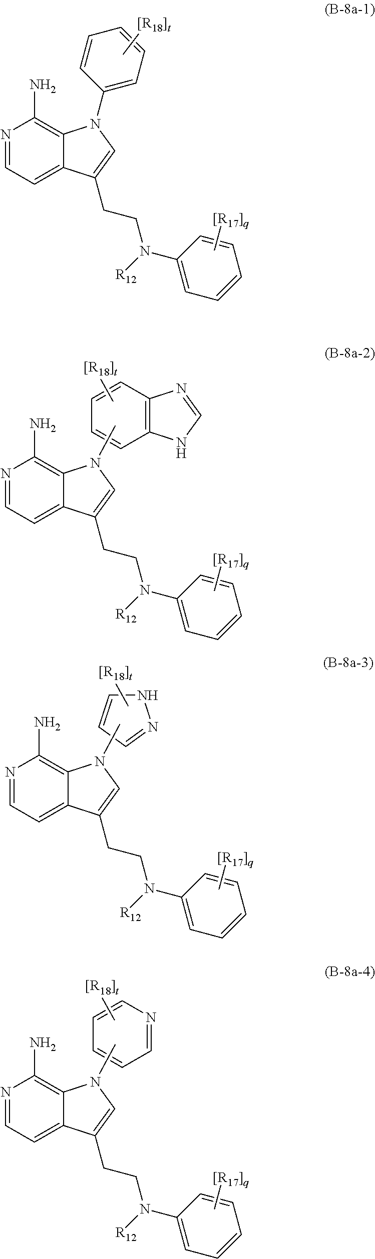

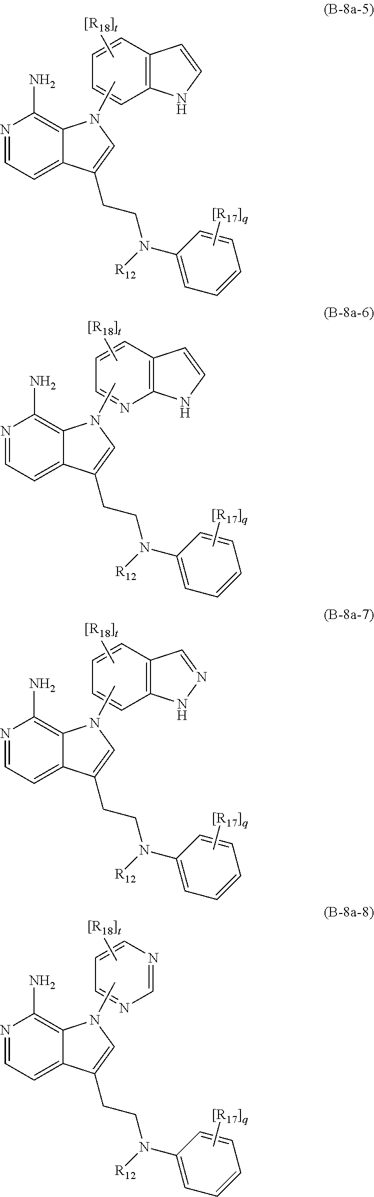

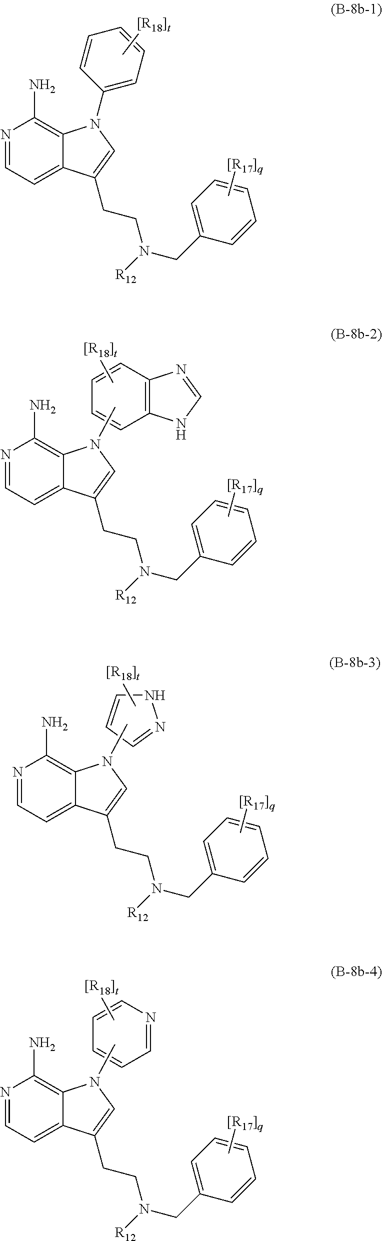

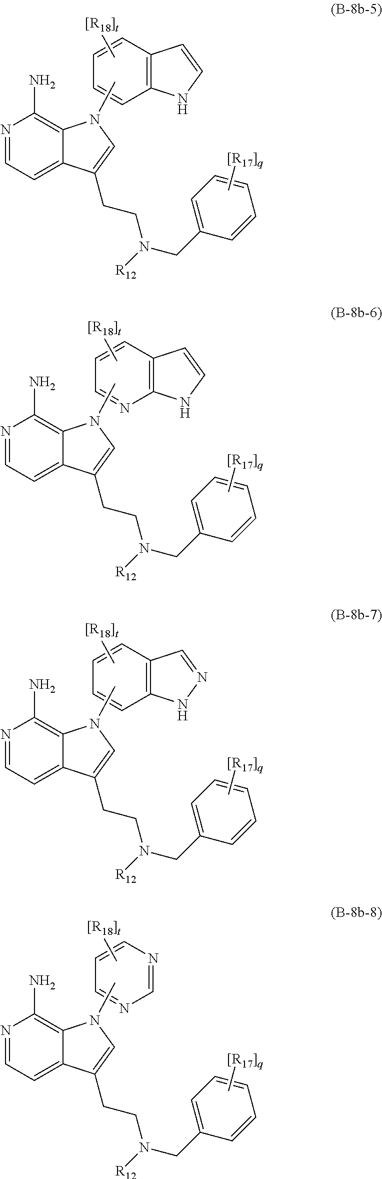

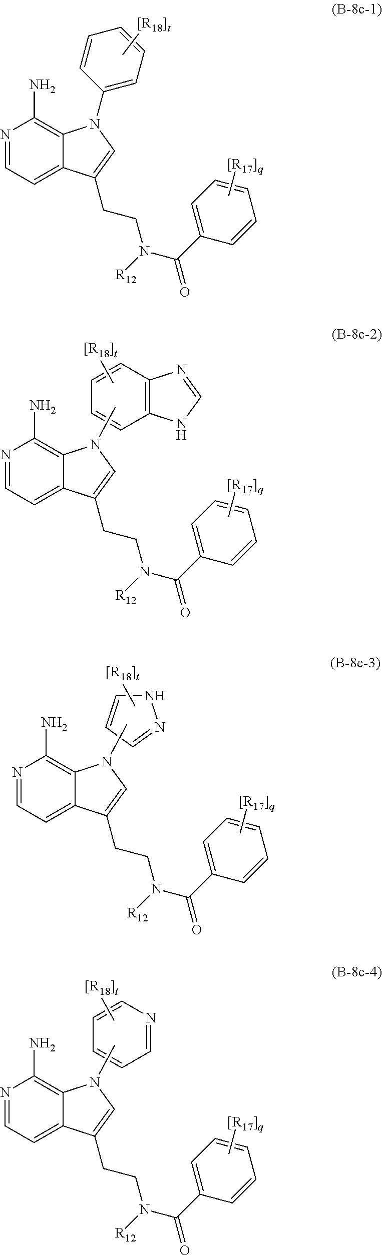

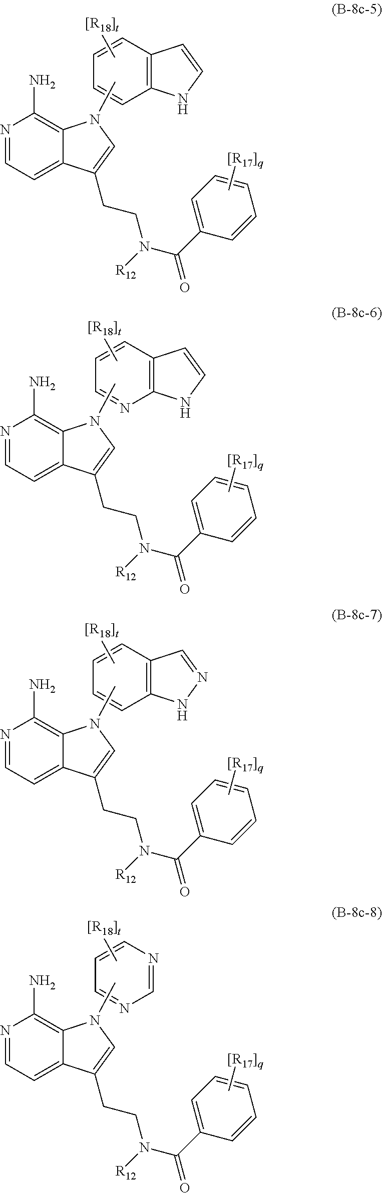

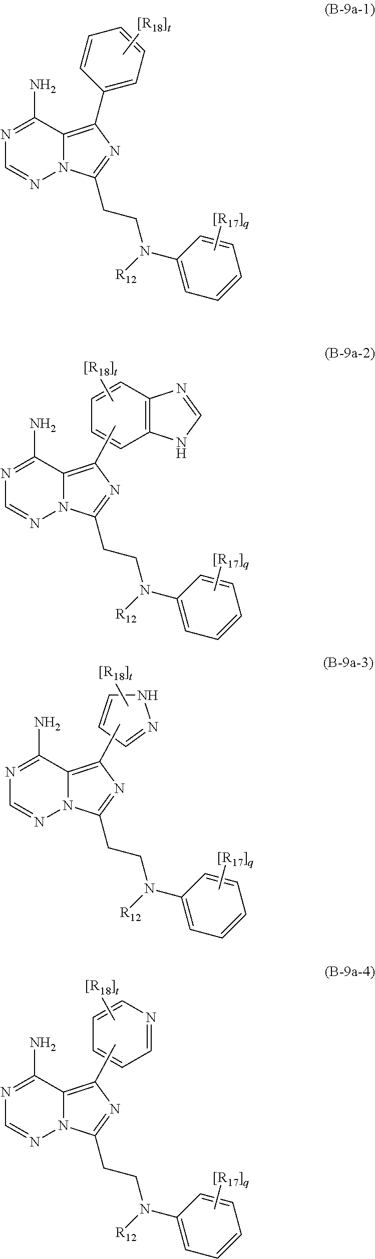

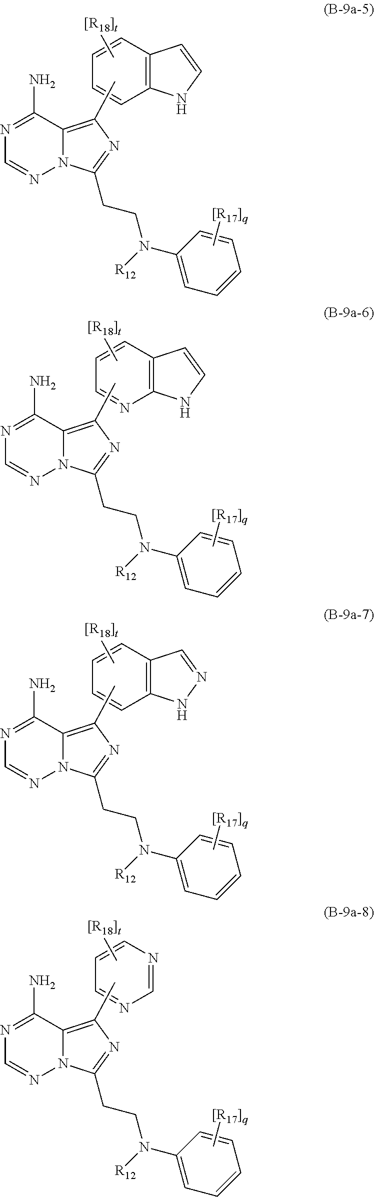

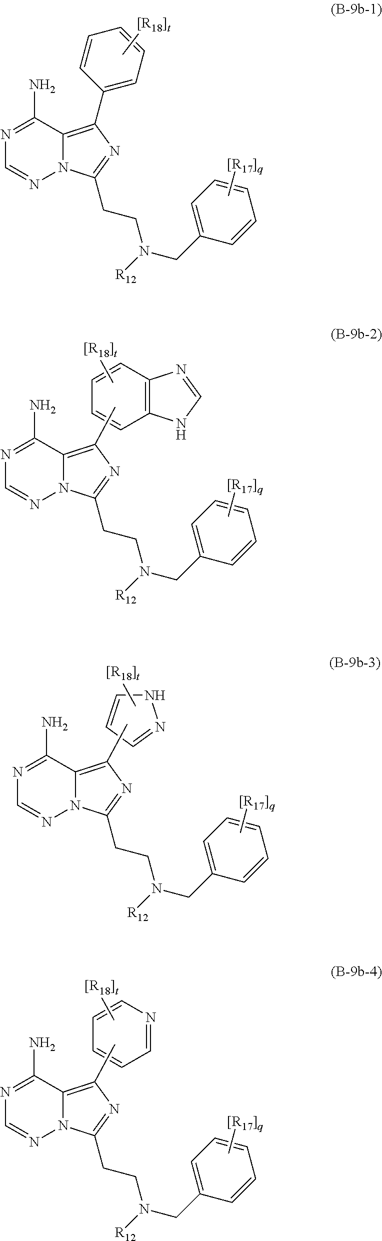

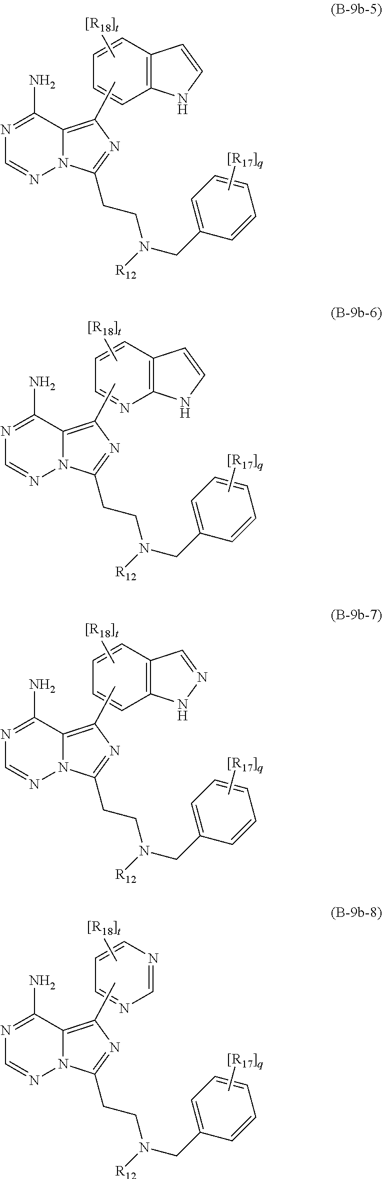

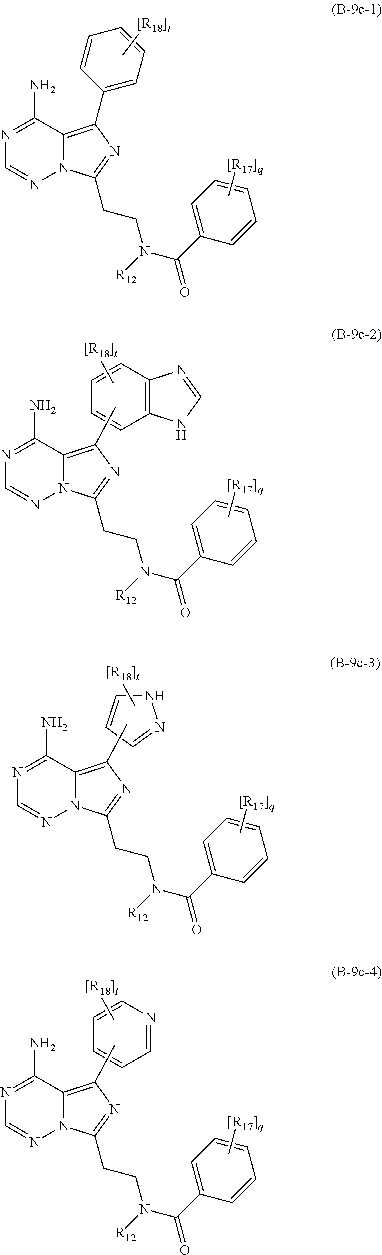

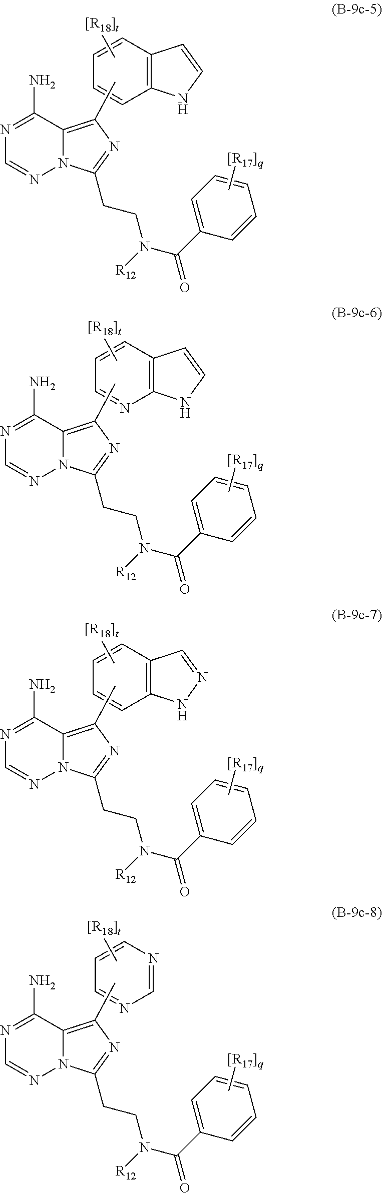

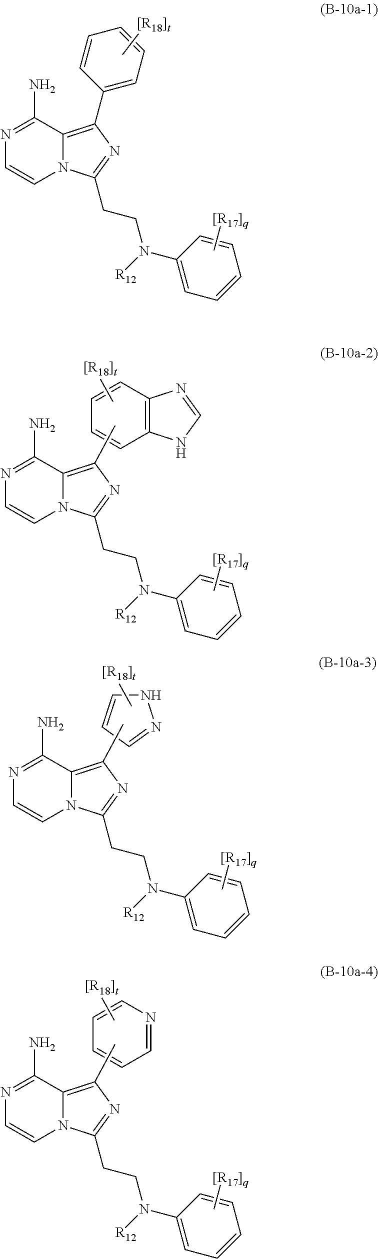

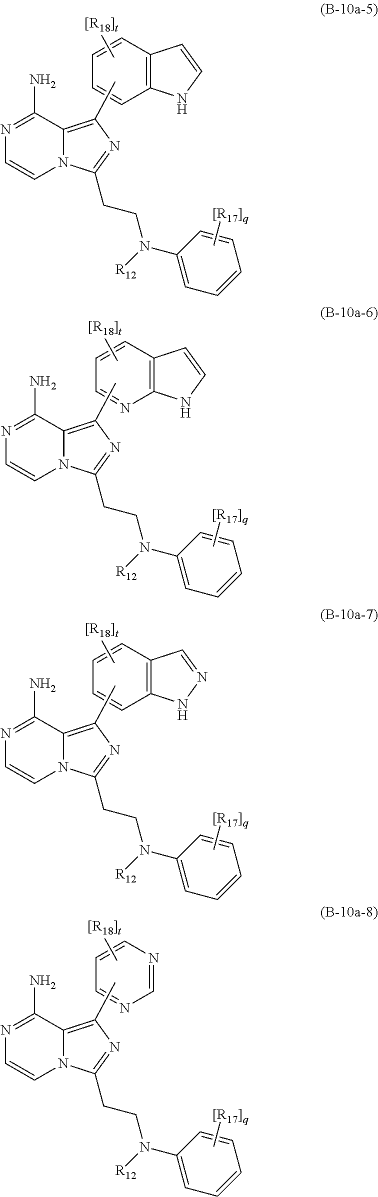

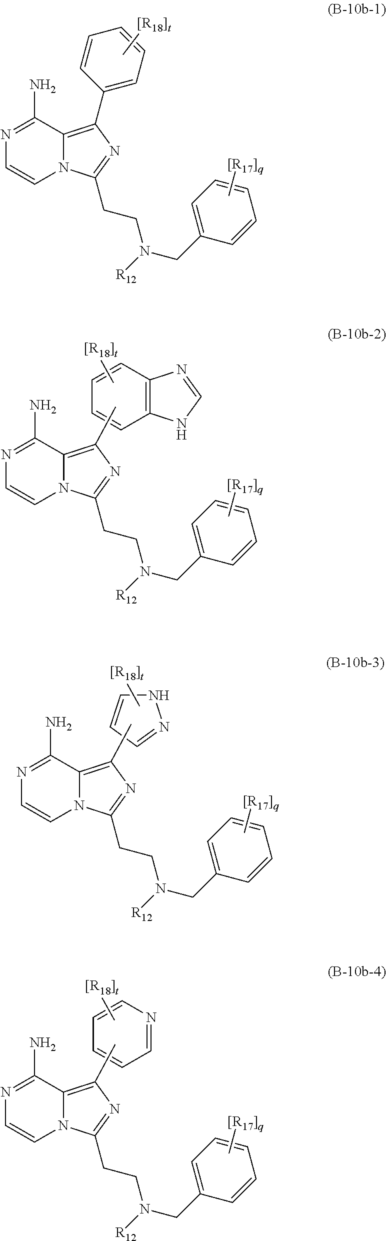

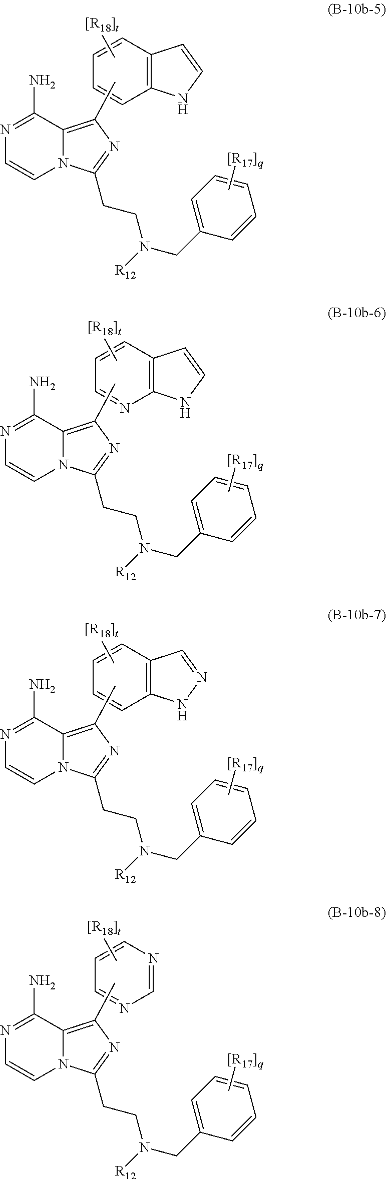

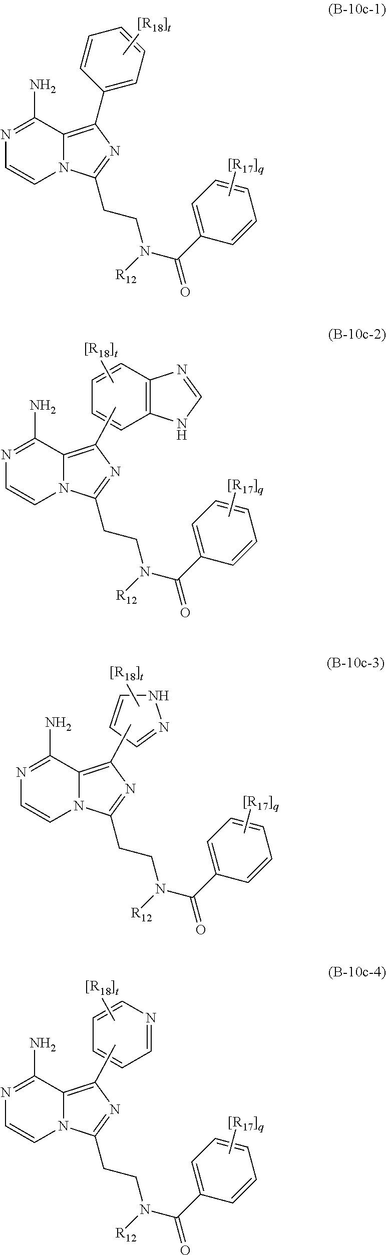

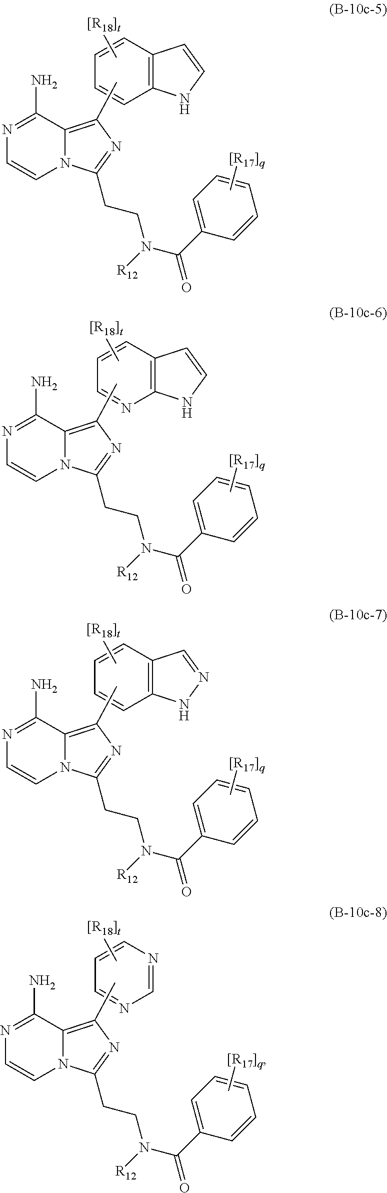

or a salt thereof; wherein q, R₁₂ and R₁₇ are as described for formulae B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, and B-10c;

each R₁₈, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₃-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₁₂, or —NHR₁₂; and

is 0 to 3.

In some embodiments of formulae B-1a-1 to B-10c-8, q is O. In some embodiments, t is 0. In some embodiments, q is 0 and t is 0. In some embodiments, q is 0 and t is 1. In some embodiments, q is 0, and t is 2. In some embodiments, q is 0, and t is 3. In some embodiments, q is 1, and t is 0. In some embodiments, q is 1, and t is 1. In some embodiments, q is 1, and t is 2. In some embodiments, q is 1, and t is 3. In some embodiments, q is 2 and t is 0. In some embodiments, q is 2 and t is 1. In some embodiments, q is 2, and t is 2. In some embodiments, q is 2, and t is 3. In some embodiments, q is 3 and t is 0. In some embodiments, q is 3 and t is 1. In some embodiments, q is 3, and t is 2. In some embodiments, q is 3, and t is 3.

In some embodiments of formulae B-1a-1 to B-10c-8, R₁₈ is hydroxyl, halo, C₁-C₈ perhaloalkyl, substituted or unsubstituted C₁-C₈ alkyl, acyl, substituted or unsubstituted amino, acylamino, aminoacyl, —R₁₂ or —NHR₁₂. In some embodiments, R₁₈ is hydroxyl. In some embodiments, R₁₈ is fluoro, chloro or bromo. In some embodiments, R₁₈ is —R₁₂ or —NHR₁₂.

In some embodiments, the compound is of formulae B-1a-1 to B-1a-8, B-1b-1 to B-1b-8, or B-1c-1 to B-1c-8, wherein q is 0, t is 0 or an integer from 1 to 3, and R₁₈ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, R₁₈ is selected from the group consisting of hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃.

In some embodiments, the compound is of formulae B-1a-1 to B-1a-8, B-1b-1 to B-1b-8, or B-1c-1 to B-1c-8, wherein q is 0, t is 2, one R₁₈ is halo, and the remaining R₁₈ is hydroxy or alkoxy. In some embodiments, t is 2, and each R₁₈ is halo. In some embodiments, t is 2, and each R₁₈ is hydroxy. In some embodiments, t is 2, and each R₁₈ is alkoxy.

In some embodiments, the compound is of formulae B-1a-1 to B-1a-8, B-1b-1 to B-1b-8, or B-1c-1 to B-1c-8, wherein q is 0, t is 3, one R₁₈ is halo, and the remaining two R₁₈ is hydroxy or alkoxy. In some embodiments, t is 3, two R₁₈ are each halo, and the remaining R₁₈ is hydroxy or alkoxy. In some embodiments, t is 3, two R₁₈ are each halo, and the remaining R₁₈ is hydroxy. In some embodiments, t is 3, one R₁₈ is cyano, one R₁₈ is halo, and the remaining R₁₈ is hydroxy or alkoxy.

In some embodiments, the compound is of formulae B-1a-1 to B-1a-8, B-1b-1 to B-1b-8, or B-1c-1 to B-1c-8, wherein q is 0, t is 2, and two vicinal R₁₈ are taken together with the carbon atoms to which they are attached to form a substituted or unsubstituted heterocycle.

In some embodiments, the compound is of formulae B-1a-1 to B-1a-8, B-1b-1 to B-1b-8, or B-1c-1 to B-1c-8, wherein q is 0, t is 3, two vicinal R₁₈ are taken together with the carbon atoms to which they are attached to form a substituted or unsubstituted heterocycle, and the remaining R₁₈ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, the remaining R₁₈ is halo, hydroxy, cyano, or methoxy.

In some embodiments, the compound is of formulae B-1a-1, B-1b-1, or B-1c-1, wherein q is 0, t is 0 or an integer from 1 to 3, and R₁₈ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, R₁₈ is selected from the group consisting of hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃.

In some embodiments, the compound is of formulae B-1a-1, B-1b-1, or B-1c-1, wherein q is 0, t is 2, one R₁₈ is halo, and the remaining R₁₈ is hydroxy or alkoxy. In some embodiments, t is 2, and each R₁₈ is halo. In some embodiments, t is 2, and each R₁₈ is hydroxy. In some embodiments, t is 2, and each R₁₈ is alkoxy.

In some embodiments, the compound is of formulae B-1a-1, B-1b-1, or B-1c-1, wherein q is 0, t is 3, one R₁₈ is halo, and the remaining two R₁₈ are each hydroxy or alkoxy. In some embodiments, t is 3, two R₁₈ are each halo, and the remaining R₁₈ is hydroxy or alkoxy. In some embodiments, t is 3, two R₁₈ are each halo, and the remaining R₁₈ is hydroxy. In some embodiments, t is 3, one R₁₈ is cyano, one R₁₈ is halo, and the remaining R₁₈ is hydroxy or alkoxy.

In some embodiments, the compound is of formulae B-1a-1, B-1b-1, or B-1c-1, wherein q is 0, t is 2, and two vicinal R₁₈ are taken together with the carbon atoms to which they are attached to form a substituted or unsubstituted heterocycle.

In some embodiments, the compound is of formulae B-1a-1, B-1b-1, or B-1c-1, wherein q is 0, t is 3, two vicinal R₁₈ are taken together with the carbon atoms to which they are attached to form a substituted or unsubstituted heterocycle, and the remaining R₁₈ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, the remaining R₁₈ is halo, hydroxy, cyano, or methoxy.

Variations of formulae (I), (II) and (III), detailed throughout, where applicable, apply to formulae B-1, B-2, B-3, B-4, B-5, B-6, B-7, B-8, B-9, and B-10, the same as if each and every variation were specifically and individually listed for formulae B-1, B-2, B-3, B-4, B-5, B-6, B-7, B-8, B-9, and B-10.

Variations of formulae B-1, B-2, B-3, B-4, B-5, B-6, B-7, B-8, B-9, and B-10, detailed throughout, where applicable, apply to formulae B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, and B-10c, the same as if each and every variation were specifically and individually listed for formulae B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, and B-10c.

Variations of formulae B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, and B-10c, detailed throughout, where applicable, apply to formulae B-1a-1, B-1a-2, B-1a-3, B-1a-4, B-1a-5, B-1a-6, B-1a-7, B-1a-8, B-1b-1, B-1b-2, B-1b-3, B-1b-4, B-1b-5, B-1b-6, B-1b-7, B-1b-8, B-1c-1, B-1c-2, B-1c-3, B-1c-4, B-1c-5, B-1c-6, B-1c-7, B-1c-8, B-2a-1, B-2a-2, B-2a-3, B-2a-4, B-2a-5, B-2a-6, B-2a-7, B-2a-8, B-2b-1, B-2b-2, B-2b-3, B-2b-4, B-2b-5, B-2b-6, B-2b-7, B-2b-8, B-2c-1, B-2c-2, B-2c-3, B-2c-4, B-2c-5, B-2c-6, B-2c-7, B-2c-8, B-3a-1, B-3a-2, B-3a-3, B-3a-4, B-3a-5, B-3a-6, B-3a-7, B-3a-8, B-3b-1, B-3b-2, B-3b-3, B-3b-4, B-3b-5, B-3b-6, B-3b-7, B-3b-8, B-3c-1, B-3c-2, B-3c-3, B-3c-4, B-3c-5, B-3c-6, B-3c-7, B-3c-8, B-4a-1, B-4a-2, B-4a-3, B-4a-4, B-4a-5, B-4a-6, B-4a-7, B-4a-8, B-4b-1, B-4b-2, B-4b-3, B-4b-4, B-4b-5, B-4b-6, B-4b-7, B-4b-8, B-4c-1, B-4c-2, B-4c-3, B-4c-4, B-4c-5, B-4c-6, B-4c-7, B-4c-8, B-5a-1, B-5a-2, B-5a-3, B-5a-4, B-5a-5, B-5a-6, B-5a-7, B-5a-8, B-5b-1, B-5b-2, B-5b-3, B-5b-4, B-5b-5, B-5b-6, B-5b-7, B-5b-8, B-5c-1, B-5c-2, B-5c-3, B-5c-4, B-5c-5, B-5c-6, B-5c-7, B-5c-8, B-6a-1, B-6a-2, B-6a-3, B-6a-4, B-6a-5, B-6a-6, B-6a-7, B-6a-8, B-6b-1, B-6b-2, B-6b-3, B-6b-4, B-6b-5, B-6b-6, B-6b-7, B-6b-8, B-6c-1, B-6c-2, B-6c-3, B-6c-4, B-6c-5, B-6c-6, B-6c-7, B-6c-8, B-7a-1, B-7a-2, B-7a-3, B-7a-4, B-7a-5, B-7a-6, B-7a-7, B-7a-8, B-7b-1, B-7b-2, B-7b-3, B-7b-4, B-7b-5, B-7b-6, B-7b-7, B-7b-8, B-7c-1, B-7c-2, B-7c-3, B-7c-4, B-7c-5, B-7c-6, B-7c-7, B-7c-8, B-8a-1, B-8a-2, B-8a-3, B-8a-4, B-8a-5, B-8a-6, B-8a-7, B-8a-8, B-8b-1, B-8b-2, B-8b-3, B-8b-4, B-8b-5, B-8b-6, B-8b-7, B-8b-8, B-8c-1, B-8c-2, B-8c-3, B-8c-4, B-8c-5, B-8c-6, B-8c-7, B-8c-8, B-9a-1, B-9a-2, B-9a-3, B-9a-4, B-9a-5, B-9a-6, B-9a-7, B-9a-8, B-9b-1, B-9b-2, B-9b-3, B-9b-4, B-9b-5, B-9b-6, B-9b-7, B-9B-9, B-9c-1, B-9c-2, B-9c-3, B-9c-4, B-9c-5, B-9c-6, B-9c-7, B-9c-8, B-10a-1, B-10a-2, B-10a-3, B-10a-4, B-10a-5, B-10a-6, B-10a-7, B-10a-8, B-10b-1, B-10b-2, B-10b-3, B-10b-4, B-10b-5, B-10b-6, B-10b-7, B-10b-8, B-10c-1, B-10c-2, B-10c-3, B-10c-4, B-10c-5, B-10c-6, B-10c-7, and B-10c-8, the same as if each and every variation were specifically and individually listed for formulae B-1a-1, B-1a-2, B-1a-3, B-1a-4, B-1a-5, B-1a-6, B-1a-7, B-1a-8, B-1b-1, B-1b-2, B-1b-3, B-1b-4, B-1b-5, B-1b-6, B-1b-7, B-1b-8, B-1c-1, B-1c-2, B-1c-3, B-1c-4, B-1c-5, B-1c-6, B-1c-7, B-1c-8, B-2a-1, B-2a-2, B-2a-3, B-2a-4, B-2a-5, B-2a-6, B-2a-7, B-2a-8, B-2b-1, B-2b-2, B-2b-3, B-2b-4, B-2b-5, B-2b-6, B-2b-7, B-2b-8, B-2c-1, B-2c-2, B-2c-3, B-2c-4, B-2c-5, B-2c-6, B-2c-7, B-2c-8, B-3a-1, B-3a-2, B-3a-3, B-3a-4, B-3a-5, B-3a-6, B-3a-7, B-3a-8, B-3b-1, B-3b-2, B-3b-3, B-3b-4, B-3b-5, B-3b-6, B-3b-7, B-3b-8, B-3c-1, B-3c-2, B-3c-3, B-3c-4, B-3c-5, B-3c-6, B-3c-7, B-3c-8, B-4a-1, B-4a-2, B-4a-3, B-4a-4, B-4a-5, B-4a-6, B-4a-7, B-4a-8, B-4b-1, B-4b-2, B-4b-3, B-4b-4, B-4b-5, B-4b-6, B-4b-7, B-4b-8, B-4c-1, B-4c-2, B-4c-3, B-4c-4, B-4c-5, B-4c-6, B-4c-7, B-4c-8, B-5a-1, B-5a-2, B-5a-3, B-5a-4, B-5a-5, B-5a-6, B-5a-7, B-5a-8, B-5b-1, B-5b-2, B-5b-3, B-5b-4, B-5b-5, B-5b-6, B-5b-7, B-5b-8, B-5c-1, B-5c-2, B-5c-3, B-5c-4, B-5c-5, B-5c-6, B-5c-7, B-5c-8, B-6a-1, B-6a-2, B-6a-3, B-6a-4, B-6a-5, B-6a-6, B-6a-7, B-6a-8, B-6b-1, B-6b-2, B-6b-3, B-6b-4, B-6b-5, B-6b-6, B-6b-7, B-6b-8, B-6c-1, B-6c-2, B-6c-3, B-6c-4, B-6c-5, B-6c-6, B-6c-7, B-6c-8, B-7a-1, B-7a-2, B-7a-3, B-7a-4, B-7a-5, B-7a-6, B-7a-7, B-7a-8, B-7b-1, B-7b-2, B-7b-3, B-7b-4, B-7b-5, B-7b-6, B-7b-7, B-7b-8, B-7c-1, B-7c-2, B-7c-3, B-7c-4, B-7c-5, B-7c-6, B-7c-7, B-7c-8, B-8a-1, B-8a-2, B-8a-3, B-8a-4, B-8a-5, B-8a-6, B-8a-7, B-8a-8, B-8b-1, B-8b-2, B-8b-3, B-8b-4, B-8b-5, B-8b-6, B-8b-7, B-8b-8, B-8c-1, B-8c-2, B-8c-3, B-8c-4, B-8c-5, B-8c-6, B-8c-7, B-8c-8, B-9a-1, B-9a-2, B-9a-3, B-9a-4, B-9a-5, B-9a-6, B-9a-7, B-9a-8, B-9b-1, B-9b-2, B-9b-3, B-9b-4, B-9b-5, B-9b-6, B-9b-7, B-9B-9, B-9c-1, B-9c-2, B-9c-3, B-9c-4, B-9c-5, B-9c-6, B-9c-7, B-9c-8, B-10a-1, B-10a-2, B-10a-3, B-10a-4, B-10a-5, B-10a-6, B-10a-7, B-10a-8, B-10b-1, B-10b-2, B-10b-3, B-10b-4, B-10b-5, B-10b-6, B-10b-7, B-10b-8, B-10c-1, B-10c-2, B-10c-3, B-10c-4, B-10c-5, B-10c-6, B-10c-7, and B-10c-8.

In some embodiments of formulae (IVa), (IVb), (Va), (Vb), (VIa) and (VIb), the compound is of structure C-1a, C-1b, C-1c, C-1d, C-1e, C-1f, C-1g, C-1 h, C-1i, C-1j, C-2a, C-2b, C-2c, C-2d, C-2e, C-2f, C-2g, C-2 h, C-2i, C-2j, C-3a, C-3b, C-3c, C-3d, C-3e, C-3f, C-3g, C-3 h, C-3i, or C-3j:

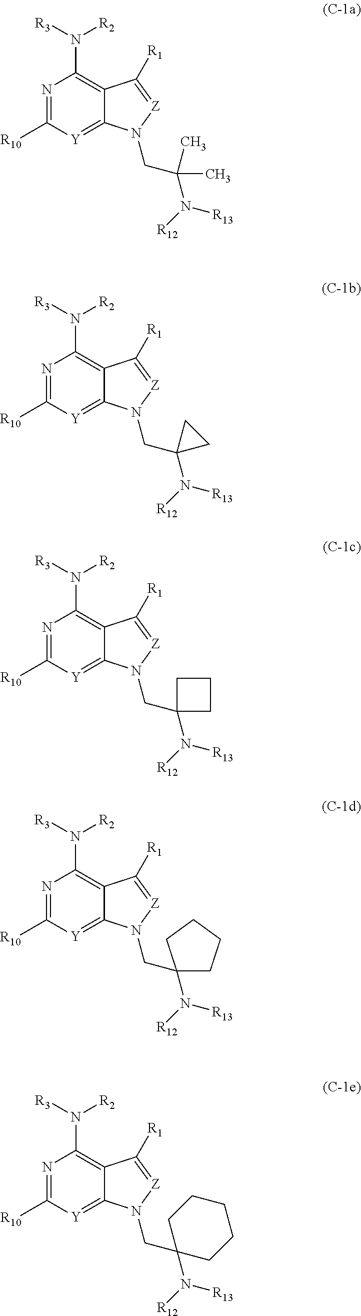

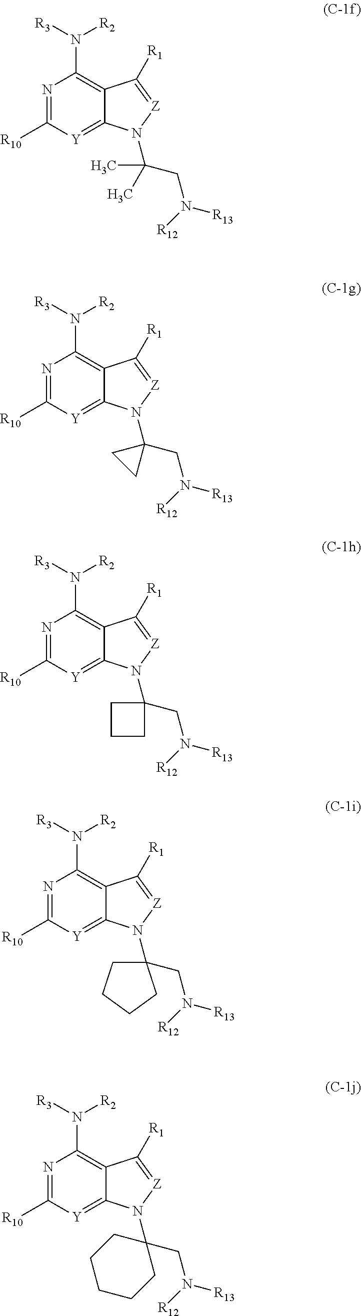

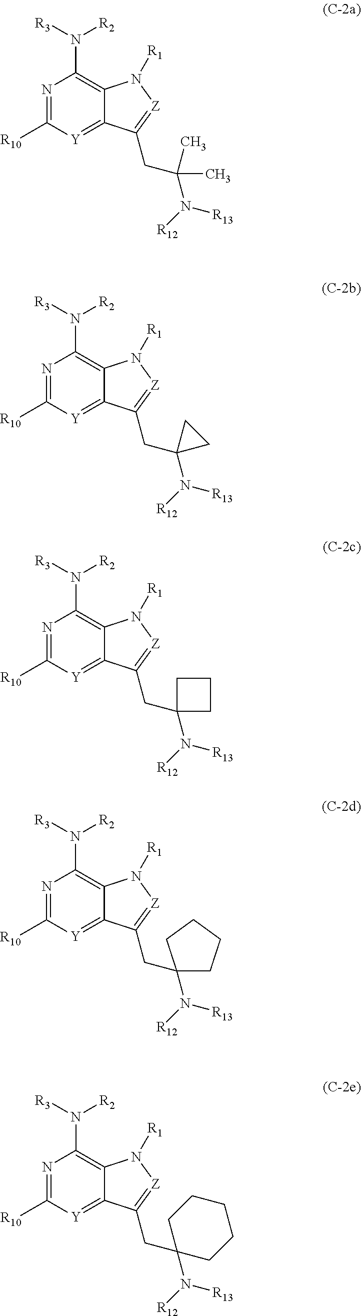

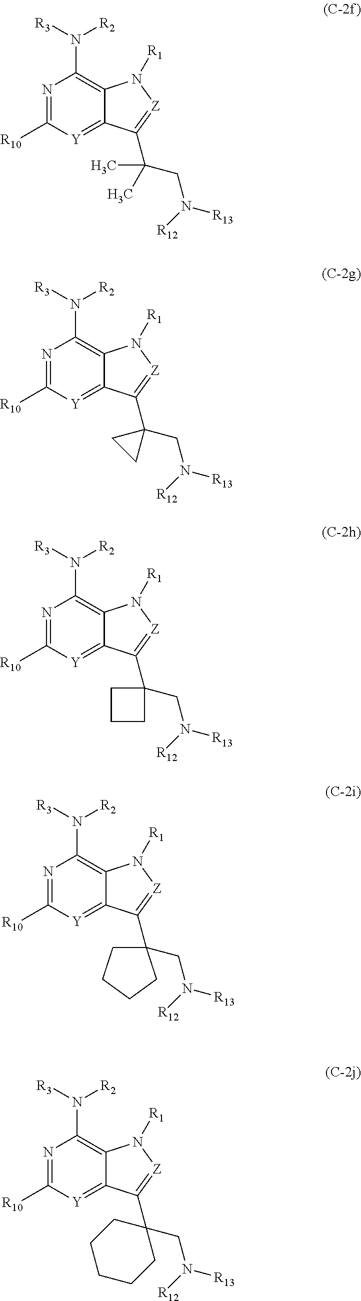

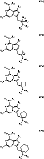

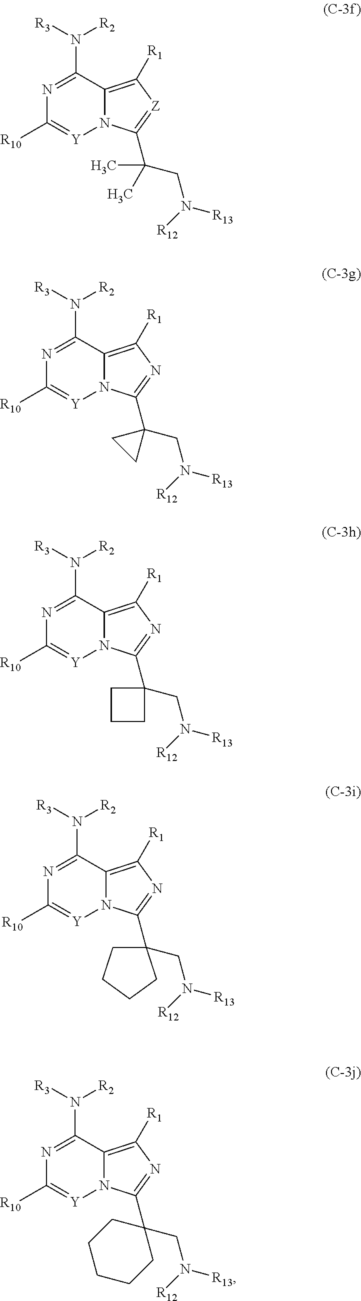

or a salt thereof; wherein R₁, R₂, R₃, R₁₀, R₁₂, and R₁₃ are as described for formulae (IVa), (IVb), (Va), (Vb), (VIa), and (VIb).

Variations of formulae (IVa), (IVb), (Va), (Vb), (VIa), and (VIb), detailed throughout, where applicable, apply to formulae C-1a, C-1b, C-1c, C-1d, C-1e, C-1f, C-1g, C-1 h, C-1i, C-1j, C-2a, C-2b, C-2c, C-2d, C-2e, C-2f, C-2g, C-2 h, C-2i, C-2j, C-3a, C-3b, C-3c, C-3d, C-3e, C-3f, C-3g, C-3 h, C-3i, and C-3j, the same as if each and every variation were specifically and individually listed for formulae C-1a, C-1b, C-1c, C-1d, C-1e, C-1f, C-1g, C-1 h, C-1i, C-1j, C-2a, C-2b, C-2c, C-2d, C-2e, C-2f, C-2g, C-2 h, C-2i, C-2j, C-3a, C-3b, C-3c, C-3d, C-3e, C-3f, C-3g, C-3 h, C-3i, and C-3j.

In some embodiments, the compound of formula (I) has the structure (D-1), (D-2), or (D-3):

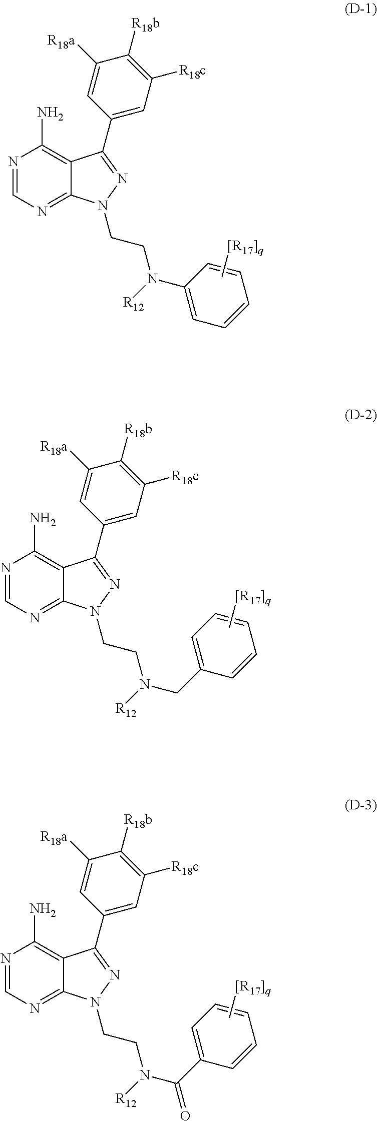

wherein R₁₂, R₁₇ and q are as described for formula (I); and each of R₁₈a, R₁₈b, and R₁₈c is independently selected from the group consisting of H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₁₂ and —NHR₁₂. In some of these embodiments, each R₁₈a, R₁₈b, and R₁₈c is independently selected from the group consisting of H, hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃. In some of these embodiments, each R₁₈a, R₁₈b, and R₁₈c is independently selected from the group consisting of H, —CF₃, hydroxyl, cyano, and halo. In some of these embodiments, each R₁₈a, R₁₈b, and R₁₈c is independently selected from the group consisting of —CF₃, hydroxyl, cyano, and halo. In some of these embodiments, each R₁₈a, R₁₈b, and R₁₈c is independently selected from the group consisting of hydroxyl, and halo.

In one embodiment, the invention relates to Compounds described in Table 1, and uses thereof.

In another embodiment, the invention relates to Compound Nos. 1, 2, 3, 4, 5, 6, 7, 8, 9, 10, 11, 12, 13, 14, 15, 16, 17, 18, 19, 20, and 21, and uses thereof.

In another embodiment, the invention relates to Compounds described in Table 2, and uses thereof.

In another embodiment, the invention relates to Compound Nos. 2.1, 2.2, 2.3, 2.4, 2.5, 2.6, 2.7, 2.8, 2.9, 2.10, 2.11, 2.12, 2.13, 2.14, 2.15, 2.16, 21.7, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, 2.100, 2.101, 2.102, 2.103, 2.104, 2.105, 2.106, 2.107, 2.108, 2.109, 2.110, 2.111, 2.112, 2.113, 2.114, 2.115, 2.116, 21.7, 2.118, 2.119, 2.120, 2.121, 2.122, 2.123, 2.124, 2.125, 2.126, 2.127, 2.128, 2.129, 2.130, 2.131, 2.132, 2.133, 2.134, 2.135, 2.136, 2.137, 2.138, 2.139, 2.140, 2.141, 2.142, 2.143, 2.144, 2.145, 2.146, 2.147, 2.148, 2.149, 2.150, 2.151, 2.152, 2.153, 2.154, 2.155, 2.156, 2.157, 2.158, 2.159, 2.160, 2.161, 2.162, 2.163, 2.164, 2.165, 2.166, 2.167, 2.168, 2.169, 2.170, 2.171, 2.172, 2.173, 2.174, 2.175, 2.176, 2.177, 2.178, 2.179, 2.180, 2.181, 2.182, 2.183, 2.184, 2.185, 2.186, 2.187, 2.188, 2.189, 2.190, 2.191, 2.192, 2.193, 2.194, 2.195, 2.196, 2.197, 2.198, 2.199, 2.200, 2.201, 2.202, 2.203, 2.204, 2.205, 2.206, 2.207, 2.208, 2.209, 2.210, 2.211, 2.212, 2.213, 2.214, 2.215, 2.216, 21.7, 2.218, 2.219, 2.220, 2.221, 2.222, 2.223, 2.224, 2.225, 2.226, 2.227, 2.228, 2.229, 2.230, 2.231, 2.232, 2.233, 2.234, 2.235, 2.236, 2.237, 2.238, 2.239, 2.240, 2.241, 2.242, 2.243, 2.244, 2.245, 2.246, 2.247, 2.248, 2.249, 2.250, 2.251, 2.252, 2.253, 2.254, 2.255, 2.256, 2.257, 2.258, 2.259, 2.260, 2.261, 2.262, 2.263, 2.264, 2.265, 2.266, 2.267, 2.268, 2.269, 2.270, 2.271, 2.272, 2.273, 2.274, 2.275, 2.276, 2.277, 2.278, 2.279, 2.280, 2.281, 2.282, 2.283, 2.284, 2.285, 2.286, 2.287, 2.288, 2.289, 2.290, 2.291, 2.292, 2.293, 2.294, 2.295, 2.296, and 2.297, and uses thereof.

Also provided are methods of using compounds described herein, including any formula detailed herein or specific compound detailed herein, in various therapeutic applications.

Representative compounds of the invention are shown in Tables 1 and 2.

TABLE 1
Representative Compounds of the Invention

| | |
|---|---|
| 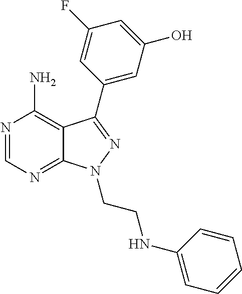 [SMILES: Nc1ncnc2c1c(-c1cc(O)cc(F)c1)nn2CCNc1ccccc1] | 1 |
| 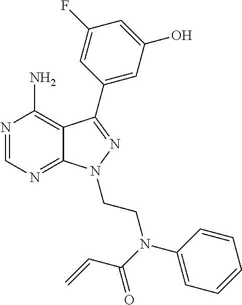 [SMILES: C=CC(=O)N(CCn1nc(-c2cc(O)cc(F)c2)c2c(N)ncnc21)c1ccccc1] | 2 |
| 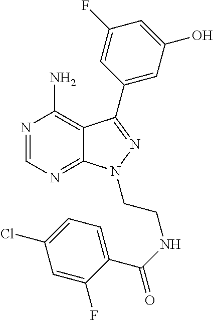 [SMILES: Nc1ncnc2c1c(-c1cc(O)cc(F)c1)nn2CCNC(=O)c1ccc(Cl)cc1F] | 3 |
| 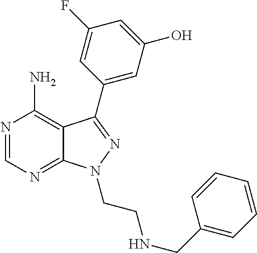 [SMILES: Nc1ncnc2c1c(-c1cc(O)cc(F)c1)nn2CCNCc1ccccc1] | 4 |
| 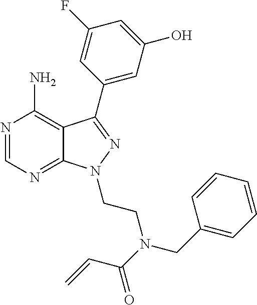 [SMILES: C=CC(=O)N(CCn1nc(-c2cc(O)cc(F)c2)c2c(N)ncnc21)Cc1ccccc1] | 5 |
| 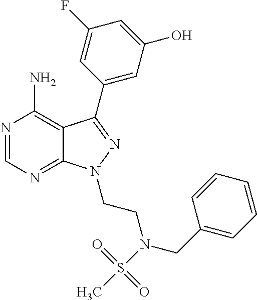 [SMILES: CS(=O)(=O)N(CCn1nc(-c2cc(O)cc(F)c2)c2c(N)ncnc21)Cc1ccccc1] | 6 |
| 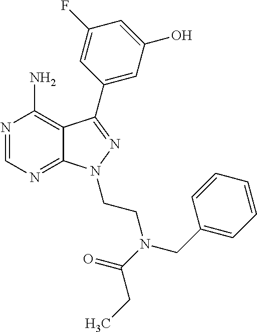 [SMILES: CCC(=O)N(CCn1nc(-c2cc(O)cc(F)c2)c2c(N)ncnc21)Cc1ccccc1] | 7 |
| 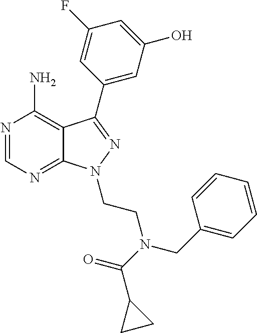 [SMILES: Nc1ncnc2c1c(-c1cc(O)cc(F)c1)nn2CCN(Cc1ccccc1)C(=O)C1CC1] | 8 |
| 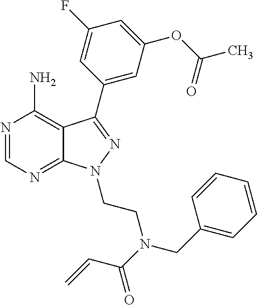 [SMILES: C=CC(=O)N(CCn1nc(-c2cc(F)cc(OC(C)=O)c2)c2c(N)ncnc21)Cc1ccccc1] | 9 |
| 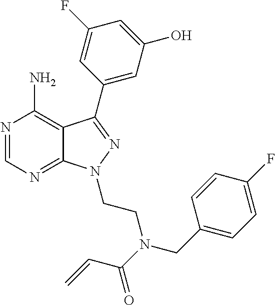 [SMILES: C=CC(=O)N(CCn1nc(-c2cc(O)cc(F)c2)c2c(N)ncnc21)Cc1ccc(F)cc1] | 10 |
| 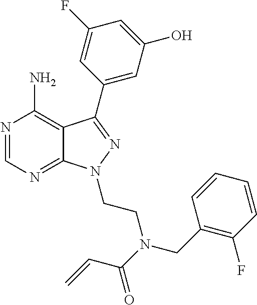 [SMILES: C=CC(=O)N(CCn1nc(-c2cc(O)cc(F)c2)c2c(N)ncnc21)Cc1ccccc1F] | 11 |
| 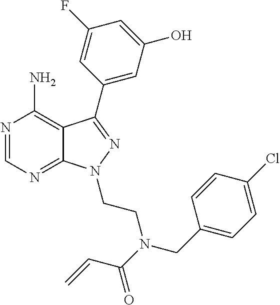 [SMILES: C=CC(=O)N(CCn1nc(-c2cc(O)cc(F)c2)c2c(N)ncnc21)Cc1ccc(Cl)cc1] | 12 |
| 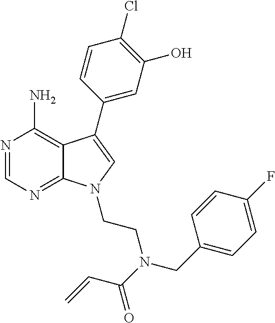 [SMILES: C=CC(=O)N(CCn1cc(-c2ccc(Cl)c(O)c2)c2c(N)ncnc21)Cc1ccc(F)cc1] | 13 |
| 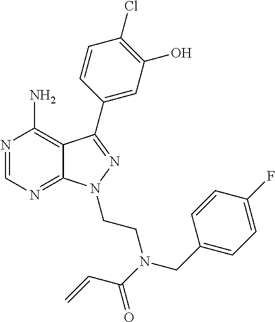 [SMILES: C=CC(=O)N(CCn1nc(-c2ccc(Cl)c(O)c2)c2c(N)ncnc21)Cc1ccc(F)cc1] | 14 |
| 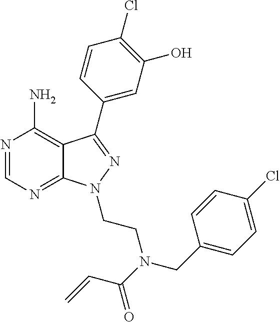 [SMILES: C=CC(=O)N(CCn1nc(-c2ccc(Cl)c(O)c2)c2c(N)ncnc21)Cc1ccc(Cl)cc1] | 15 |
| 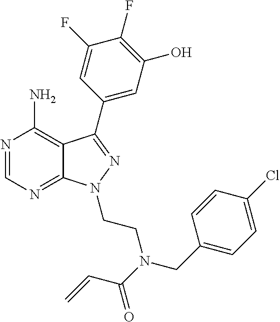 [SMILES: C=CC(=O)N(CCn1nc(-c2cc(O)c(F)c(F)c2)c2c(N)ncnc21)Cc1ccc(Cl)cc1] | 16 |
| 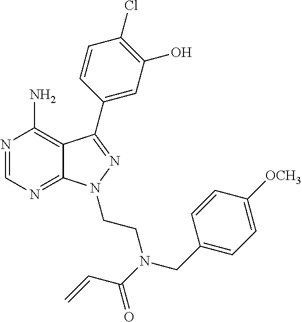 [SMILES: C=CC(=O)N(CCn1nc(-c2ccc(Cl)c(O)c2)c2c(N)ncnc21)Cc1ccc(OC)cc1] | 17 |
| 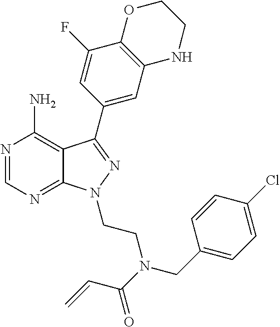 [SMILES: C=CC(=O)N(CCn1nc(-c2cc(F)c3c(c2)NCCO3)c2c(N)ncnc21)Cc1ccc(Cl)cc1] | 18 |
| 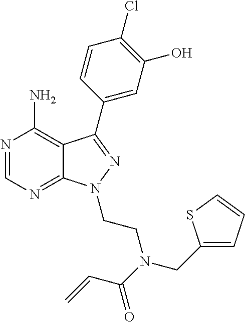 [SMILES: C=CC(=O)N(CCn1nc(-c2ccc(Cl)c(O)c2)c2c(N)ncnc21)Cc1cccs1] | 19 |
| 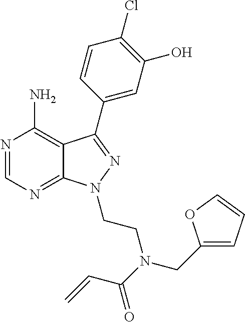 [SMILES: C=CC(=O)N(CCn1nc(-c2ccc(Cl)c(O)c2)c2c(N)ncnc21)Cc1ccco1] | 20 |
| 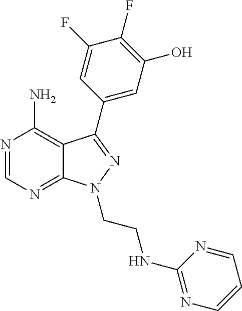 [SMILES: Nc1ncnc2c1c(-c1cc(O)c(F)c(F)c1)nn2CCNc1ncccn1] | 21 |
| 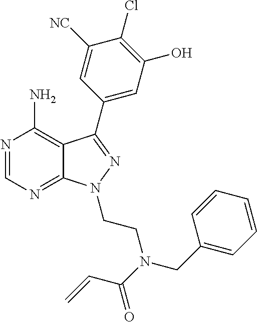 [SMILES: C=CC(=O)N(CCn1nc(-c2cc(O)c(Cl)c(C#N)c2)c2c(N)ncnc21)Cc1ccccc1] | 22 |
| 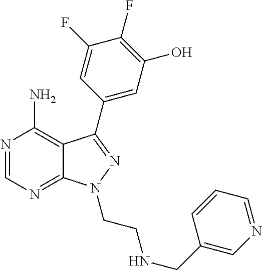 [SMILES: Nc1ncnc2c1c(-c1cc(O)c(F)c(F)c1)nn2CCNCc1cccnc1] | 23 |
| 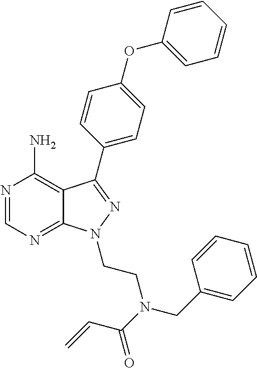 [SMILES: C=CC(=O)N(CCn1nc(-c2ccc(Oc3ccccc3)cc2)c2c(N)ncnc21)Cc1ccccc1] | 24 |

TABLE 2
Compound of the Application

| | |
|---|---|
| 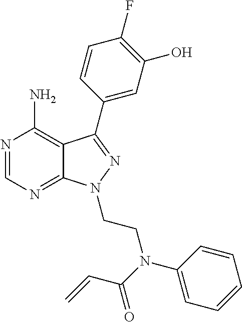 [SMILES: C=CC(=O)N(CCn1nc(-c2ccc(F)c(O)c2)c2c(N)ncnc21)c1ccccc1] | 2.1 |
| 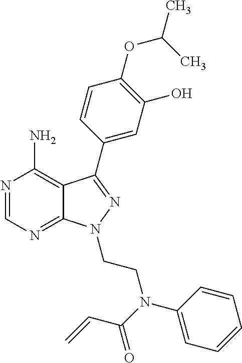 [SMILES: C=CC(=O)N(CCn1nc(-c2ccc(OC(C)C)c(O)c2)c2c(N)ncnc21)c1ccccc1] | 2.2 |
| 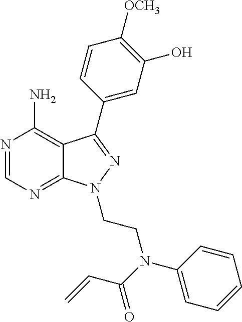 [SMILES: C=CC(=O)N(CCn1nc(-c2ccc(OC)c(O)c2)c2c(N)ncnc21)c1ccccc1] | 2.3 |
| 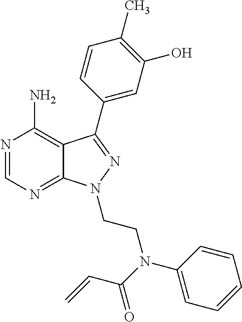 [SMILES: C=CC(=O)N(CCn1nc(-c2ccc(C)c(O)c2)c2c(N)ncnc21)c1ccccc1] | 2.4 |
| 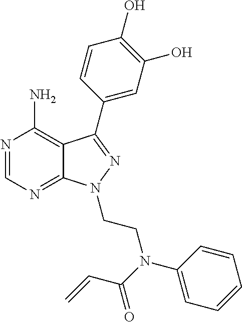 [SMILES: C=CC(=O)N(CCn1nc(-c2ccc(O)c(O)c2)c2c(N)ncnc21)c1ccccc1] | 2.5 |
| 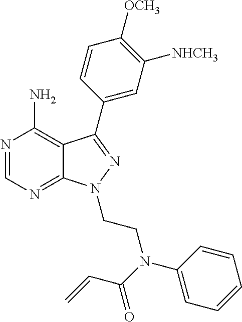 [SMILES: C=CC(=O)N(CCn1nc(-c2ccc(OC)c(NC)c2)c2c(N)ncnc21)c1ccccc1] | 2.6 |
| 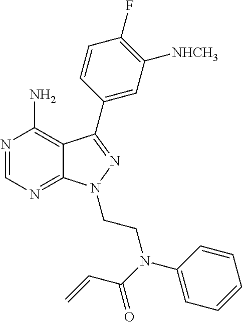 [SMILES: C=CC(=O)N(CCn1nc(-c2ccc(F)c(NC)c2)c2c(N)ncnc21)c1ccccc1] | 2.7 |
| 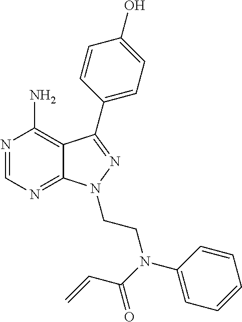 [SMILES: C=CC(=O)N(CCn1nc(-c2ccc(O)cc2)c2c(N)ncnc21)c1ccccc1] | 2.8 |
| 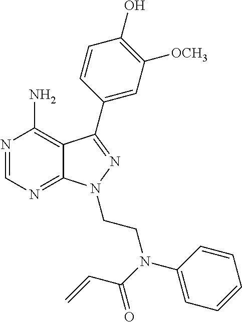 [SMILES: C=CC(=O)N(CCn1nc(-c2ccc(O)c(OC)c2)c2c(N)ncnc21)c1ccccc1] | 2.9 |
| 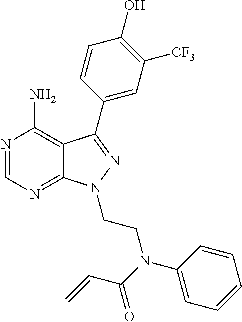 [SMILES: C=CC(=O)N(CCn1nc(-c2ccc(O)c(C(F)(F)F)c2)c2c(N)ncnc21)c1ccccc1] | 2.10 |
| 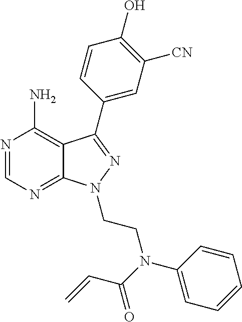 [SMILES: C=CC(=O)N(CCn1nc(-c2ccc(O)c(C#N)c2)c2c(N)ncnc21)c1ccccc1] | 2.11 |
| 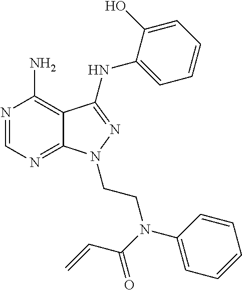 [SMILES: C=CC(=O)N(CCn1nc(Nc2ccccc2O)c2c(N)ncnc21)c1ccccc1] | 2.12 |
| 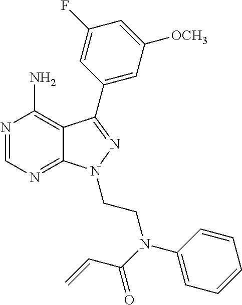 [SMILES: C=CC(=O)N(CCn1nc(-c2cc(F)cc(OC)c2)c2c(N)ncnc21)c1ccccc1] | 2.13 |
| 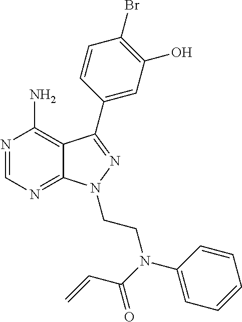 [SMILES: C=CC(=O)N(CCn1nc(-c2ccc(Br)c(O)c2)c2c(N)ncnc21)c1ccccc1] | 2.14 |
| 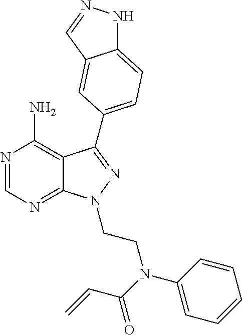 [SMILES: C=CC(=O)N(CCn1nc(-c2ccc3[nH]ncc3c2)c2c(N)ncnc21)c1ccccc1] | 2.15 |
| 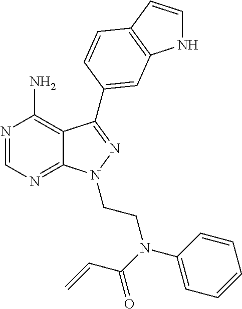 [SMILES: C=CC(=O)N(CCn1nc(-c2ccc3cc[nH]c3c2)c2c(N)ncnc21)c1ccccc1] | 2.16 |
| 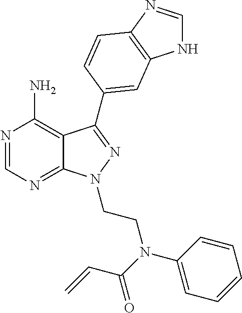 [SMILES: C=CC(=O)N(CCn1nc(-c2ccc3nc[nH]c3c2)c2c(N)ncnc21)c1ccccc1] | 2.17 |
| 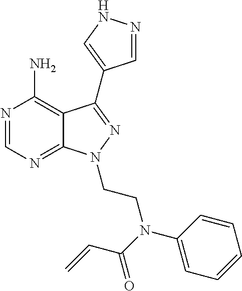 [SMILES: C=CC(=O)N(CCn1nc(-c2cn[nH]c2)c2c(N)ncnc21)c1ccccc1] | 2.18 |
| 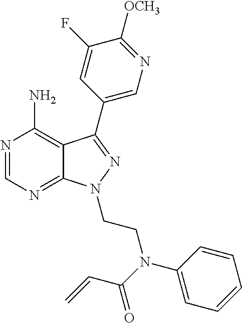 [SMILES: C=CC(=O)N(CCn1nc(-c2cnc(OC)c(F)c2)c2c(N)ncnc21)c1ccccc1] | 2.19 |
| 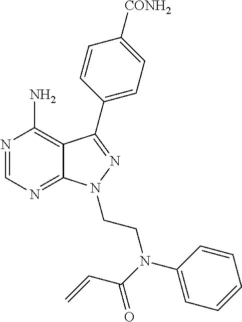 [SMILES: C=CC(=O)N(CCn1nc(-c2ccc(C(N)=O)cc2)c2c(N)ncnc21)c1ccccc1] | 2.20 |
| 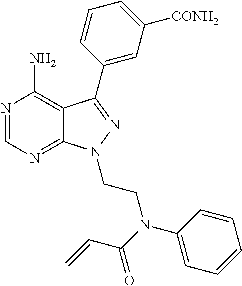 [SMILES: C=CC(=O)N(CCn1nc(-c2cccc(C(N)=O)c2)c2c(N)ncnc21)c1ccccc1] | 2.21 |
| 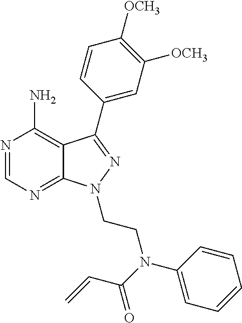 [SMILES: C=CC(=O)N(CCn1nc(-c2ccc(OC)c(OC)c2)c2c(N)ncnc21)c1ccccc1] | 2.22 |
| 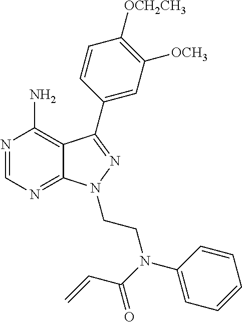 [SMILES: C=CC(=O)N(CCn1nc(-c2ccc(OCC)c(OC)c2)c2c(N)ncnc21)c1ccccc1] | 2.23 |
| 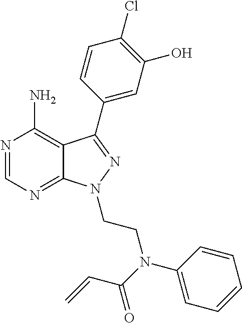 [SMILES: C=CC(=O)N(CCn1nc(-c2ccc(Cl)c(O)c2)c2c(N)ncnc21)c1ccccc1] | 2.24 |
| 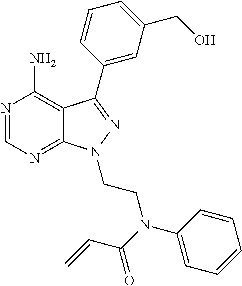 [SMILES: C=CC(=O)N(CCn1nc(-c2cccc(CO)c2)c2c(N)ncnc21)c1ccccc1] | 2.25 |
| 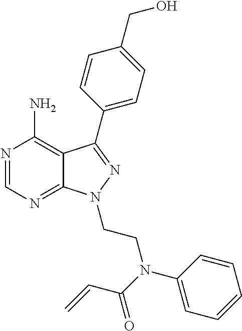 [SMILES: C=CC(=O)N(CCn1nc(-c2ccc(CO)cc2)c2c(N)ncnc21)c1ccccc1] | 2.26 |
| 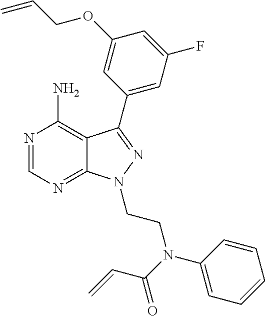 [SMILES: C=CCOc1cc(F)cc(-c2nn(CCN(C(=O)C=C)c3ccccc3)c3ncnc(N)c23)c1] | 2.27 |
| 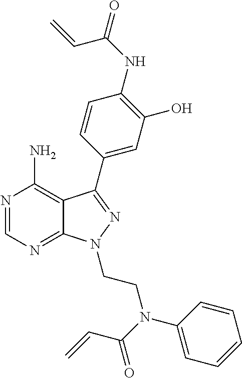 [SMILES: C=CC(=O)Nc1ccc(-c2nn(CCN(C(=O)C=C)c3ccccc3)c3ncnc(N)c23)cc1O] | 2.28 |
| 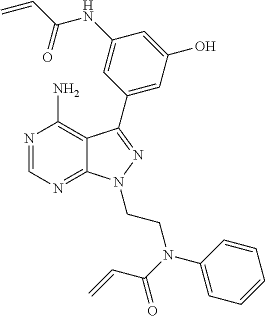 [SMILES: C=CC(=O)Nc1cc(O)cc(-c2nn(CCN(C(=O)C=C)c3ccccc3)c3ncnc(N)c23)c1] | 2.29 |
| 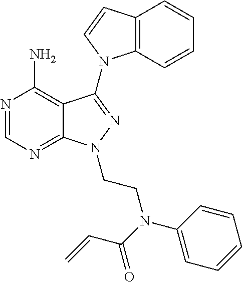 [SMILES: C=CC(=O)N(CCn1nc(-n2ccc3ccccc32)c2c(N)ncnc21)c1ccccc1] | 2.30 |
| 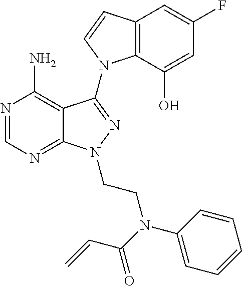 [SMILES: C=CC(=O)N(CCn1nc(-n2ccc3cc(F)cc(O)c32)c2c(N)ncnc21)c1ccccc1] | 2.31 |
| 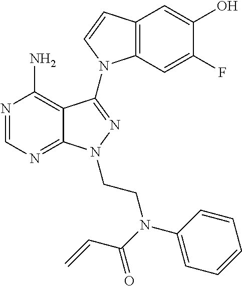 [SMILES: C=CC(=O)N(CCn1nc(-n2ccc3cc(O)c(F)cc32)c2c(N)ncnc21)c1ccccc1] | 2.32 |
| 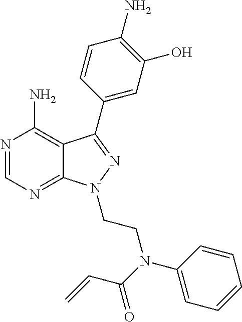 [SMILES: C=CC(=O)N(CCn1nc(-c2ccc(N)c(O)c2)c2c(N)ncnc21)c1ccccc1] | 2.33 |
| 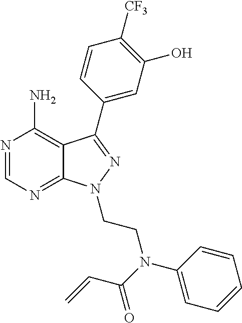 [SMILES: C=CC(=O)N(CCn1nc(-c2ccc(C(F)(F)F)c(O)c2)c2c(N)ncnc21)c1ccccc1] | 2.34 |
| 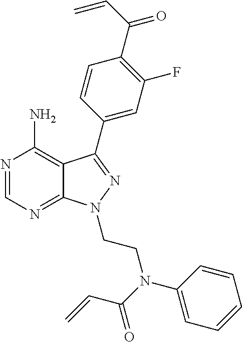 [SMILES: C=CC(=O)c1ccc(-c2nn(CCN(C(=O)C=C)c3ccccc3)c3ncnc(N)c23)cc1F] | 2.35 |
| 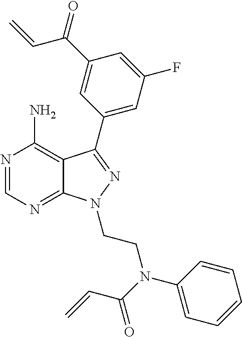 [SMILES: C=CC(=O)c1cc(F)cc(-c2nn(CCN(C(=O)C=C)c3ccccc3)c3ncnc(N)c23)c1] | 2.36 |
| 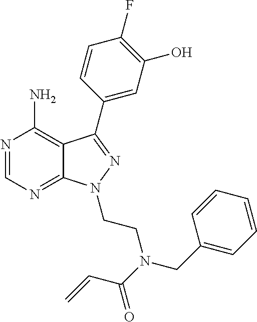 [SMILES: C=CC(=O)N(CCn1nc(-c2ccc(F)c(O)c2)c2c(N)ncnc21)Cc1ccccc1] | 2.37 |
| 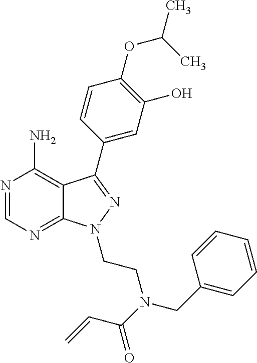 [SMILES: C=CC(=O)N(CCn1nc(-c2ccc(OC(C)C)c(O)c2)c2c(N)ncnc21)Cc1ccccc1] | 2.38 |
| 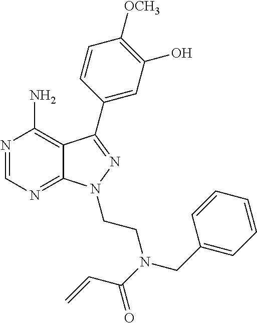 [SMILES: C=CC(=O)N(CCn1nc(-c2ccc(OC)c(O)c2)c2c(N)ncnc21)Cc1ccccc1] | 2.39 |
| 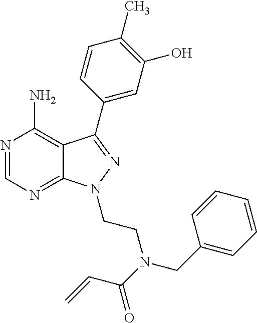 [SMILES: C=CC(=O)N(CCn1nc(-c2ccc(C)c(O)c2)c2c(N)ncnc21)Cc1ccccc1] | 2.40 |
| 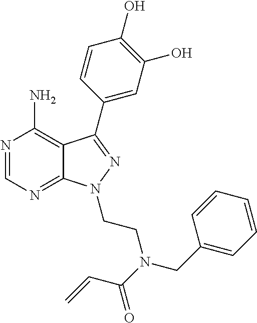 [SMILES: C=CC(=O)N(CCn1nc(-c2ccc(O)c(O)c2)c2c(N)ncnc21)Cc1ccccc1] | 2.41 |
| 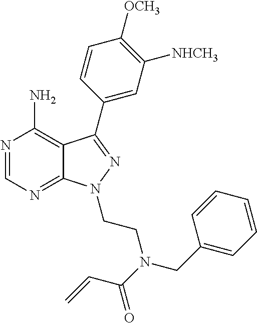 [SMILES: C=CC(=O)N(CCn1nc(-c2ccc(OC)c(NC)c2)c2c(N)ncnc21)Cc1ccccc1] | 2.42 |
| 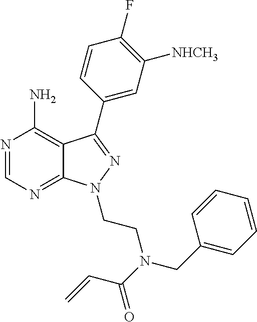 [SMILES: C=CC(=O)N(CCn1nc(-c2ccc(F)c(NC)c2)c2c(N)ncnc21)Cc1ccccc1] | 2.43 |
| 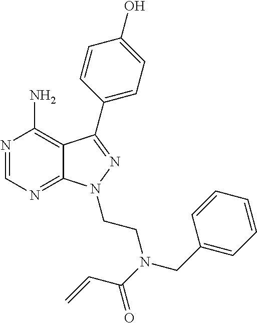 [SMILES: C=CC(=O)N(CCn1nc(-c2ccc(O)cc2)c2c(N)ncnc21)Cc1ccccc1] | 2.44 |
| 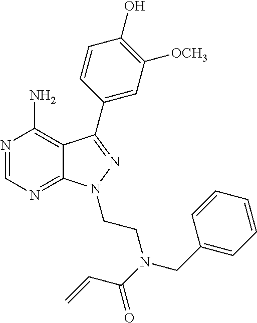 [SMILES: C=CC(=O)N(CCn1nc(-c2ccc(O)c(OC)c2)c2c(N)ncnc21)Cc1ccccc1] | 2.45 |
| 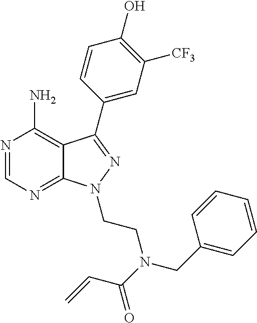 [SMILES: C=CC(=O)N(CCn1nc(-c2ccc(O)c(C(F)(F)F)c2)c2c(N)ncnc21)Cc1ccccc1] | 2.46 |
| 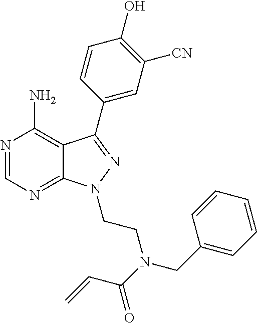 [SMILES: C=CC(=O)N(CCn1nc(-c2ccc(O)c(C#N)c2)c2c(N)ncnc21)Cc1ccccc1] | 2.47 |
| 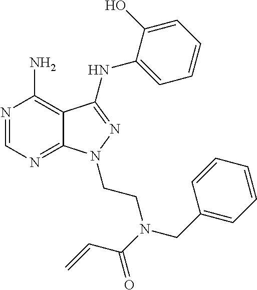 [SMILES: C=CC(=O)N(CCn1nc(Nc2ccccc2O)c2c(N)ncnc21)Cc1ccccc1] | 2.48 |
| 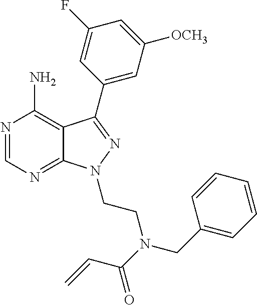 [SMILES: C=CC(=O)N(CCn1nc(-c2cc(F)cc(OC)c2)c2c(N)ncnc21)Cc1ccccc1] | 2.49 |
| 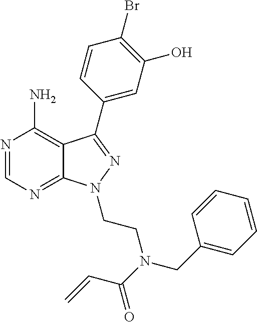 [SMILES: C=CC(=O)N(CCn1nc(-c2ccc(Br)c(O)c2)c2c(N)ncnc21)Cc1ccccc1] | 2.50 |
| 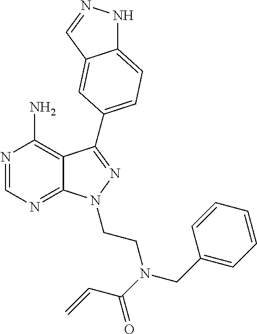 [SMILES: C=CC(=O)N(CCn1nc(-c2ccc3[nH]ncc3c2)c2c(N)ncnc21)Cc1ccccc1] | 2.51 |
| 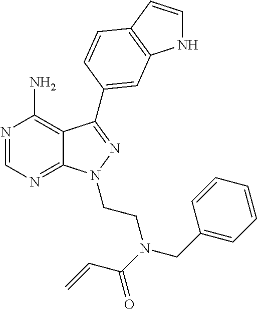 [SMILES: C=CC(=O)N(CCn1nc(-c2ccc3cc[nH]c3c2)c2c(N)ncnc21)Cc1ccccc1] | 2.52 |
| 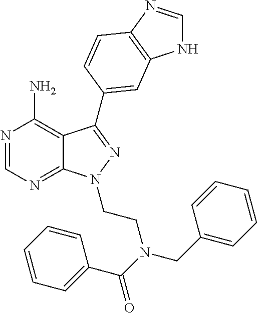 [SMILES: Nc1ncnc2c1c(-c1ccc3nc[nH]c3c1)nn2CCN(Cc1ccccc1)C(=O)c1ccccc1] | 2.53 |
| 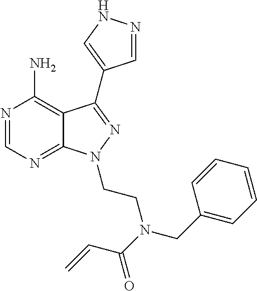 [SMILES: C=CC(=O)N(CCn1nc(-c2cn[nH]c2)c2c(N)ncnc21)Cc1ccccc1] | 2.54 |
| 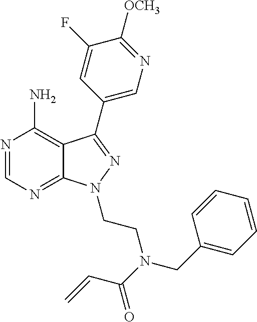 [SMILES: C=CC(=O)N(CCn1nc(-c2cnc(OC)c(F)c2)c2c(N)ncnc21)Cc1ccccc1] | 2.55 |
| 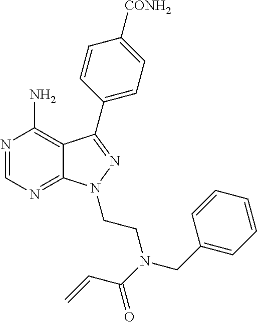 [SMILES: C=CC(=O)N(CCn1nc(-c2ccc(C(N)=O)cc2)c2c(N)ncnc21)Cc1ccccc1] | 2.56 |
| 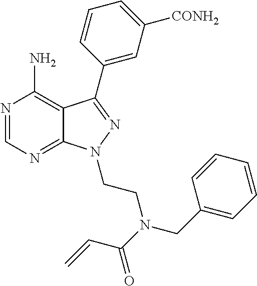 [SMILES: C=CC(=O)N(CCn1nc(-c2cccc(C(N)=O)c2)c2c(N)ncnc21)Cc1ccccc1] | 2.57 |
| 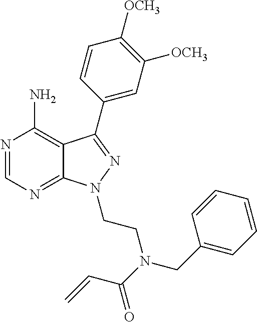 [SMILES: C=CC(=O)N(CCn1nc(-c2ccc(OC)c(OC)c2)c2c(N)ncnc21)Cc1ccccc1] | 2.58 |
| 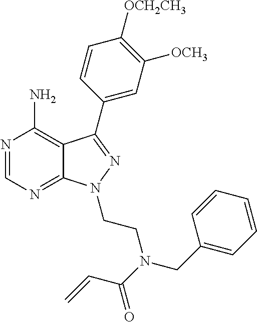 [SMILES: C=CC(=O)N(CCn1nc(-c2ccc(OCC)c(OC)c2)c2c(N)ncnc21)Cc1ccccc1] | 2.59 |
| 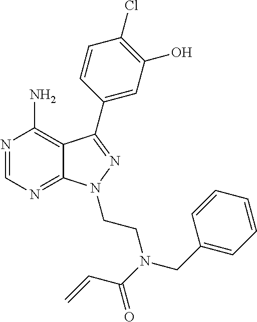 [SMILES: C=CC(=O)N(CCn1nc(-c2ccc(Cl)c(O)c2)c2c(N)ncnc21)Cc1ccccc1] | 2.60 |
| 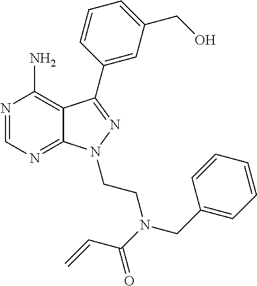 [SMILES: C=CC(=O)N(CCn1nc(-c2cccc(CO)c2)c2c(N)ncnc21)Cc1ccccc1] | 2.61 |
| 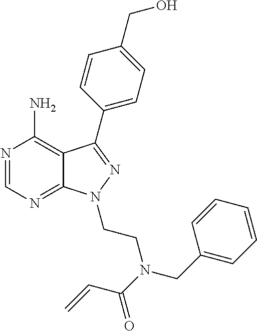 [SMILES: C=CC(=O)N(CCn1nc(-c2ccc(CO)cc2)c2c(N)ncnc21)Cc1ccccc1] | 2.62 |
| 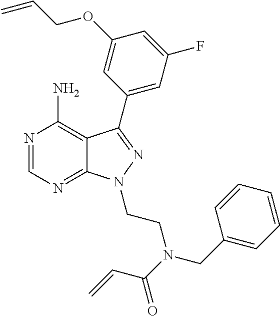 [SMILES: C=CCOc1cc(F)cc(-c2nn(CCN(Cc3ccccc3)C(=O)C=C)c3ncnc(N)c23)c1] | 2.63 |
| 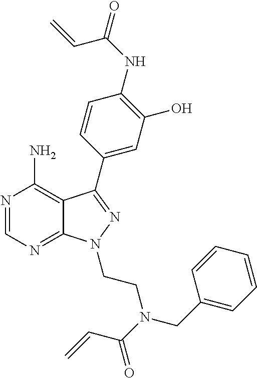 [SMILES: C=CC(=O)Nc1ccc(-c2nn(CCN(Cc3ccccc3)C(=O)C=C)c3ncnc(N)c23)cc1O] | 2.64 |
| 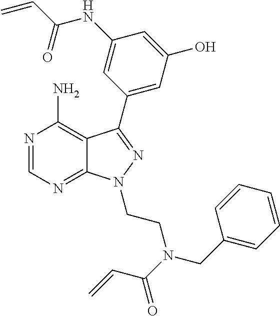 [SMILES: C=CC(=O)Nc1cc(O)cc(-c2nn(CCN(Cc3ccccc3)C(=O)C=C)c3ncnc(N)c23)c1] | 2.65 |
| 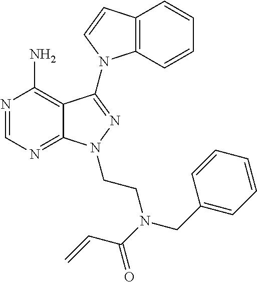 [SMILES: C=CC(=O)N(CCn1nc(-n2ccc3ccccc32)c2c(N)ncnc21)Cc1ccccc1] | 2.66 |
| 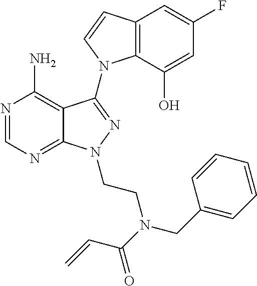 [SMILES: C=CC(=O)N(CCn1nc(-n2ccc3cc(F)cc(O)c32)c2c(N)ncnc21)Cc1ccccc1] | 2.67 |
| 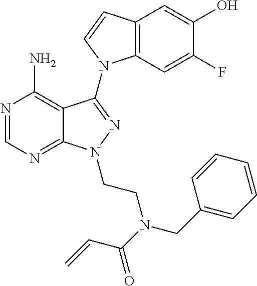 [SMILES: C=CC(=O)N(CCn1nc(-n2ccc3cc(O)c(F)cc32)c2c(N)ncnc21)Cc1ccccc1] | 2.68 |
| 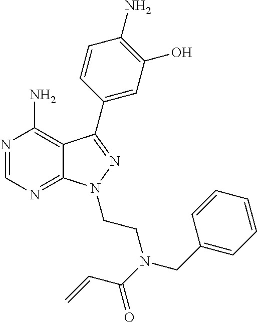 [SMILES: C=CC(=O)N(CCn1nc(-c2ccc(N)c(O)c2)c2c(N)ncnc21)Cc1ccccc1] | 2.69 |
| 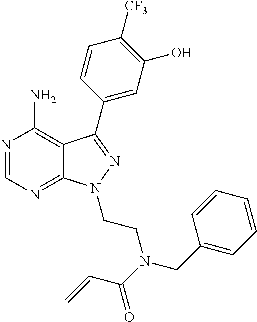 [SMILES: C=CC(=O)N(CCn1nc(-c2ccc(C(F)(F)F)c(O)c2)c2c(N)ncnc21)Cc1ccccc1] | 2.70 |
| 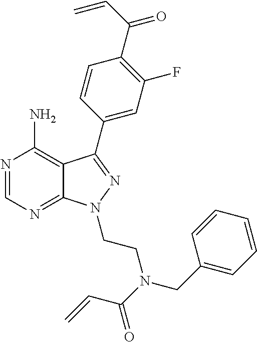 [SMILES: C=CC(=O)c1ccc(-c2nn(CCN(Cc3ccccc3)C(=O)C=C)c3ncnc(N)c23)cc1F] | 2.71 |
| 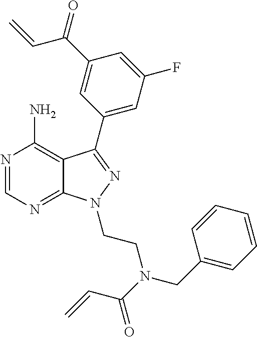 [SMILES: C=CC(=O)c1cc(F)cc(-c2nn(CCN(Cc3ccccc3)C(=O)C=C)c3ncnc(N)c23)c1] | 2.72 |
| 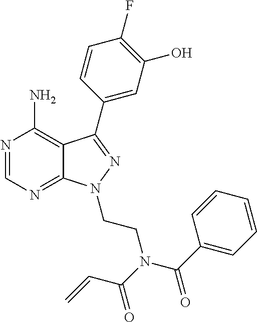 [SMILES: C=CC(=O)N(CCn1nc(-c2ccc(F)c(O)c2)c2c(N)ncnc21)C(=O)c1ccccc1] | 2.73 |
| 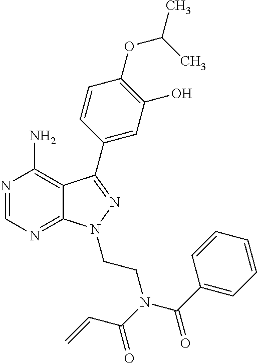 [SMILES: C=CC(=O)N(CCn1nc(-c2ccc(OC(C)C)c(O)c2)c2c(N)ncnc21)C(=O)c1ccccc1] | 2.74 |
| 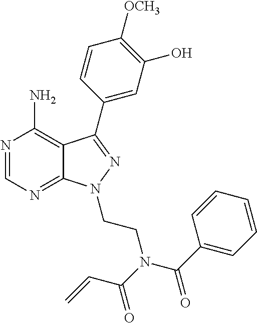 [SMILES: C=CC(=O)N(CCn1nc(-c2ccc(OC)c(O)c2)c2c(N)ncnc21)C(=O)c1ccccc1] | 2.75 |
| 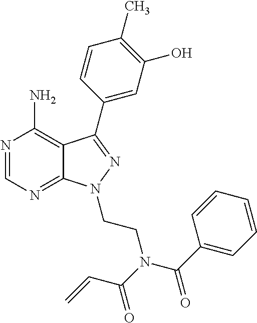 [SMILES: C=CC(=O)N(CCn1nc(-c2ccc(C)c(O)c2)c2c(N)ncnc21)C(=O)c1ccccc1] | 2.76 |
| 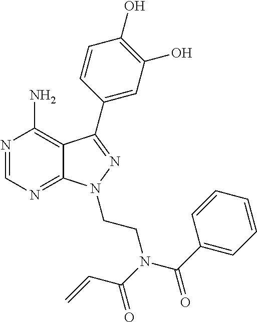 [SMILES: C=CC(=O)N(CCn1nc(-c2ccc(O)c(O)c2)c2c(N)ncnc21)C(=O)c1ccccc1] | 2.77 |
| 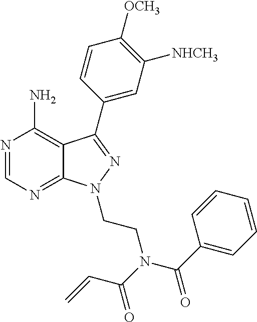 [SMILES: C=CC(=O)N(CCn1nc(-c2ccc(OC)c(NC)c2)c2c(N)ncnc21)C(=O)c1ccccc1] | 2.78 |
| 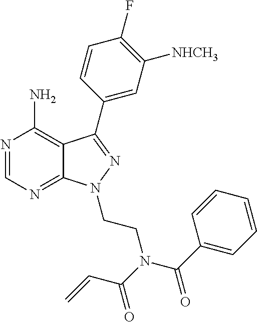 [SMILES: C=CC(=O)N(CCn1nc(-c2ccc(F)c(NC)c2)c2c(N)ncnc21)C(=O)c1ccccc1] | 2.79 |
| 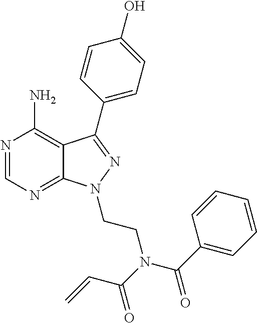 [SMILES: C=CC(=O)N(CCn1nc(-c2ccc(O)cc2)c2c(N)ncnc21)C(=O)c1ccccc1] | 2.80 |
| 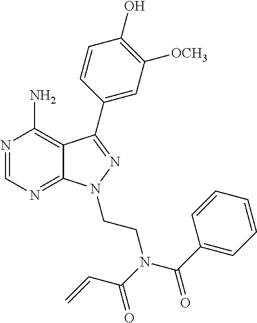 [SMILES: C=CC(=O)N(CCn1nc(-c2ccc(O)c(OC)c2)c2c(N)ncnc21)C(=O)c1ccccc1] | 2.81 |
| 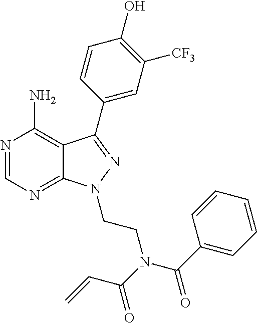 [SMILES: C=CC(=O)N(CCn1nc(-c2ccc(O)c(C(F)(F)F)c2)c2c(N)ncnc21)C(=O)c1ccccc1] | 2.82 |
| 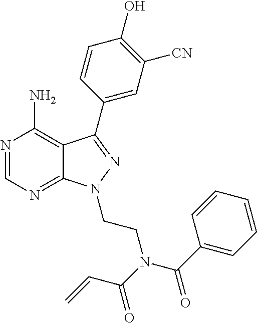 [SMILES: C=CC(=O)N(CCn1nc(-c2ccc(O)c(C#N)c2)c2c(N)ncnc21)C(=O)c1ccccc1] | 2.83 |
| 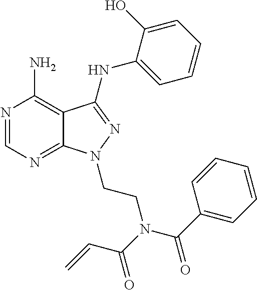 [SMILES: C=CC(=O)N(CCn1nc(Nc2ccccc2O)c2c(N)ncnc21)C(=O)c1ccccc1] | 2.84 |
| 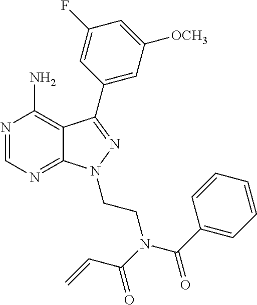 [SMILES: C=CC(=O)N(CCn1nc(-c2cc(F)cc(OC)c2)c2c(N)ncnc21)C(=O)c1ccccc1] | 2.85 |
| 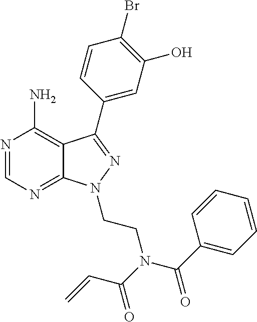 [SMILES: C=CC(=O)N(CCn1nc(-c2ccc(Br)c(O)c2)c2c(N)ncnc21)C(=O)c1ccccc1] | 2.86 |
| 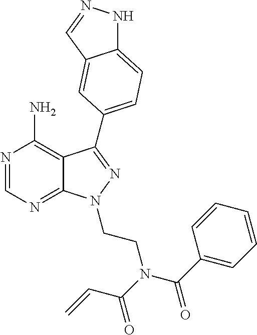 [SMILES: C=CC(=O)N(CCn1nc(-c2ccc3[nH]ncc3c2)c2c(N)ncnc21)C(=O)c1ccccc1] | 2.87 |
| 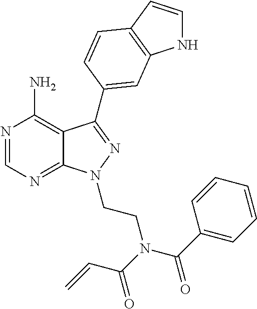 [SMILES: C=CC(=O)N(CCn1nc(-c2ccc3cc[nH]c3c2)c2c(N)ncnc21)C(=O)c1ccccc1] | 2.88 |
| 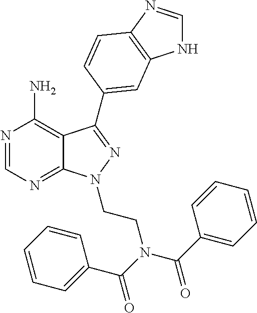 [SMILES: Nc1ncnc2c1c(-c1ccc3nc[nH]c3c1)nn2CCN(C(=O)c1ccccc1)C(=O)c1ccccc1] | 2.89 |
| 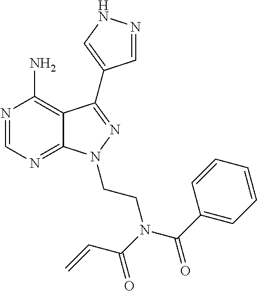 [SMILES: C=CC(=O)N(CCn1nc(-c2cn[nH]c2)c2c(N)ncnc21)C(=O)c1ccccc1] | 2.90 |
| 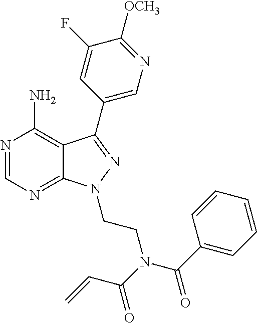 [SMILES: C=CC(=O)N(CCn1nc(-c2cnc(OC)c(F)c2)c2c(N)ncnc21)C(=O)c1ccccc1] | 2.91 |
| 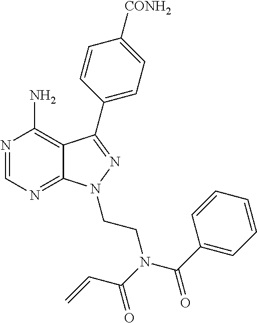 [SMILES: C=CC(=O)N(CCn1nc(-c2ccc(C(N)=O)cc2)c2c(N)ncnc21)C(=O)c1ccccc1] | 2.92 |
| 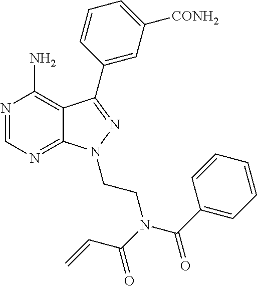 [SMILES: C=CC(=O)N(CCn1nc(-c2cccc(C(N)=O)c2)c2c(N)ncnc21)C(=O)c1ccccc1] | 2.93 |
| 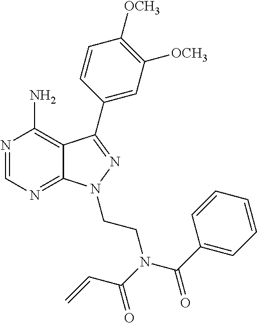 [SMILES: C=CC(=O)N(CCn1nc(-c2ccc(OC)c(OC)c2)c2c(N)ncnc21)C(=O)c1ccccc1] | 2.94 |
| 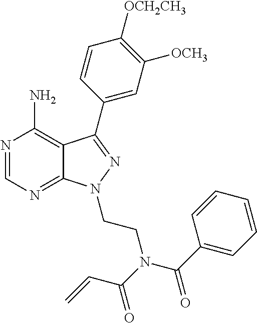 [SMILES: C=CC(=O)N(CCn1nc(-c2ccc(OCC)c(OC)c2)c2c(N)ncnc21)C(=O)c1ccccc1] | 2.95 |
| 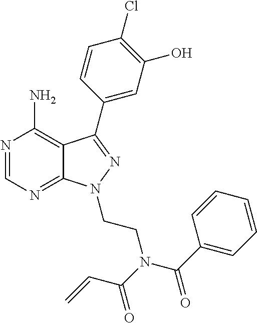 [SMILES: C=CC(=O)N(CCn1nc(-c2ccc(Cl)c(O)c2)c2c(N)ncnc21)C(=O)c1ccccc1] | 2.96 |
| 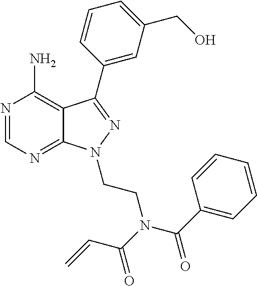 [SMILES: C=CC(=O)N(CCn1nc(-c2cccc(CO)c2)c2c(N)ncnc21)C(=O)c1ccccc1] | 2.97 |
| 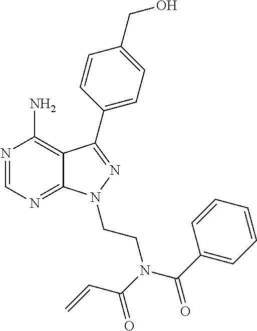 [SMILES: C=CC(=O)N(CCn1nc(-c2ccc(CO)cc2)c2c(N)ncnc21)C(=O)c1ccccc1] | 2.98 |
| 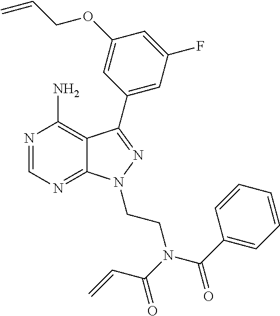 [SMILES: C=CCOc1cc(F)cc(-c2nn(CCN(C(=O)C=C)C(=O)c3ccccc3)c3ncnc(N)c23)c1] | 2.99 |
| 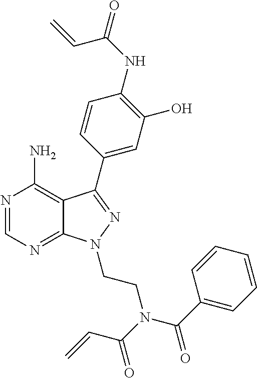 [SMILES: C=CC(=O)Nc1ccc(-c2nn(CCN(C(=O)C=C)C(=O)c3ccccc3)c3ncnc(N)c23)cc1O] | 2.100 |
| 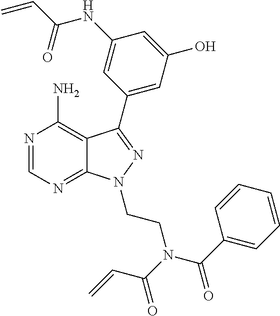 [SMILES: C=CC(=O)Nc1cc(O)cc(-c2nn(CCN(C(=O)C=C)C(=O)c3ccccc3)c3ncnc(N)c23)c1] | 2.101 |
| 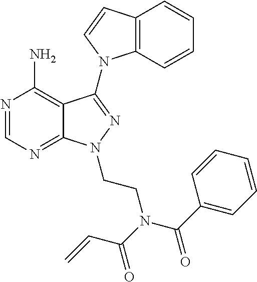 [SMILES: C=CC(=O)N(CCn1nc(-n2ccc3ccccc32)c2c(N)ncnc21)C(=O)c1ccccc1] | 2.102 |
| 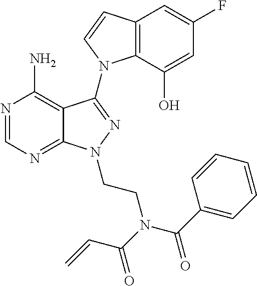 [SMILES: C=CC(=O)N(CCn1nc(-n2ccc3cc(F)cc(O)c32)c2c(N)ncnc21)C(=O)c1ccccc1] | 2.103 |
| 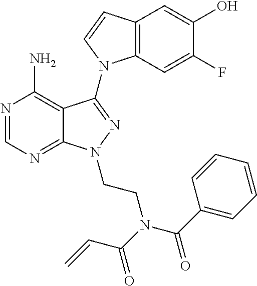 [SMILES: C=CC(=O)N(CCn1nc(-n2ccc3cc(O)c(F)cc32)c2c(N)ncnc21)C(=O)c1ccccc1] | 2.104 |
| 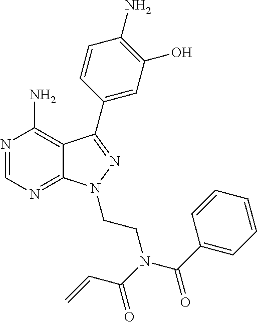 [SMILES: C=CC(=O)N(CCn1nc(-c2ccc(N)c(O)c2)c2c(N)ncnc21)C(=O)c1ccccc1] | 2.105 |
| 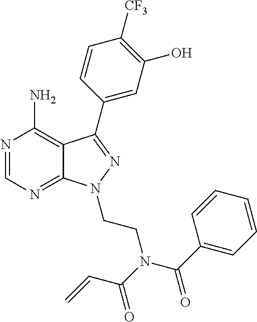 [SMILES: C=CC(=O)N(CCn1nc(-c2ccc(C(F)(F)F)c(O)c2)c2c(N)ncnc21)C(=O)c1ccccc1] | 2.106 |
| 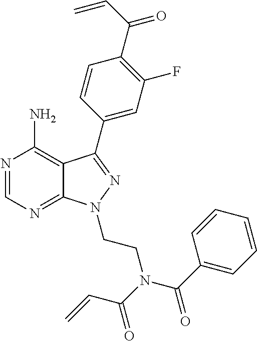 [SMILES: C=CC(=O)c1ccc(-c2nn(CCN(C(=O)C=C)C(=O)c3ccccc3)c3ncnc(N)c23)cc1F] | 2.107 |
| 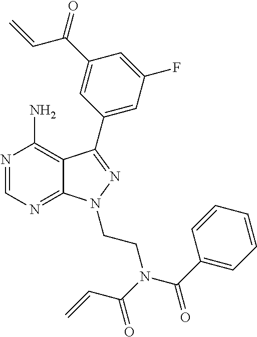 [SMILES: C=CC(=O)c1cc(F)cc(-c2nn(CCN(C(=O)C=C)C(=O)c3ccccc3)c3ncnc(N)c23)c1] | 2.108 |
| 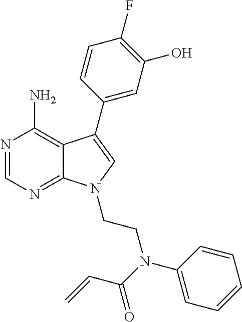 [SMILES: C=CC(=O)N(CCn1cc(-c2ccc(F)c(O)c2)c2c(N)ncnc21)c1ccccc1] | 2.109 |
| 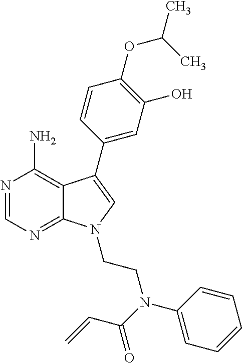 [SMILES: C=CC(=O)N(CCn1cc(-c2ccc(OC(C)C)c(O)c2)c2c(N)ncnc21)c1ccccc1] | 2.110 |
| 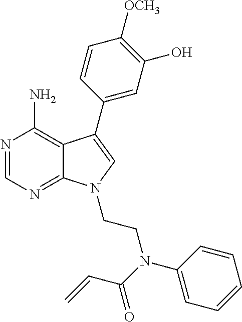 [SMILES: C=CC(=O)N(CCn1cc(-c2ccc(OC)c(O)c2)c2c(N)ncnc21)c1ccccc1] | 2.111 |
| 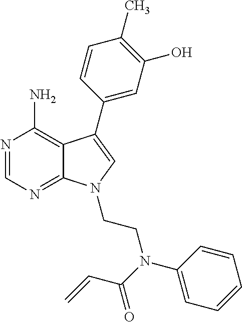 [SMILES: C=CC(=O)N(CCn1cc(-c2ccc(C)c(O)c2)c2c(N)ncnc21)c1ccccc1] | 2.112 |
| 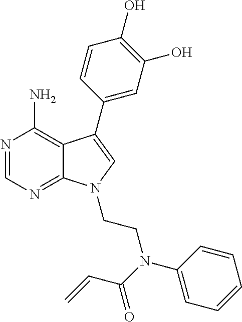 [SMILES: C=CC(=O)N(CCn1cc(-c2ccc(O)c(O)c2)c2c(N)ncnc21)c1ccccc1] | 2.113 |
| 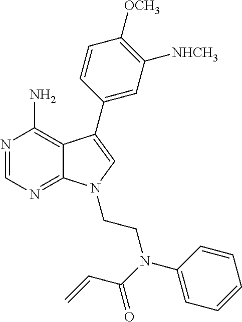 [SMILES: C=CC(=O)N(CCn1cc(-c2ccc(OC)c(NC)c2)c2c(N)ncnc21)c1ccccc1] | 2.114 |
| 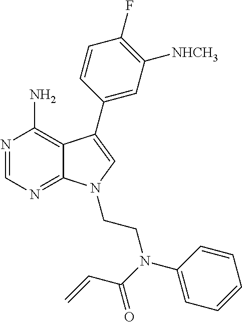 [SMILES: C=CC(=O)N(CCn1cc(-c2ccc(F)c(NC)c2)c2c(N)ncnc21)c1ccccc1] | 2.115 |
| 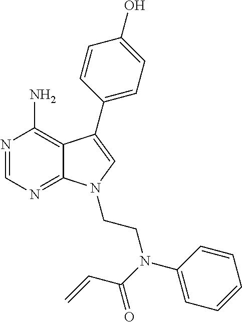 [SMILES: C=CC(=O)N(CCn1cc(-c2ccc(O)cc2)c2c(N)ncnc21)c1ccccc1] | 2.116 |
| 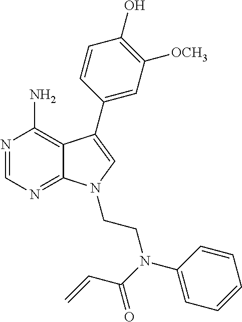 [SMILES: C=CC(=O)N(CCn1cc(-c2ccc(O)c(OC)c2)c2c(N)ncnc21)c1ccccc1] | 2.117 |
| 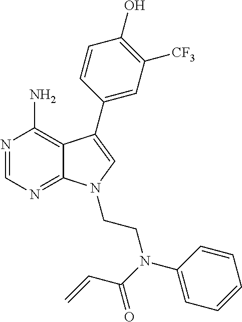 [SMILES: C=CC(=O)N(CCn1cc(-c2ccc(O)c(C(F)(F)F)c2)c2c(N)ncnc21)c1ccccc1] | 2.118 |
| 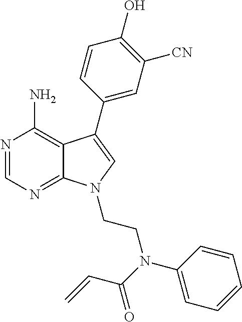 [SMILES: C=CC(=O)N(CCn1cc(-c2ccc(O)c(C#N)c2)c2c(N)ncnc21)c1ccccc1] | 2.119 |
| 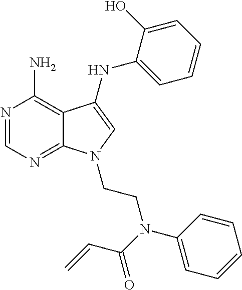 [SMILES: C=CC(=O)N(CCn1cc(Nc2ccccc2O)c2c(N)ncnc21)c1ccccc1] | 2.120 |
| 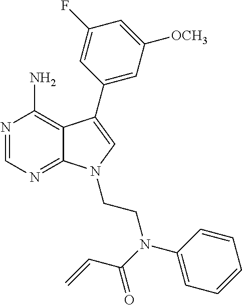 [SMILES: C=CC(=O)N(CCn1cc(-c2cc(F)cc(OC)c2)c2c(N)ncnc21)c1ccccc1] | 2.121 |
| 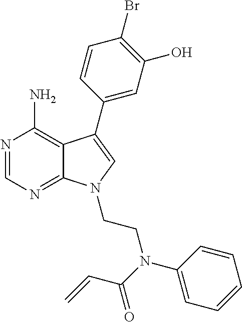 [SMILES: C=CC(=O)N(CCn1cc(-c2ccc(Br)c(O)c2)c2c(N)ncnc21)c1ccccc1] | 2.122 |
| 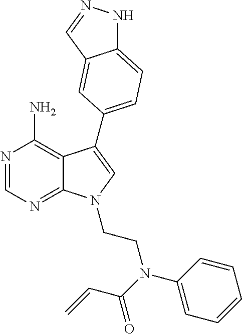 [SMILES: C=CC(=O)N(CCn1cc(-c2ccc3[nH]ncc3c2)c2c(N)ncnc21)c1ccccc1] | 2.123 |
| 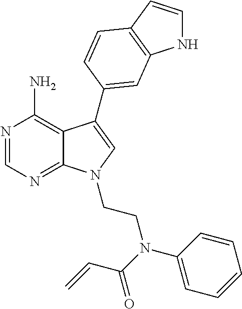 [SMILES: C=CC(=O)N(CCn1cc(-c2ccc3cc[nH]c3c2)c2c(N)ncnc21)c1ccccc1] | 2.124 |
| 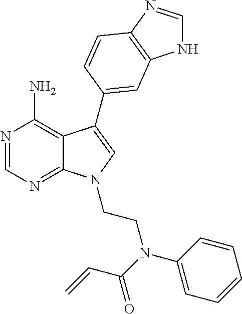 [SMILES: C=CC(=O)N(CCn1cc(-c2ccc3nc[nH]c3c2)c2c(N)ncnc21)c1ccccc1] | 2.125 |
| 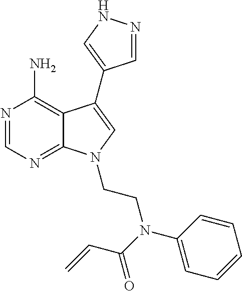 [SMILES: C=CC(=O)N(CCn1cc(-c2cn[nH]c2)c2c(N)ncnc21)c1ccccc1] | 2.126 |
| 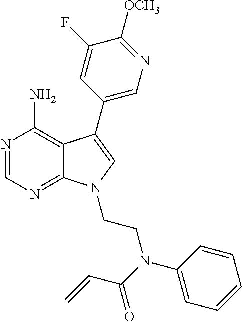 [SMILES: C=CC(=O)N(CCn1cc(-c2cnc(OC)c(F)c2)c2c(N)ncnc21)c1ccccc1] | 2.127 |
| 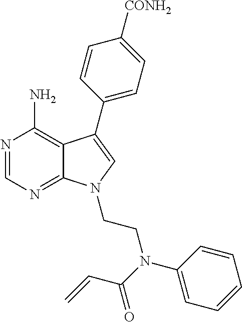 [SMILES: C=CC(=O)N(CCn1cc(-c2ccc(C(N)=O)cc2)c2c(N)ncnc21)c1ccccc1] | 2.128 |
| 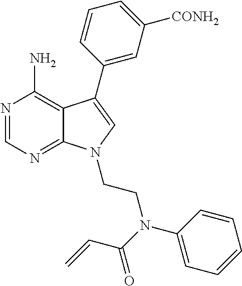 [SMILES: C=CC(=O)N(CCn1cc(-c2cccc(C(N)=O)c2)c2c(N)ncnc21)c1ccccc1] | 2.129 |
| 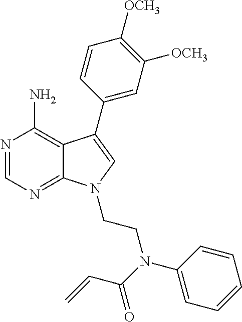 [SMILES: C=CC(=O)N(CCn1cc(-c2ccc(OC)c(OC)c2)c2c(N)ncnc21)c1ccccc1] | 2.130 |
| 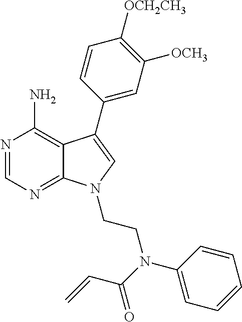 [SMILES: C=CC(=O)N(CCn1cc(-c2ccc(OCC)c(OC)c2)c2c(N)ncnc21)c1ccccc1] | 2.131 |
| 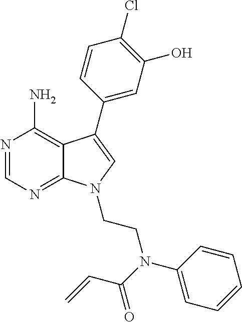 [SMILES: C=CC(=O)N(CCn1cc(-c2ccc(Cl)c(O)c2)c2c(N)ncnc21)c1ccccc1] | 2.132 |
| 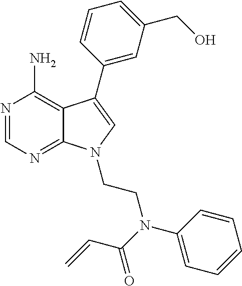 [SMILES: C=CC(=O)N(CCn1cc(-c2cccc(CO)c2)c2c(N)ncnc21)c1ccccc1] | 2.133 |
| 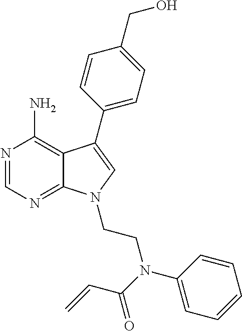 [SMILES: C=CC(=O)N(CCn1cc(-c2ccc(CO)cc2)c2c(N)ncnc21)c1ccccc1] | 2.134 |
| 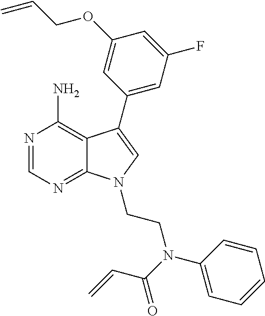 [SMILES: C=CCOc1cc(F)cc(-c2cn(CCN(C(=O)C=C)c3ccccc3)c3ncnc(N)c23)c1] | 2.135 |
| 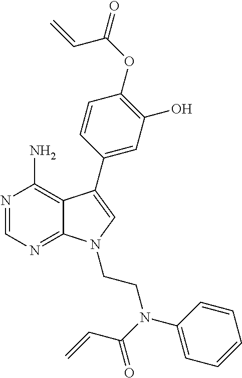 [SMILES: C=CC(=O)Oc1ccc(-c2cn(CCN(C(=O)C=C)c3ccccc3)c3ncnc(N)c23)cc1O] | 2.136 |
| 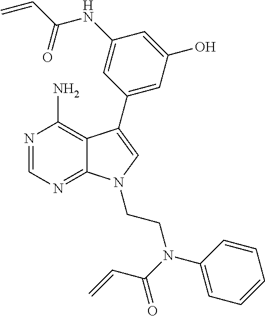 [SMILES: C=CC(=O)Nc1cc(O)cc(-c2cn(CCN(C(=O)C=C)c3ccccc3)c3ncnc(N)c23)c1] | 2.137 |
| 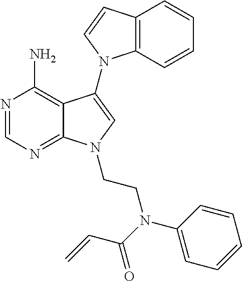 [SMILES: C=CC(=O)N(CCn1cc(-n2ccc3ccccc32)c2c(N)ncnc21)c1ccccc1] | 2.138 |
| 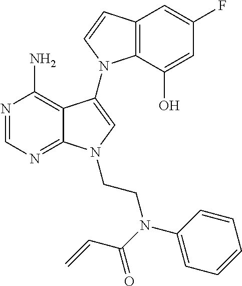 [SMILES: C=CC(=O)N(CCn1cc(-n2ccc3cc(F)cc(O)c32)c2c(N)ncnc21)c1ccccc1] | 2.139 |
| 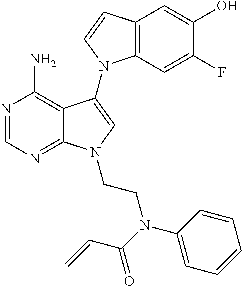 [SMILES: C=CC(=O)N(CCn1cc(-n2ccc3cc(O)c(F)cc32)c2c(N)ncnc21)c1ccccc1] | 2.140 |
| 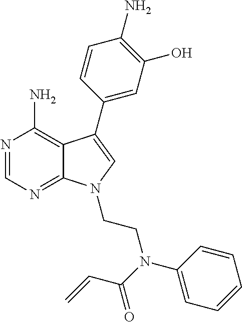 [SMILES: C=CC(=O)N(CCn1cc(-c2ccc(N)c(O)c2)c2c(N)ncnc21)c1ccccc1] | 2.141 |
| 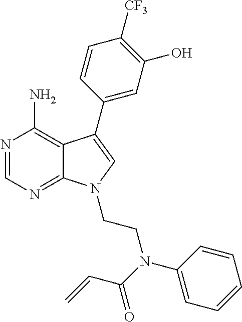 [SMILES: C=CC(=O)N(CCn1cc(-c2ccc(C(F)(F)F)c(O)c2)c2c(N)ncnc21)c1ccccc1] | 2.142 |
| 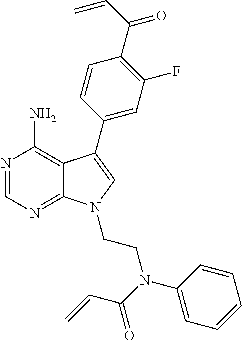 [SMILES: C=CC(=O)c1ccc(-c2cn(CCN(C(=O)C=C)c3ccccc3)c3ncnc(N)c23)cc1F] | 2.143 |
| 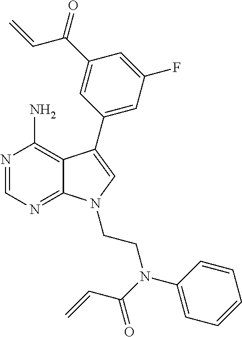 [SMILES: C=CC(=O)c1cc(F)cc(-c2cn(CCN(C(=O)C=C)c3ccccc3)c3ncnc(N)c23)c1] | 2.144 |
| 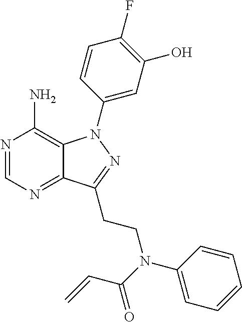 [SMILES: C=CC(=O)N(CCc1nn(-c2ccc(F)c(O)c2)c2c(N)ncnc12)c1ccccc1] | 2.145 |
| 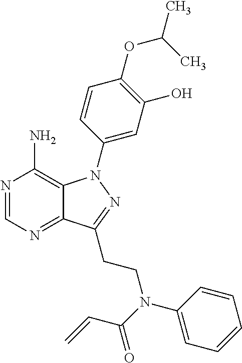 [SMILES: C=CC(=O)N(CCc1nn(-c2ccc(OC(C)C)c(O)c2)c2c(N)ncnc12)c1ccccc1] | 2.146 |
| 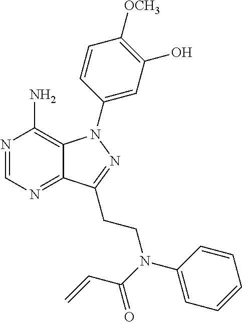 [SMILES: C=CC(=O)N(CCc1nn(-c2ccc(OC)c(O)c2)c2c(N)ncnc12)c1ccccc1] | 2.147 |
| 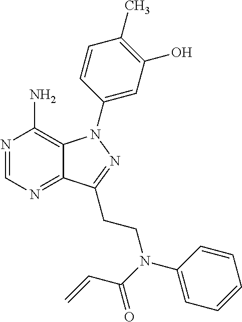 [SMILES: C=CC(=O)N(CCc1nn(-c2ccc(C)c(O)c2)c2c(N)ncnc12)c1ccccc1] | 2.148 |
| 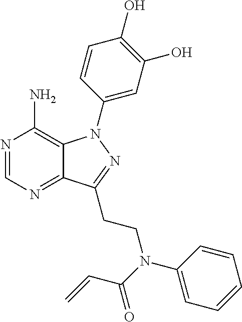 [SMILES: C=CC(=O)N(CCc1nn(-c2ccc(O)c(O)c2)c2c(N)ncnc12)c1ccccc1] | 2.149 |
| 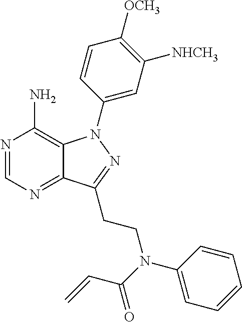 [SMILES: C=CC(=O)N(CCc1nn(-c2ccc(OC)c(NC)c2)c2c(N)ncnc12)c1ccccc1] | 2.150 |
| 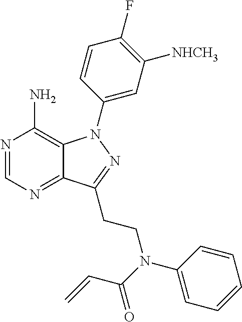 [SMILES: C=CC(=O)N(CCc1nn(-c2ccc(F)c(NC)c2)c2c(N)ncnc12)c1ccccc1] | 2.151 |
| 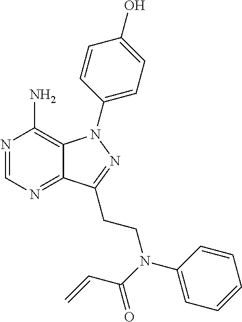 [SMILES: C=CC(=O)N(CCc1nn(-c2ccc(O)cc2)c2c(N)ncnc12)c1ccccc1] | 2.152 |
| 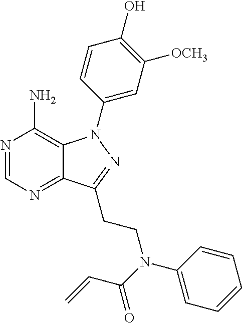 [SMILES: C=CC(=O)N(CCc1nn(-c2ccc(O)c(OC)c2)c2c(N)ncnc12)c1ccccc1] | 2.153 |
| 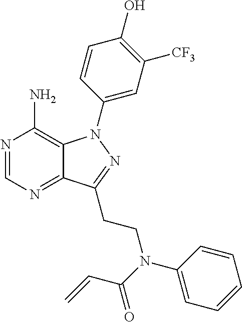 [SMILES: C=CC(=O)N(CCc1nn(-c2ccc(O)c(C(F)(F)F)c2)c2c(N)ncnc12)c1ccccc1] | 2.154 |
| 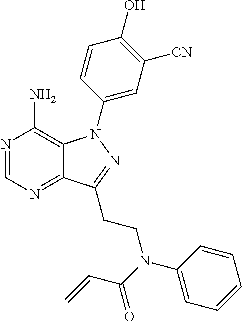 [SMILES: C=CC(=O)N(CCc1nn(-c2ccc(O)c(C#N)c2)c2c(N)ncnc12)c1ccccc1] | 2.155 |
| 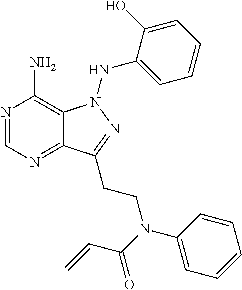 [SMILES: C=CC(=O)N(CCc1nn(Nc2ccccc2O)c2c(N)ncnc12)c1ccccc1] | 2.156 |
| 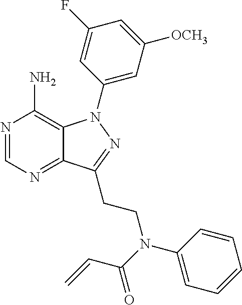 [SMILES: C=CC(=O)N(CCc1nn(-c2cc(F)cc(OC)c2)c2c(N)ncnc12)c1ccccc1] | 2.157 |
| 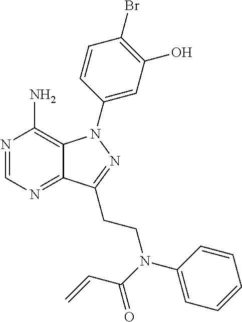 [SMILES: C=CC(=O)N(CCc1nn(-c2ccc(Br)c(O)c2)c2c(N)ncnc12)c1ccccc1] | 2.158 |
| 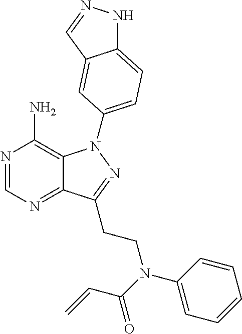 [SMILES: C=CC(=O)N(CCc1nn(-c2ccc3[nH]ncc3c2)c2c(N)ncnc12)c1ccccc1] | 2.159 |
| 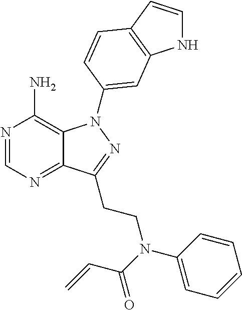 [SMILES: C=CC(=O)N(CCc1nn(-c2ccc3cc[nH]c3c2)c2c(N)ncnc12)c1ccccc1] | 2.160 |
| 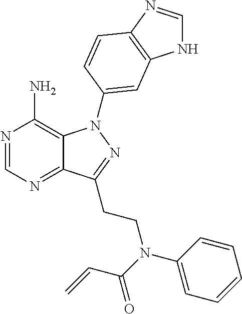 [SMILES: C=CC(=O)N(CCc1nn(-c2ccc3nc[nH]c3c2)c2c(N)ncnc12)c1ccccc1] | 2.161 |
| 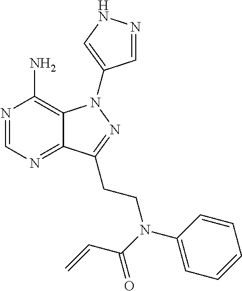 [SMILES: C=CC(=O)N(CCc1nn(-c2cn[nH]c2)c2c(N)ncnc12)c1ccccc1] | 2.162 |
| 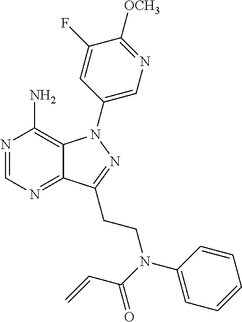 [SMILES: C=CC(=O)N(CCc1nn(-c2cnc(OC)c(F)c2)c2c(N)ncnc12)c1ccccc1] | 2.163 |
| 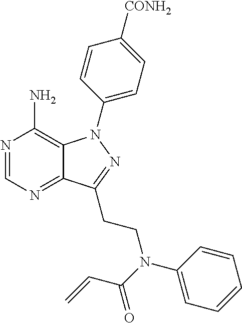 [SMILES: C=CC(=O)N(CCc1nn(-c2ccc(C(N)=O)cc2)c2c(N)ncnc12)c1ccccc1] | 2.164 |
| 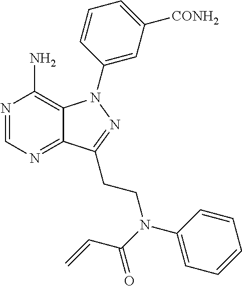 [SMILES: C=CC(=O)N(CCc1nn(-c2cccc(C(N)=O)c2)c2c(N)ncnc12)c1ccccc1] | 2.165 |
| 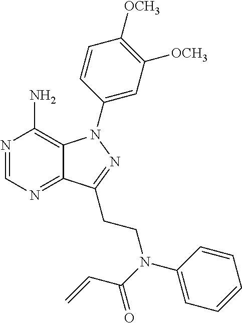 [SMILES: C=CC(=O)N(CCc1nn(-c2ccc(OC)c(OC)c2)c2c(N)ncnc12)c1ccccc1] | 2.166 |
| 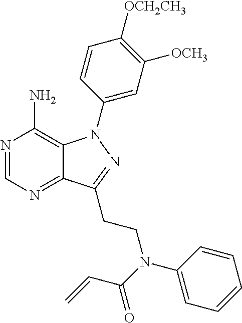 [SMILES: C=CC(=O)N(CCc1nn(-c2ccc(OCC)c(OC)c2)c2c(N)ncnc12)c1ccccc1] | 2.167 |
| 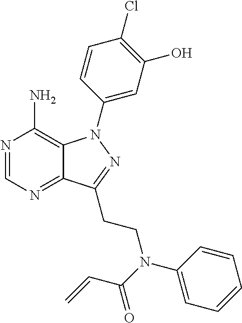 [SMILES: C=CC(=O)N(CCc1nn(-c2ccc(Cl)c(O)c2)c2c(N)ncnc12)c1ccccc1] | 2.168 |
| 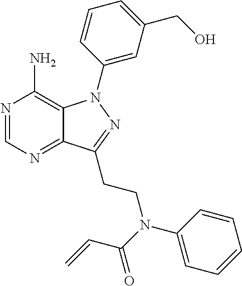 [SMILES: C=CC(=O)N(CCc1nn(-c2cccc(CO)c2)c2c(N)ncnc12)c1ccccc1] | 2.169 |
| 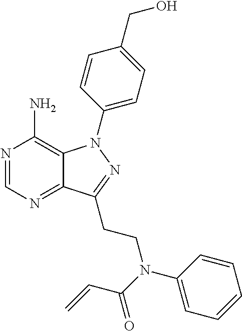 [SMILES: C=CC(=O)N(CCc1nn(-c2ccc(CO)cc2)c2c(N)ncnc12)c1ccccc1] | 2.170 |
| 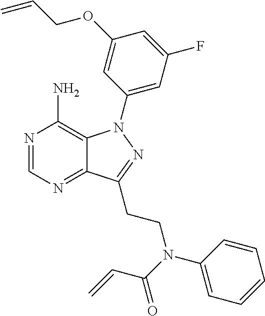 [SMILES: C=CCOc1cc(F)cc(-n2nc(CCN(C(=O)C=C)c3ccccc3)c3ncnc(N)c32)c1] | 2.171 |
| 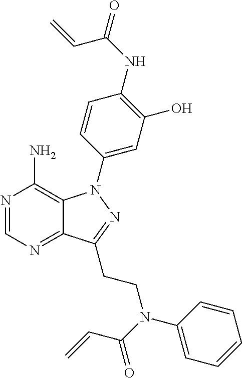 [SMILES: C=CC(=O)Nc1ccc(-n2nc(CCN(C(=O)C=C)c3ccccc3)c3ncnc(N)c32)cc1O] | 2.172 |
| 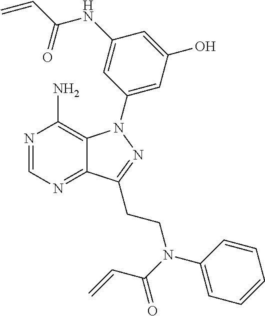 [SMILES: C=CC(=O)Nc1cc(O)cc(-n2nc(CCN(C(=O)C=C)c3ccccc3)c3ncnc(N)c32)c1] | 2.173 |
| 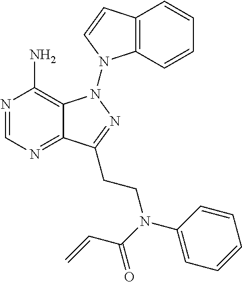 [SMILES: C=CC(=O)N(CCc1nn(-n2ccc3ccccc32)c2c(N)ncnc12)c1ccccc1] | 2.174 |
| 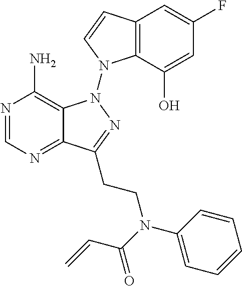 [SMILES: C=CC(=O)N(CCc1nn(-n2ccc3cc(F)cc(O)c32)c2c(N)ncnc12)c1ccccc1] | 2.175 |
| 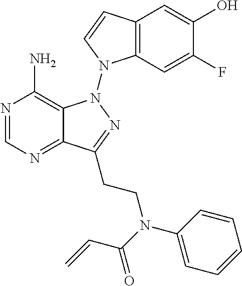 [SMILES: C=CC(=O)N(CCc1nn(-n2ccc3cc(O)c(F)cc32)c2c(N)ncnc12)c1ccccc1] | 2.176 |
| 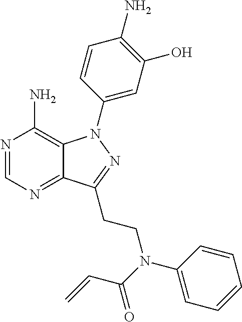 [SMILES: C=CC(=O)N(CCc1nn(-c2ccc(N)c(O)c2)c2c(N)ncnc12)c1ccccc1] | 2.177 |
| 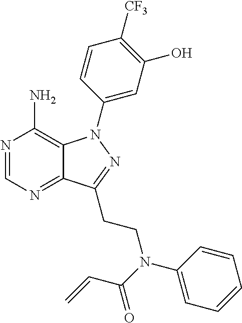 [SMILES: C=CC(=O)N(CCc1nn(-c2ccc(C(F)(F)F)c(O)c2)c2c(N)ncnc12)c1ccccc1] | 2.178 |
| 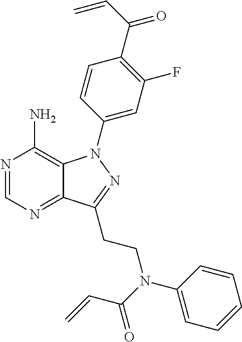 [SMILES: C=CC(=O)c1ccc(-n2nc(CCN(C(=O)C=C)c3ccccc3)c3ncnc(N)c32)cc1F] | 2.179 |
| 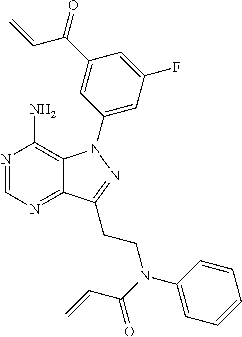 [SMILES: C=CC(=O)c1cc(F)cc(-n2nc(CCN(C(=O)C=C)c3ccccc3)c3ncnc(N)c32)c1] | 2.180 |
| 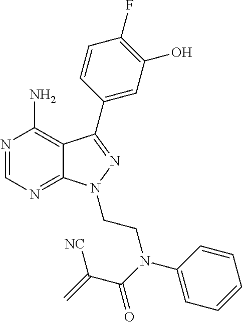 [SMILES: C=C(C#N)C(=O)N(CCn1nc(-c2ccc(F)c(O)c2)c2c(N)ncnc21)c1ccccc1] | 2.181 |
| 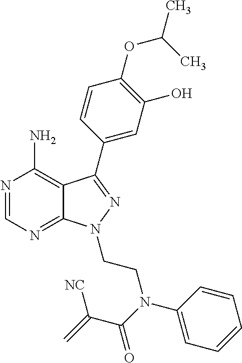 [SMILES: C=C(C#N)C(=O)N(CCn1nc(-c2ccc(OC(C)C)c(O)c2)c2c(N)ncnc21)c1ccccc1] | 2.182 |
| 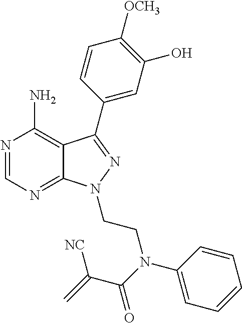 [SMILES: C=C(C#N)C(=O)N(CCn1nc(-c2ccc(OC)c(O)c2)c2c(N)ncnc21)c1ccccc1] | 2.183 |
| 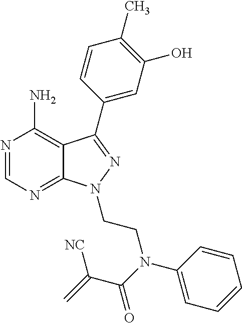 [SMILES: C=C(C#N)C(=O)N(CCn1nc(-c2ccc(C)c(O)c2)c2c(N)ncnc21)c1ccccc1] | 2.184 |
| 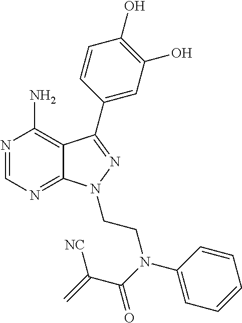 [SMILES: C=C(C#N)C(=O)N(CCn1nc(-c2ccc(O)c(O)c2)c2c(N)ncnc21)c1ccccc1] | 2.185 |
| 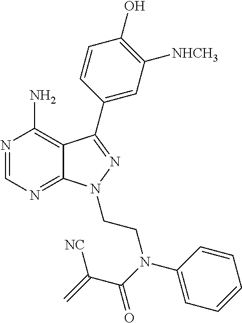 [SMILES: C=C(C#N)C(=O)N(CCn1nc(-c2ccc(O)c(NC)c2)c2c(N)ncnc21)c1ccccc1] | 2.186 |
| 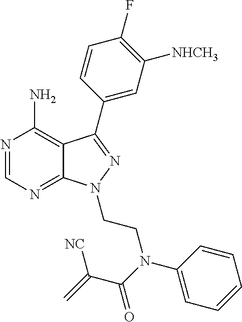 [SMILES: C=C(C#N)C(=O)N(CCn1nc(-c2ccc(F)c(NC)c2)c2c(N)ncnc21)c1ccccc1] | 2.187 |
| 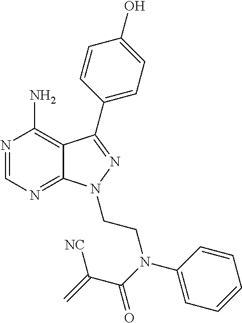 [SMILES: C=C(C#N)C(=O)N(CCn1nc(-c2ccc(O)cc2)c2c(N)ncnc21)c1ccccc1] | 2.188 |
| 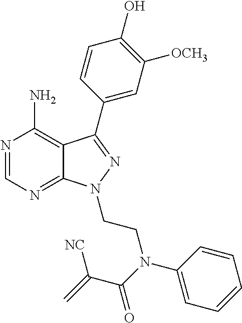 [SMILES: C=C(C#N)C(=O)N(CCn1nc(-c2ccc(O)c(OC)c2)c2c(N)ncnc21)c1ccccc1] | 2.189 |
| 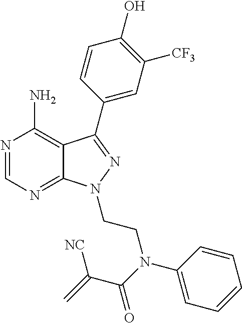 [SMILES: C=C(C#N)C(=O)N(CCn1nc(-c2ccc(O)c(C(F)(F)F)c2)c2c(N)ncnc21)c1ccccc1] | 2.190 |
| 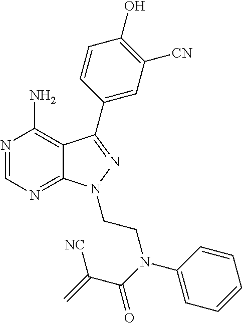 [SMILES: C=C(C#N)C(=O)N(CCn1nc(-c2ccc(O)c(C#N)c2)c2c(N)ncnc21)c1ccccc1] | 2.191 |
| 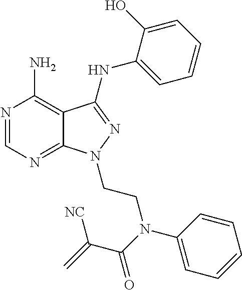 [SMILES: C=C(C#N)C(=O)N(CCn1nc(Nc2ccccc2O)c2c(N)ncnc21)c1ccccc1] | 2.192 |
| 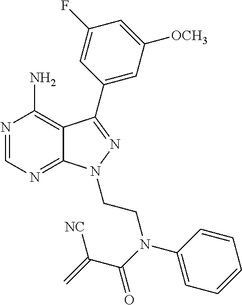 [SMILES: C=C(C#N)C(=O)N(CCn1nc(-c2cc(F)cc(OC)c2)c2c(N)ncnc21)c1ccccc1] | 2.193 |
| 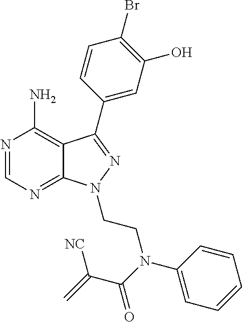 [SMILES: C=C(C#N)C(=O)N(CCn1nc(-c2ccc(Br)c(O)c2)c2c(N)ncnc21)c1ccccc1] | 2.194 |
| 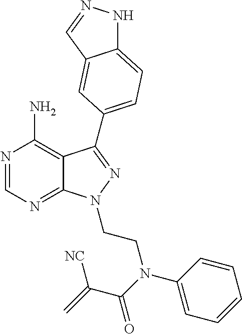 [SMILES: C=C(C#N)C(=O)N(CCn1nc(-c2ccc3[nH]ncc3c2)c2c(N)ncnc21)c1ccccc1] | 2.195 |
| 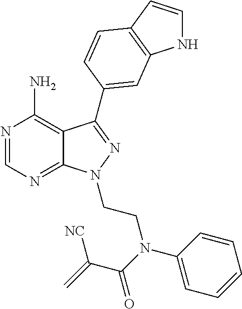 [SMILES: C=C(C#N)C(=O)N(CCn1nc(-c2ccc3cc[nH]c3c2)c2c(N)ncnc21)c1ccccc1] | 2.196 |
| 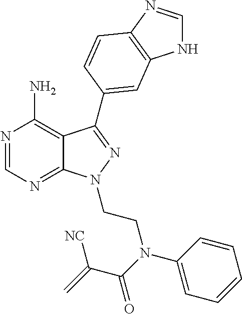 [SMILES: C=C(C#N)C(=O)N(CCn1nc(-c2ccc3nc[nH]c3c2)c2c(N)ncnc21)c1ccccc1] | 2.197 |
| 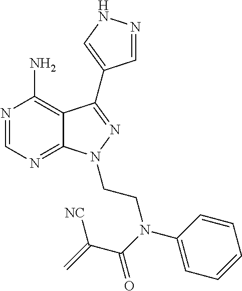 [SMILES: C=C(C#N)C(=O)N(CCn1nc(-c2cn[nH]c2)c2c(N)ncnc21)c1ccccc1] | 2.198 |
| 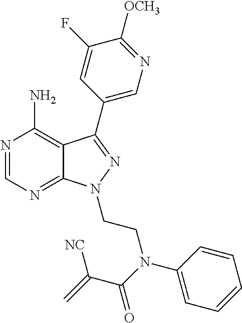 [SMILES: C=C(C#N)C(=O)N(CCn1nc(-c2cnc(OC)c(F)c2)c2c(N)ncnc21)c1ccccc1] | 2.199 |
| 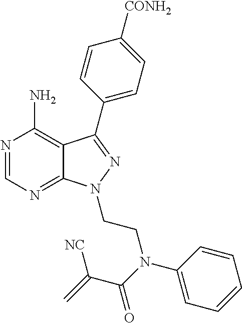 [SMILES: C=C(C#N)C(=O)N(CCn1nc(-c2ccc(C(N)=O)cc2)c2c(N)ncnc21)c1ccccc1] | 2.200 |
| 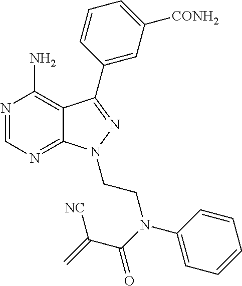 [SMILES: C=C(C#N)C(=O)N(CCn1nc(-c2cccc(C(N)=O)c2)c2c(N)ncnc21)c1ccccc1] | 2.201 |
| 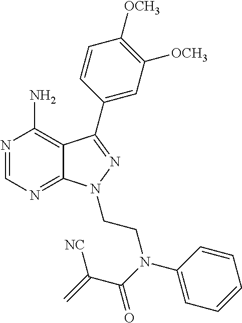 [SMILES: C=C(C#N)C(=O)N(CCn1nc(-c2ccc(OC)c(OC)c2)c2c(N)ncnc21)c1ccccc1] | 2.202 |
| 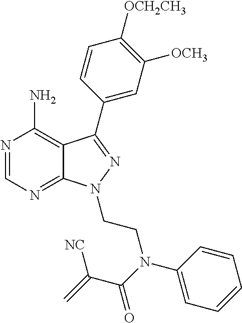 [SMILES: C=C(C#N)C(=O)N(CCn1nc(-c2ccc(OCC)c(OC)c2)c2c(N)ncnc21)c1ccccc1] | 2.203 |
| 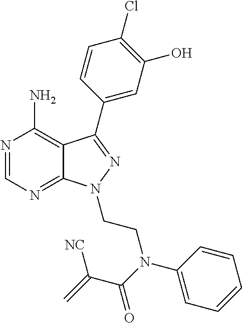 [SMILES: C=C(C#N)C(=O)N(CCn1nc(-c2ccc(Cl)c(O)c2)c2c(N)ncnc21)c1ccccc1] | 2.204 |
| 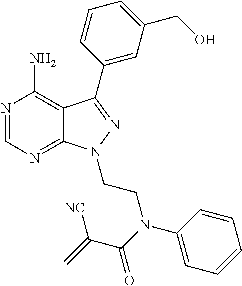 [SMILES: C=C(C#N)C(=O)N(CCn1nc(-c2cccc(CO)c2)c2c(N)ncnc21)c1ccccc1] | 2.205 |
| 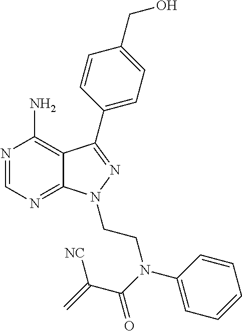 [SMILES: C=C(C#N)C(=O)N(CCn1nc(-c2ccc(CO)cc2)c2c(N)ncnc21)c1ccccc1] | 2.206 |
| 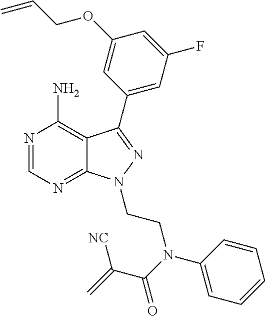 [SMILES: C=CCOc1cc(F)cc(-c2nn(CCN(C(=O)C(=C)C#N)c3ccccc3)c3ncnc(N)c23)c1] | 2.207 |
| 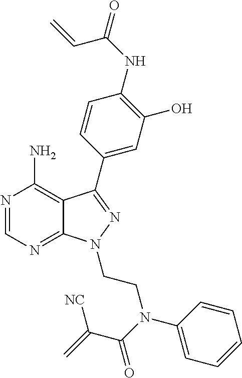 [SMILES: C=CC(=O)Nc1ccc(-c2nn(CCN(C(=O)C(=C)C#N)c3ccccc3)c3ncnc(N)c23)cc1O] | 2.208 |
| 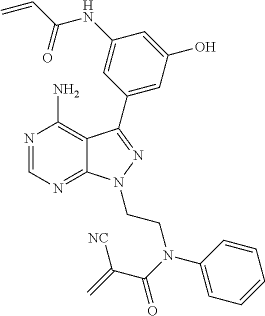 [SMILES: C=CC(=O)Nc1cc(O)cc(-c2nn(CCN(C(=O)C(=C)C#N)c3ccccc3)c3ncnc(N)c23)c1] | 2.209 |
| 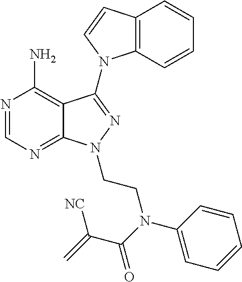 [SMILES: C=C(C#N)C(=O)N(CCn1nc(-n2ccc3ccccc32)c2c(N)ncnc21)c1ccccc1] | 2.210 |
| 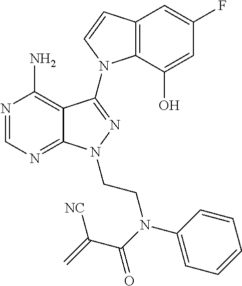 [SMILES: C=C(C#N)C(=O)N(CCn1nc(-n2ccc3cc(F)cc(O)c32)c2c(N)ncnc21)c1ccccc1] | 2.211 |
| 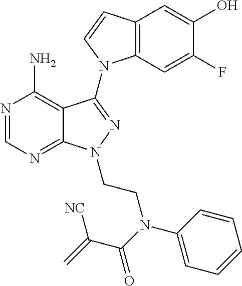 [SMILES: C=C(C#N)C(=O)N(CCn1nc(-n2ccc3cc(O)c(F)cc32)c2c(N)ncnc21)c1ccccc1] | 2.212 |
| 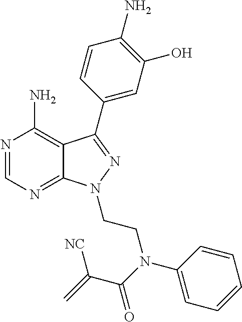 [SMILES: C=C(C#N)C(=O)N(CCn1nc(-c2ccc(N)c(O)c2)c2c(N)ncnc21)c1ccccc1] | 2.213 |
| 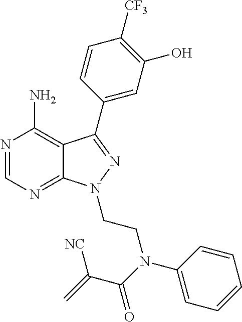 [SMILES: C=C(C#N)C(=O)N(CCn1nc(-c2ccc(C(F)(F)F)c(O)c2)c2c(N)ncnc21)c1ccccc1] | 2.214 |
| 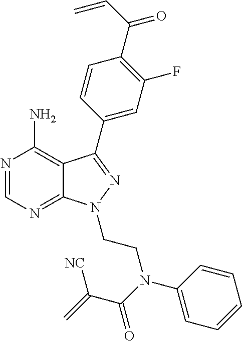 [SMILES: C=CC(=O)c1ccc(-c2nn(CCN(C(=O)C(=C)C#N)c3ccccc3)c3ncnc(N)c23)cc1F] | 2.215 |
| 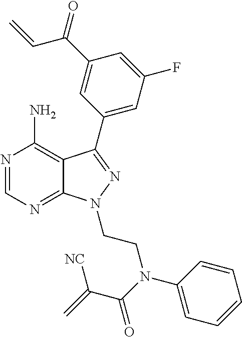 [SMILES: C=CC(=O)c1cc(F)cc(-c2nn(CCN(C(=O)C(=C)C#N)c3ccccc3)c3ncnc(N)c23)c1] | 2.216 |
| 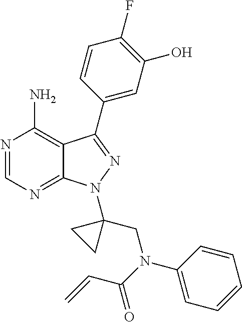 [SMILES: C=CC(=O)N(CC1(n2nc(-c3ccc(F)c(O)c3)c3c(N)ncnc32)CC1)c1ccccc1] | 2.217 |
| 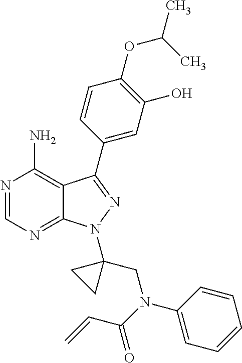 [SMILES: C=CC(=O)N(CC1(n2nc(-c3ccc(OC(C)C)c(O)c3)c3c(N)ncnc32)CC1)c1ccccc1] | 2.218 |
| 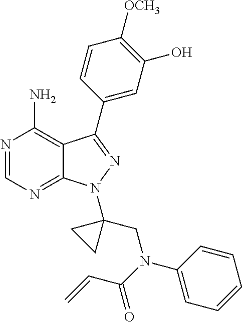 [SMILES: C=CC(=O)N(CC1(n2nc(-c3ccc(OC)c(O)c3)c3c(N)ncnc32)CC1)c1ccccc1] | 2.219 |
| 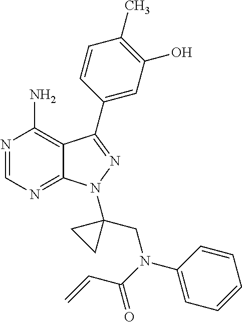 [SMILES: C=CC(=O)N(CC1(n2nc(-c3ccc(C)c(O)c3)c3c(N)ncnc32)CC1)c1ccccc1] | 2.220 |
| 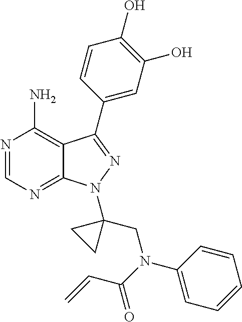 [SMILES: C=CC(=O)N(CC1(n2nc(-c3ccc(O)c(O)c3)c3c(N)ncnc32)CC1)c1ccccc1] | 2.221 |
| 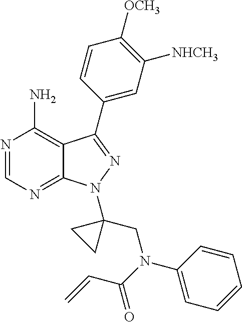 [SMILES: C=CC(=O)N(CC1(n2nc(-c3ccc(OC)c(NC)c3)c3c(N)ncnc32)CC1)c1ccccc1] | 2.222 |
| 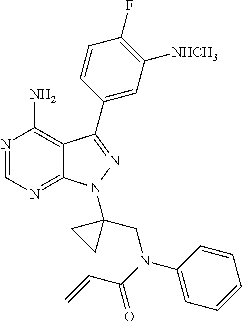 [SMILES: C=CC(=O)N(CC1(n2nc(-c3ccc(F)c(NC)c3)c3c(N)ncnc32)CC1)c1ccccc1] | 2.223 |
| 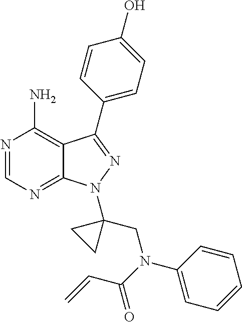 [SMILES: C=CC(=O)N(CC1(n2nc(-c3ccc(O)cc3)c3c(N)ncnc32)CC1)c1ccccc1] | 2.224 |
| 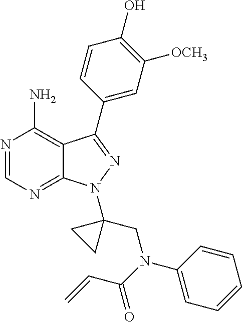 [SMILES: C=CC(=O)N(CC1(n2nc(-c3ccc(O)c(OC)c3)c3c(N)ncnc32)CC1)c1ccccc1] | 2.225 |
| 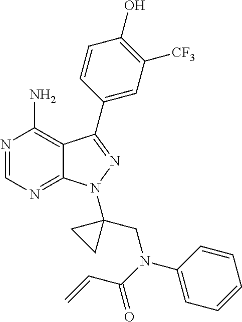 [SMILES: C=CC(=O)N(CC1(n2nc(-c3ccc(O)c(C(F)(F)F)c3)c3c(N)ncnc32)CC1)c1ccccc1] | 2.226 |
| 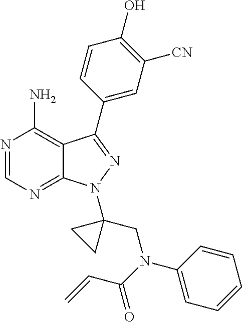 [SMILES: C=CC(=O)N(CC1(n2nc(-c3ccc(O)c(C#N)c3)c3c(N)ncnc32)CC1)c1ccccc1] | 2.227 |
| 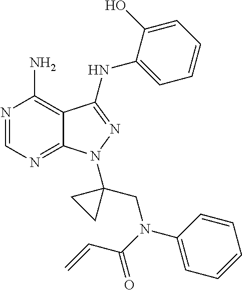 [SMILES: C=CC(=O)N(CC1(n2nc(Nc3ccccc3O)c3c(N)ncnc32)CC1)c1ccccc1] | 2.228 |
| 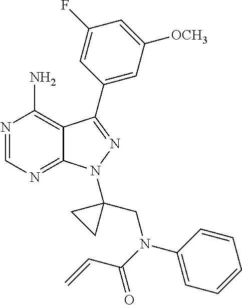 [SMILES: C=CC(=O)N(CC1(n2nc(-c3cc(F)cc(OC)c3)c3c(N)ncnc32)CC1)c1ccccc1] | 2.229 |
| 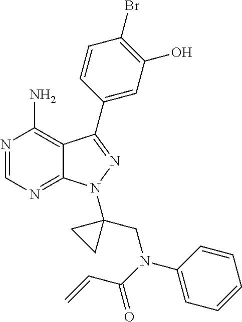 [SMILES: C=CC(=O)N(CC1(n2nc(-c3ccc(Br)c(O)c3)c3c(N)ncnc32)CC1)c1ccccc1] | 2.230 |
| 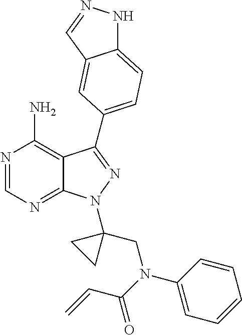 [SMILES: C=CC(=O)N(CC1(n2nc(-c3ccc4[nH]ncc4c3)c3c(N)ncnc32)CC1)c1ccccc1] | 2.231 |
| 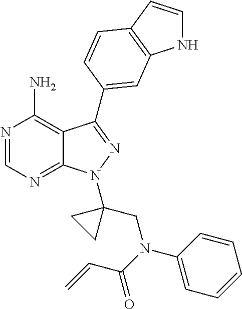 [SMILES: C=CC(=O)N(CC1(n2nc(-c3ccc4cc[nH]c4c3)c3c(N)ncnc32)CC1)c1ccccc1] | 2.232 |
| 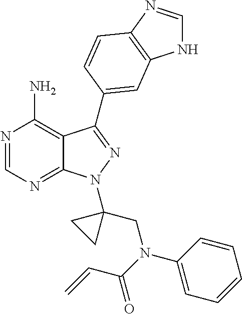 [SMILES: C=CC(=O)N(CC1(n2nc(-c3ccc4nc[nH]c4c3)c3c(N)ncnc32)CC1)c1ccccc1] | 2.233 |
| 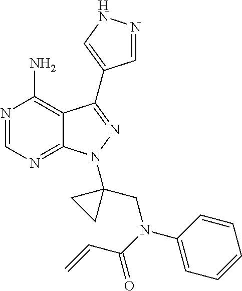 [SMILES: C=CC(=O)N(CC1(n2nc(-c3cn[nH]c3)c3c(N)ncnc32)CC1)c1ccccc1] | 2.234 |
| 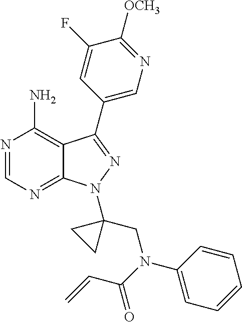 [SMILES: C=CC(=O)N(CC1(n2nc(-c3cnc(OC)c(F)c3)c3c(N)ncnc32)CC1)c1ccccc1] | 2.235 |
| 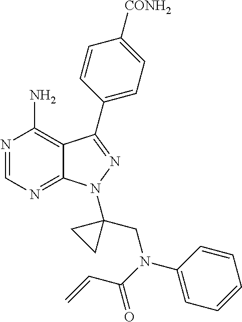 [SMILES: C=CC(=O)N(CC1(n2nc(-c3ccc(C(N)=O)cc3)c3c(N)ncnc32)CC1)c1ccccc1] | 2.236 |
| 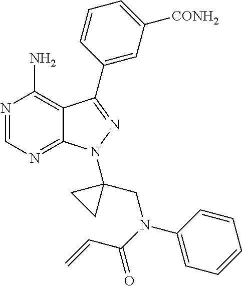 [SMILES: C=CC(=O)N(CC1(n2nc(-c3cccc(C(N)=O)c3)c3c(N)ncnc32)CC1)c1ccccc1] | 2.237 |
| 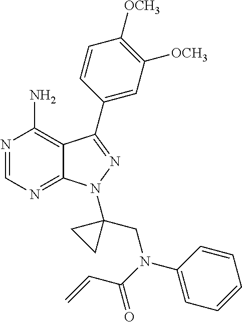 [SMILES: C=CC(=O)N(CC1(n2nc(-c3ccc(OC)c(OC)c3)c3c(N)ncnc32)CC1)c1ccccc1] | 2.238 |
| 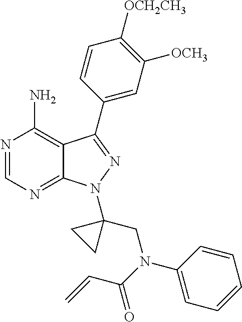 [SMILES: C=CC(=O)N(CC1(n2nc(-c3ccc(OCC)c(OC)c3)c3c(N)ncnc32)CC1)c1ccccc1] | 2.239 |
| 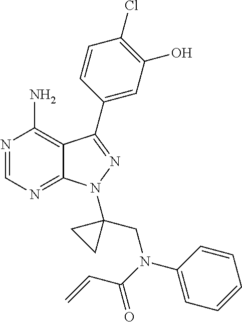 [SMILES: C=CC(=O)N(CC1(n2nc(-c3ccc(Cl)c(O)c3)c3c(N)ncnc32)CC1)c1ccccc1] | 2.240 |
| 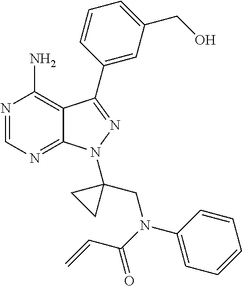 [SMILES: C=CC(=O)N(CC1(n2nc(-c3cccc(CO)c3)c3c(N)ncnc32)CC1)c1ccccc1] | 2.241 |
| 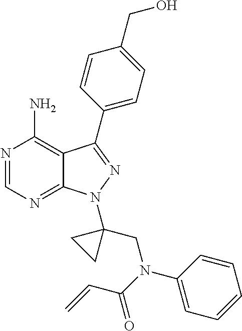 [SMILES: C=CC(=O)N(CC1(n2nc(-c3ccc(CO)cc3)c3c(N)ncnc32)CC1)c1ccccc1] | 2.242 |
| 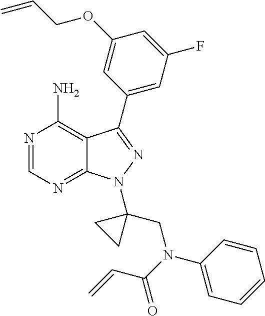 [SMILES: C=CCOc1cc(F)cc(-c2nn(C3(CN(C(=O)C=C)c4ccccc4)CC3)c3ncnc(N)c23)c1] | 2.243 |
| 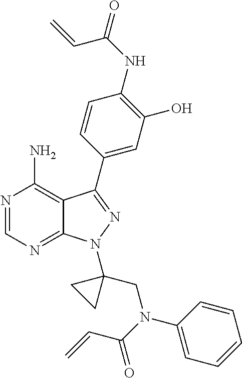 [SMILES: C=CC(=O)Nc1ccc(-c2nn(C3(CN(C(=O)C=C)c4ccccc4)CC3)c3ncnc(N)c23)cc1O] | 2.244 |
| 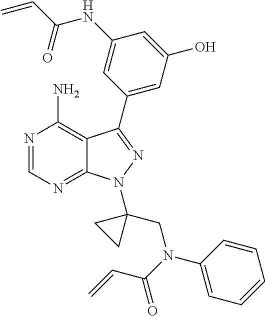 [SMILES: C=CC(=O)Nc1cc(O)cc(-c2nn(C3(CN(C(=O)C=C)c4ccccc4)CC3)c3ncnc(N)c23)c1] | 2.245 |
| 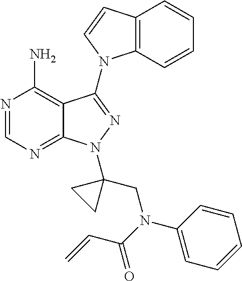 [SMILES: C=CC(=O)N(CC1(n2nc(-n3ccc4ccccc43)c3c(N)ncnc32)CC1)c1ccccc1] | 2.246 |
| 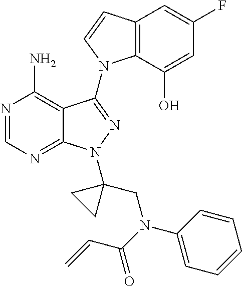 [SMILES: C=CC(=O)N(CC1(n2nc(-n3ccc4cc(F)cc(O)c43)c3c(N)ncnc32)CC1)c1ccccc1] | 2.247 |
| 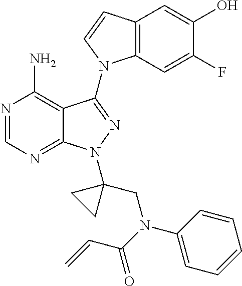 [SMILES: C=CC(=O)N(CC1(n2nc(-n3ccc4cc(O)c(F)cc43)c3c(N)ncnc32)CC1)c1ccccc1] | 2.248 |
| 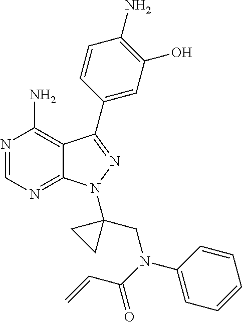 [SMILES: C=CC(=O)N(CC1(n2nc(-c3ccc(N)c(O)c3)c3c(N)ncnc32)CC1)c1ccccc1] | 2.249 |
| 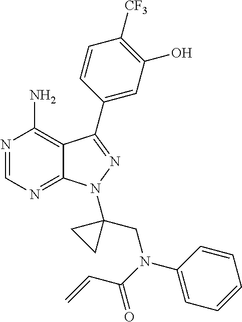 [SMILES: C=CC(=O)N(CC1(n2nc(-c3ccc(C(F)(F)F)c(O)c3)c3c(N)ncnc32)CC1)c1ccccc1] | 2.250 |
| 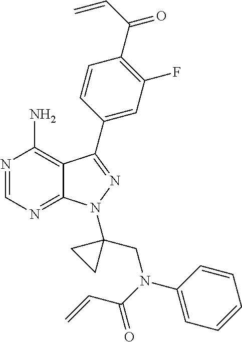 [SMILES: C=CC(=O)c1ccc(-c2nn(C3(CN(C(=O)C=C)c4ccccc4)CC3)c3ncnc(N)c23)cc1F] | 2.251 |
| 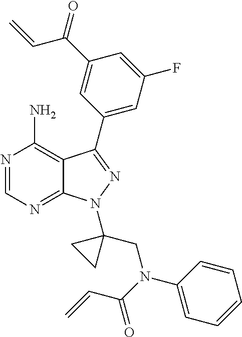 [SMILES: C=CC(=O)c1cc(F)cc(-c2nn(C3(CN(C(=O)C=C)c4ccccc4)CC3)c3ncnc(N)c23)c1] | 2.252 |
| 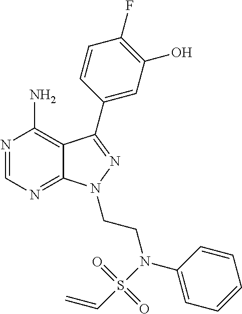 [SMILES: C=CS(=O)(=O)N(CCn1nc(-c2ccc(F)c(O)c2)c2c(N)ncnc21)c1ccccc1] | 2.253 |
| 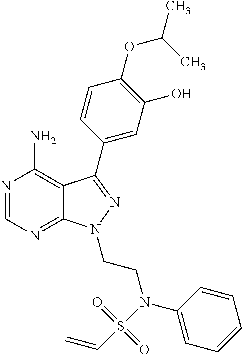 [SMILES: C=CS(=O)(=O)N(CCn1nc(-c2ccc(OC(C)C)c(O)c2)c2c(N)ncnc21)c1ccccc1] | 2.254 |
| 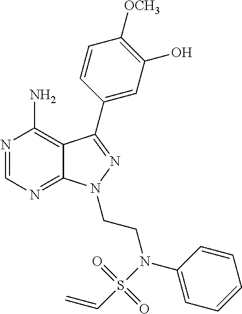 [SMILES: C=CS(=O)(=O)N(CCn1nc(-c2ccc(OC)c(O)c2)c2c(N)ncnc21)c1ccccc1] | 2.255 |
| 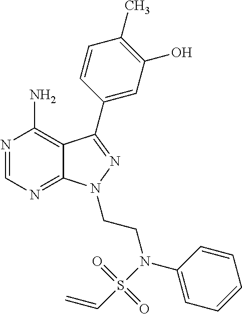 [SMILES: C=CS(=O)(=O)N(CCn1nc(-c2ccc(C)c(O)c2)c2c(N)ncnc21)c1ccccc1] | 2.256 |
| 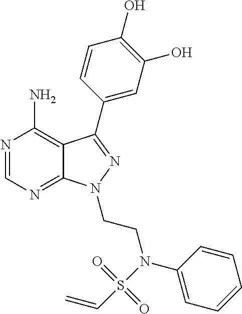 [SMILES: C=CS(=O)(=O)N(CCn1nc(-c2ccc(O)c(O)c2)c2c(N)ncnc21)c1ccccc1] | 2.257 |
| 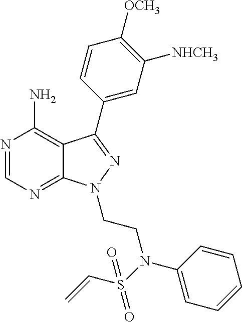 [SMILES: C=CS(=O)(=O)N(CCn1nc(-c2ccc(OC)c(NC)c2)c2c(N)ncnc21)c1ccccc1] | 2.258 |
| 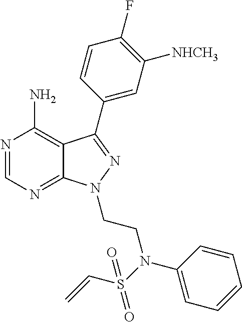 [SMILES: C=CS(=O)(=O)N(CCn1nc(-c2ccc(F)c(NC)c2)c2c(N)ncnc21)c1ccccc1] | 2.259 |
| 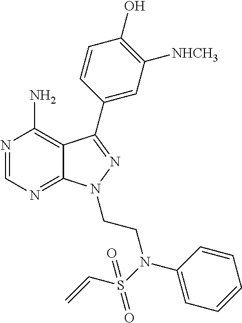 [SMILES: C=CS(=O)(=O)N(CCn1nc(-c2ccc(O)c(NC)c2)c2c(N)ncnc21)c1ccccc1] | 2.260 |
| 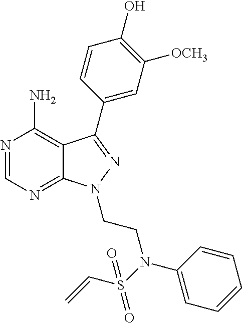 [SMILES: C=CS(=O)(=O)N(CCn1nc(-c2ccc(O)c(OC)c2)c2c(N)ncnc21)c1ccccc1] | 2.261 |
| 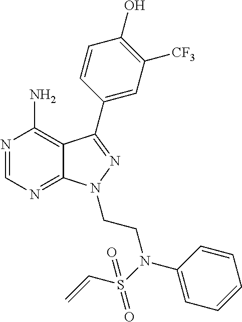 [SMILES: C=CS(=O)(=O)N(CCn1nc(-c2ccc(O)c(C(F)(F)F)c2)c2c(N)ncnc21)c1ccccc1] | 2.262 |
| 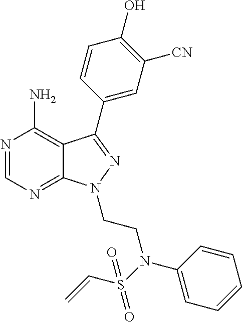 [SMILES: C=CS(=O)(=O)N(CCn1nc(-c2ccc(O)c(C#N)c2)c2c(N)ncnc21)c1ccccc1] | 2.263 |
| 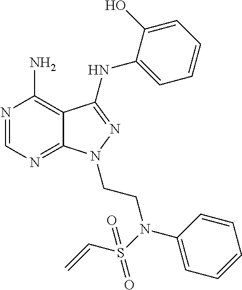 [SMILES: C=CS(=O)(=O)N(CCn1nc(Nc2ccccc2O)c2c(N)ncnc21)c1ccccc1] | 2.264 |
| 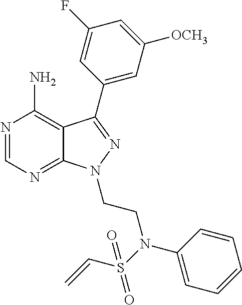 [SMILES: C=CS(=O)(=O)N(CCn1nc(-c2cc(F)cc(OC)c2)c2c(N)ncnc21)c1ccccc1] | 2.265 |
| 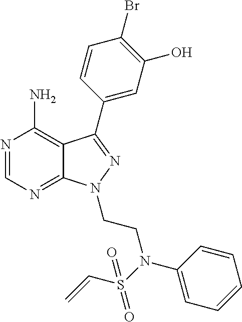 [SMILES: C=CS(=O)(=O)N(CCn1nc(-c2ccc(Br)c(O)c2)c2c(N)ncnc21)c1ccccc1] | 2.266 |
| 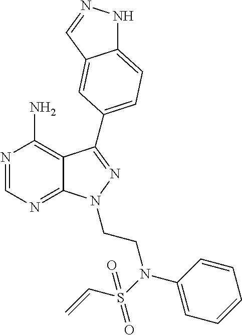 [SMILES: C=CS(=O)(=O)N(CCn1nc(-c2ccc3[nH]ncc3c2)c2c(N)ncnc21)c1ccccc1] | 2.267 |
| 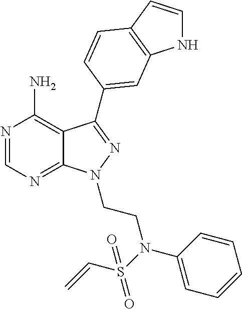 [SMILES: C=CS(=O)(=O)N(CCn1nc(-c2ccc3cc[nH]c3c2)c2c(N)ncnc21)c1ccccc1] | 2.268 |
| 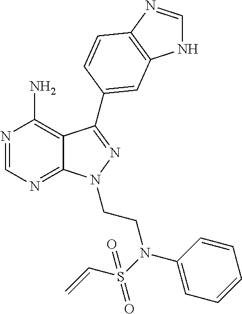 [SMILES: C=CS(=O)(=O)N(CCn1nc(-c2ccc3nc[nH]c3c2)c2c(N)ncnc21)c1ccccc1] | 2.269 |
| 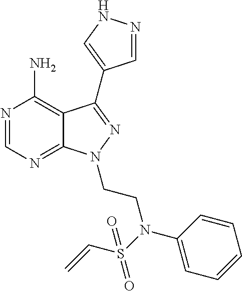 [SMILES: C=CS(=O)(=O)N(CCn1nc(-c2cn[nH]c2)c2c(N)ncnc21)c1ccccc1] | 2.270 |
| 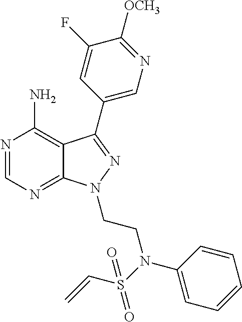 [SMILES: C=CS(=O)(=O)N(CCn1nc(-c2cnc(OC)c(F)c2)c2c(N)ncnc21)c1ccccc1] | 2.271 |
| 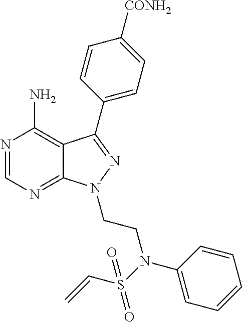 [SMILES: C=CS(=O)(=O)N(CCn1nc(-c2ccc(C(N)=O)cc2)c2c(N)ncnc21)c1ccccc1] | 2.272 |
| 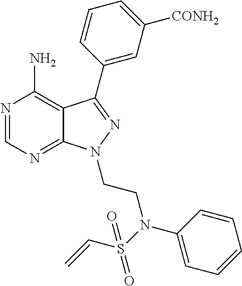 [SMILES: C=CS(=O)(=O)N(CCn1nc(-c2cccc(C(N)=O)c2)c2c(N)ncnc21)c1ccccc1] | 2.273 |
| 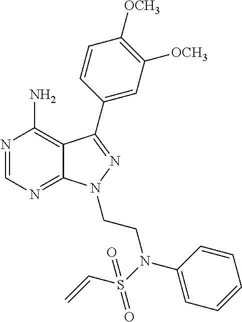 [SMILES: C=CS(=O)(=O)N(CCn1nc(-c2ccc(OC)c(OC)c2)c2c(N)ncnc21)c1ccccc1] | 2.274 |
| 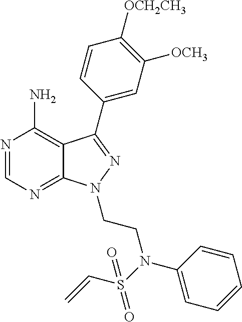 [SMILES: C=CS(=O)(=O)N(CCn1nc(-c2ccc(OCC)c(OC)c2)c2c(N)ncnc21)c1ccccc1] | 2.275 |
| 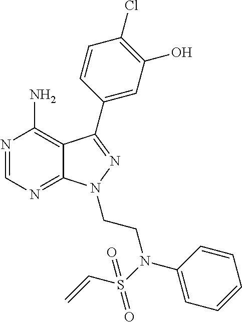 [SMILES: C=CS(=O)(=O)N(CCn1nc(-c2ccc(Cl)c(O)c2)c2c(N)ncnc21)c1ccccc1] | 2.276 |
| 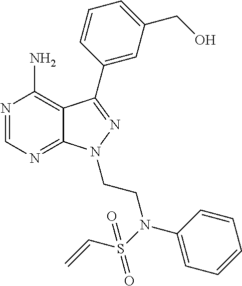 [SMILES: C=CS(=O)(=O)N(CCn1nc(-c2cccc(CO)c2)c2c(N)ncnc21)c1ccccc1] | 2.277 |
| 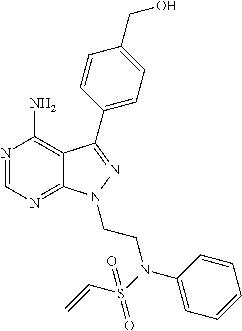 [SMILES: C=CS(=O)(=O)N(CCn1nc(-c2ccc(CO)cc2)c2c(N)ncnc21)c1ccccc1] | 2.278 |
| 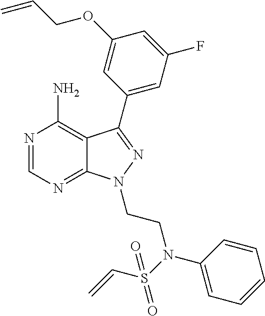 [SMILES: C=CCOc1cc(F)cc(-c2nn(CCN(c3ccccc3)S(=O)(=O)C=C)c3ncnc(N)c23)c1] | 2.279 |
| 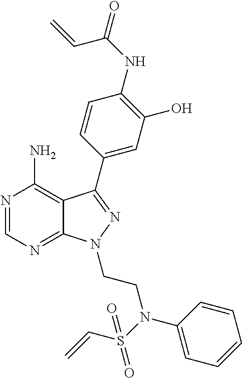 [SMILES: C=CC(=O)Nc1ccc(-c2nn(CCN(c3ccccc3)S(=O)(=O)C=C)c3ncnc(N)c23)cc1O] | 2.280 |
| 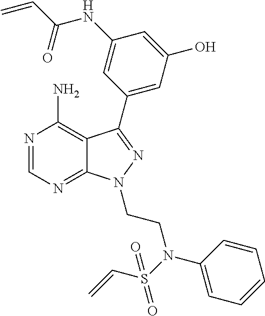 [SMILES: C=CC(=O)Nc1cc(O)cc(-c2nn(CCN(c3ccccc3)S(=O)(=O)C=C)c3ncnc(N)c23)c1] | 2.281 |
| 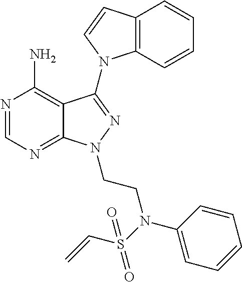 [SMILES: C=CS(=O)(=O)N(CCn1nc(-n2ccc3ccccc32)c2c(N)ncnc21)c1ccccc1] | 2.282 |
| 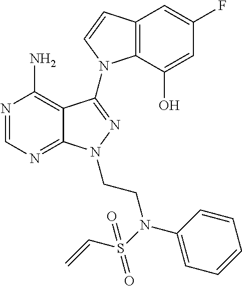 [SMILES: C=CS(=O)(=O)N(CCn1nc(-n2ccc3cc(F)cc(O)c32)c2c(N)ncnc21)c1ccccc1] | 2.283 |
| 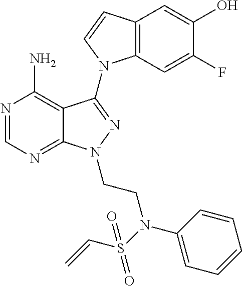 [SMILES: C=CS(=O)(=O)N(CCn1nc(-n2ccc3cc(O)c(F)cc32)c2c(N)ncnc21)c1ccccc1] | 2.284 |
| 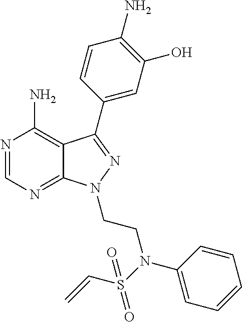 [SMILES: C=CS(=O)(=O)N(CCn1nc(-c2ccc(N)c(O)c2)c2c(N)ncnc21)c1ccccc1] | 2.285 |
| 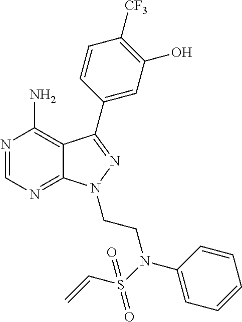 [SMILES: C=CS(=O)(=O)N(CCn1nc(-c2ccc(C(F)(F)F)c(O)c2)c2c(N)ncnc21)c1ccccc1] | 2.286 |
| 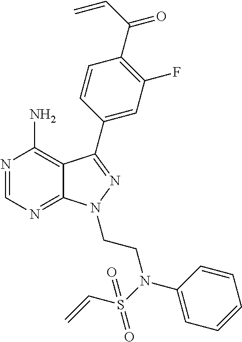 [SMILES: C=CC(=O)c1ccc(-c2nn(CCN(c3ccccc3)S(=O)(=O)C=C)c3ncnc(N)c23)cc1F] | 2.287 |
| 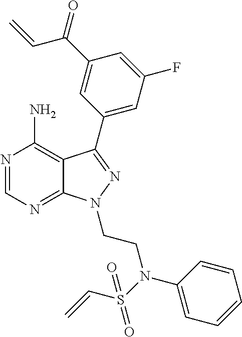 [SMILES: C=CC(=O)c1cc(F)cc(-c2nn(CCN(c3ccccc3)S(=O)(=O)C=C)c3ncnc(N)c23)c1] | 2.288 |
| 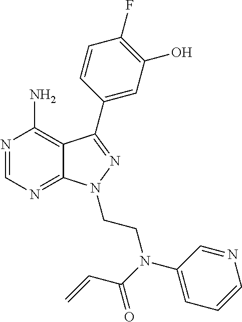 [SMILES: C=CC(=O)N(CCn1nc(-c2ccc(F)c(O)c2)c2c(N)ncnc21)c1cccnc1] | 2.289 |
| 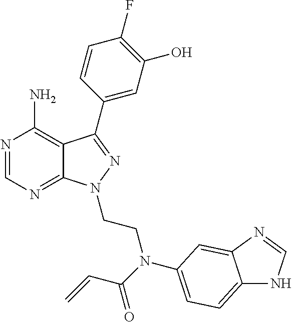 [SMILES: C=CC(=O)N(CCn1nc(-c2ccc(F)c(O)c2)c2c(N)ncnc21)c1ccc2[nH]cnc2c1] | 2.290 |
| 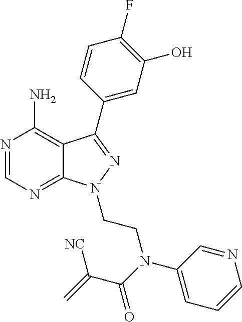 [SMILES: C=C(C#N)C(=O)N(CCn1nc(-c2ccc(F)c(O)c2)c2c(N)ncnc21)c1cccnc1] | 2.291 |
| 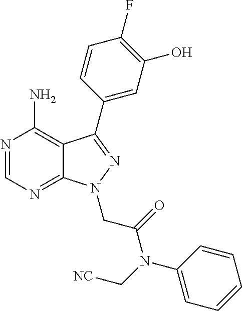 [SMILES: N#CCN(C(=O)Cn1nc(-c2ccc(F)c(O)c2)c2c(N)ncnc21)c1ccccc1] | 2.292 |
| 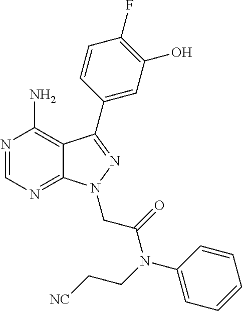 [SMILES: N#CCCN(C(=O)Cn1nc(-c2ccc(F)c(O)c2)c2c(N)ncnc21)c1ccccc1] | 2.293 |
| 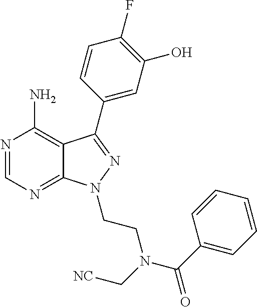 [SMILES: N#CCN(CCn1nc(-c2ccc(F)c(O)c2)c2c(N)ncnc21)C(=O)c1ccccc1] | 2.294 |
| 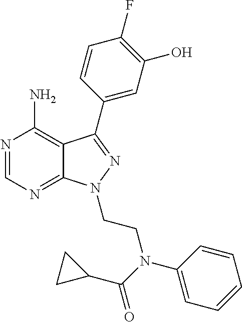 [SMILES: Nc1ncnc2c1c(-c1ccc(F)c(O)c1)nn2CCN(C(=O)C1CC1)c1ccccc1] | 2.295 |
| 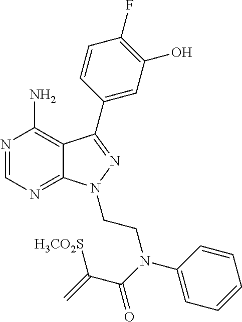 | 2.296 |
| 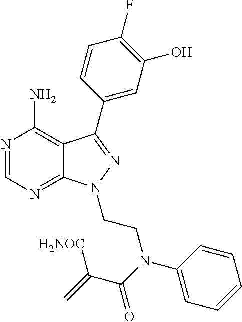 [SMILES: C=C(C(N)=O)C(=O)N(CCn1nc(-c2ccc(F)c(O)c2)c2c(N)ncnc21)c1ccccc1] | 2.297 |

Representative examples of compounds detailed herein, including intermediates and final compounds according to the invention are depicted in the tables below. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the invention embraces all salts and solvates of the compounds depicted here, as well as the non-salt and non-solvate form of the compound, as is well understood by the skilled artisan. In some embodiments, the salts of the compounds of the invention are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted.

The invention also includes any or all of the stereochemical forms, including any enantiomeric or diastereomeric forms of the compounds described. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. Taking compound 1 as an example, a composition of substantially pure compound 1 intends a composition that contains no more than 35% impurity, wherein the impurity denotes a compound other than compound 1 or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other embodiments, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the invention embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

### General Description of Biological Assays

The binding properties of compounds disclosed herein to a panel of kinases, including BTK and PI3Kδ may be determined. Binding properties may be assessed by methods known in the art, such as competitive binding assays, or kinase assays. In one variation, compounds are assessed by the kinase assays detailed herein. Other assays are known to those skilled in the art, such as, for example, those presented and discussed in US2013/0165395A1 (for BTK), and US2012/0053166A1 (for PI3Kδ). Compounds disclosed herein may also be tested in cell-based assays or in in vivo models for further characterization.

### Therapeutic Uses and Methods

Compounds and compositions provided herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

While not being bound by theory, it is believed that the effects of the compounds presented herein may be attributed to the ability of the compounds to inhibit both BTK kinase and PI3Kδ kinase, the result being a reduction or elimination of signaling in the BCR pathway, and reduction in downstream events of that pathway. Compounds that negatively regulate both BTK and PI3Kδ expression or activity can be used as dual BTK-PI3Kδ inhibitors in the methods of the invention. In one aspect, the compounds may have a synergistic effect. In another aspect, the compounds may have an additive effect. In another aspect, the compounds may inhibit just one of the BTK and PI3Kδ kinases. In another aspect, the compounds may inhibit one of the BTK and PI3Kδ kinases and also block the resistance mechanism of the remaining kinase. In another aspect, the compounds may block the resistance mechanisms of both BTK and PI3Kδ kinases.

The compounds of the invention are inhibitors of kinase activity, in particular BTK and PI3Kδ activity. Compounds which are BTK and/or PI3Kδ inhibitors may be used in the treatment of disorders wherein the underlying pathology is (at least in part) attributable to inappropriate BTK and/or PI3Kδ activity. “Inappropriate BTK and/or PI3Kδ activity” refers to any kinase activity that deviates from normal BTK and/or PI3Kδ activity expected in a particular patient, and which may take the form of, for instance, an abnormal increase in activity, or an aberration in the timing and/or control of BTK and/or PI3Kδ activity. Such inappropriate activity may result then, for example, from overexpression or mutation of the protein kinase(s) leading to inappropriate or uncontrolled activation. Accordingly, in another aspect, the invention is directed to methods of treating such disorders. In some aspects, provided herein are methods for treating diseases impacted by a resistance to BTK inhibition. In some aspects, provided herein are methods for treating diseases impacted by a resistance to PI3Kδ inhibition.

In some aspects, provided herein is a method for utilizing compounds of the invention as a monotherapy to treat both BTK-mediated disorders and PI3Kδ-mediated disorders. In some aspects, the monotherapy can overcome resistance to treatment of either a BTK-mediated disorder alone, or a PI3Kδ-mediated disorder alone. In some aspects, the monotherapy can negate the need for combination treatment with a second therapeutic agent previously required to overcome resistance to treatment. In some aspects, provided is a method to treat a subject that is resistant or has developed a resistance to therapeutic agents.

In some aspects, presented herein is a method for suppressing a function of basophils and/or mast cells, and thereby enabling treatment of diseases or disorders characterized by excessive or undesirable basophil and/or mast cell activity. A compound of the invention can be used that inhibits the expression or activity of both BTK and PI3Kδ in the basophils and/or mast cells.

In some aspects, provided herein is a method for suppressing osteoclastogenesis. A compound of the invention can be used that delays the onset and/or development of bone loss arising from osteoclast-associated bone disorders, such as for example bone metastasis, osteoarthritis and rheumatoid arthritis.

Compounds provided herein may be used in a method of delaying the onset and/or development of a disease or condition associated with excessive or undesirable basophil and/or mast cell activity, or with basophil and/or mast cell dysfunction. The compounds provided herein may be used in a method of delaying the onset of a disease or condition that is responsive to a decrease in basophil and/or mast cell activity. The compounds as provided herein may also be used in a method of delaying the onset and/or development of any indications presented below.

In some aspects, compounds presented herein selectively inhibit PI3Kδ over related PI3K isoforms. The advantage of a PI3Kδ selective inhibitor which targets cells mediating inflammation and cancer cells, wherein potential clinical indications include cancer, rheumatoid arthritis, asthma, allergies and COPD, is that treatment is well tolerated, and side effects like hyperinsulinemia are avoided. In some aspects, compounds of the invention provide therapeutic benefits to treating hematologic malignancies without adversely affecting insulin signaling.

In some aspects, presented herein are methods of selectively inhibiting PI3Kδ. In other aspects, presented herein are methods of inhibiting PI3Kβ and/or PI3Kγ.

In some aspects, the compounds presented herein inhibit both Btk kinase and PI3Kδ kinase activity with an in vitro IC₅₀ of less than 10 μM. (e.g., less than 1 μM, less than 0.5 μM, less than 0.4 μM, less than 0.3 μM, less than 0.1 μM, less than 0.08 μM, less than 0.06 μM, less than 0.05 μM, less than 0.04 μM, less than 0.03 μM, less than 0.02 μM, less than 0.01 μM, less than 0.008 μM, less than 0.006 μM, less than 0.005 μM, less than 0.004 μM, less than 0.003 μM, less than 0.002 μM, or less than 0.001 μM. In some aspects, the compounds inhibit both Btk kinase and PI3Kδ kinase activity with an in vitro IC₅₀ of less than 0.1 μM. In some aspects, the compounds inhibit both Btk kinase and PI3Kδ kinase activity with an in vitro IC₅₀ of less than 0.05 μM.

Compounds provided herein, such as the dual BTK and PI3Kδ kinase inhibitors provided herein, are expected to find use in therapy, particularly in disease indications resulting from an inappropriate activation of the BCR pathway, B-cell malignancies, or diseases otherwise benefiting from inhibition of BTK or PI3Kδ activity.

In one aspect, provided herein is a method for treating diseases impacted by one or more of BTK and/or PI3Kδ kinases, in a subject in need thereof by administering to the subject thereof a composition containing a therapeutically effective amount of a compound having a structure presented herein.

In another aspect, the invention includes a method for suppressing a function of basophils and/or mast cells, and thereby enabling treatment of diseases or disorders characterized by excessive or undesirable basophil and/or mast cell activity. According to the method, a compound of the invention can be used that selectively inhibits the expression or activity of BTK or PI3Kδ in the basophils and/or mast cells. Preferably, the method employs an inhibitor in an amount sufficient to inhibit stimulated histamine release by the basophils and/or mast cells.

In some aspects, the subject in need is suffering from an autoimmune disease, a heteroimmune condition, an inflammatory disease, cancer, a thromboembolic disorder, a respiratory disease.

In some aspects, the autoimmune disease includes, but is not limited to, inflammatory bowel disease, arthritis, lupus, inflammatory bowel disease (including Crohn's disease and ulcerative colitis), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behyet's disease, chronic fatigue, dysautonomia, endometriosis, interstitial cystitis, neuromyotonia, scleroderma, vulvodynia, allergic rhinitis, asthma and COPD.

In some aspects, the heteroimmune condition or disease includes, but is not limited to, graft versus host disease, transplantation, transfusion, anaphylaxis, allergy, type I hypersensitivity, allergic conjunctivitis, allergic rhinitis, and atopic dermatitis.

In some aspects, the inflammatory disease includes, but is not limited to, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some aspects, the cancer is associated with abnormal BTK or PI3Kδ activity compared to activity in a subject without cancer. In some aspects, the cancer is a B-cell proliferative disorder, and includes, but is not limited to, diffuse large B cell lymphoma, follicular lymphoma, chronic lymphocytic lymphoma, chronic lymphocytic leukemia (CLL), B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, lymphomatoid granulomatosis, acute lymphocytic leukemia (ALL), acute myeloid leukemia (AML), small lymphocytic lymphoma (SLL), multiple myeloma (MM), non-Hodgkin's lymphoma (NHL), and mantle cell lymphoma (MCL). In some aspects, the cancer is B-cell or T-cell ALL. In some aspects, the cancer is Hodgkin's lymphoma. In some aspects, the cancer is breast, lung, colon, prostate or ovarian cancer. In some aspects, lymphoma is a mature (peripheral) B-cell neoplasm. In specific embodiments, the mature B-cell neoplasm is selected from the group consisting of B-cell chronic lymphocytic leukemia/small lymphocytic lymphoma; B-cell prolymphocytic leukemia; Lymphoplasmacytic lymphoma; Marginal zone lymphoma, such as Splenic marginal zone B-cell lymphoma (+/− villous lymphocytes), Nodal marginal zone lymphoma (+/monocytoid B-cells), and Extranodal marginal zone B-cell lymphoma of mucosa-associated lymphoid tissue (MALT) type; Hairy cell leukemia; Plasma cell myeloma/plasmacytoma; Follicular lymphoma, follicle center; Mantle cell lymphoma; Diffuse large cell B-cell lymphoma (including Mediastinal large B-cell lymphoma, Intravascular large B-cell lymphoma, and Primary effusion lymphoma); and Burkitt's lymphoma/Burkitt's cell leukemia.

In some aspects, the thromboembolic disorder includes, but is not limited to, myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some aspects, the inflammatory disease includes, but is not limited to, asthma, inflammatory bowel disease (including Crohn's disease and ulcerative colitis), appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some aspects, the respiratory disease is asthma. In some aspects, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child onset asthma, adult-onset asthma, cough-variant asthma, occupational asthma, steroid-resistant asthma, and seasonal asthma.

In some aspects, provided herein are methods for treating the diseases presented above, by administering to a subject in need thereof a composition containing a therapeutically effective amount of a compound that forms a covalent bond with one or both of BTK and/or PI3Kδ. In some aspects, the compound forms a covalent bound with the activated form of BTK and/or PI3Kδ. In some aspects, the compound irreversibly inhibits one or both of BTK and/or PI3Kδ to which it is covalently bound. In some aspects, the compound forms a covalent bond with a cysteine residue on one or both of BTK and/or PI3Kδ.

In some aspects, the subject is refractory to chemotherapy treatment, or in relapse after treatment with chemotherapy. In some aspects, the subject is a de novo patient. In some aspects, the method comprises reducing the level of BTK and/or PI3Kδ activity in said patient. In some aspects, the subject is a human subject.

In one aspect, the methods presented herein comprise administering to an individual (e.g., in a human) a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In one aspect are provided methods for treating autoimmune diseases in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In another aspect are provided methods for treating a heteroimmune condition in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In another aspect are provided methods for treating an inflammatory disease in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In another aspect are provided methods for treating cancer in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a therapeutically effective amount of compound provided herein, or a pharmaceutically acceptable salt thereof; a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In some embodiments, the present application provides a method for the treatment of cancer or for the treatment of an autoimmune disease to a patient comprising the administration of a therapeutically effective amount of a Btk inhibitor to a patient in need thereof, wherein the Btk inhibitor is a compound of the formulae (I), (II) or (III), (IVa), (IVb), (Va), (Vb), (VIa), (VIb), or any one of a compound of the formula B-1 to B-10, B-1a to B-10c, B-1a-1 to B-10c-8, or C-1a to C-3j; or a compound of Table 1 or 2, or a pharmaceutically acceptable salt thereof. In some embodiments the method, the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma. In some embodiments of the method, the autoimmune disease is selected from the group consisting of rheumatoid arthritis and systemic lupus erythematosis.

In some embodiments, the present application provides a method for the treatment of cancer or for the treatment of an autoimmune disease to a patient comprising the administration of a therapeutically effective amount of a PI3Kδ inhibitor to a patient in need thereof, wherein the PI3Kδ inhibitor is a compound of the formulae (I), (II) or (III), (IVa), (IVb), (Va), (Vb), (VIa), (VIb), or any one of a compound of the formula B-1 to B-10, B-1a to B-10c, B-1a-1 to B-10c-8, or C-1a to C-3j; or a compound of Table 1 or 2, or a pharmaceutically acceptable salt thereof. In some embodiments of the method, the cancer is selected from the group consisting of chronic lymphocytic leukemia, mantle cell lymphoma, B cell non Hodgkin lymphoma, multiple myeloma and acute myeloid lymphoma. In some embodiments of the method, the autoimmune disease is selected from the group consisting of rheumatoid arthritis, allergic asthma and myocardial infarction.

In some embodiments, the present application provides a method for the treatment of cancer or for the treatment of an autoimmune disease to a patient comprising the administration of a therapeutically effective amount of a dual Btk and PI3Kδ inhibitor to a patient in need thereof, wherein the dual Btk and PI3Kδ inhibitor is a compound of the formulae (I), (II) or (III), (IVa), (IVb), (Va), (Vb), (VIa), (VIb), or any one of a compound of the formula B-1 to B-10, B—1a to B-10c, B-1a-1 to B-10c-8, or C-1a to C-3j; or a compound of Table 1 or 2, or a pharmaceutically acceptable salt thereof. In some embodiments of the method, the cancer is selected from the group consisting of chronic lymphocytic leukemia, mantle cell lymphoma, multiple myeloma and B cell non Hodgkin lymphoma. In some embodiments of the method, the autoimmune disease is rheumatoid arthritis.

Additionally provided is a method for treating a tumor comprising contacting the tumor with an effective amount of one or more compounds provided herein, or a salt thereof. In one aspect of the method, a compound or salt thereof is administered to an individual in need of tumor treatment. In one aspect, the treatment results in a reduction of the tumor size. In another aspect, the treatment slows or prevents tumor growth and/or metastasis.

Any of the methods of treatment provided herein may be used to treat a primary tumor. Any of the methods of treatment provided herein may also be used to treat a metastatic cancer (that is, cancer that has metastasized from the primary tumor). Any of the methods of treatment provided herein may be used to treat cancer at an advanced stage. Any of the methods of treatment provided herein may be used to treat cancer at a locally advanced stage. Any of the methods of treatment provided herein may be used to treat early stage cancer. Any of the methods of treatment provided herein may be used to treat cancer in remission. In some of the embodiments of any of the methods of treatment provided herein, the cancer has reoccurred after remission. In some embodiments of any of the methods of treatment provided herein, the cancer is progressive cancer.

In another aspect are provided methods for treating a thromboembolic disorder in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In another aspect are provided methods for treating a respiratory disease in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt (such as a pharmaceutically acceptable salt) of any of the foregoing.

In each of the above embodiments, aspects and variations, the application also provides compounds of the formulae (I), (II), (III), (IVa), (IVb), (Va), (Vb), (VIa), (VIb), B-1 to B-10, B-1a to B-10c, B-1a-1 to B-10c-8, or C-1a to C-3j; or a compound of Table 1 or 2, or an isomer thereof, or a salt thereof, for use in a method of treating cancer or for the treatment of an autoimmune disease. In one aspect of the above, the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma.

### Dosing and Method of Administration

The dose of a compound administered to an individual (such as a human) may vary with the particular compound or salt thereof, the method of administration, and the particular indication being treated. The amount should be sufficient to produce a desirable response, such as a therapeutic or prophylactic response against the disease. In some embodiments, the amount of the compound or salt thereof is a therapeutically effective amount. In some embodiments, the amount of the compound or salt thereof is a prophylactically effective amount. In some embodiments, the amount of compound or salt thereof is below the level that induces a toxicological effect (e.g., an effect above a clinically acceptable level of toxicity) or is at a level where a potential side effect can be controlled or tolerated when the composition is administered to the individual.

Methods as provided herein may comprise administering to an individual a pharmacological composition that contains an effective amount of a compound and a pharmaceutically acceptable carrier. The effective amount of the compound may in one aspect be a dose of between about 0.01 and about 100 mg/kg.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein.

Articles of manufacture comprising a compound of the invention, or a salt or solvate thereof, in a suitable container are provided. The container may be a vial, jar, ampoule, preloaded syringe, i.v. bag, and the like.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants.

Any of the compounds described herein can be formulated in a tablet in any dosage form described, for example, a compound as described herein or a pharmaceutically acceptable salt thereof can be formulated as a 10 mg tablet.

The compound may be administered to an individual in accordance with an effective dosing regimen for a desired period of time or duration, such as at least about one month, at least about 2 month, at least about 3 month, at least about 6 month, or at least about 12 months or longer, which in some embodiments may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent (e.g., once daily dosing for 7 days followed by no doses for 7 days, repeated for any 14 day time period, such as about 2 month, about 4 month, about 6 months or more). Any of the dosing frequencies can employ any of the compounds described herein together with any of the dosages described herein.

Compositions comprising a compound provided herein are also described. In one variation, the composition comprises a compound and a pharmaceutically acceptable carrier or excipient. In another variation, a composition of substantially pure compound is provided.

### Kits

Kits comprising a compound of the invention, or a salt or solvate thereof, and suitable packaging are provided. In one embodiment, a kit further comprises instructions for use. In one aspect, a kit comprises a compound of the invention, or a salt or solvate thereof, and instructions for use of the compounds in the treatment of a disease or condition for which a reduction in basophil and/or mast cell is expected to be or is beneficial.

The invention further provides kits for carrying out the methods of the invention, which comprises one or more compounds described herein or a pharmacological composition comprising a compound described herein. The kits may employ any of the compounds disclosed herein. In one variation, the kit employs a compound described herein or a pharmaceutically acceptable salt thereof.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 month, 4 month, 5 month, 7 month, 8 month, 9 month, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### General Synthetic Methods

The compounds of the invention may be prepared by a number of processes as generally described below and more specifically in the Examples hereinafter. In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

The following abbreviations are used herein: thin layer chromatography (TLC); hour (h); minute (min); second (sec); ethanol (EtOH); dimethylsulfoxide (DMSO); N,N-dimethylformamide (DMF); trifluoroacetic acid (TFA); tetrahydrofuran (THF); molar (M); Normal (N); aqueous (aq.); methanol (MeOH); dichloromethane (DCM); ethyl acetate (EtOAc); Retention factor (Rf); room temperature (RT).

Compounds detailed herein may be prepared by those of skill in the art by referral to the General Methods and Examples described below. Synthetic routes to bicyclic heterocyclic compounds analogous to compounds C, N, S, W and AC, in the General Methods below will be familiar to those skilled in the art or readily available in the published literature. As typical examples, routes to functionalized pyrazolopyrimidines, pyridopyrimidines, and pyrazolopyridines are presented in General Methods 1 to 5.

### General Method 1

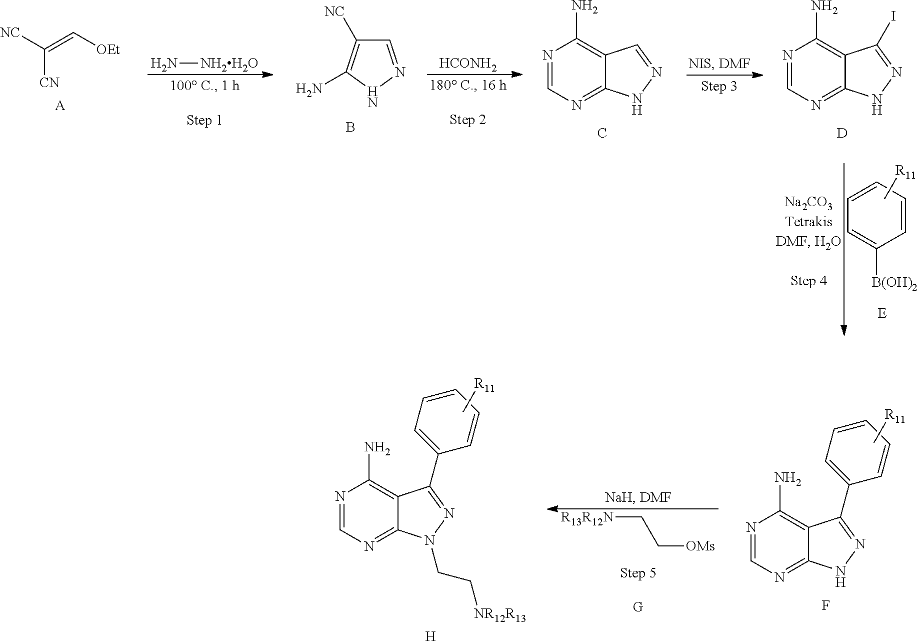

In certain examples of formula (I) provided herein, compounds of the type J can be prepared from starting materials of the type A. This dinitrile A can be treated with hydrazine, under standard pyrazole-forming conditions of step 1 to provide compound B which when treated with formamide in step 2 provides the pyrazolopyrimidine C. Functionalization of the 3-position in step 3 with an appropriate halogenating reagent, such as N-iodosuccinimide (NIS) in this case, provides the 3-iodo derivative D. Other halogenating agents can be envisioned for this step. Treatment of D with an appropriately functionalized boronic acid E under Suzuki coupling conditions of step 4, affords the coupled product F. Treatment of amine F with appropriately functionalized aminoethyl-mesylate G under basic conditions in final step 5 allows coupling at the 1-position of the pyrazolopyrimidine, to yield the final product H. Other labile groups such as tosylates and other leaving groups of reagent G will be familiar to those skilled in the art.

### General Method 2

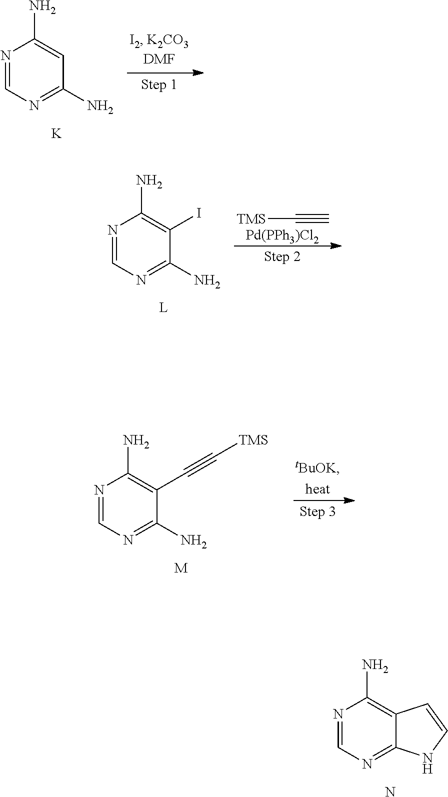

In other examples of formula (I), pyrrolopyrimidines of the type N can be prepared from diaminopyrimidines K. Iodination of compound K under mildly basic conditions of step 1 allows for the iodo intermediate L which, when treated with trimethylsilyl-acetylene under Palladium-mediated coupling conditions of step 2 provides coupled compound M. Base-mediated cyclization of the acetylene in step 3 yields the pyrrolopyrimidine product N. This compound N can be further utilized in a similar fashion to compound C of General Method 1.

### General Method 3

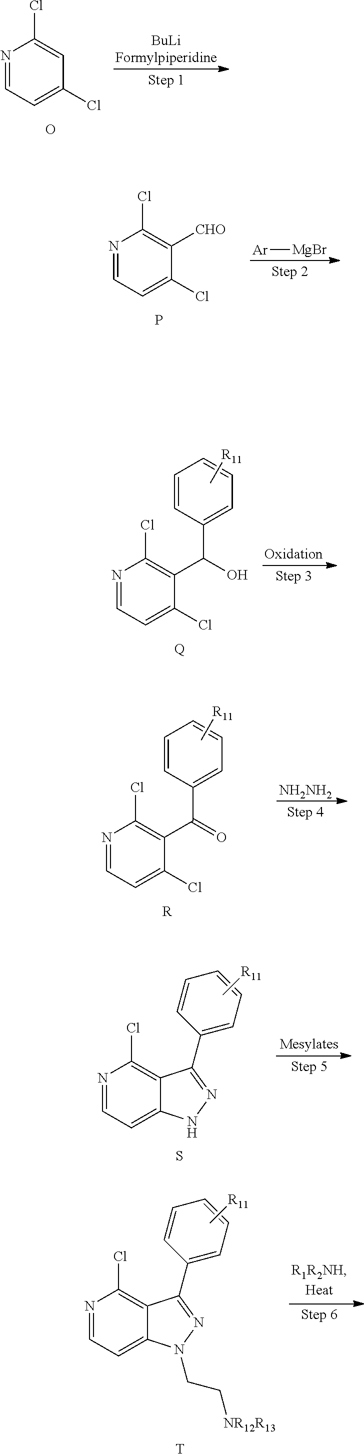

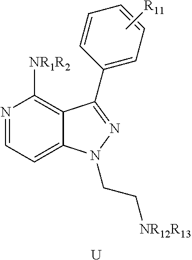

In other examples of formula (I), subjecting 2,4-dichloropyridine 0 to formylation conditions in step 1 provides the aldehyde intermediate P which, when treated in step 2 with appropriately functionalized aryl metallic reagent or an aryl Grignard reagent, yields the coupled alcohol Q. Oxidation of the alcohol to the ketone under standard conditions in step 3 affords compound R which, when treated with hydrazine in step 4 gives the pyrazolopyridine S. The free amine group can be coupled in step 5 with various mesylates as described in General Method 1 to give compound T, followed finally by conversion of the chloro group to the desired amine in step 6, to give the desired final product U.

### General Method 4

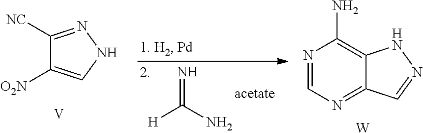

In certain examples of formula (II) provided herein, compounds of the type W can be prepared from starting materials of the type V. Hydrogenation of the nitro pyrazolecarbonitrile V, followed by heating with formamidine acetate provides the pyrazolopyrimidine-7-amine W.

### General Method 5

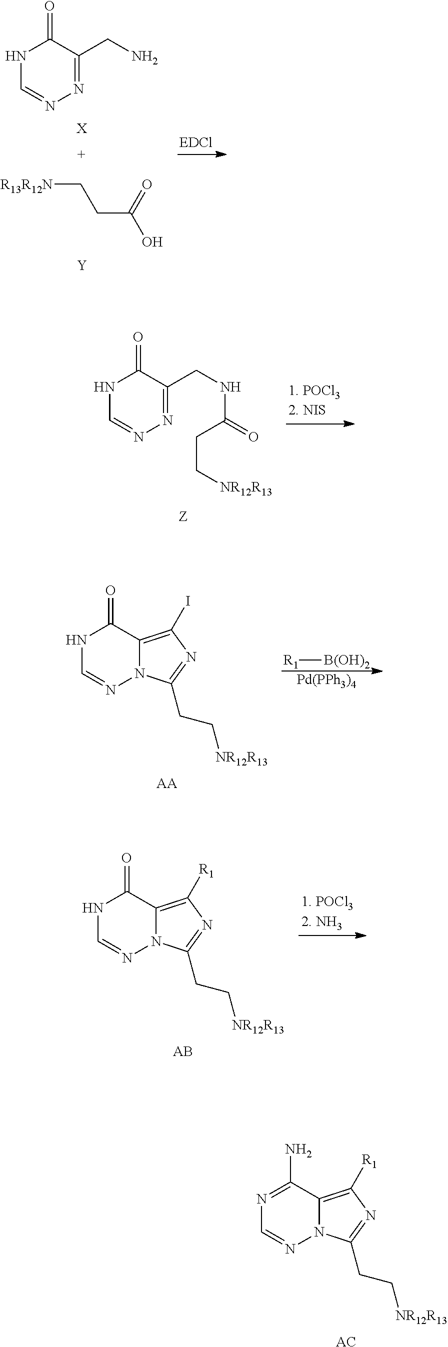

In certain examples of formula (III) provided herein, compounds of the type AC can be prepared from starting materials of the type X. Aminomethyl-triazinone X, described in patent publication US2008/0076921A1, can be coupled with appropriately functionalized carboxylic acid Y, under standard coupling conditions to give amide Z. Chlorination of Z with POCl₃ results in ring closure to yield the imidazotriazinone which, upon treatment with NIS results in the iodinated derivative AA. Coupling of the iodo compound AA with an appropriately functionalized boronic acid R₁—B(OH)₂, under Suzuki coupling conditions, yields the coupled product AB, the pyrimidone moiety of which can be converted to the amino pyrimidine product following well-versed chlorination-amination steps to afford the final product AC.

The following Examples are provided to illustrate but not to limit the invention.

All references disclosed herein are incorporated herein by reference in their entireties.

### EXAMPLES

### Example 1

### Preparation of Compound No. 1

### Synthesis of 3-(4-amino-1-(2-(phenylamino)ethyl)-1H-pyrazolo [3,4-d]pyrimidin-3-yl)-5-fluorophenol

### Step-1: Synthesis of 5-amino-1H-pyrazole-4-carbonitrile

To 2-(ethoxymethylene)propanedinitrile (20 g, 163.80 mmol) was added hydrazine hydrate (15.9 mL, 327.60 mmol) dropwise at 0° C. and the resultant reaction mixture was heated at 100° C. in a closed reagent bottle for 1 h. The reaction was monitored by TLC. After completion, the reaction mixture was cooled to RT and water (50 mL) was added. The product was extracted using EtOAc (3×200 mL). The combined organic layer was dried over anhydrous sodium sulfate and concentrated under reduced pressure to obtain 15 g of 5-amino-1H-pyrazole-4-carbonitrile as a light brown solid.

### Step-2: Synthesis of 1H-pyrazolo[3,4-d]pyrimidin-4-amine

To 5-amino-1H-pyrazole-4-carbonitrile (15 g, 138.76 m mol) was added formamide (75 mL) under nitrogen atmosphere and the reaction mixture was heated at 180° C. overnight. The reaction was monitored by TLC. After completion of reaction, the mixture was cooled to 0° C. and water (50 mL) was added, whereupon a precipitate formed. The precipitate was collected by filtration and dried to obtain 13.1 g of 1H-pyrazolo[3, 4-d]pyrimidin-4-amine as a yellow solid.

### Step-3: Synthesis of 3-iodo-1H-pyrazolo[3,4-d]pyrimidin-4-amine

To a suspension of 1H-pyrazolo[3, 4-d]pyrimidin-4-amine (7.5 g, 55.50 mmol) in anhydrous DMF (50 mL) was added N-iodosuccinimide (49 g, 222.0 mmol) portionwise under a nitrogen atmosphere. The reaction mixture was heated at 80° C. for 2 h. The reaction was monitored by TLC. After completion of reaction, the mixture was cooled to RT and diluted with EtOAc (400 mL). Saturated aq. sodium thiosulfate solution (100 mL) was added, whereupon a precipitate formed. The precipitate was collected by filtration, washed with additional water, and diethyl ether, and dried to obtain 14 g of 3-iodo-1H-pyrazolo[3,4-d]pyrimidin-4-amine as an off-white solid.

### Step-4: Synthesis of 3-(3-fluoro-5-methoxyphenyl)-1H-pyrazolo[3,4-d]pyrimidin-4-amine

To a solution of 3-iodo-1H-pyrazolo[3,4-d]pyrimidin-4-amine (700 mg, 2.68 mmol) in DMF (12 mL) was added 3-fluoro-5-methoxyphenylboronic acid (546.9 mg, 3.21 mmol) at RT. A solution of Na₂CO₃ (852.6 mg, 8.04 mmol) in H₂O (10 mL) was added to the reaction mixture followed by addition of Pd(PPh₃)₄ (309.8 mg, 0.268 mmol) at RT, and the resultant reaction mixture was heated at 100° C. for 16 h. The reaction was monitored by TLC and LCMS. After completion of reaction, the mixture was filtered through a celite bed. The filtrate obtained was diluted with EtOAc (300 mL). The organic layer was washed with water (2×150 mL), dried over sodium sulfate and concentrated. The crude compound was purified by column chromatography (silica gel, 100-200), eluting with 4% MeOH/DCM to afford 225 mg of 3-(3-fluoro-5-methoxyphenyl)-1H-pyrazolo[3,4-d]pyrimidin-4-amine as a light brown solid.

### Step-5: Synthesis of 2-(phenylamino)ethanol

Aniline (5 g, 53.68 mmol) and 2-bromoethanol (6.03g, 48.31 mmol) were charged in a reaction bottle. The mixture was heated at 80° C. for 1 h, and the reaction monitored by TLC and NMR. The reaction mixture was allowed to come to RT and basified with a saturated solution of sodium bicarbonate. The mixture was extracted with EtOAc (2×100 mL). The combined organic layer was dried over anhydrous sodium sulfate and concentrated under reduced pressure. The crude product was purified on silica column (#100-200) using 0-30% EtOAc: hexane as eluent to give 3 g of 2-(phenylamino)ethanol.

### Step-6: Synthesis of 2-(phenylamino) ethyl methanesulfonate

2-(Phenylamino) ethanol (5) (2.8g, 20.4 mL) was charged in DCM (30 mL). Triethylamine (5.6 mL, 40.8 mmol) was added to the reaction mixture and the mixture was cooled to 0° C. Mesyl chloride (1.6 mL, 20.4 mmol) was added dropwise and the mixture was stirred at the same temperature for 30 min. The reaction was monitored by TLC and water (30 mL) was then added. The DCM layer was isolated and the aqueous layer was further washed with DCM (2×30 mL). The combined organic layer was dried over anhydrous sodium sulfate and concentrated under reduced pressure to obtain the crude product. The crude product was purified on silica column (#100-200) using 0-30% EtOAc:hexane as eluant to give 2.3 g of 2-(phenylamino)ethyl methanesulfonate.

### Step-7: Synthesis of 3-(3-fluoro-5-methoxyphenyl)-1-(2-(phenylamino)ethyl)-1H-pyrazolo[3,4-d]pyrimidin-4-amine

3-(3-Fluoro-5-methoxyphenyl)-1H-pyrazolo[3,4-d]pyrimidin-4-amine (300 mg, 1.15 mmol) was charged in DMF (2 mL), and the reaction mixture cooled to 0° C. Sodium hydride (55 mg, 1.38 mmol) was added. After 10 min, 2-(phenylamino)ethyl methanesulfonate (298 mg, 1.38 mmol) was added dropwise. The reaction mixture was allowed to come to RT and stirred at RT overnight. The reaction was monitored by TLC and LCMS. Water (10 mL) was added and the mixture was extracted with EtOAc (2×50 mL). The combined organic layer was washed with water (3×25 mL) and dried over anhydrous sodium sulfate. The solvent was evaporated under reduced pressure and the crude product was triturated with hexane. The solid obtained was filtered off and mother liquor was concentrated to get 201 mg of 3-(3-fluoro-5-methoxyphenyl)-1-(2-(phenylamino)ethyl)-1H-pyrazolo[3,4-d]pyrimidin-4-amine.

### Step-8: Synthesis of 3-(4-amino-1-(2-(phenylamino)ethyl)-1H-pyrazolo[3,4-d]pyrimidin-3-yl)-5-fluorophenol

3-(3-Fluoro-5-methoxyphenyl)-1-(2-(phenylamino)ethyl)-1H-pyrazolo [3,4-d]pyrimidin-4-amine (25 mg, 0.066 mmol) was charged in DCM (1 mL) and the reaction mixture was cooled to 0° C. A 1M solution of boron tribromide (0.2 mL, 0.198 mmol) was added dropwise. The reaction mixture was allowed to come to RT and stirred at RT for 4 h. The reaction was monitored by TLC and LCMS. Saturated sodium bicarbonate was added and the mixture extracted with EtOAc (3×25 mL). The combined organic layer was dried over anhydrous sodium sulfate and concentrated under reduced pressure and purified using reverse phase HPLC to get (12 mg) 3-(4-amino-1-(2-(phenylamino)ethyl)-1H-pyrazolo[3,4-d]pyrimidin-3-yl)-5-fluorophenol. ¹H NMR (400 MHz, CD₃OD) δ (ppm): 8.23 (s, 1H), 6.98 (m, 2H), 6.90 (s, 1H), 6.85 (d, J=9.1 Hz, 1H), 6.65 (d, J=10.7 Hz, 1H), 6.52 (m, 3H), 4.56 (t, J=6.1 Hz, 3H), 3.66 (t, J=6.2 Hz, 2H).

### Example 2

### Preparation of Compound No. 2

### Synthesis of N-(2-(4-amino-3-(3-fluoro-5-hydroxyphenyl)-1H-pyrazolo[3,4-d]pyrimidin-1-yl)ethyl)-N-phenylacrylamide

### Step-1: Synthesis of 3-(4-Amino-1-(2-(phenylamino)ethyl)-1H-pyrazolo[3,4-d]pyrimidin-3-yl)-5-fluorophenol

See Example 1.

### Step-2: Synthesis of N-(2-(4-amino-3-(3-fluoro-5-hydroxyphenyl)-1H-pyrazolo[3,4-d]pyrimidin-1-yl)ethyl)-N-phenylacrylamide

3-(4-Amino-1-(2-(phenylamino)ethyl)-1H-pyrazolo[3,4-d]pyrimidin-3-yl)-5-fluorophenol (25 mg, 0.066 mmol) was charged in DCM (1 mL) and triethylamine (0.004 mL, 0.03 mmol) was added. The reaction mixture was cooled to 0° C. and a 1M solution of acryloyl chloride (0.068 mL, 0.03 mmol) was added. The reaction mixture was allowed to come to RT and stirred at RT for 4 h. A saturated solution of sodium bicarbonate was added and the DCM layer was extracted. The aqueous layer was washed with DCM (2×25 mL). The combined DCM layer was dried over anhydrous sodium sulfate and concentrated under vacuum. The crude product was purified using reverse phase HPLC to get 3.64 mg of N-(2-(4-amino-3-(3-fluoro-5-hydroxyphenyl)-1H-pyrazolo[3,4-d]pyrimidin-1-yl)ethyl)-N-phenylacrylamide. ¹H NMR (400 MHz, CD₃OD) δ (ppm): 8.22 (s, 1H), 7.26 (m, 3H), 7.03 (m, 2H), 6.82 (s, 1H), 6.73 (d, J=9.0 Hz, 1H), 6.64 (d, J=10.6 Hz, 1H), 6.22-5.98 (m, 2H), 5.51 (d, J=10.2 Hz, 1H), 4.66 (t, J=5.4 Hz, 3H), 4.45-4.24 (m, 3H).

### Example 3

### Preparation of Compound No. 3

### Synthesis of N-(2-(4-amino-3-(3-fluoro-5-hydroxyphenyl)-1H-pyrazolo[3,4-d]pyrimidin-1-yl)ethyl)-2-chloro-4-fluorobenzamide

### Step-1: Synthesis of 3-iodo-1H-pyrazolo[3,4-d]pyrimidin-4-amine

See Example 1.

### Step-2: Synthesis of tert-butyl (2-hydroxyethyl)carbamate

To 2-aminoethanol (5 g, 81.94 mmol) in THF (60 mL) was added sodium carbonate (17.3 g, 163.88 mmol) dissolved in water at RT. The reaction was allowed to stir at RT for 10 min. Di-tert-butyl dicarbonate (19.6 g, 90.13 mmol) was added to the reaction mixture at 0° C. The reaction mixture was brought to RT and allowed to stir at RT overnight. The reaction was monitored by NMR. To the reaction mixture was added water (40 mL) and the mixture extracted with EtOAc (2×400 mL). The combined organic layer was washed with water (100 mL), brine (100 mL), dried over sodium sulfate, and concentrated under reduced pressure to give 12.1 g of desired product as a pale yellow compound.

### Step-3: Synthesis of 2-((tert-butoxycarbonyl)amino)ethyl methanesulfonate

To tert-butyl N-(2-hydroxyethyl)carbamate (12 g, 50.14 mmol) in DCM (75 mL) was added triethylamine (35.23 mL, 250.74 mmol) at 0° C. The reaction was brought to RT and allowed to stir at RT for 15 min. Mesyl chloride (5.8 mL, 75.2 mmol) was added to the reaction mixture at 0° C. dropwise. The reaction was brought to RT slowly and allowed to stir at RT for 2 h. The reaction was monitored by NMR. The reaction was quenched with water (100 mL) and the reaction mixture extracted with the addition of more DCM (800 mL). The organic layer was washed with an aq. solution of citric acid (100 mL), brine (100 mL), dried over sodium sulfate and concentrated under reduced pressure to give crude product. The crude product was purified by silica gel (#100-200) column chromatography, eluting with 20% EtOAc/Hexane to give 12.2 g of desired product as a pale yellow liquid compound.

### Step-4: Synthesis of tert-butyl (2-(4-amino-3-iodo-1H-pyrazolo[3,4-d]pyrimidin-1-yl)ethyl)carbamate

To 3-iodo-1H-pyrazolo[3,4-d]pyrimidin-4-amine (1g, 3.83 mmol) in DMF (18 mL) was added sodium hydride (305 mg, 7.66 mmol) at 0° C. and the reaction mixture was brought to RT and allowed to stir at RT for 30 min. After 30 min, 2-(tert-butoxycarbonylamino)ethyl methanesulfonate (1.83 g, 7.66 mmol) dissolved in DMF (2 mL) was added to the reaction mixture at 0° C. The reaction mixture was immediately heated to 100° C. for 1 h. The reaction was monitored by TLC and LCMS. The reaction was quenched by addition of ice cold water (20 mL) at 0° C. and the reaction mixture extracted with EtOAc (2×250 mL). The combined organic layer was washed with aqueous solution of ammonium chloride (75 mL), water (3×75 mL), brine (75 mL), dried over sodium sulfate and concentrated under reduced pressure to give 1.1g of desired compound.

### Step-5: Synthesis of 1-(2-aminoethyl)-3-iodo-M-pyrazolo[3,4-d]pyrimidin-4-amine

To (ethyl N-[2-(4-amino-3-iodo-pyrazolo[3,4-d]pyrimidin-1-yl)ethyl]carbamate (500 mg, 1.64 mmol) in DCM was added TFA (1.5 mL) at 0° C. dropwise, and the reaction was allowed to run at RT for 1 h. The reaction was monitored by TLC. On completion, the mixture was evaporated under reduced pressure to give crude product which was purified by washing with pentane to give 510 mg of product as a creamish solid compound.

### Step-6: Synthesis of N-(2-(4-amino-3-iodo-1H-pyrazolo[3,4-d]pyrimidin-1-yl)ethyl)-2-chloro-4-fluorobenzamide

To 2-chloro-4-fluoro-benzoic acid in DMF (8 mL) were added PyBOP (670 mg, 1.28 mmol) and DIPEA (0.6 mL, 3.43 mmol) and the reaction was allowed to stir at RT for 10 min. 1-(2-Aminoethyl)-3-iodo-pyrazolo[3,4-d]pyrimidin-4-amine (538 mg, 1.28 mmol) dissolved in DMF (2 mL) was added to the reaction mixture. The reaction was allowed to stir at RT overnight. The reaction was monitored by LCMS. On completion, water (20 mL) was added to the reaction mixture and the reaction mixture was extracted with EtOAc (2×100 mL). The combined organic layer was washed with water (3×25 mL), brine (50 mL), dried over sodium sulfate and concentrated under reduced pressure to give crude product. This crude product was purified by precipitating in presence of acetone as solvent giving 275 mg of desired compound as an off white solid.

### Step-7: Synthesis of N-(2-(4-amino-3-(3-fluoro-5-methoxyphenyl)-1H-pyrazolo[3,4-d]pyrimidin-1-yl)ethyl)-2-chloro-4-fluorobenzamide

To N-[2-(4-amino-3-iodo-pyrazolo[3,4-d]pyrimidin-1-yl)ethyl]-2-chloro-4-fluoro-benzamide (250 mg, 0.54 mmol) and (3-fluoro-5-methoxy-phenyl)boronic acid (138 mg, 0.81 mmol) in DMF (5 mL) was added sodium carbonate (115 mg, 1.08 mmol) dissolved in water (5 mL), and the reaction was heated at 100° C. overnight. The reaction was monitored by LCMS and TLC. On completion, water (10 mL) was added to the reaction mixture and the reaction mixture extracted with EtOAc (2×100 mL). The combined organic layer was washed with water (2×25 mL), brine (30 mL), dried over sodium sulfate and concentrated under reduced pressure to give 240 mg crude product which was used in the next reaction without further purification.

### Step-8: Synthesis of N-(2-(4-amino-3-(3-fluoro-5-hydroxyphenyl)-1H-pyrazolo[3,4-d]pyrimidin-1-yl)ethyl)-2-chloro-4-fluorobenzamide

To a suspension of N-[2-[4-amino-3-(3-fluoro-5-methoxy-phenyl)pyrazolo[3,4-d]pyrimidin-1-yl]ethyl]-2-chloro-4-fluoro-benzamide (90 mg, 0.196 mmol) in DCM (4 mL) was added BBr₃ (1M in DCM) (0.9 mL, 0.98 mmol) at 0° C. and the reaction was allowed to stir at RT for 2 h. The reaction was monitored by LCMS. On completion reaction was quenched with an aqueous solution of sodium bicarbonate (3 mL) and the reaction mixture was extracted with the addition of more DCM (100 mL). The organic layer was washed with water (20 mL), brine (15 mL), dried over sodium sulfate and concentrated under reduced pressure to give crude product. The crude product was purified by preparative HPLC to afford 14.1 mg of off white solid compound as the TFA salt. ¹H NMR (400 MHz, CD₃OD) δ (ppm): 8.36 (s, 1H), 7.38 (dd, J=8.6, 6.0 Hz, 1H), 7.22 (dd, J=8.8, 2.5 Hz, 1H), 7.02 (td, J=8.4, 2.5 Hz, 1H), 6.97-6.82 (m, 2H), 6.69 (dt, J=10.6, 2.4 Hz, 1H), 4.69 (t, J=5.5 Hz, 2H), 3.91 (t, J=5.5 Hz, 2H).

### Example 4

### Preparation of Compound No. 4

Synthesis of 3-{4-amino-1-[2-(benzylamino)ethyl]-1H-pyrazolo[3,4-d]pyrimidin-3-yl}-5-fluorophenol

### Step-1: Synthesis of 5-amino-1H-pyrazole-4-carbonitrile

Ethoxymethylenemalonitrile (20 g, 163.8 mmol) was charged in a flask at 0° C. and hydrazine hydrate (16 mL, 327.6 mmol) was added dropwise. After completion of addition, the reaction mixture was heated at 100° C. for 0.5 h. Ice cold water (10 mL) was added and the reaction mixture was extracted in EtOAc (5×100 mL). The reaction mixture was dried over anhydrous sodium sulfate and concentrated to get 10.9 g of 5-amino-1H-pyrazole-4-carbonitrile.

### Step-2: Synthesis of 1H-pyrazolo[3,4-d]pyrimidin-4-amine

5-Amino-1H-pyrazole-4-carbonitrile (10.9 g, 100.9 mmol) and formamide (40 mL, 10 fold) were charged in a flask and the reaction mixture was heated at 180° C. overnight. Ice cold water (10 mL) was added and the solid obtained was filtered and dried under vacuum to get 7.6 g of 1H-pyrazolo[3,4-d]pyrimidin-4-amine.

### Step-3: Synthesis of 3-iodo-1H-pyrazolo[3,4-d]pyrimidin-4-amine

1H-Pyrazolo[3,4-d]pyrimidin-4-amine (2 g, 14.8 mol) was charged in DMF (12 mL) and to this was added N-iodosuccinimide (6.3 g, 28.6 mmol). The reaction mixture was heated at 100° C. for 4 h. The solid obtained was filtered off and washed with cold EtOH (15 mL). The solid obtained was dried under vacuum to get (4 g) 3-iodo-1H-pyrazolo [3, 4-d]pyrimidin-4-amine.

### Step-4: Synthesis of 3-(3-fluoro-5-methoxyphenyl)-1H-pyrazolo[3,4-d]pyrimidin-4-amine

3-Iodo-1H-pyrazolo [3, 4-d]pyrimidin-4-amine (3 g, 11.45 mmol), tetrakistriphenylphosphinepalladium (0) (1.3 g, 1.145 mmol) was charged in DME (60 mL) and the mixture purged with nitrogen for 5 min. Potassium carbonate (1.5 g, 11.45 mmol) and 3-fluoro-5-methoxyphenylboronic acid (5.4 g, 34.55 mmol) were added. Water (60 mL) was added and the mixture again purged with nitrogen for 5 min. The reaction mixture was stirred at 100° C. for 24 h. The reaction was monitored by TLC and LCMS. Then reaction mixture was acidified with 2M HCl and the aqueous layer was separated with EtOAc (3×100 mL). The aqueous layer was basified with saturated sodium carbonate solution and the solid obtained was filtered off. The solid obtained was washed with water (10 mL) and dried under vacuum to get 1.3 g of 3-(3-fluoro-5-methoxyphenyl)-1H-pyrazolo[3,4-d]pyrimidin-4-amine.

### Step-5: Synthesis of 2-(benzylamino)ethyl methanesulfonate

2-(Benzyl amino)ethanol (500 mg, 3.3 mmol) was charged in DCM (20 mL). Triethylamine (0.91 mL, 6.61 mmol) was added and the mixture was cooled to 0° C. Methane sulphonyl chloride (0.12 mL, 1.65 mmol) was added at dropwise and the reaction mixture was stirred at the same temperature for 30 min. The reaction was monitored by TLC, and then water (15 mL) was added. The DCM layer was extracted and the aqueous layer was further extracted with DCM (2×30 mL). The combined organic layer was dried over anhydrous sodium sulfate and concentrated under reduced pressure. The crude product was purified on silica column (#100-200) using 0-30% EtOAc:hexane as eluent to give 255 mg of 2-(benzylamino)ethyl methanesulfonate (5a) and also get 115 mg of 2-(N-benzylmethylsulfonamido)ethyl methanesulfonate.

### Step-6: Synthesis of 1-(2-(benzylamino)ethyl)-3-(3-fluoro-5-methoxyphenyl)-1H-pyrazolo[3,4-d]pyrimidin-4-amine

3-(3-Fluoro-5-methoxyphenyl)-1H-pyrazolo[3,4-d]pyrimidin-4-amine (25 mg, 0.19 mmol) was charged in DMF (2 mL) and potassium carbonate (104.8 mg, 0.76 mmol) was added. The reaction mixture was heated at 80° C. for 5 min under nitrogen and N-benzyl-2-chloroethanamine (65 mg, 0.38 mmol). The reaction mixture was heated at 80° C. overnight. The reaction was monitored by TLC and LCMS and allowed to come to RT. Water (10 mL) was added and the mixture was extracted with EtOAc (2×50 mL). The combined organic layer was washed with water (3×25 mL) and dried over anhydrous sodium sulfate. The solvent was evaporated under reduced pressure and the crude product was triturated with hexane. The solid obtained was filtered off and mother liquor was concentrated to get 35 mg of 1-(2 (benzylamino)ethyl)-3-(3-fluoro-5-methoxyphenyl)-1H-pyrazolo[3,4-d]pyrimidin-4-amine.

### Step-7: Synthesis of 3-(4-amino-1-(2-(benzylamino)ethyl)-1H-pyrazolo[3,4-d]pyrimidin-3-yl)-5-fluorophenol

1-(2-(Benzylamino)ethyl)-3-(3-fluoro-5-methoxyphenyl)-1H-pyrazolo[3,4-d]pyrimidin-4-amine (30 mg, 0.076 mmol) was charged in DCM (3 mL) and the reaction mixture was cooled to 0° C. A 1M solution of Boron tribromide in DCM (0.23 mL, 0.23 mmol) was added dropwise. The reaction mixture was allowed to come to RT and stirred at RT for 4 h. The reaction was monitored by TLC and LCMS. Saturated sodium bicarbonate was added and the mixture was extracted with EtOAc (3×25 mL). The combined organic layer was dried over anhydrous sodium sulfate and concentrated under reduced pressure and purified using reverse phase HPLC to get 3.52 mg of 3-(4-amino-1-(2-(benzylamino)ethyl)-1H-pyrazolo[3,4-d]pyrimidin-3-yl)-5-fluorophenol. ¹H NMR (400 MHz, CD₃OD) δ (ppm): 8.34 (s, 1H), 7.53-7.42 (m, 5H), 7.04-6.84 (m, 2H), 6.70 (d, J=10.6 Hz, 1H), 4.80 (t, J=5.8 Hz, 2H), 4.34 (s, 2H), 3.68 (t, J=5.7 Hz, 2H).

### Example 5

### Preparation of Compound No. 5

### Synthesis of N-(2-(4-amino-3-(3-fluoro-5-hydroxyphenyl)-1H-pyrazolo[3,4-d]pyrimidin-1-yl)ethyl)-N-benzylacrylamide

3-(4-Amino-1-(2-(phenylamino)ethyl)-1H-pyrazolo[3,4-d]pyrimidin-3-yl)-5-fluorophenol (15 mg, 0.04 mmol) was charged in DCM (1 mL) and triethylamine (0.005 mL, 0.02 mmol) were added. The reaction mixture was cooled to 0° C. and a 1M solution of acryloyl chloride in DCM (0.04 mL, 0.02 mmol) was added. The reaction mixture was allowed to come to RT and stirred at for 4 h. A saturated solution of sodium bicarbonate was added and the DCM layer was extracted. The aqueous layer was washed with DCM (2×25 mL). The combined DCM layer was dried over anhydrous sodium sulfate and concentrated under vacuum. The crude product was purified using reverse phase HPLC to get (2.16 mg) of N-(2-(4-amino-3-(3-fluoro-5-hydroxyphenyl)-1H-pyrazolo[3,4-d]pyrimidin-1-yl)ethyl)-N-benzylacrylamide. ¹H NMR (400 MHz, CD₃OD) δ (ppm): 8.32 (s, 1H), 7.28-7.19 (m, 5H), 7.10 (d, J=7.1 Hz, 1H), 6.90-6.87 (m, 1H), 6.85-6.67 (m, 1H), 6.70-6.65 (m, 0.5H), 6.60-6.41 (m, 0.5H), 6.15 (d, 0.5H), 6.01 (d, 0.5H), 5.67 (d, J=10.5 Hz, 0.5H), 5.49 (d, J=10.5 Hz, 0.5H), 4.68 (m, 2H), 4.60 (s, 1H), 3.99 (m, 2H).

### Example 6

### Preparation of Compound No. 6

### Synthesis of N-[2-[4-amino-3-(3-fluoro-5-hydroxy-phenyl)pyrazolo[3,4-d]pyrimidin-1-yl]ethyl]-N-benzyl-methanesulfonamide

### Step-1: Synthesis of 5-amino-1H-pyrazole-4-carbonitrile

Ethoxymethylenemalonitrile (20 g, 163.8 mmol) was charged in a flask at 0° C. and hydrazine hydrate (16 mL, 327.6 mmol) was added dropwise. After completion of addition, the reaction mixture was heated at 100° C. for 0.5 h. Ice cold water (10 mL) was added and the reaction mixture was extracted in EtOAc (5×100 mL). The reaction mixture was dried over anhydrous sodium sulfate and concentrated to get 10.9 g of 5-amino-1H-pyrazole-4-carbonitrile.

### Step-2: Synthesis of 1H-pyrazolo[3,4-d]pyrimidin-4-amine

5-Amino-1H-pyrazole-4-carbonitrile (10.9 g. 100.9 mmol) and formamide (40 mL, 10 fold) was charged in a flask and the reaction mixture was heated at 180° C. overnight. Ice cold water (10 mL) was added and the solid obtained was filtered and dried under vacuum to get 7.6 g of 1H-pyrazolo[3,4-d]pyrimidin-4-amine.

### Step-3: Synthesis of 3-iodo-1H-pyrazolo[3,4-d]pyrimidin-4-amine

1H-Pyrazolo[3,4-d]pyrimidin-4-amine (2 g, 14.8 mol) was charged in DMF (12 mL) and added N-iodosuccinimide (6.3 g, 28.6 mmol). The reaction mixture was heated at 100° C. for 4 h. The solid obtained was filtered off and washed with cold EtOH (15 mL). The solid obtained was dried under vacuum to get (4 g) 3-iodo-1H-pyrazolo [3, 4-d]pyrimidin-4-amine.

### Step-4: Synthesis of 3-(3-fluoro-5-methoxyphenyl)-1H-pyrazolo[3,4-d]pyrimidin-4-amine

3-Iodo-1H-pyrazolo [3, 4-d]pyrimidin-4-amine (3 g, 11.45 mmol), tetrakistriphenylphosphinepalladium (0) (1.3 g, 1.145 mmol) was charged in DME (60 mL) and the mixture purged with nitrogen for 5 min. Potassium carbonate (1.5 g, 11.45 mmol) and 3-fluoro-5-methoxyphenylboronic acid (5.4 g, 34.55 mmol) was added. Water (60 mL) was added and again purged with nitrogen for 5 min. The reaction mixture was stirred at 100° C. for 24 h. The reaction was monitored by TLC and LCMS. Then the reaction mixture was acidified with 2M HCl and the aqueous layer was separated with EtOAc (3×100 mL). The aqueous layer was basified with saturated sodium carbonate solution and the solid obtained was filtered off. The solid obtained was washed with water (10 mL) and dried under vacuum to get 1.3 g of 3-(3-fluoro-5-methoxyphenyl)-1H-pyrazolo[3,4-d]pyrimidin-4-amine.

### Step-5: Synthesis of 2-(benzylamino)ethyl methanesulfonate

2-(Benzylamino)ethanol (500 mg, 3.3 mmol) was charged in DCM (20 mL). Triethylamine (0.91 mL, 6.61 mmol) was added and the reaction mixture was cooled to 0° C. Methane sulphonyl chloride (0.12 mL, 1.65 mmol) was added dropwise and the reaction mixture was stirred at the same temperature for 30 min. The reaction was monitored by TLC and water (15 mL) was added. The DCM layer was extracted and the aqueous layer was further extracted with DCM (2×30 mL). The combined organic layer was dried over anhydrous sodium sulfate and concentrated under reduced pressure. The crude product was purified on silica column (#100-200) using 0-30% EtOAc: hexane as eluent to give 255 mg of 2-(benzylamino)ethyl methanesulfonate and 115 mg of 2-(N-benzylmethylsulfonamido)ethyl methanesulfonate.

### Step-6: Synthesis of 1-(2-(benzylamino)ethyl)-3-(3-fluoro-5-methoxyphenyl)-1H-pyrazolo[3,4-d]pyrimidin-4-amine

3-(3-Fluoro-5-methoxyphenyl)-1H-pyrazolo[3,4-d]pyrimidin-4-amine (25 mg, 0.19 mmol) was charged in DMF (2 mL) and potassium carbonate (104.8 mg, 0.76 mmol) was added. The reaction mixture was heated at 80° C. for 5 min under nitrogen and 2-(N-benzylmethylsulfonamido) ethyl methanesulfonate (32 mg, 0.10 mmol). The reaction mixture was heated at 80° C. overnight. The reaction was monitored by TLC and LCMS and allowed to come to RT. Water (10 mL) was added and the mixture was extracted with EtOAc (2×10 mL). The combined organic layer was washed with water (3×15 mL) and dried over anhydrous sodium sulfate. The solvent was evaporated under reduced pressure to get 27 mg of 1-(2-(benzylamino) ethyl)-3-(3-fluoro-5-methoxyphenyl)-1H-pyrazolo [3, 4-d]pyrimidin-4-amine.

### Step-7: Synthesis of N-[2-[4-amino-3-(3-fluoro-5-hydroxy-phenyl)pyrazolo[3,4-d]pyrimidin-1-yl]ethyl]-N-benzyl-methanesulfonamide

1-(2-(Benzylamino)ethyl)-3-(3-fluoro-5-methoxyphenyl)-1H-pyrazolo[3,4-d]pyrimidin-4-amine (25 mg, 0.053 mmol) was charged in DCM (2 mL) and the reaction mixture was cooled to 0° C. A 1M solution of boron tribromide in DCM (0.15 mL, 0.15 mmol) was added dropwise. The reaction mixture was allowed to come to RT and stirred at RT for 4 h. The reaction was monitored by TLC and LCMS. Saturated sodium bicarbonate was added and the mixture extracted with EtOAc (3×25 mL). The combined organic layer was dried over anhydrous sodium sulfate and concentrated under reduced pressure and purified using reverse phase HPLC to get 3.52 mg of 3-(4-amino-1-(2-(benzylamino)ethyl)-1H-pyrazolo[3,4-d]pyrimidin-3-yl)-5-fluorophenol. ¹H NMR (400 MHz, CD₃OD) δ (ppm): 8.34 (s, 1H), 7.18 (m, 5H), 6.83 (d, J=10.6 Hz, 1H) 6.66 (d, J=10.6 Hz, 1H), 4.75 (s, 2H), 4.53 (t, J=5.7 Hz, 2H), 3.76 (t, J=5.7, 2H), 3.3 (s, 3H).

### Example 7

### Preparation of Compound No. 7

### Synthesis of N-{2-[4-amino-3-(3-fluoro-5-hydroxyphenyl)-1H-pyrazolo[3,4-d]pyrimidin-1-yl]ethyl}-N-benzylpropanamide

To a stirred solution of 3-[4-amino-1-[2-(benzylamino)ethyl]pyrazolo[3,4-d]pyrimidin-3-yl]-5-fluoro-phenol TFA salt (150 mg, 0.247 mmol) in DCM (5 mL) and DMF (0.5 mL) was added triethylamine (25 mg, 0.247 mmol) at 0° C. under nitrogen atmosphere. The reaction mixture was stirred for 5 min then propanoyl chloride (22 mg, 0.247 mmol) was added. The reaction mixture was stirred at for 15 min. The reaction was monitored by TLC and LCMS. After completion of reaction, the reaction mixture was diluted with water (10 mL) and extracted with DCM (2×25 mL). The combined DCM layer was washed with water (2×25 mL) and brine solution (25 mL). The organic layer was dried over anhydrous sodium sulfate and concentrated under vacuum. The crude product was purified using reverse phase combiflash chromatography to get N-[2-[4-amino-3-(3-fluoro-5-hydroxy-phenyl)pyrazolo[3,4-d]pyrimidin-1-yl]ethyl]-N-benzyl-propanamide (28.5 mg). This was dissolved in ethanolic HCl (5 mL) and then dried to get N-[2-[4-amino-3-(3-fluoro-5-hydroxy-phenyl)pyrazolo[3,4-d]pyrimidin-1-yl]ethyl]-N-benzyl-propanamide (19.5 mg) as the HCl salt (20 mg). ¹H NMR (400 MHz, Methanol-d4) δ (ppm): 8.42 (s, 1H), 7.34-7.19 (m, 3H), 7.17-7.09 (m, 2H), 6.95-6.86 (m, 2H), 6.73 (ddt, J=10.2, 7.1, 2.2 Hz, 1H), 4.68 (t, J=5.2 Hz, 2H), 4.57 (s, 2H), 3.93 (q, J=5.3 Hz, 2H), 2.39-2.22 (m, 2H), 1.04-0.96 (t, J=7.5 Hz, 3H). LCMS: 435 (M+1).

### Example 8

### Preparation of Compound No. 8

### Synthesis of N-{2-[4-amino-3-(3-fluoro-5-hydroxyphenyl)-1H-pyrazolo[3,4-d]pyrimidin-1-yl]ethyl}-N-benzylcyclopropanecarboxamide

To a stirred solution of 3-[4-amino-1-[2-(benzylamino)ethyl]pyrazolo[3,4-d]pyrimidin-3-yl]-5-fluoro-phenol TFA salt (150 mg, 0.247 mmol) in DCM (5 mL) and DMF (0.5 mL) was added triethylamine (25 mg, 0.247 mmol) at 0° C. under nitrogen atmosphere. The reaction mixture was stirred for 5 min at the same temperature then to it was added cyclopropanecarbonyl chloride (25 mg, 0.247 mmol). The resultant reaction mixture was stirred at 0° C. for 15 min. The progress of reaction was monitored by TLC and LCMS. After completion of reaction, the reaction mixture was diluted with water (10 mL) and the product was extracted with DCM (2×25 mL). The combined DCM layer was washed with water (2×25 mL) and brine solution (25 mL). The organic layer was dried over anhydrous sodium sulfate and concentrated under vacuum to obtain crude product which was purified by reverse phase combiflash chromatography to get N-[2-[4-amino-3-(3-fluoro-5-hydroxy-phenyl)pyrazolo [3,4-d]pyrimidin-1-yl]ethyl]-N-benzyl-cyclopropanecarboxamide (28.5 mg). This was dissolved ethanolic HCl (5 mL) and then dried to get N-[2-[4-amino-3-(3-fluoro-5-hydroxy-phenyl)pyrazolo [3,4-d]pyrimidin-1-yl]ethyl]-N-benzyl-cyclopropanecarboxamide as the HCl salt (29.1 mg). ¹H NMR (400 MHz, Methanol-d4) δ (ppm): 8.41 (s, 1H), 7.32-7.10 (m, 5H), 6.98-6.82 (m, 2H), 6.73-6.64 (m, 1H), 4.78 (s, 2H), 4.68 (t, J=5.4 Hz, 1H), 4.58 (t, J=5.4 Hz, 1H), 4.12 (t, J=5.5 Hz, 1H), 3.96 (t, J=5.4 Hz, 1H), 1.83 (m, 1H), 0.79-0.61 (m, 4H). LCMS: 447 (M+1).

### Example 9

### Preparation of Compound No. 9

### Synthesis of 3-{4-amino-1-[2-(N-benzylprop-2-enamido)ethyl]-1H-pyrazolo [3,4-d]pyrimidin-3-yl}-5-fluorophenyl acetate

To a stirred solution of 3-N-[2-[4-amino-3-(3-fluoro-5-hydroxy-phenyl)pyrazolo [3,4-d]pyrimidin-1-yl]ethyl]-N-benzyl-prop-2-enamide (200 mg, 0.461 mmol) in DCM (8 mL) was added triethylamine (47 mg, 0.461 mmol) at 0° C. under nitrogen atmosphere and the resultant reaction mixture was stirred for 5 min then to this was added acetyl bromide (57 mg, 0.461 mmol). The reaction mixture was stirred at for 5 min. The progress of reaction was monitored by TLC and LCMS. After completion of reaction, the reaction mixture was diluted with water (10 mL) and the product was extracted with DCM (2×25 mL). The combined DCM layer was washed with water (2×25 mL) and brine solution (25 mL), dried over anhydrous sodium sulfate and concentrated under vacuum to get a crude product which was purified using reverse phase HPLC to get [3-[4-amino-1-[2-[benzyl(prop-2-enoyl)amino]ethyl]pyrazolo [3,4-d]pyrimidin-3-yl]-5-fluoro-phenyl]acetate (60.10 mg). ¹H NMR (400 MHz, Chloroform-d) δ (ppm): 8.37 (s, 1H), 7.38-7.27 (m, 5H), 7.25-7.18 (m, 2H), 7.11-6.83 (m, 2H), 6.46 (td, J=16.6, 10.2 Hz, 1H), 6.40-6.17 (m, 1H), 5.78 (d, J=12.2 Hz, 2H), 5.60 (ddd, J=29.8, 10.3, 2.2 Hz, 1H), 4.81-4.64 (m, 2H), 4.59 (t, J=6.3 Hz, 1H), 4.40 (s, 1H), 3.98 (t, J=5.9 Hz, 1H), 3.85 (t, J=6.4 Hz, 1H), 2.36 (s, 3H). LCMS: 475 (M+1).

### Example 10

### Preparation of Compound No. 10

### Synthesis of N-{2-[4-amino-3-(3-fluoro-5-hydroxyphenyl)-1H-pyrazolo [3,4-d]pyrimidin-1-yl]ethyl}-N-[(4-fluorophenyl)methyl]prop-2-enamide

To a stirred solution of (3-[4-amino-1-[2-[(fluorophenyl)methylamino]ethyl]pyrazolo[3,4-d]pyrimidin-3-yl]-5-fluoro-phenol (250 mg, 0.63 mmol) in DCM (9 mL) and DMF (1 mL) was added triethylamine (63 mg, 0.63 mmol) at 0° C. and reaction allowed to stir at 0° C. for 1 minute. After 1 min. acryloyl chloride (57 mg, 0.63 mmol) was added at 0° C. and reaction allowed to stir at 0° C. for 2 min. The reaction was monitored by LCMS. On completion of reaction, water (5 mL) was added to the reaction mixture and the mixture extracted by addition of more of DCM. The organic layer was dried over sodium sulfate and concentrated under reduced pressure to give a crude compound. The crude compound was purified by preparative HPLC giving N-[2-[4-amino-3-(3-fluoro-5-hydroxy-phenyl)pyrazolo[3,4-d]pyrimidin-1-yl]ethyl]-N-[(4-fluorophenyl)methyl]prop-2-enamide (27.1 mg). The pure compound was dissolved in THF (2 mL) and methanesulfonic acid (11 mg, 2 equivalent) was added. The reaction was allowed to stir at RT for 1 h. The reaction mixture was concentrated under reduced pressure and triturated with diethyl ether to give pure product (28.2 mg) as the dimesylate salt, as an off white solid. ¹H NMR (400 MHz, Methanol-d4) δ (ppm): 8.41 (d, J=7.4 Hz, 1H), 7.27 (m, 1H), 7.17 (t, J=6.9 Hz, 1H), 7.01 (m, 2H), 6.96-6.80 (m, 2H), 6.79-6.58 (m, 1H), 6.46 (dd, J=16.6, 10.5 Hz, 1H), 6.14 (d, J=15.7 Hz, 1H), 6.04 (d, J=16.4 Hz, 1H), 5.69 (d, J=10.5 Hz, 1H), 5.53 (d, J=10.2 Hz, 1H), 4.71 (t, J=5.5 Hz, 2H), 4.66 (s, 2H), 4.00 (m, 2H), 2.74 (s, 6H).

### Example 11

### Preparation of Compound No. 11

### Synthesis of N-{2-[4-amino-3-(3-fluoro-5-hydroxyphenyl)-1H-pyrazolo[3,4-d]pyrimidin-1-yl]ethyl}-N-[(2-fluorophenyl)methyl]prop-2-enamide

### Step 1: Synthesis of 2-[(2-fluorophenyl)methylamino]ethanol

A stirred solution of 2-aminoethanol (10 g, 0.08 mol) and 2-fluorobenzaldehyde (5.9 g, 0.096 mol) in methanol (80 mL) was allowed to stir for 45 min at RT. After 45 min sodium borohydride (1.824 g, 0.128 mol) was added and allowed to stir for 15 min. After 15 min, the reaction mixture was quenched with ice cold water (150 mL) followed by extraction with EtOAc (3×100 mL). The combined organic layer was dried over anhydrous sodium sulfate, and concentrated under reduced pressure to give 2-[(2-fluorophenyl)methylamino]ethanol (10 g).

### Step 2: Synthesis of tert-butyl N-[(2-fluorophenyl)methyl]-N-(2-hydroxyethyl)carbamate

To a stirred solution of 2-[(2-fluorophenyl)methylamino]ethanol (10 g, 0.06 mol) in THF (40 mL) was added a sodium carbonate (12.72 g, 0.12 mol) solution in water (40 mL), followed by addition of BOC anhydride (15.28 mL, 0.066 mol). After 3 h stirring at RT, the reaction mixture was quenched with water (200 mL) followed by extraction with EtOAc (1×600 mL). The organic layer was washed with water (3×500 mL) dried over anhydrous sodium sulfate, and concentrated under reduced pressure to give tert-butyl N-[(2-fluorophenyl)methyl]-N-(2-hydroxyethyl)carbamate (16 g).

### Step 3: Synthesis of 2-[tert-butoxycarbonyl-[(2-fluorophenyl)methyl]amino]ethyl methanesulfonate

To a stirred solution of tert-butyl N-[(2-fluorophenyl)methyl]-N-(2-hydroxyethyl)carbamate (5 g, 0.018 mol) in DCM (20 mL) at 0° C. was added TEA (10.34 mL, 0.072 mol) and allowed to stir for 15 min. Mesyl chloride was added to it dropwise. The reaction mixture was brought to RT. The progress of reaction was followed by TLC. After 3 h, the reaction mixture was quenched with cold water (50 mL) followed by extraction with EtOAc (3×100 mL). The combined organic layer was dried over anhydrous sodium sulfate, and concentrated under reduced pressure to 2-[tert-butoxycarbonyl-[(2-fluorophenyl)methyl]amino]ethyl methanesulfonate (5.8 g).

Step 4: Synthesis of tert-butyl N-[2-(4-amino-3-iodo-pyrazolo[3,4-d]pyrimidin-1-yl)ethyl]N-[(2-fluorophenyl)methyl]carbamate

To a stirred solution of 3-iodo-1H-pyrazolo[3,4-d]pyrimidin-4-amine (100 mg, 0.38 mmol)) in DMF (2.5 mL) was added cesium carbonate (248 mg, 0.76 mmol) and stirred for 45 min at RT. After 45 min, 2-[tert-butoxycarbonyl-[(2-fluorophenyl)methyl]amino]ethyl methanesulfonate (265 mg, 0.76 mmol) in DMF (2.5 mL) was added followed by TBAI (29 mg, 0.76 mmol). The reaction mixture was stirred for 2 h at 100° C. After completion of reaction, the mixture was quenched with water (50 mL), followed by extraction with EtOAc (3×30 mL). The combined organic layers were washed with brine (100 mL) dried over anhydrous sodium sulfate, concentrated under reduced pressure to give a crude product which was purified over silica gel (100-200#) using 90% EtOAc/Hexane as eluent to give tert-butyl N-[2-(4-amino-3-iodo-pyrazolo[3,4-d]pyrimidin-1-yl)ethyl]-N-[(2-fluorophenyl)methyl]carbamate (965 mg).

### Step 5: Synthesis of tert-butyl N-[2-[4-amino-3-(3-fluoro-5-hydroxy-phenyl)pyrazolo[3,4-d]pyrimidin-1-yl]ethyl]-N-[(2-fluorophenyl)methyl]carbamate

To a stirred solution of tert-butyl N-[2-(4-amino-3-iodo-pyrazolo[3,4-d]pyrimidin-1-yl)ethyl]-N-[(2-fluorophenyl)methyl]carbamate (865 mg, 1.689 mmol) and (3-fluoro-5-hydroxy-phenyl)boronic acid (342 mg, 2.19 mmol) in DMF (8 mL) was added a solution of sodium carbonate (537 mg, 5.067 mmol) in water (8 mL) and Pd(PPh₃)₄ (195 mg, 0.1689 mmol) at RT. The reaction mixture was heated at 110° C. for 5 h. After completion of reaction, the mixture was allowed to cool to RT followed by quenching with water (70 mL), and extraction with EtOAc (3×50 mL). The combined organic layers were washed with brine (150 mL) dried over anhydrous sodium sulfate, then concentrated under reduced pressure to give a crude product, which was purified over silica (100-200#) using 70% EtoAc/Hexane as eluent to give tert-butyl N-[2-[4-amino-3-(3-fluoro-5-hydroxy-phenyl)pyrazolo[3,4-d]pyrimidin-1-yl]ethyl]-N-[(2-fluorophenyl)methyl]carbamate (865 mg).

### Step 6: Synthesis of 3-[4-amino-1-[2-[(2-fluorophenyl)methylamino]ethyl]pyrazolo[3,4-d]pyrimidin-3-yl]-5-fluoro-phenol

To a stirred solution of tert-butyl N-[2-[4-amino-3-(3-fluoro-5-hydroxy-phenyl)pyrazolo[3,4-d]pyrimidin-1-yl]ethyl]-N-[(2-fluorophenyl)methyl]carbamate (865 mg) in DCM (9 mL) was added TFA (3 mL) at 0° C. After addition, the reaction mixture was brought to RT. The progress of reaction was monitored by TLC. After completion of reaction, the mixture was concentrated under reduced pressure to give 3-[4-amino-1-[2-[(2-fluorophenyl)methylamino]ethyl]pyrazolo[3,4-d]pyrimidin-3-yl]-5-fluoro-phenol (550 mg).

### Step 7: Synthesis of N-[2-[4-amino-3-(3-fluoro-5-hydroxy-phenyl)pyrazolo[3,4-d]pyrimidin-1-yl]ethyl]-N-[(2-fluorophenyl)methyl]prop-2-enamide

To a stirred solution of 3-[4-amino-1-[2-[(2-fluorophenyl)methylamino]ethyl]pyrazolo[3,4-d]pyrimidin-3-yl]-5-fluoro-phenol (200 mg, 0.32 mmol) in DCM (9 mL) was added acryloyl chloride (29 mg 0.32 mL) at 0° C. and allowed to stir for 10 min. After completion of reaction, the mixture was quenched with ice cold water (50 mL), and extracted with EtOAc (3×30 mL). The combined organic layers were dried over anhydrous sodium sulfate, and concentrated under reduced pressure to give a crude product, which was purified by reverse phase HPLC to give N-[2-[4-amino-3-(3-fluoro-5-hydroxy-phenyl)pyrazolo[3,4-d]pyrimidin-1-yl]ethyl]-N-[(2-fluorophenyl)methyl]prop-2-enamide (47 mg). N-[2-[4-amino-3-(3-fluoro-5-hydroxy-phenyl)pyrazolo[3,4-d]pyrimidin-1-yl]ethyl]-N-[(2-fluorophenyl) methyl]prop-2-enamide (35 mg) was dissolved in THF (2 mL) followed by addition of methanesulfonic acid (15 mg) and allowed to stir for 1 h. The reaction mixture was concentrated under reduced pressure and washed with diethyl ether to give N-[2-[4-amino-3-(3-fluoro-5-hydroxy-phenyl)pyrazolo[3,4-d]pyrimidin-1-yl]ethyl]-N-[(2-fluorophenyl)methyl]prop-2-enamide mesylate salt (32 mg). ¹H NMR (400 MHz, Methanol-d4) δ (ppm): 8.41 (d, J=12.8 Hz, 1H), 7.28 (q, J=11.0, 8.9 Hz, 1H), 7.09 (td, J=20.5, 8.9 Hz, 2H), 6.96-6.83 (m, 2H), 6.78-6.64 (m, 2H), 6.15-5.98 (m, 1H), 5.70-5.50 (m, 1H), 4.72 (d, J=9.5 Hz, 4H), 4.06 (s, 1H), 3.99 (t, J=5.4 Hz, 1H), 2.71 (s, 3H). LCMS (M+1): 467.

### Example 12

### Preparation of Compound No. 12

### Synthesis of N-{2-[4-amino-3-(3-fluoro-5-hydroxyphenyl)-1H-pyrazolo[3,4-d]pyrimidin-1-yl]ethyl}-N-[(4-chlorophenyl)methyl]prop-2-enamide

To a stirred solution of 3-[4-amino-1-[2-[(4-chlorophenyl)methylamino]ethyl]pyrazolo[3,4-d]pyrimidin-3-yl]-5-fluoro-phenol (164 mg, 0.397 mmol) in DCM (5 mL) was cooled to 0° C. and to it was added acryloyl chloride (39.5 mg, 0.437 mmol) dropwise and stirred for 15 min. After 15 min, the reaction mixture was quenched with ice cold water (20 mL), and extracted with EtOAc (3×15 mL). The combined organic layer was washed with brine (60 mL), dried over anhydrous sodium sulfate, and concentrated under reduced pressure to give a crude product, which was purified by reverse HPLC to give N-[2-[4-amino-3-(3-fluoro-5-hydroxy-phenyl)pyrazolo[3,4-d]pyrimidin-1-yl]ethyl]-N-[(4-chlorophenyl)methyl]prop-2-enamide (35 mg). This compound was dissolved in THF followed by addition of methanesulfonic acid (15 mg) and allowed to stir for 1 h. The reaction mixture was concentrated under reduced pressure and washed with ether to give N-[2-[4-amino-3-(3-fluoro-5-hydroxy-phenyl)pyrazolo[3,4-d]pyrimidin-1-yl]ethyl]-N-[(4-chlorophenyl)methyl]prop-2-enamide bis mesylate salt (30 mg). ¹H NMR (400 MHz, Methanol-d4) δ (ppm): 8.40 (d, J=4.1 Hz, 1H), 7.26 (d, J=8.3 Hz, 2H), 7.20 (d, J=8.1 Hz, 1H), 7.11 (d, J=8.1 Hz, 1H), 6.95-6.85 (m, 2H), 6.78-6.67 (m, 1H), 6.67-6.50 (m, 1H), 6.16-6.07 (m, 1H), 5.70-5.56 (m, 1H), 4.71 (s, 2H), 4.66 (s, 2H), 4.10-3.98 (m, 2H), 2.70 (s, 6H).

### Example 13

### Preparation of Compound No. 13

### Synthesis of N-{2-[4-amino-5-(4-chloro-3-hydroxyphenyl)-7H-pyrrolo[2,3-d]pyrimidin-7-yl]ethyl}-N-[(4-fluorophenyl)methyl]prop-2-enamide

### Step-1: Synthesis of tert-butyl N-[2-(4-amino-5-bromo-pyrrolo[2,3-d]pyrimidin-7-yl)ethyl]carbamate

Cs₂CO₃ (8.75 g, 26.855 mmol) was added to a solution of 5-bromo-7H-pyrrolo[2,3-d]pyrimidin-4-amine (2.6 g, 12.206 mmol) in DMF (25 mL). The reaction was stirred at RT for 1 h, followed by addition of TBAI (902 mg, 2.441 mmol) and 2-(tert-butoxycarbonyl amino)ethyl methanesulfonate (6.42 g, 26.830 mmol). The reaction was heated at 70° C. for 3 h, monitored by TLC. The mixture was diluted with water (100 mL) and extracted with EtOAc (3×120 mL). The combined organic layer was washed with water (2×300 mL) and brine (100 mL), dried over anhydrous sodium sulfate and concentrated under reduced pressure to afford a crude product, which was purified by column chromatography (silica-gel, 100-200 mesh) using 2% MeOH in DCM as eluent to obtain 1.2 g of tert-butyl N-[2-(4-amino-5-bromo-pyrrolo[2,3-d]pyrimidin-7-yl)ethyl]carbamate, as an off-white solid.

### Step-2: Synthesis of tert-butyl N-[2-[4-amino-5-(4-chloro-3-hydroxy-phenyl)pyrrolo[2,3-d]pyrimidin-7-yl]ethyl]carbamate

A solution of Na₂CO₃ (491 mg, 4.633 mmol) in water (10 mL) was added to a solution of (4-chloro-3-hydroxy-phenyl)boronic acid (402 mg, 2.318 mmol) in DMF (10 mL). The reaction mixture was purged with nitrogen for 10 minutes, followed by addition of tert-butyl N-[2-(4-amino-5-bromo-pyrrolo[2,3-d]pyrimidin-7-yl)ethyl]carbamate (550 mg, 1.543 mmol) and Pd(PPh₃)₄ (107 mg, 0.092 mmol) and again purged for 2 minutes. The reaction was heated at 70° C. for 3 h, monitored by TLC. The reaction was diluted with water (100 mL) and extracted with EtOAc (2×120 mL). The combined organic layer was washed with water (2×200 mL) and brine (60 mL), dried over anhydrous sodium sulfate and concentrated under reduced pressure to afford a crude product, which was purified by column chromatography (silica-gel, 100-200 mesh) using 3.5% MeOH in DCM as eluent to obtain 300 mg of tert-butyl N-[2-[4-amino-5-(4-chloro-3-hydroxy-phenyl)pyrrolo[2,3-d]pyrimidin-7-yl]ethyl]carbamate as an off-white solid.

### Step-3: Synthesis of tert-butyl 5-[4-amino-7-(2-aminoethyl)pyrrolo[2,3-d]pyrimidin-5-yl]-2-chloro-phenol

TFA (3 mL) was added dropwise at 0° C. to a solution of tert-butyl N-[2-[4-amino-5-(4-chloro-3-hydroxy-phenyl)pyrrolo[2,3-d]pyrimidin-7-yl]ethyl]carbamate (350 mg, 0.755 mmol) in DCM (9 mL). The reaction was allowed to warm to RT and stirred for 90 min, monitored by TLC. The reaction was concentrated under reduced pressure to obtain a solid residue, which was dissolved in satd. aq. NaHCO₃ (50 mL) and extracted with EtOAc (4×60 mL). The organic layer was concentrated under reduced pressure and washed with DCM (10 mL) to obtain 250 mg of 5-[4-amino-7-(2-aminoethyl)pyrrolo[2,3-d]pyrimidin-5-yl]-2-chloro-phenol as a yellow solid.

### Step-4: Synthesis of 5-[4-amino-7-[2-[(4-fluorophenyl)methylamino]ethyl]pyrrolo[2,3-d]pyrimidin-5-yl]-2-chloro-phenol

A solution of 5-[4-amino-7-(2-aminoethyl)pyrrolo[2,3-d]pyrimidin-5-yl]-2-chloro-phenol (400 mg, 1.316 mmol) and 4-fluorobenzaldehyde (163 mg, 1.313 mmol) in acetonitrile (60 mL) was stirred at RT for 1 h. To this was added sodium triacetoxy borohydride (560 mg, 2.641 mmol) and glacial acetic acid (2 mL). The reaction was stirred at RT overnight, monitored by TLC. The reaction was concentrated under reduced pressure to obtain a solid residue that was dissolved in aq. saturated NaHCO₃ solution (100 mL) and extracted with EtOAc (12×25 mL). The organic layer was dried over anhydrous sodium sulfate and concentrated under reduced pressure to afford a crude product, which was purified by column chromatography (silica-gel, 100-200 mesh) using 5% MeOH in DCM as eluent to obtain 160 mg of 5-[4-amino-7-[2-[(4-fluorophenyl)methylamino]ethyl]pyrrolo[2,3-d]pyrimidin-5-yl]-2-chloro-phenol as an off-white solid.

### Step-5: Synthesis of N-[2-[4-amino-5-(4-chloro-3-hydroxy-phenyl)pyrrolo[2,3-d]pyrimidin-7-yl]ethyl]-N-[(4-fluorophenyl)methyl]prop-2-enamide

Acryloyl chloride (39 mg, 0.430 mmol) in DCM (1 mL) was added to a solution of 5-[4-amino-7-[2-[(4-fluorophenyl)methylamino]ethyl]pyrrolo[2,3-d]pyrimidin-5-yl]-2-chloro-phenol (160 mg, 0.388 mmol) in DCM (7 mL) at 0° C. The progress of the reaction was monitored by TLC. The reaction was quenched with water (20 mL) and extracted with EtOAc (2×30 mL). The organic layer was washed with brine (20 mL) dried over anhydrous sodium sulfate and concentrated under reduced pressure to afford a crude product, which was purified by reverse phase preparative HPLC to obtain N-[2-[4-amino-5-(4-chloro-3-hydroxy-phenyl)pyrrolo[2,3-d]pyrimidin-7-yl]ethyl]-N-[(4-fluorophenyl)methyl]prop-2-enamide (18 mg) as a white solid, treated with methanesulfonic acid (4 mg) in THF to obtain N-[2-[4-amino-5-(4-chloro-3-hydroxy-phenyl)pyrrolo[2,3-d]pyrimidin-7-yl]ethyl]-N-[(4-fluorophenyl)methyl]prop-2-enamide mesylate salt (19 mg) (a white solid). ¹H NMR (400 MHz, Methanol-d4) δ (ppm): 8.31 (d, J=17.9 Hz, 1H), 7.44 (m, 2H), 7.29 (d, J=6.1 Hz, 1H), 7.18-7.11 (m, 1H), 7.03 (m, 3H), 6.93 (d, J=8.4 Hz, 1H), 6.69 (dd, J=16.6, 10.4 Hz, 0.5H), 6.37 (dd, J=16.6, 10.5 Hz, 0.5H), 6.16 (d, J=16.1 Hz, 0.5H), 6.12 (d, J=16.1 Hz, 0.5H), 5.70 (d, J=10.4 Hz, 0.5H), 5.48 (d, J=10.5 Hz, 0.5H), 4.65 (d, J=17.0 Hz, 2H), 4.55 (t, J=5.8 Hz, 2H), 3.90 (t, J=5.7 Hz, 2H), 2.70 (s, 3H). LCMS−(M+1): 393.7.

### Example 14

### Preparation of Compound No. 14

### Synthesis of N-{2-[4-amino-3-(4-chloro-3-hydroxyphenyl)-1H-pyrazolo[3,4-d]pyrimidin-1-yl]ethyl}-N-[(4-fluorophenyl)methyl]prop-2-enamide

To a stirred solution of N-(2-(4-amino-3-(4-chloro-3-hydroxyphenyl)-1H-pyrazolo[3,4-d]pyrimidin-1-yl)ethyl)-N-(4-fluorobenzyl)acrylamide (100 mg, 0.24 mmol) in DCM (4 mL) was slowly added acryloyl chloride (25 mg, 0.264 mmol) dissolved in DCM (1 mL) at 0° C. The reaction mixture was allowed to stir at 0° C. for 5 min. The reaction was monitored by TLC. After completion, the reaction was quenched with ice-cold water and extracted with EtOAc (70 mL). The organic layer was washed with water (30 mL), brine (30 mL), dried over sodium sulfate and concentrated. The crude compound was purified by reverse phase HPLC to obtain N-(2-(4-amino-3-(4-chloro-3-hydroxyphenyl)-1H-pyrazolo[3,4-d]pyrimidin-1-yl)ethyl)-N-(4-fluorobenzyl)acrylamide (12 mg). The compound obtained was dissolved in THF (8 mL) and to it was added 2 eq. of methanesulfonic acid and stirred for 1 h at RT. The reaction mixture was then concentrated under reduced pressure and triturated with pentane (2 mL), ether (2 mL) to obtain N-(2-(4-amino-3-(4-chloro-3-hydroxyphenyl)-1H-pyrazolo[3,4-d]pyrimidin-1-yl)ethyl)-N-(4-fluorobenzyl) acrylamide (16 mg) as the mesylate salt (an off-white solid). ¹H NMR (400 MHz, Methanol-d4) δ (ppm): 8.40 (d, J=7.1 Hz, 1H), 7.53 (t, J=7.5 Hz, 1H), 7.24 (d, J=15.5 Hz, 2H), 7.13 (dd, J=17.6, 10.7 Hz, 2H), 7.01 (t, J=8.6 Hz, 2H), 6.66-6.45 (m, 1H), 6.45-6.13 (m, 1H), 5.68-4.71 (m, 1H), 4.71 (t, J=5.5 Hz, 2H), 4.66 (s, 2H), 4.10-3.93 (m, 2H).

### Example 15

### Preparation of Compound No. 15

### Synthesis of N-{2-[4-amino-3-(4-chloro-3-hydroxyphenyl)-1H-pyrazolo[3,4-d]pyrimidin-1-yl]ethyl}-N-[(4-chlorophenyl)methyl]prop-2-enamide

5-(4-Amino-1-(2-(4-chlorobenzylamino) ethyl)-1H-pyrazolo[3,4-d]pyrimidin-3-yl)-2-chlorophenol (100 mg, 0.23 mmol) was charged in DCM (10 mL). The reaction mixture was cooled to 0° C., then a solution of acryloyl chloride (21 mg, 0.23 mmol) in DCM (1 mL) was added dropwise with constant stirring. The reaction mixture was stirred for 5 min at the same temperature and monitored by TLC and LC-MS. Ice water (10 mL) was added and the mixture extracted with EtOAc (3×100 mL). The combined organic layer was dried over anhydrous sodium sulfate and concentrated under reduced pressure to get a crude product. The crude product was purified through reverse phase HPLC to obtain N-(2-(4-amino-3-(4-chloro-3-hydroxyphenyl)-1H-pyrazolo[3,4-d]pyrimidin-1-yl)ethyl)-N-(4-chlorobenzyl) acrylamide (20 mg) as the free base. The compound obtained was dissolved in THF (10 mL) and added 2 eq. of methanesulfonic acid and stirred for 1 h at RT. The reaction mixture was then concentrated under reduced pressure and triturated with pentane (2 mL), ether (2 mL) to obtain N-(2-(4-amino-3-(4-chloro-3-hydroxyphenyl)-1H-pyrazolo[3,4-d]pyrimidin-1-yl)ethyl)-N-(4-chlorobenzyl) acrylamide (22 mg) as the mesylate salt (an off-white solid). ¹H NMR (400 MHz, Methanol-d4) δ (ppm): 8.40 (d, J=5.3 Hz, 1H), 7.60-7.48 (m, 1H), 7.30-7.16 (m, 4H), 7.11 (t, J=8.2 Hz, 2H), 6.65-6.50 (m, 1H), 6.16-6.06 (m, 1H), 5.70-5.56 (m, 1H), 4.71 (d, J=5.3 Hz, 2H), 4.67 (d, J=14.0 Hz, 2H), 4.04 (dt, J=14.4, 5.3 Hz, 2H).

### Example 16

### Preparation of Compound No. 16

### Synthesis of N-{2-[4-amino-3-(3,4-difluoro-5-hydroxyphenyl)-1H-pyrazolo[3,4-d]pyrimidin-1-yl]ethyl}-N-[(4-chlorophenyl)methyl]prop-2-enamide

### Step-1: Synthesis of 1-(2-aminoethyl)-3-iodo-pyrazolo[3,4-d]pyrimidin-4-amine

Tert-butyl N-[2-(4-amino-3-iodo-pyrazolo[3,4-d]pyrimidin-1-yl)ethyl]carbamate (1 g, 2.46 mmol, 1 eq.) was suspended in 30 mL of DCM then at 0° C. TFA (10 mL) was added dropwise and the mixture stirred for 15 min at 0° C. then at RT for 2 h. The reaction was monitored by TLC and ¹H-NMR. The mixture was evaporated, then the residue dissolved in 50 mL of ethanol and solid sodium carbonate added (up to pH=7-8). The mixture was stirred for 30 min, then filtered, and the ethanol layer evaporated to give the desired compound (700 mg) which was used as such for the next step of synthesis.

### Step-2: Synthesis of 1-[2-[(4-chlorophenyl)methylamino]ethyl]-3-iodo-pyrazolo[3,4-d]pyrimidin-4-amine

1-(2-Aminoethyl)-3-iodo-pyrazolo[3,4-d]pyrimidin-4-amine (500 mg, 1.64 mmol, 1 eq.) and 4-chloro benzaldehyde (210 mg, 1 eq.) were stirred in 30 mL of methanol at RT for 1 h. The mixture was cooled to 0° C. and NaBH₃CN (207 mg, 2 eq.) and acetic acid (1.47 g, 15 eq.) were added and the mixture stirred overnight. The reaction was monitored by TLC and LCMS. The methanol was evaporated under reduced pressure and the residue extracted with EtOAc (3×100 mL), then dried over anhydrous sodium sulfate, and concentrated under reduced pressure. The crude product was purified by silica gel (100-200) column chromatography by using MeOH/DCM (0-10%) as eluent system to get pure compound (155 mg) as a solid.

### Step-3: Synthesis of N-[2-(4-amino-3-iodo-pyrazolo[3,4-d]pyrimidin-1-yl)ethyl]-N-[(4-chlorophenyl)methyl]prop-2-enamide

1-[2-[(4-Chlorophenyl)methylamino]ethyl]-3-iodo-pyrazolo[3,4-d]pyrimidin-4-amine (155 mg, 0.362 mmol, 1 eq.) was suspended in 10 mL of DCM maintained at 0° C., then acryloyl chloride (32.5 mg, 1 eq.) was added and the mixture stirred for 10 min at 0° C. The reaction was monitored by TLC and LCMS. The reaction was quenched by 50 mL of chilled water and 5 mL of saturated NaHCO₃ solution, then extracted with DCM (3×100 mL), and dried by anhydrous sodium sulfate then concentrated under reduced pressure to give the product as a viscous solid (150 mg), which was directly used as such for the next step of synthesis.

### Step-4: Synthesis of N-[2-[4-amino-3-(3,4-difluoro-5-hydroxy-phenyl)pyrazolo[3,4-d]pyrimidin-1-yl]ethyl]-N-[(4-chlorophenyl)methyl]prop-2-enamide

N-[2-(4-Amino-3-iodo-pyrazolo[3,4-d]pyrimidin-1-yl)ethyl]-N-[(4-chlorophenyl)methyl]prop-2-enamide (150 mg, 0.31 mmol, 1 eq.) along with (3,4-difluoro-5-hydroxy-phenyl)boronic acid (81 mg, 1.5 eq.) and sodium carbonate (82 mg, 2.5 eq. in 1 mL water) were charged in 8 mL of acetonitrile in a 25 mL glass bottle, then Pd(PPh₃)₄ (35 mg, 0.1 eq.) was added, and the mixture heated to 100° C. for 2 h. The reaction progression was monitored by LCMS. The reaction mixture was allowed to come at RT; water (100 mL) was added, and then extracted with EtOAc (3×100 mL). The combined organic layer was washed with brine (50 mL), dried over anhydrous sodium sulfate and concentrated under reduced pressure to obtain a crude product, which was purified by reverse phase HPLC to afford N-[2-[4-amino-3-(3,4-difluoro-5-hydroxy-phenyl)pyrazolo[3,4-d]pyrimidin-1-yl]ethyl]-N-[(4-chlorophenyl)methyl]prop-2-enamide (13 mg) as a white solid. ¹H NMR (400 MHz, Methanol-d4) δ (ppm): 8.24 (d, J=13.1 Hz, 1H), 7.28 (d, J=8.3 Hz, 1H), 7.22 (d, J=7.5 Hz, 2H), 7.10-6.97 (m, 3H), 6.58 (dd, J=16.7-6.37 (m, J=16.6, 10.5 Hz, 1H), 6.15-5.98 (m, J=16.3 Hz, 1H), 5.66-5.45 (m, J=10.6 Hz, 1H), 4.66 (s, 2H), 4.60 (t, J=14.6, 8.8 Hz, 2H), 4.54 (s, 2H), 3.94 (t, J=5.6 Hz, 2H). 485 (M+1).

### Example 17

### Preparation of Compound No. 17

### Synthesis of N-{2-[4-amino-3-(4-chloro-3-hydroxyphenyl)-1H-pyrazolo[3,4-d]pyrimidin-1-yl]ethyl}-N-[(4-methoxyphenyl)methyl]prop-2-enamide

To a stirred solution of 5-[4-amino-1-[2-[(4-methoxyphenyl) methylamino]ethyl]pyrazolo[3,4-d]pyrimidin-3-yl]-2-chloro-phenol (100 mg, 0.24 mmol) in DCM (5 mL) was added acryloyl chloride (21.3 mg, 0.24 mmol) at 0° C. and the reaction mixture was allowed to stir for 5 min. After completion of reaction, the reaction mixture was quenched with ice cold water (15 mL), and extracted with EtOAc (3×70 mL). The combined organic layers were washed with brine (70 mL), and concentrated under reduced pressure to give a crude product, which was purified by reverse HPLC to give N-[2-[4-amino-3-(4-chloro-3-hydroxy-phenyl)pyrazolo[3,4-d]pyrimidin-1-yl]ethyl]-N-[(4-methoxyphenyl)methyl]prop-2-enamide (36 mg). The product was mixed with methanesulfonic acid and stirred for 1 h to give N-[2-[4-amino-3-(4-chloro-3-hydroxy-phenyl)pyrazolo[3,4-d]pyrimidin-1-yl]ethyl]-N-[(4-methoxyphenyl) methyl]prop-2-enamide (40 mg) as the mesylate salt (a brown solid). ¹H NMR (400 MHz, Methanol-d4) δ (ppm): 8.39 (d, J=9.6 Hz, 1H), 7.58-7.46 (m, 1H), 7.22 (s, 1H), 7.18-7.07 (m, 2H), 7.02 (d, J=8.3 Hz, 1H), 6.79 (t, J=9.9 Hz, 3H), 6.74-6.45 (m, 1H), 6.16-6.06 (m, 1H), 5.70-5.51 (m, 1H), 4.70 (s, 2H), 4.58 (d, J=13.7 Hz, 2H), 3.99 (s, 2H), 3.74 (d, J=6.6 Hz, 3H), 2.71 (s, 9H).

### Example 18

### Preparation of Compound No. 18

### Synthesis of N-{2-[4-amino-3-(8-fluoro-3,4-dihydro-2H-1,4-benzoxazin-6-yl)-1H-pyrazolo [3,4-d]pyrimidin-1-yl]ethyl}-N-[(4-chlorophenyl)methyl]prop-2-enamide

### Step-1: Synthesis of 1-(2-aminoethyl)-3-iodo-pyrazolo[3,4-d]pyrimidin-4-amine

tert-butyl N-[2-(4-Amino-3-iodo-pyrazolo[3,4-d]pyrimidin-1-yl)ethyl]carbamate (1g, 2.46 mmol, 1 eq.) was suspended in 30 mL of DCM at 0° C., and TFA (10 mL) was added dropwise, and the mixture stirred for 15 min at 0° C. then at RT for 2 h. The reaction was monitored by TLC and ¹H-NMR. Upon completion, the DCM was evaporated, and the residue dissolved in 50 mL of ethanol and solid sodium carbonate added (up to pH=7-8). The mixture was stirred for 30 min, then filtered and the ethanol evaporated to give the desired compound (700 mg) which was used as such for the next step of synthesis.

### Step-2: Synthesis of 1-[2-[(4-chlorophenyl)methylamino]ethyl]-3-iodo-pyrazolo[3,4-d]pyrimidin-4-amine

1-(2-Aminoethyl)-3-iodo-pyrazolo[3,4-d]pyrimidin-4-amine (500 mg, 1.64 mmol, 1 eq.) and 4-chloro benzaldehyde (210 mg, 1 eq.) were stirred in 30 mL of methanol at RT for 1 h. Then the mixture was cooled to 0° C., and NaBH₃CN (207 mg, 2 eq.) and acetic acid (1.47 g, 15 eq.) were added and the mixture stirred overnight. The reaction was monitored by TLC and LCMS. The methanol was evaporated under reduced pressure and the residue extracted with EtOAc (3×100 mL), then dried by anhydrous sodium sulfate, and concentrated under reduced pressure. The residue was purified by silica gel (100-200) column chromatography by using MeOH/DCM (0-10%) as eluent system to get pure compound (155 mg) as a solid product.

### Step-3: Synthesis of N-[2-(4-amino-3-iodo-pyrazolo[3,4-d]pyrimidin-1-yl)ethyl]-N-[(4-chlorophenyl)methyl]prop-2-enamide

1-[2-[(4-Chlorophenyl)methylamino]ethyl]-3-iodo-pyrazolo[3,4-d]pyrimidin-4-amine (155 mg, 0.362 mmol, 1 eq.) was suspended in 10 mL of DCM maintained at 0° C. then acryloyl chloride (32.5 mg, 1 eq.) was added and the mixture stirred for 10 min at 0° C. The reaction was monitored by TLC and LCMS. The reaction was quenched by 50 mL of chilled water and 5 mL of saturated NaHCO₃ solution, then extracted with DCM (3×100 mL), and dried by anhydrous sodium sulfate and concentrated under reduced pressure to give the product as a viscous solid (150 mg), which was directly used as such for the next step of synthesis.

### Step-4: Synthesis of N-[2-[4-amino-3-(8-fluoro-3,4-dihydro-2H-1,4-benzoxazin-6-yl)pyrazolo [3,4-d]pyrimidin-1-yl]ethyl]-N-[(4-chlorophenyl)methyl]prop-2-enamide

N-[2-(4-Amino-3-iodo-pyrazolo[3,4-d]pyrimidin-1-yl)ethyl]-N-[(4-chlorophenyl)methyl]prop-2-enamide (400 mg, 0.82 mmol, 1 eq.) along with 8-fluoro-6-(4,4,5,5-tetramethyl-1,3,2-dioxaborolan-2-yl)-3,4-dihydro-2H-1,4-benzoxazine (300 mg, 1.3 eq.) and sodium carbonate (219 mg, 2.5 eq. in 2 mL water) were charged in 8 mL of DMF in a 25 mL glass bottle, then Pd(PPh₃)₄ (95.7 mg, 0.1 eq.) were added, and the reaction mixture heated to 100° C. for 3 h. The reaction progression was monitored by LCMS. The reaction mixture was allowed to come RT, water (100 mL) was added, and the mixture extracted with EtOAc (3×100 mL). The combined organic layer was washed with brine (50 mL), dried over anhydrous sodium sulfate and concentrated under reduced pressure to obtain a crude product, which was purified by reverse phase HPLC to afford N-[2-[4-amino-3-(8-fluoro-3,4-dihydro-2H-1,4-benzoxazin-6-yl)pyrazolo[3,4-d]pyrimidin-1-yl]ethyl]-N-[4-chlorophenyl)methyl]prop-2-enamide (60 mg) as a white solid, as the free base. ¹H NMR (400 MHz, DMSO-d6) δ (ppm): 8.20 (d, J=7.7 Hz, 1H), 7.33 (m, 2H), 7.23 (d, J=8.1 Hz, 1H), 7.07 (d, J=8.1 Hz, 1H), 6.66 (s, 1H), 6.59 (dd, J=11.2, 2.2 Hz, 1H), 6.12 (d, J=16.3 Hz, 1H), 5.86 (d, J=16.4 Hz, 1H), 5.39 (d, J=10.7 Hz, 1H), 4.58 (s, 2H), 4.50 (dd, J=16.5, 10.8 Hz, 4H), 4.22 (t, J=4.3 Hz, 2H), 3.79 (d, J=5.5 Hz, 2H). 508 (M+1).

### Example 19

### Preparation of Compound No. 19

### Synthesis of N-{2-[4-amino-3-(4-chloro-3-hydroxyphenyl)-1H-pyrazolo[3,4-d]pyrimidin-1-yl]ethyl}-N-(thiophen-3-ylmethyl)prop-2-enamide

To a stirred solution of 5-(4-amino-1-(2-(thiophen-3-ylmethylamino)ethyl)-1H-pyrazolo[3,4-d]pyrimidin-3-yl)-2-chlorophenol (130 mg, 0.32 mmol) in DCM (15 mL) was added acryloyl chloride (30 mg, 0.32 mmol) dropwise at 0° C. and the resultant reaction mixture was allowed to stir for 10 min. The reaction was monitored by TLC. After completion, the reaction mixture was quenched with ice cold water (15 mL), and extracted with EtOAc (3×70 mL). The combined organic layers were washed with brine (70 mL), dried over sodium sulfate and concentrated under reduced pressure to give a crude product, which was purified by preparative HPLC to give N-(2-(4-amino-3-(4-chloro-3-hydroxyphenyl)-1H-pyrazolo[3,4-d]pyrimidin-1-yl)ethyl)-N-(thiophen-3-ylmethyl) acrylamide (13 mg). The product obtained was dissolved in THF (2 mL), methane sulfonic acid (3.51 mg, 0.036 mmol) was added, and the mixture stirred for 1 h. The reaction mixture was concentrated, washed with diethyl ether (2×2 mL) and lyophilized to give of N-(2-(4-amino-3-(4-chloro-3-hydroxyphenyl)-1H-pyrazolo[3,4-d]pyrimidin-1-yl)ethyl)-N-(thiophen-3-ylmethyl) acrylamide (15 mg) as the mesylate salt (an off-white solid). ¹H NMR (400 MHz, Methanol-d4) δ (ppm): 8.40 (d, J=8.8 Hz, 1H), 7.52 (t, J=7.6 Hz, 1H), 7.34 (q, J=5.9, 5.3 Hz, 1H), 7.28-7.20 (m, 1H), 7.17-7.08 (m, 2H), 6.99 (d, J=5.0 Hz, 1H), 6.91 (d, J=4.9 Hz, 1H), 6.70-6.42 (m, 1H), 6.17-5.99 (m, 1H), 5.69-5.52 (m, 1H), 4.74-4.62 (m, 4H), 4.01 (t, J=5.4 Hz, 2H), 2.71 (s, 3H).

### Example 20

### Preparation of Compound No. 20

### Synthesis of N-{2-[4-amino-3-(4-chloro-3-hydroxyphenyl)-1H-pyrazolo[3,4-d]pyrimidin-1-yl]ethyl}-N-(furan-2-ylmethyl)prop-2-enamide

To a stirred solution of 5-(4-amino-1-(2-(furan-2-ylmethylamino)ethyl)-1H-pyrazolo[3,4-d]pyrimidin-3-yl)-2-chlorophenol (120 mg, 0.311 mmol) in DCM (5 mL) was added acryloyl chloride (28.2 mg, 0.311 mmol) dropwise at 0° C. and the resultant reaction mixture was allowed to stir for 10 min. The reaction was monitored by TLC. After completion, the reaction mixture was quenched with ice cold water (15 mL), extracted with EtOAc (3×70 mL). The combined organic layers were washed with brine (70 mL), dried over sodium sulfate and concentrated under reduced pressure to give a crude product, which was purified by preparative HPLC to give of N-(2-(4-amino-3-(4-chloro-3-hydroxyphenyl)-1H-pyrazolo[3,4-d]pyrimidin-1-yl)ethyl)-N-(furan-2-ylmethyl)acrylamide (8 mg). The product obtained was dissolved in THF (2 mL), methane sulfonic acid (3.51 mg, 0.036 mmol) was added to it and the mixture stirred for 1 h. The reaction mixture was concentrated, washed with diethyl ether (2×2 mL) and lyophilized to give of N-(2-(4-amino-3-(4-chloro-3-hydroxyphenyl)-1H-pyrazolo[3,4-d]pyrimidin-1-yl)ethyl)-N-(furan-2-ylmethyl) acrylamide (9.2 mg) as the mesylate salt (an off-white solid). ¹H NMR (400 MHz, Methanol-d4) δ (ppm): 8.36 (d, J=6.0 Hz, 1H), 7.51 (d, J=8.2 Hz, 1H), 7.41 (d, J=20.0 Hz, 1H), 7.23 (s, 1H), 7.12 (dd, J=8.1, 2.0 Hz, 1H), 6.78-6.23 (m, 1H), 6.36 (m, 2H), 6.08-5.98 (m, 1H), 5.69-5.48 (m, 1H), 4.68 (s, 2H), 4.58 (s, 2H), 3.97 (d, J=5.6 Hz, 2H), 2.71 (s, 6H).

### Example 21

### Preparation of Compound No. 21

### Synthesis of 5-(4-amino-1-{2-[(pyrimidin-2-yl)amino]ethyl}-1H-pyrazolo[3,4-d]pyrimidin-3-yl)-2,3-difluorophenol

To a solution of the 5-(4-amino-1-(2-aminoethyl)-1H-pyrazolo [3, 4-d]pyrimidin-3-yl)-2, 3-difluorophenol (100 mg, 0.326 mmol) in ethanol (1 mL), were added 2-chloro pyrimidine (56 mg, 0.49 mmol), sodium bicarbonate (54 mg, 0.652 mmol) and water (1 mL). Then the mixture was heated at 90° C. for 18 h. The progress of reaction was monitored by TLC and LCMS. The mixture was concentrated and the organic layer was extracted with EtOAc (2×100 mL). The combined organic layer was dried over anhydrous sodium sulfate and concentrated under reduced pressure. The crude compound was purified by reverse phase HPLC to obtain 5-(4-amino-1-(6, 7-dihydro-5H-cyclopenta[b]pyridin-5-yl)-1H-pyrazolo [3, 4-d]pyrimidin-3-yl)-2, 3-difluorophenol (7 mg) as a free base. The product was dissolved in 2 mL of ethanolic-HCl and kept at RT for 20 min. The reaction mixture was then concentrated under reduced pressure and lyophilized to obtain 5-(4-amino-1-(6, 7-dihydro-5H-cyclopenta[b]pyridin-5-yl)-1H-pyrazolo [3, 4-d]pyrimidin-3-yl)-2, 3-difluorophenol (7.5 mg) as the HCl salt (an off-white solid). ¹H NMR (400 MHz, Methanol-d4) δ (ppm): 8.38 (s, 1H), 7.02 (m, 3H), 6.98 (m, 2H), 4.80-4.71 (m, 2H), 4.10 (t, J=5.2 Hz, 2H).

### Example 22

### Preparation of Compound No. 22

### Synthesis of N-{2-[4-amino-3-(3-cyano-4-fluoro-5-hydroxyphenyl)-1H-pyrazolo[3,4-d]pyrimidin-1-yl]ethyl}-N-benzylprop-2-enamide

To a stirred solution of 5-[4-amino-1-[2-(benzylamino)ethyl]pyrazolo[3,4-d]pyrimidin-3-yl]-2-fluoro-3-hydroxy-benzonitrile (110 mg, 0.27 mmol) in DCM (6 mL) was added acryloyl chloride (24.2 mg, 0.2729 mmol) in DCM (1 mL) at 0° C. dropwise and the resultant reaction mixture was stirred at 0° C. for 5 min. The reaction was monitored by TLC and LCMS. After completion, the reaction mixture was slowly quenched with ice water (15 mL) and extracted with EtOAc (100 mL). The organic layer was washed with water (40 mL), brine (30 mL), dried over sodium sulfate and concentrated. The crude product was purified by preparative HPLC to afford N-[2-[4-amino-3-(3-cyano-4-fluoro-5-hydroxy-phenyl) pyrazolo[3,4-d]pyrimidin-1-yl]ethyl]-N-benzyl-prop-2-enamide (21 mg) as a free base (an off-white solid). ¹H NMR (400 MHz, Methanol-d4): δ (ppm): 8.24 (d, J=14.5 Hz, 1H), 7.42 (d, J=7.9 Hz, 1H), 7.24 (m, 5H), 7.06 (d, J=7.1 Hz, 1H), 6.60-6.40 (m, 1H), 6.14-5.99 (m, 1H), 5.65-5.46 (m, 1H), 4.67-4.51 (m, 4H), 4.51 (m, 4H), 3.93 (dt, J=12.1, 5.4 Hz, 2H).

### Example 23

### Preparation of Compound No. 23

### Synthesis of 5-(4-amino-1-{2-[(pyridin-3-ylmethyl)amino]ethyl}-1H-pyrazolo[3,4-d]pyrimidin-3-yl)-2,3-difluorophenol

### Step-1: Synthesis of 3-iodo-1-[2-(3-pyridylmethylamino)ethyl]pyrazolo[3,4-d]pyrimidin-4-amine

To a stirred solution of 1-(2-aminoethyl)-3-iodo-pyrazolo[3,4-d]pyrimidin-4-amine (360 mg, 1.184 mmol) in methanol (10 mL) was added 3-pyridinecarboxaldehyde (126 mg, 1.184 mmol). The reaction mixture was allowed to stir at RT for 2 h. Then, sodium cyanoborohydride (148 mg, 2.368 mmol) was added to the reaction mixture at RT and the mixture was stirred at RT for 1 h. The reaction was monitored by TLC and LCMS. After completion of reaction, the mixture was concentrated under reduced pressure. The residue obtained was diluted with water (80 mL) and extracted with EtOAc (3×100 mL). The combined organic layer was dried over sodium sulfate and concentrated to obtain 3-iodo-1-[2-(3-pyridylmethylamino)ethyl]pyrazolo[3,4-d]pyrimidin-4-amine (400 mg) as a yellow sticky solid which was taken to the next step without further purification.

### Step-2: Synthesis of 5-[4-amino-1-[2-(3-pyridylmethylamino)ethyl]pyrazolo[3,4-d]pyrimidin-3-yl]-2,3-difluoro-phenol

To a stirred solution of 3-iodo-1-[2-(3-pyridylmethylamino)ethyl]pyrazolo[3,4-d]pyrimidin-4-amine (400 mg, 1.0126 mmol) in DMF (5 mL) was added, 3,4-difluoro-5-hydroxyphenyl boronic acid (264 mg, 1.518 mmol) and stirred for 2 min. Then a solution of sodium carbonate (322 mg, 3.037 mmol) in water (5 mL) was added followed by addition of Pd(PPh₃)₄ (81 mg, 0.07 mmol) and the resultant reaction mixture was heated at 90° C. for 16 h. The reaction was monitored by TLC and LC-MS. After completion, the reaction mixture was diluted with water (60 mL) and extracted with EtOAc (3×100 mL). The combined organic layer was dried over anhydrous sodium sulfate and concentrated. The crude product was purified by reverse phase preparative HPLC to obtain 5-[4-amino-1-[2-(3-pyridylmethylamino)ethyl]pyrazolo[3,4-d]pyrimidin-3-yl]-2,3-difluoro-phenol (30 mg) as an off-white solid. This product (10 mg) was dissolved in 2M Ethanolic-HCl (4 mL) and stirred at RT for 20 min. The reaction mixture was concentrated under reduced pressure and lyophilized to obtain 5-[4-amino-1-[2-(3-pyridylmethylamino) ethyl]pyrazolo[3,4-d]pyrimidin-3-yl]-2,3-difluoro-phenol (10.5 mg) as the HCl salt (an off-white solid). ¹H NMR (400 MHz, Methanol-d4) δ (ppm): 8.97 (s, 1H), 8.84 (s, 1H), 8.54 (d, J=8.1 Hz, 1H), 8.47 (s, 1H), 7.98-7.89 (m, 1H), 7.10 (dt, J=6.3, 4.0 Hz, 2H), 4.92 (t, J=5.7 Hz, 2H), 4.55 (s, 2H), 3.81 (t, J=5.7 Hz, 2H). Mass: M+1=398.

### Example 24

### Preparation of Compound No. 24

### Synthesis of N-{2-[4-amino-3-(4-phenoxyphenyl)-1H-pyrazolo[3,4-d]pyrimidin-1-yl]ethyl}-N-benzylprop-2-enamide

To a stirred solution of 1-[2-(benzylamino)ethyl]-3-(4-phenoxyphenyl)pyrazolo [3,4-d]pyrimidin-4-amine (110 mg, 0.2522 mmol) in DCM (4 ml) added acryloyl chloride (20 mg, 0.2522 mmol) in DCM (1 mL) at 0° C. The reaction mixture was stirred at 0° C. for 5 min. The reaction was monitored with TLC. After completion of reaction, the mixture was diluted with EtOAc (150 mL) and washed with water (2×50 mL). The organic layer was dried over sodium sulfate and concentrated under reduced pressure. The crude product was purified by reverse phase preparative HPLC to obtain N-[2-[4-amino-3-(4-phenoxyphenyl)pyrazol o[3,4-d]pyrimidin-1-yl]ethyl]-N-benzyl-prop-2-enamide (28.82 mg) as the free base (an off-white solid). ¹H NMR (400 MHz, Methanol-d4) δ (ppm): 8.22 (d, J=15.5 Hz, 1H), 7.63 (dd, J=8.4, 6.2 Hz, 2H), 7.39 (t, J=7.8 Hz, 2H), 7.31-7.00 (m, 10H), 6.58-6.38 (m, 1H), 6.15-5.98 (m, 1H), 5.63-5.43 (m, 1H), 4.69-4.55 (m, 4H), 3.90 (dt, J=17.9, 5.6 Hz, 2H). Mass: M+1=395.

### Example P1

### Preparation of Compound Nos. 2.1 to 2.297, and Stereoisomers Thereof

Compound Nos. 2.1 to 2.297, and stereoisomers thereof, can be prepared in an analogous fashion to the compounds described herein using appropriately functionalized starting materials and reagents known to those skilled in the art.

### Example B1

### BTK and PI3Kδ Kinase Profiling of Compounds of the Invention

Compounds of the invention were profiled in a 10 dose IC₅₀ panel against BTK and PI3Kδ, both provided by Reaction Biology Corporation.

BTK assay: In vitro profiling protein kinases was performed using the “HotSpot” assay platform. Briefly, specific kinase/substrate pairs along with required cofactors were prepared in reaction buffer. Compounds were prepared as 10 mM stock solutions in DMSO. Compounds were delivered into the reaction, followed 15-20 minutes later by addition of a mixture of ATP (Sigma, St. Louis Mo.) and ³³P ATP (Perkin Elmer, Waltham Mass.) to a final concentration of 10 μM. Reactions were carried out at RT for 120 min, followed by spotting of the reactions onto P81 ion exchange filter paper (Whatman Inc., Piscataway, N.J.). Unbound phosphate was removed by extensive washing of filters in 0.75% Phosphoric acid. After subtraction of background derived from control reactions containing inactive enzyme, kinase activity data was expressed as the percent of remaining kinase activity in test samples compared to vehicle (DMSO) reactions. ICso values and curve fits were obtained using Prism (GraphPad Software). Both the percent inhibition values @ 1 and 0.1 μM, as well as IC₅₀ values are presented in Table B1. Buffer Conditions: 20 mM Hepes (pH 7.5), 10 mM MgCl₂, 1 mM EGTA, 0.02% Brij35, 0.02 mg/mL BSA, 0.1 mM Na₃VO₄, 2 mM DTT, 1% DMSO.

PI3Kδ assay: The PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI3-Kinase (h) activity displaces biotin-PIP3 from the complex resulting in a loss of energy transfer and thus a decrease in signal. This is a 3-step reaction: First, the kinase reaction with PIP2 substrate is carried out in the presence of ATP (10 μM), and the reaction is quenched with stop Solution, and then finally detect by adding Detection Mixture followed by incubation (10 min). Reaction procedure: 1) Prepare substrate in freshly prepared Reaction Buffer. 2) Deliver kinase into the substrate solution and gently mix. 3) Deliver compounds in 100% DMSO (1 μM) into the kinase reaction mixture by Acoustic technology (Echo550; nanoliter range), incubate for 10 min at RT. 4) Deliver ATP into the reaction mixture to initiate the reaction. 5) Incubate for 30 min at 30° C. 6) Quench the reaction with Stop Solution. 7) Add Detection Mixture, and incubate for overnight. 8) Measure HTRF: Ex=320 nm, ratio of Em=615 nm and Em=665 nm. The emission ratio is converted into μM PIP3 production based on PIP3 standard curves. The nonlinear regression to obtain the standard curve and IC₅₀ values are performed using Graphpad Prism software. Both the percent inhibition values @ 1 and 0.1 μM, as well as IC₅₀ values are presented in Table B1. Assay Buffer: HEPES 50 mM (pH7.0), NaN3 0.02%, BSA 0.01%, Orthovanadate 0.1 mM, 1% DMSO. Detection buffer: HEPES 10 mM (pH7.0), BSA 0.02%, KF 0.16 M, EDTA 4 mM. Substrate: 10 μM PIP2 substrate (PI(4,5)P2).

TABLE B1
Kinase activity data - BTK & PI3Kδ kinases

| | BTK @ | BTK @ | BTK @ | BTK | PI3Kδ | PI3Kδ @ | PI3Kδ @ | PI3Kδ |
|---|---|---|---|---|---|---|---|---|
| Cpd. | 1 μM | 0.1 μM | 0.01 μM | IC₅₀ | @ 1 μM | 0.1 μM | 0.01 μM | IC₅₀ |
| No | (% Inh) | (% Inh) | (% Inh) | (μM) | (% Inh) | (% Inh) | (% Inh) | (μM) |
| 1 | | | | 0.167 | | | | 0.0100 |
| 2 | | 94.11 | | 0.0039 | | 39.1 | | 0.0516 |
| 3 | | | | 4.78 | | | | 0.023 |
| 4 | | 14.84 | −1.37 | | | 68.02 | 1.78 | |
| 5 | | 94.00 | | 0.0037 | | 70.27 | | 0.0130 |
| 6 | | | | | | | | |
| 7 | 20.62 | 19.86 | | | 96.31 | 83.49 | | |
| 8 | 12.24 | 9.54 | | | 86.1 | 69.99 | | |
| 9 | 91.59 | 47.27 | | | 85.17 | 46.98 | | |
| 10 | 98.32 | 95.44 | | 0.0030 | 89.02 | 67.15 | | 0.0128 |
| 11 | 97.23 | 93.8 | | 0.0074 | 82.42 | 54.24 | | 0.0320 |
| 12 | | 94.42 | 62.82 | 0.0004 | | 66.69 | 37.69 | 0.0097 |
| 13 | | 98.61 | 95.56 | | | 43.03 | 14.04 | |
| 14 | | 96.09 | 86.55 | | | 54.97 | 24.83 | |
| 15 | | 92.14 | 48.54 | | | 31.13 | 7.57 | |
| 16 | | 93.83 | 63.43 | 0.0027 | | 73.01 | 48.34 | 0.0124 |
| 17 | | 96.16 | 90.34 | | | 41.09 | 13.58 | |
| 18 | | 93.14 | 62.5 | | | 23.39 | 20.79 | |
| 19 | | 96.21 | 93.67 | | | 75.6 | 38.92 | |
| 20 | | 95.43 | 90.34 | | | 40.59 | 17.08 | |
| 21 | | 37.14 | 9.64 | | | 56.63 | 8.59 | |
| 22 | | 50.99 | 5.36 | | | 86.41 | 52.6 | |
| 23 | | 60.45 | 12.74 | | | 66.72 | 3.31 | |
| 24 | | 96.71 | 96.52 | | | 0.74 | −32.29 | |

### Example B2

### PI3Kδ Isoform Profiling of Compounds of the Invention

Compounds of the invention were profiled in a 10 dose IC₅₀ panel against PI3Kα, PI3Kβ, and PI3Kγ kinases, provided by Reaction Biology Corporation, applied to the PI3Kδ protocol provided in Example B1. Both the percent inhibition values @ 0.3 μM, as well as IC₅₀ values are presented in Table B2.

TABLE B2
Kinase Activity Data: Related PI3K isoforms: PI3Kα, PI3Kβ, and PI3Kγ.

| Com- | PI3Kα | PI3Kα | PI3Kβ | PI3Kβ | PI3Kγ | PI3Kγ |
|---|---|---|---|---|---|---|
| pound | @ 0.3 μM | IC₅₀ | @ 0.3 μM | IC₅₀ | @ 0.3 μM | IC₅₀ |
| No. | (% Inh) | (μM) | (% Inh) | (μM) | (% Inh) | (μM) |
| 1 | 50.5 | | 62.02 | | 49.73 | |
| 2 | 20.11 | 4.21 | 30.43 | 5.28 | −2.49 | 9.07 |
| 5 | 26.41 | 3.32 | 45.77 | 3.41 | 34.91 | 10.50 |
| 10 | | 0.504 | 16.32 | | −3.88 | |
| 11 | | 3.22 | 41.01 | | 21.45 | |
| 12 | | 0.869 | 31.34 | | 21.48 | |
| 16 | | 0.29 | 25.24 | | 14.63 | |

### Example B3

### BMX/ETK, ITK and TEC Kinase Profiling of Compounds of the Invention

Compounds of the invention were profiled in a 10 dose IC₅₀ panel against BMX/ETK, ITK and TEC kinases, provided by Reaction Biology Corporation, applied to the BTK protocol provided in Example B1. Both the percent inhibition values @ 0.3 μM, as well as IC₅₀ values are presented in Table B3.

TABLE B3
Kinase Activity Data: BMX/ETK, ITK and TEC kinases

| | | BMX/ | | | | |
|---|---|---|---|---|---|---|
| Com- | BMX/ETK | ETK | ITK | ITK | TEC | TEC |
| pound | @ 0.3 μM | IC₅₀ | @ 0.3 μM | IC₅₀ | @ 0.3 μM | IC₅₀ |
| No. | (% Inh) | (μM) | (% Inh) | (μM) | (% Inh) | (μM) |
| 1 | 43.50 | | 0.06 | | −1.13 | |
| 2 | 98.46 | 1.03 | 50.67 | 19.9 | 72.61 | 6.43 |
| 5 | 92.53 | 4.30 | 10.25 | >100 | 48.81 | 25.20 |
| 10 | 92.98 | | 30.53 | | 66.14 | |
| 11 | 93.49 | | 34.69 | | 68.69 | |
| 12 | 94.20 | | 62.54 | | 71.61 | |
| 16 | 97.17 | | 8.93 | | 74.99 | |

### Example B4

### B Cell Activation Assay

1 Female mouse approximately 2 months old was euthanized with CO₂ and the spleen removed. The tissue was homogenized with a polished glass microscope slide. The cells were collected in 5 mL complete medium (RPMI+10% FBS+pen/strep), placed in a 15 mL falcon tube and centrifuged at 500×g for 5 min. The supernatant was discarded and the pellet was resuspended in 3 mL of ACK lysis buffer (NH₄Cl 0.17M, KHCO₃ 10 mM, EDTA-Na₂ 0.1 mM, pH 7.2), incubated at 37° C. for 5 min. 2 mL complete medium was added and the mixture centrifuged at 500×g for 5 min. The supernatant was discarded and the pellet resuspended in 1 mL complete medium. The mixture was passed through a 40 μM cell strainer and collect in 50 mL falcon tube. Enough medium was added to have a total of 10 mL. The cells were counted and seeded in 24 or 48-well plate in 1 mL complete medium at 10⁶ cells/mL. The plate was placed in a 37° C. incubator for 15 min. The cells were treated for 30 min at 37° C. with 100 nM test compound dissolved in 100% DMSO. 3 μL IgD (Accurate chemical and scientific corp.) was added at a final concentration of 3 μg/mL and the mixture incubated at 37° C. for 4 h. The cells were collected in 1.5 mL eppendorf tubes, centrifuged at 500×g for 5 min and then resuspended in 100 μL of PBS+2% FBS. The following antibodies were added: α-B220 PE (pharmingen #553090), α-CD69 APC (pharmingen #60689), live/dead fixable aqua dead cell stain kit (life technologies # L34957) all at 1:300, and the mixture incubated for 15 min at 4° C. in the dark. To this was added 900 μL of PBS+2% FBS and the mixture centrifuged at 500×g for 5 min. The supernatant was removed and the pellet resuspended in 300 μL of PBS+2% FBS and mixed well. The mixture was transferred to a cytometer tube. The samples were run in the FACS Canto II cytometer and quantified by the number of CD69+B220+ cells as a percentage of the total number of B220+ alive cells. The results are displayed both as % inhibition and as IC₅₀ values, in Table B4.

TABLE B4

| | CD69 | CD69 IC₅₀ |
|---|---|---|
| Compound No. | @ 100 nM (% Inh) | (μM) |
| 1 | 74.46 ± 6.72 (n = 3) | 0.0551 ± 0.0383 (n = 3) |
| 2 | −113.90 ± 72.17 (n = 5) | |
| 3 | 12.77 ± 6.00 (n = 6) | |
| 4 | 39.15 ± 7.85 (n = 3) | |
| 5 | 12.48 ± 22.30 (n = 10) | |
| 7 | 66.50 ± 5.85 (n = 3) | 0.0819 ± 0.0065 (n = 3) |
| 10 | −23.69 ± 39.70 (n = 5) | |
| 11 | −23.69 ± 39.70 (n = 5) | |
| 12 | −44.67 ± 53.58 (n = 5) | |
| 13 | −164.66 ± 63.05 (n = 3) | |
| 14 | −135.21 ± 55.75 (n = 3) | |
| 15 | −103.29 ± 45.35 (n = 3) | |
| 16 | −44.81 ± 29.39 (n = 3) | |
| 17 | −61.13 ± 47.14 (n = 3) | |
| 18 | −7.39 ± 6.26 (n = 3) | |
| 19 | −99.28 ± 22.26 (n = 3) | |
| 20 | −82.03 ± 14.71 (n = 3) | |
| 21 | 18.24 ± 2.92 (n = 3) | |
| 22 | 27.53 ± 5.77 (n = 3) | |
| 23 | 39.66 ± 10.24 (n = 3) | |
| 24 | 55.81 ± 9.10 (n = 4) | |

### Example B5

### Cell Anti-Proliferation Assay

Compounds of the invention are profiled in a B-lymphocyte anti-proliferation assay using SUDHL6 cells. On Day 0, SUDHL cells (7,500) are plated in RPMI media with FBS medium in a 96-well plate. On Day 1, Compounds of the invention are charged on top of the plated cells with a 1% DMSO final concentration. On Day 3, [³H]Thymidine is added at 100 nCi/well. On Day 4, the cells are harvested using a cell harvester on a GF/C filter mat and dried in a hot air oven. Cell readout is obtained using a scintillation counter, and the activity of the compound is determined as an IC₅₀ value.

### Example B6

### Collagen Antibody-Induced Arthritis Model

The efficacy of compounds of the invention in autoimmune diseases such as arthritis can be assessed by preclinical in-vivo models including, for example, collagen antibody-induced arthritis, provided by MD Biosciences, Inc. Balb/c, DBA/1, or C57BI/6 strains of mice are used, with positive controls of Dexamethasone or Enbrel, over a period of 10-18 days. Readouts include body weight, clinical signs, arthritis score, hind paw thickness, histology, cytokine analysis, and gene expression.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of the formula (I): 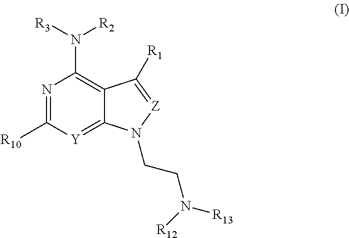
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
Z is N or CR₉;
R₉ is H, or is selected from the group consisting of halo, CN, CF₃, —C(═O)NH₂, and substituted or unsubstituted C₁-C₆ alkyl;
R₁₀ is H, or is selected from the group consisting of halo, —NHR₂, and substituted or unsubstituted C₁-C₆ alkyl;
R₁₂ is selected from the group consisting of H, —[C₁-C₆ alkyl]-CN, 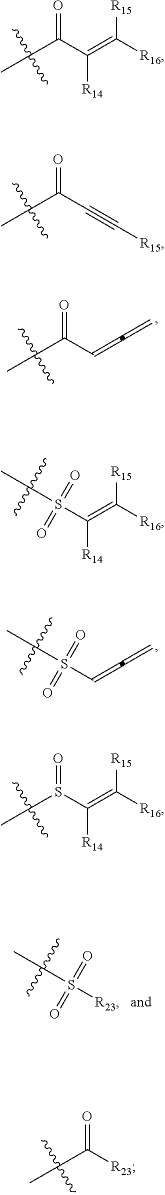
R₁₃ is selected from the group consisting of substituted or unsubstituted aryl, substituted or unsubstituted heteroaryl, —C(═O)-substituted aryl, —C(═O)-unsubstituted aryl, —CH₂— substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;
R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H, or are independently selected from the group consisting of substituted or unsubstituted C₁-C₆ alkyl, substituted or unsubstituted cycloalkyl, substituted or unsubstituted heterocyclyl, and substituted or unsubstituted amino; and
R₂₃ is substituted or unsubstituted C₁-C₆ alkyl or substituted or unsubstituted C₃-C₆ cycloalkyl.

2. A compound of the formula (II): 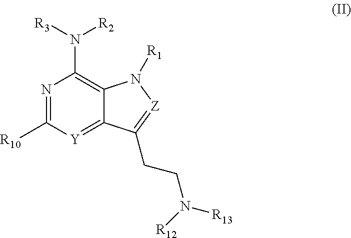
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
Z is N or CR₉;
R₉ is H, or is selected from the group consisting of halo, CN, CF₃, —C(═O)NH₂, and substituted or unsubstituted C₁-C₆ alkyl;
R₁₀ is H, or is selected from the group consisting of halo, —NHR₂, and substituted or unsubstituted C₁-C₆ alkyl;
R₁₂ is H, or is selected from the group consisting of —[C₁-C₆ alkyl]-CN, 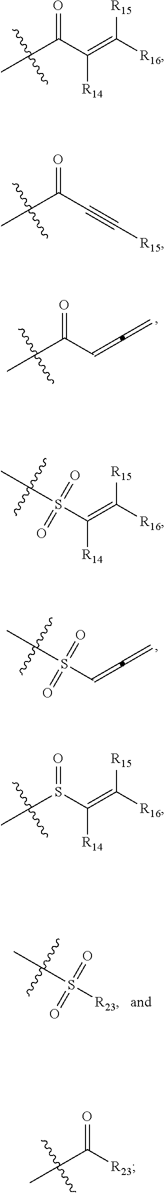
R₁₃ is selected from the group consisting of a substituted or unsubstituted aryl, substituted or unsubstituted heteroaryl, —C(═O)-substituted aryl, —C(═O)-unsubstituted aryl, —CH₂— substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;
R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H, or are independently selected from the group consisting of substituted or unsubstituted C₁-C₆ alkyl, substituted or unsubstituted cycloalkyl, substituted or unsubstituted heterocyclyl, and substituted or unsubstituted amino; and
R₂₃ is substituted or unsubstituted C₁-C₆ alkyl or substituted or unsubstituted C₃-C₆ cycloalkyl.

3. A compound of the formula (III): 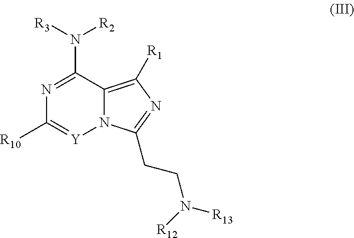
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
R₁₀ is H, or is selected from the group consisting of halo, —NHR₂, and substituted or unsubstituted C₁-C₆ alkyl;
R₁₂ is H, or is selected from the group consisting of —[C₁-C₆ alkyl]-CN, 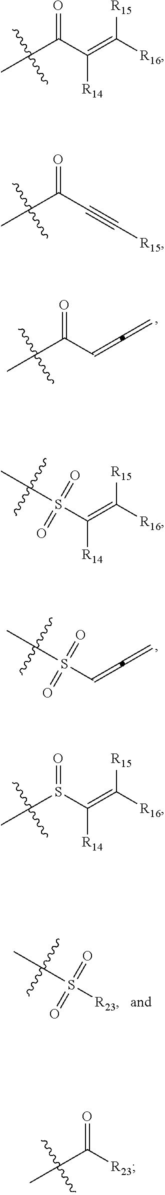
R₁₃ is selected from the group consisting of substituted or unsubstituted aryl, substituted or unsubstituted heteroaryl, —C(═O)-substituted aryl, —C(═O)-unsubstituted aryl, —CH₂— substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;
R₁₄ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H, are independently selected from the group consisting of substituted or unsubstituted C₁-C₆ alkyl, substituted or unsubstituted cycloalkyl, substituted or unsubstituted heterocyclyl, and substituted or unsubstituted amino; and
R₂₃ is substituted or unsubstituted C₁-C₆ alkyl or substituted or unsubstituted C₃-C₆ cycloalkyl.

4. A compound of the formulae (IVa) and (IVb): 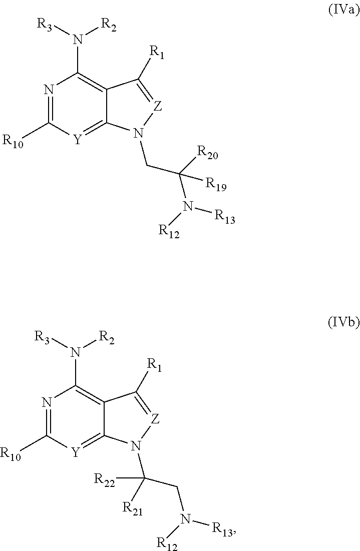
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
Z is N or CR₉;
R₉ is H, or is selected from the group consisting of halo, CN, CF₃, —C(═O)NH₂, and substituted or unsubstituted C₁-C₆ alkyl;
R₁₀ is H, or is selected from the group consisting of halo, and substituted or unsubstituted C₁-C₆ alkyl;
R₁₂ is selected from the group consisting of H, —[C₁-C₆ alkyl]-CN, 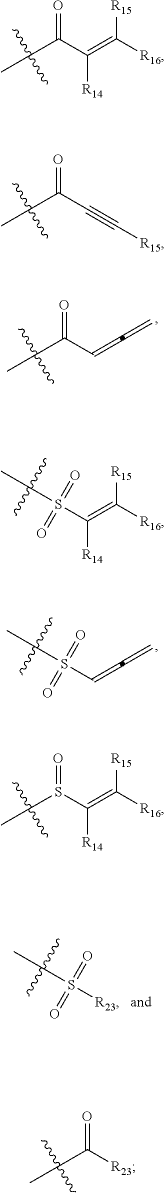
R₁₃ is selected from the group consisting of substituted or unsubstituted aryl, substituted or unsubstituted heteroaryl, —C(═O)-substituted aryl, —C(═O)-unsubstituted aryl, —CH₂— substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;
R₁₄ is H or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H or selected from the group consisting of substituted or unsubstituted C₁-C₆ alkyl, substituted or unsubstituted cycloalkyl, substituted or unsubstituted heterocyclyl, and substituted or unsubstituted amino;
R₁₉ and R₂₀ are both unsubstituted C₁-C₆ alkyl, or are taken together with the carbon atom to which they are attached to form an unsubstituted C₃-C₈ cycloalkyl or carbonyl;
R₂₁ and R₂₂ are both unsubstituted C₁-C₆ alkyl, or are taken together with the carbon atom to which they are attached to form an unsubstituted C₃-C₈ cycloalkyl or carbonyl; and
R₂₃ is substituted or unsubstituted C₁-C₆ alkyl or substituted or unsubstituted C₃-C₆ cycloalkyl;
with the provision that for formula (IVb), when both Y and Z are N; R₂, R₃ and R₁₀ are each H;
and R₂₁ and R₂₂ are each methyl; then R₁₂ is other than H.

5. A compound of the formulae (Va) and (Vb): 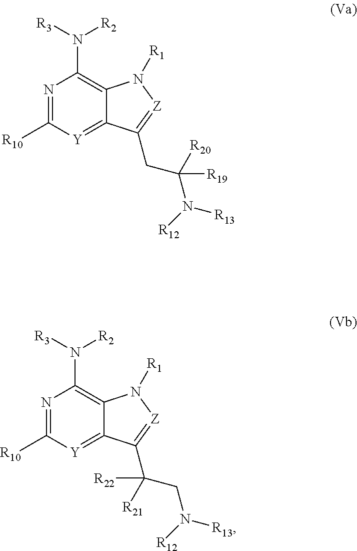
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
Z is N or CR₉;
R₉ is H, or is selected from the group consisting of halo, CN, CF₃, —C(═O)NH₂, and substituted or unsubstituted C₁-C₆ alkyl;
R₁₀ is H, or is selected from the group consisting of halo, —NHR₂, and substituted or unsubstituted C₁-C₆ alkyl;
R₁₂ is selected from the group consisting of H, —[C₁-C₆ alkyl]-CN, 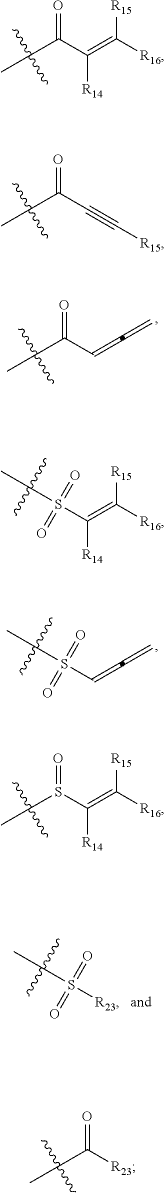
R₁₃ is selected from the group consisting of substituted or unsubstituted aryl, substituted or unsubstituted heteroaryl, —C(═O)-substituted aryl, —C(═O)-unsubstituted aryl, —CH₂— substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl, and a substituted or unsubstituted heterocyclyl;
R₁₄ is H or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;
R₁₉ and R₂₀ are both unsubstituted C₁-C₆ alkyl, or are taken together with the carbon atom to which they are attached to form an unsubstituted C₃-C₈ cycloalkyl or carbonyl;
R₂₁ and R₂₂ are both unsubstituted C₁-C₆ alkyl; or are taken together with the carbon atom to which they are attached to form an unsubstituted C₃-C₈ cycloalkyl or carbonyl; and
R₂₃ is substituted or unsubstituted C₁-C₆ alkyl or substituted or unsubstituted C₃-C₆ cycloalkyl.

6. A compound of the formulae (VIa) and (VIb): 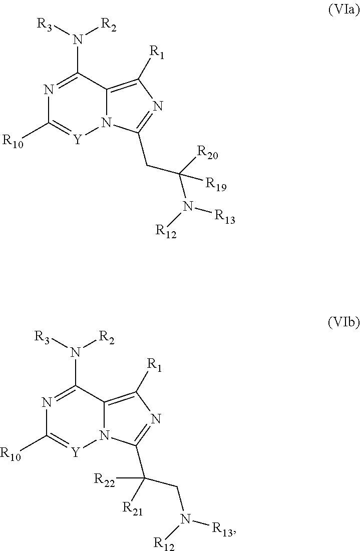
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
Y is CH or N;
R₁₀ is H, or is selected from the group consisting of halo, —NHR₂, and substituted or unsubstituted C₁-C₆ alkyl;
R₁₂ is selected from the group consisting of H, —[C₁-C₆ alkyl]-CN, 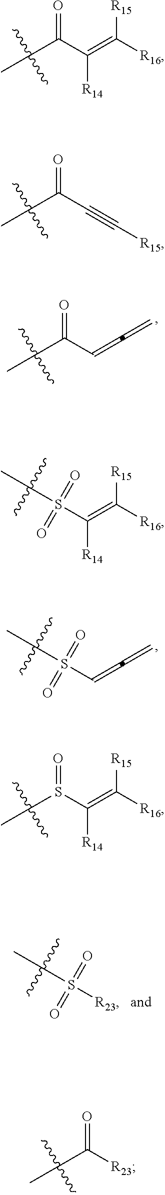
R₁₃ is substituted or unsubstituted aryl, substituted or unsubstituted heteroaryl, —C(═O)-substituted aryl, —C(═O)-unsubstituted aryl, —CH₂-substituted aryl, or —CH₂-unsubstituted aryl, —C(═O)-substituted heteroaryl, —C(═O)-unsubstituted heteroaryl, —CH₂-substituted heteroaryl, —CH₂-unsubstituted heteroaryl, substituted or unsubstituted cycloalkyl and a substituted or unsubstituted heterocyclyl;
R₁₄ is H or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₂₃, —C(═O)OR₂₃, and —C(═O)NH₂;
R₁₅ and R₁₆ are each independently H or selected from the group consisting of substituted or unsubstituted C₁-C₆ alkyl, substituted or unsubstituted cycloalkyl, substituted or unsubstituted heterocyclyl, and substituted or unsubstituted amino;
R₁₉ and R₂₀ are both unsubstituted C₁-C₆ alkyl, or are taken together with the carbon atom to which they are attached to form an unsubstituted C₃-C₈ cycloalkyl or carbonyl;
R₂₁ and R₂₂ are both unsubstituted C₁-C₆ alkyl; or are taken together with the carbon atom to which they are attached to form an unsubstituted C₃-C₈ cycloalkyl or carbonyl; and
R₂₃ is substituted or unsubstituted C₁-C₆ alkyl.

7. The compound of the claim 1 wherein R₁ is selected from the group consisting of substituted or unsubstituted C₁-C₆ alkyl, substituted or unsubstituted C₂-C₆ alkenyl, substituted or unsubstituted aryl, substituted or unsubstituted heteroaryl, substituted or unsubstituted cycloalkyl, or substituted or unsubstituted heterocyclyl.

8. The compound of the claim 1 wherein R₁ is selected from the group consisting of phenyl, substituted phenyl, pyridyl or substituted pyridyl, pyrimidyl or substituted pyrimidyl, cyclohexyl or substituted cyclohexyl, indolyl or substituted indolyl, substituted or unsubstituted pyrrolopyridinyl and a substituted or unsubstituted indazolyl.

9. The compound of the claim 1 wherein R₁ is phenyl substituted with two or more substituents selected from the group consisting of hydroxyl, halo, perhaloalkyl, alkoxy, phenoxy, aminoacyl, substituted or unsubstituted amino, substituted or unsubstituted C₁-C₆ alkyl, cyano and allyloxy.

10. The compound of claim 9, wherein R₁ is selected from the group consisting of a substituted or unsubstituted pyridin-2-yl, substituted or unsubstituted pyridin-3-yl, substituted or unsubstituted pyridin-4-yl, substituted or unsubstituted benzimidazol-2-yl, substituted or unsubstituted benzimidazol-4-yl, substituted or unsubstituted benzimidazol-5-yl, substituted or unsubstituted pyrazol-3-yl and substituted or unsubstituted pyrazol-4-yl.

11. The compound of claim 9, wherein R₁ is selected from the group consisting of a substituted or unsubstituted indol-1-yl, indol-2-yl, indol-3-yl, indol-4-yl, indol-5-yl, indol-6-yl, or indol-7-yl, pyrrolopyridin-2-yl, substituted or unsubstituted pyrrolopyridin-3-yl, substituted or unsubstituted pyrrolopyridin-4-yl, substituted or unsubstituted pyrrolopyridin-5-yl, or substituted or unsubstituted pyrrolopyridin-6-yl.

12. The compound of claim 9, wherein R₁ is selected from the group consisting of substituted or unsubstituted indazol-3-yl, substituted or unsubstituted indazol-4-yl, substituted or unsubstituted indazol-5-yl, substituted or unsubstituted indazol-6-yl, substituted or unsubstituted indazol-7-yl, substituted or unsubstituted pyrimidin-2-yl, substituted or unsubstituted pyrimidin-4-yl and substituted or unsubstituted pyrimidin-5-yl.

13. The compound of claim 1, wherein R₂ and R₃ are each H, R₂ is H and R₃ is C₁-C₆ alkyl or R₂ is H and R₃ is methyl.

14. The compound of claim 1, wherein Y is CH or N.

15. The compound of claim 1, wherein Z is CR₉ or Z is N.

16. The compound of claim 1, wherein Y is CH and Z is N, Y is N and Z is N, Y is CH and Z is CR₉ or Y is N and Z is CR₉.

17. The compound of claim 1, wherein R₁₂ is selected from the group consisting of

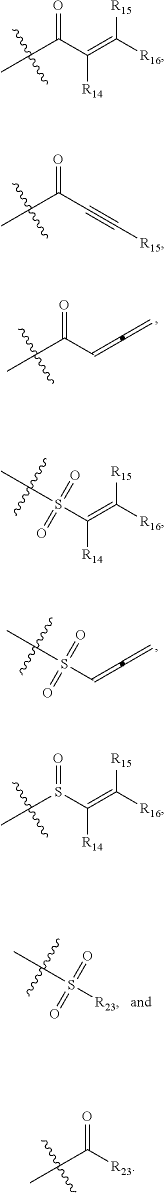

18. The compound of claim 17, wherein R₁₄ is H or is selected from the group consisting of CN, halo, —NO₂, CF₃, —SO₂CH₃, —SO₂NH₂, —C(═O)R₂₃, —C(═O)OR₂₃ and —C(═O)NH₂.

19. The compound of claim 1, wherein R₁₃ is substituted or unsubstituted phenyl.

20. The compound of claim 1, wherein R₁₃ is selected from the group consisting of substituted or unsubstituted benzoyl, substituted or unsubstituted benzyl, substituted or unsubstituted cyclopentyl and substituted or unsubstituted cyclohexyl.

21. The compound of claim 1, wherein R₁₃ is selected from the group consisting of substituted or unsubstituted pyridinyl, substituted or unsubstituted pyrimidinyl, substituted or unsubstituted pyridinyl and substituted or unsubstituted benzimidazolyl.

22. The compound of claim 21, wherein R₁₉ and R₂₀ are each methyl, R₁₉ and R₂₀ are taken together with the carbon to which they are attached to form a cyclopropyl, a cyclobutyl, a cyclopentyl, a cyclohexyl, or R₁₉ and R₂₀ are taken together with the carbon to which they are attached to form a carbonyl.

23. The compound of claim 22, wherein R₂₁ and R₂₂ are each methyl.

24. The compound of claim 22, wherein R₂₁ and R₂₂ are taken together with the carbon to which they are attached to form a cyclopropyl, a cyclobutyl, a cyclopentyl or a cyclohexyl, or wherein R₂₁ and R₂₂ are taken together with the carbon to which they are attached to form a carbonyl.

25. The compound of claim 1 wherein the compound is of the structure B-1, B-2, B-3, B-4, B-5, B-6, B-7, B-8, B-9, or B-10: 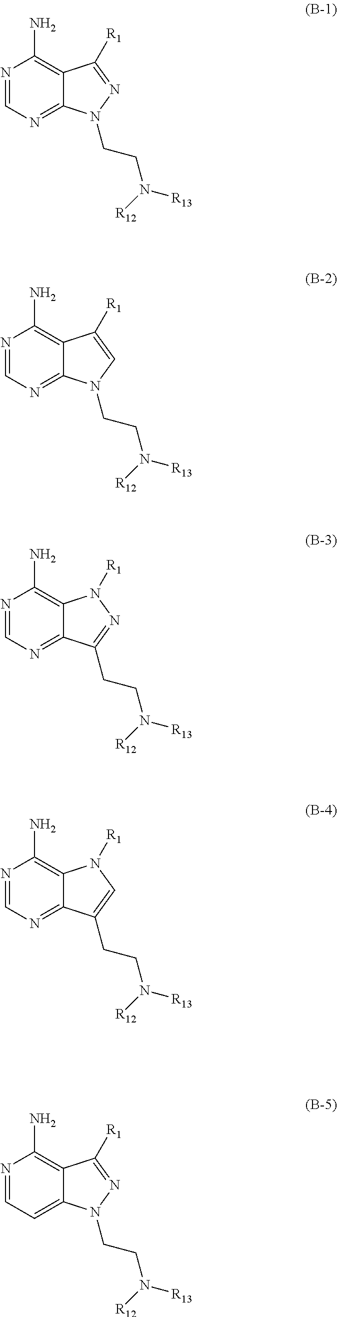 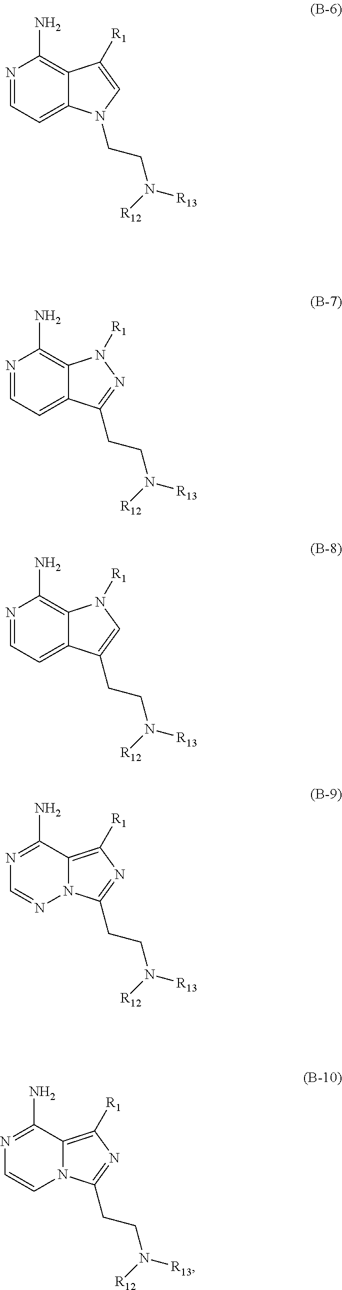
or a salt thereof; wherein R₁, R₁₂ and R₁₃ are as described for formulae (I), (II), and (III).

26. The compound of claim 25, wherein the compound is of the structure B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, or B-10c: 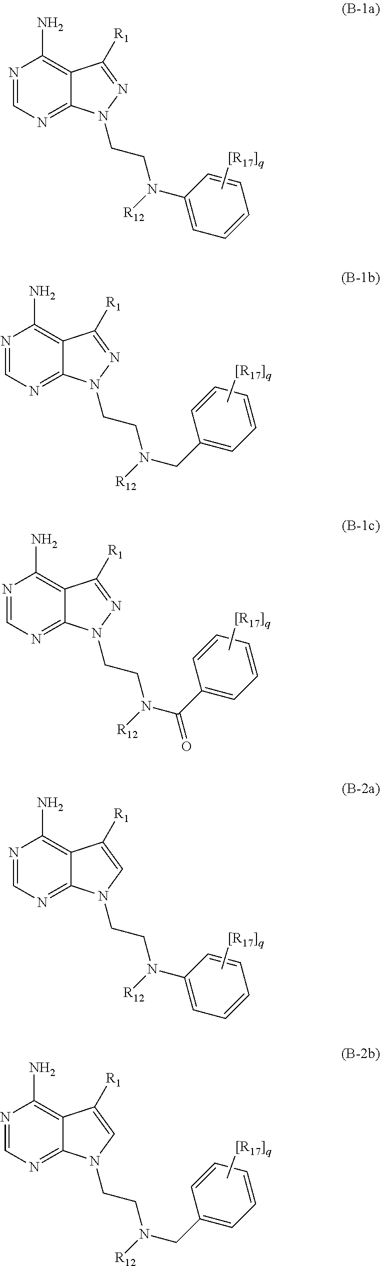 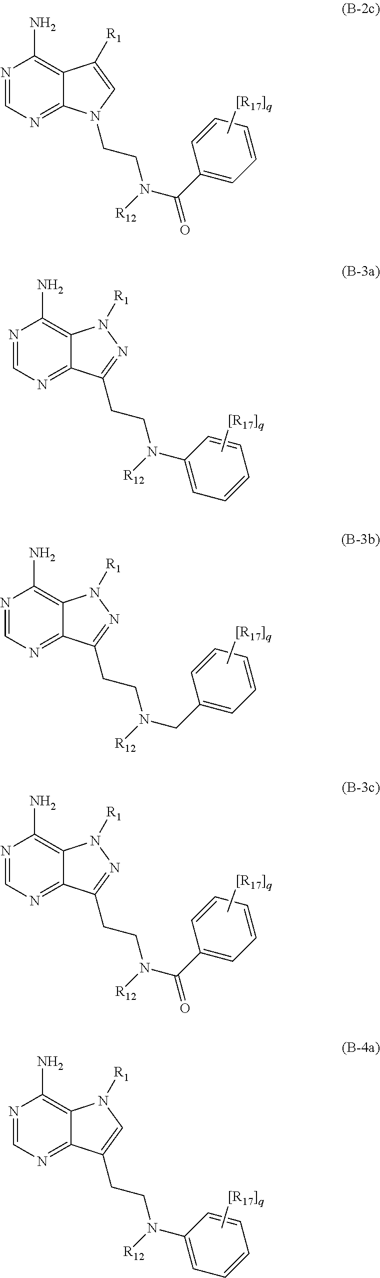 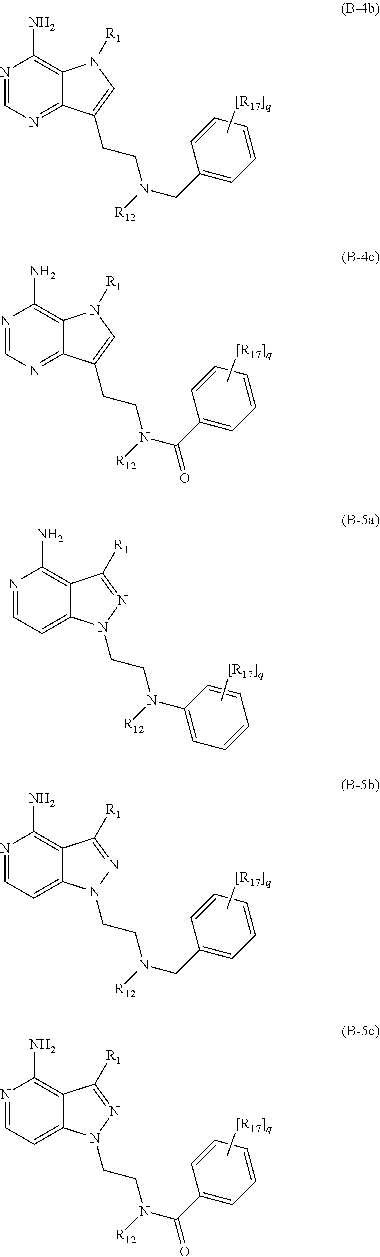 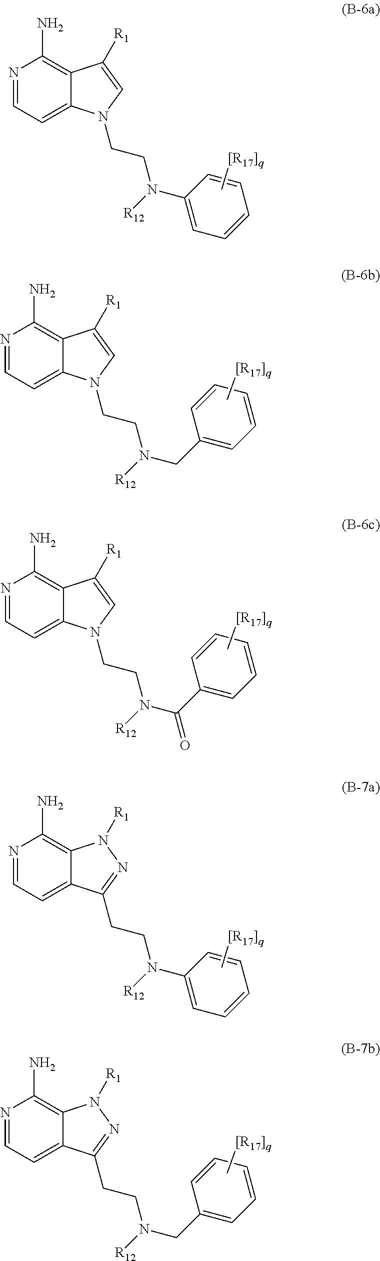 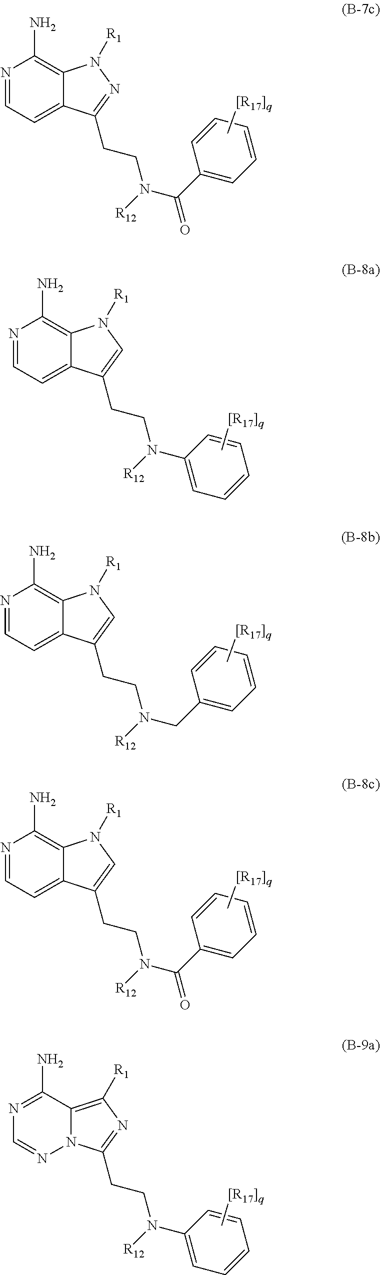 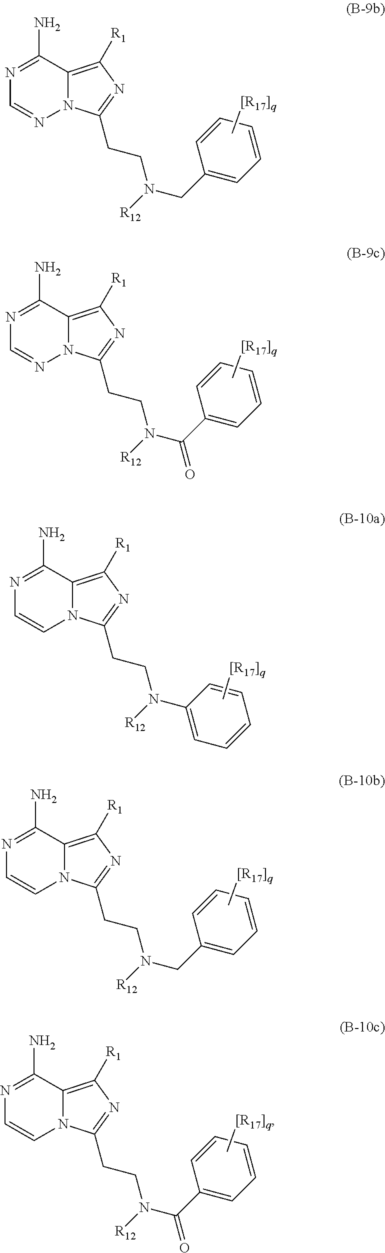
or a salt thereof; wherein R₁ and R₁₂ are as described for formulae B-1, B-2, B-3, B-4, B-5, B-6, B-7, B-8, B-9, and B-10;
each R₁₇, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₃-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, or acyl; and
q is 0 to 3.

27. The compound of claim 26, wherein the compound is of the structure B-1a-1, B-1a-2, B-1a-3, B-1a-4, B-1a-5, B-1a-6, B-1a-7, B-1a-8, B-1b-1, B-1b-2, B-1b-3, B-1b-4, B-1b-5, B-1b-6, B-1b-7, B-1b-8, B-1c-1, B-1c-2, B-1c-3, B-1c-4, B-1c-5, B-1c-6, B-1c-7, B-1c-8, B-2a-1, B-2a-2, B-2a-3, B-2a-4, B-2a-5, B-2a-6, B-2a-7, B-2a-8, B-2b-1, B-2b-2, B-2b-3, B-2b-4, B-2b-5, B-2b-6, B-2b-7, B-2b-8, B-2c-1, B-2c-2, B-2c-3, B-2c-4, B-2c-5, B-2c-6, B-2c-7, B-2c-8, B-3a-1, B-3a-2, B-3a-3, B-3a-4, B-3a-5, B-3a-6, B-3a-7, B-3a-8, B-3b-1, B-3b-2, B-3b-3, B-3b-4, B-3b-5, B-3b-6, B-3b-7, B-3b-8, B-3c-1, B-3c-2, B-3c-3, B-3c-4, B-3c-5, B-3c-6, B-3c-7, B-3c-8, B-4a-1, B-4a-2, B-4a-3, B-4a-4, B-4a-5, B-4a-6, B-4a-7, B-4a-8, B-4b-1, B-4b-2, B-4b-3, B-4b-4, B-4b-5, B-4b-6, B-4b-7, B-4b-8, B-4c-1, B-4c-2, B-4c-3, B-4c-4, B-4c-5, B-4c-6, B-4c-7, B-4c-8, B-5a-1, B-5a-2, B-5a-3, B-5a-4, B-5a-5, B-5a-6, B-5a-7, B-5a-8, B-5b-1, B-5b-2, B-5b-3, B-5b-4, B-5b-5, B-5b-6, B-5b-7, B-5b-8, B-5c-1, B-5c-2, B-5c-3, B-5c-4, B-5c-5, B-5c-6, B-5c-7, B-5c-8, B-6a-1, B-6a-2, B-6a-3, B-6a-4, B-6a-5, B-6a-6, B-6a-7, B-6a-8, B-6b-1, B-6b-2, B-6b-3, B-6b-4, B-6b-5, B-6b-6, B-6b-7, B-6b-8, B-6c-1, B-6c-2, B-6c-3, B-6c-4, B-6c-5, B-6c-6, B-6c-7, B-6c-8, B-7a-1, B-7a-2, B-7a-3, B-7a-4, B-7a-5, B-7a-6, B-7a-7, B-7a-8, B-7b-1, B-7b-2, B-7b-3, B-7b-4, B-7b-5, B-7b-6, B-7b-7, B-7b-8, B-7c-1, B-7c-2, B-7c-3, B-7c-4, B-7c-5, B-7c-6, B-7c-7, B-7c-8, B-8a-1, B-8a-2, B-8a-3, B-8a-4, B-8a-5, B-8a-6, B-8a-7, B-8a-8, B-8b-1, B-8b-2, B-8b-3, B-8b-4, B-8b-5, B-8b-6, B-8b-7, B-8b-8, B-8c-1, B-8c-2, B-8c-3, B-8c-4, B-8c-5, B-8c-6, B-8c-7, B-8c-8, B-9a-1, B-9a-2, B-9a-3, B-9a-4, B-9a-5, B-9a-6, B-9a-7, B-9a-8, B-9b-1, B-9b-2, B-9b-3, B-9b-4, B-9b-5, B-9b-6, B-9b-7, B-9B-9, B-9c-1, B-9c-2, B-9c-3, B-9c-4, B-9c-5, B-9c-6, B-9c-7, B-9c-8, B-10a-1, B-10a-2, B-10a-3, B-10a-4, B-10a-5, B-10a-6, B-10a-7, B-10a-8, B-10b-1, B-10b-2, B-10b-3, B-10b-4, B-10b-5, B-10b-6, B-10b-7, B-10b-8, B-10c-1, B-10c-2, B-10c-3, B-10c-4, B-10c-5, B-10c-6, B-10c-7, or B-10c-8: 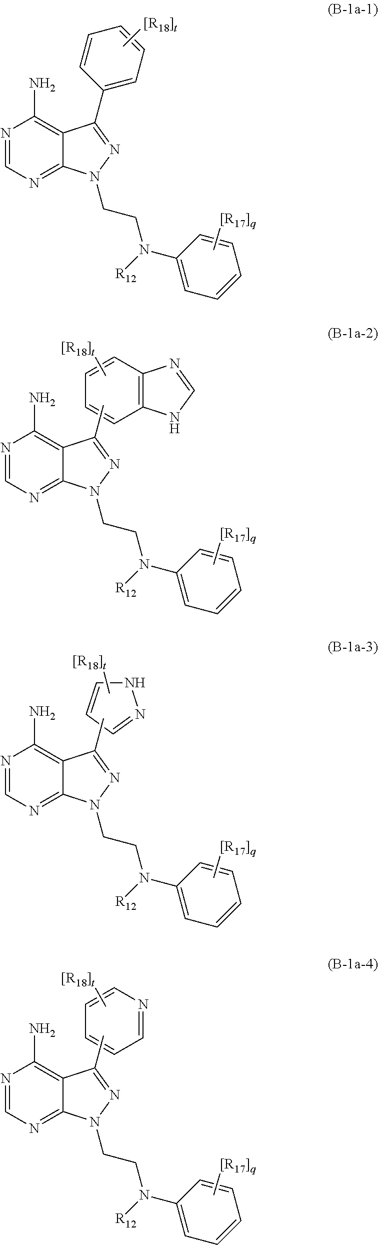 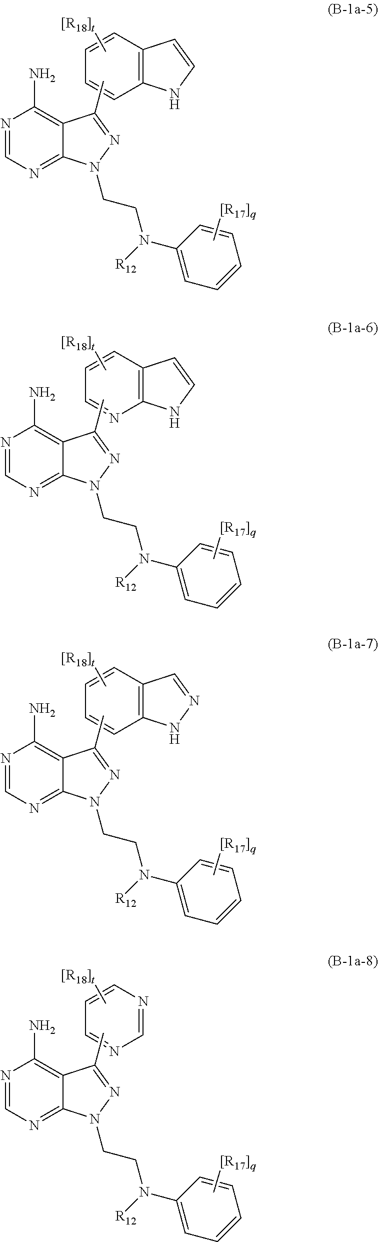 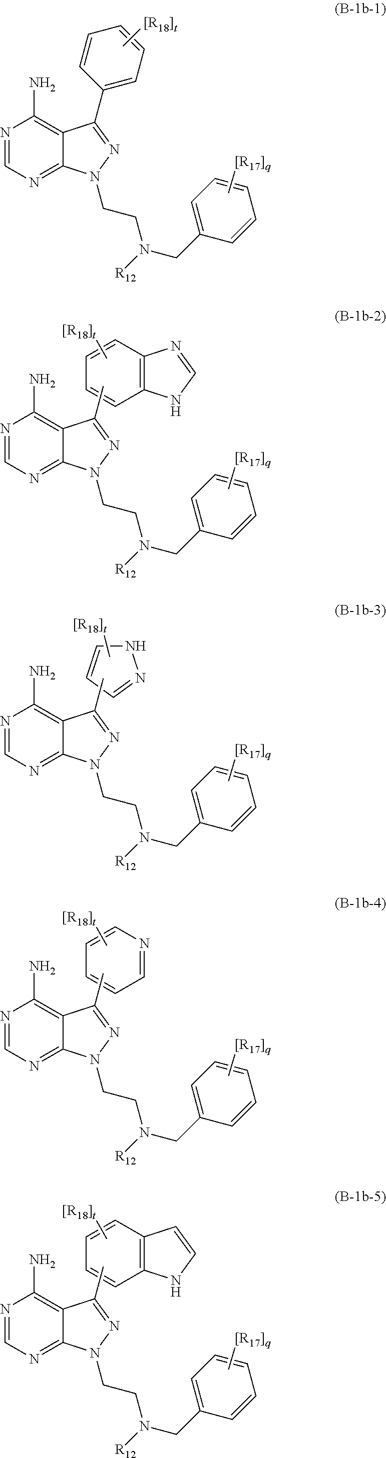 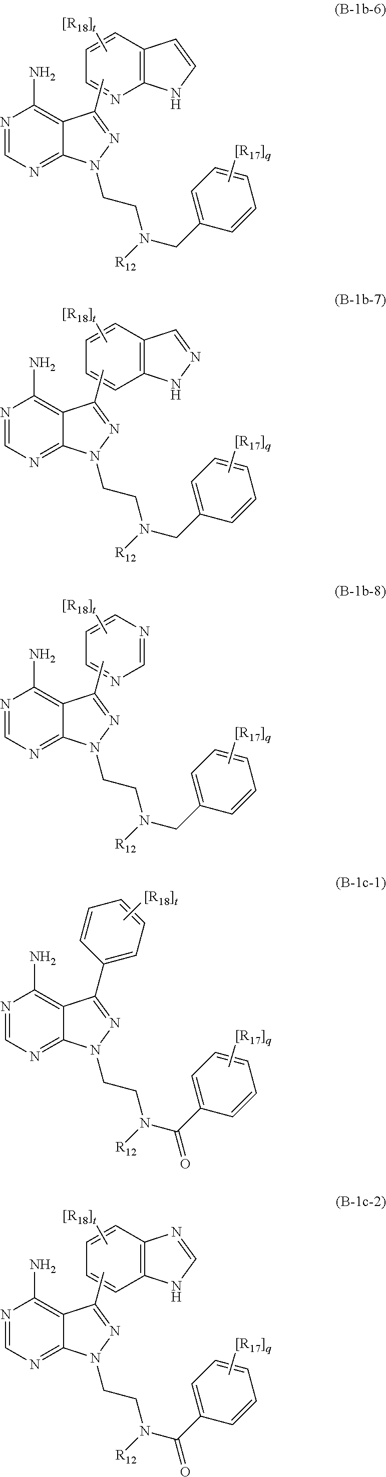 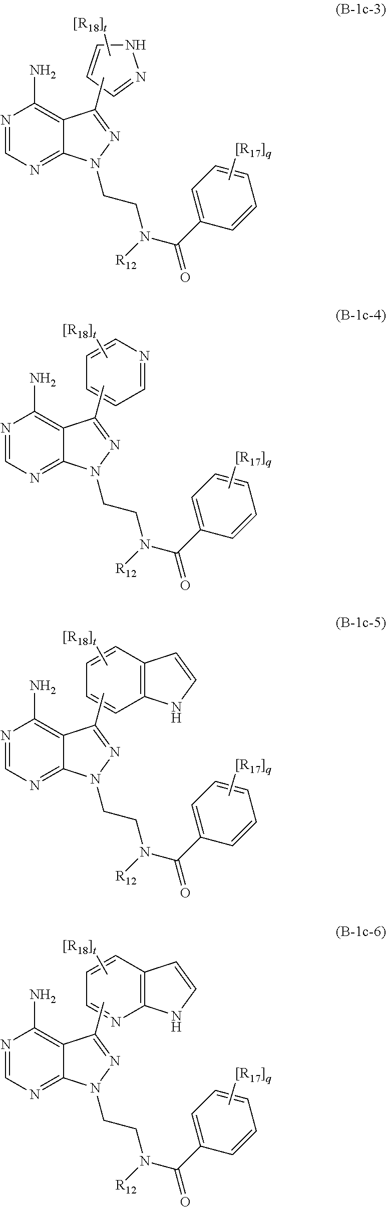 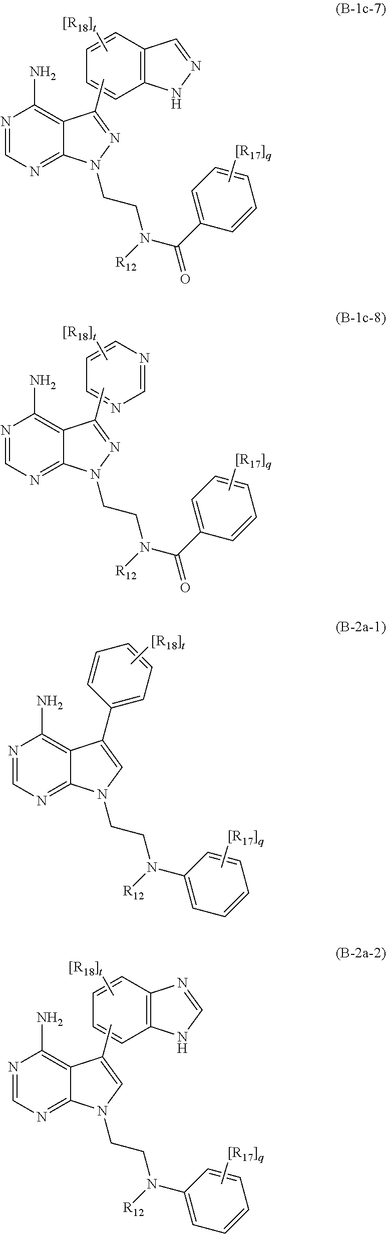 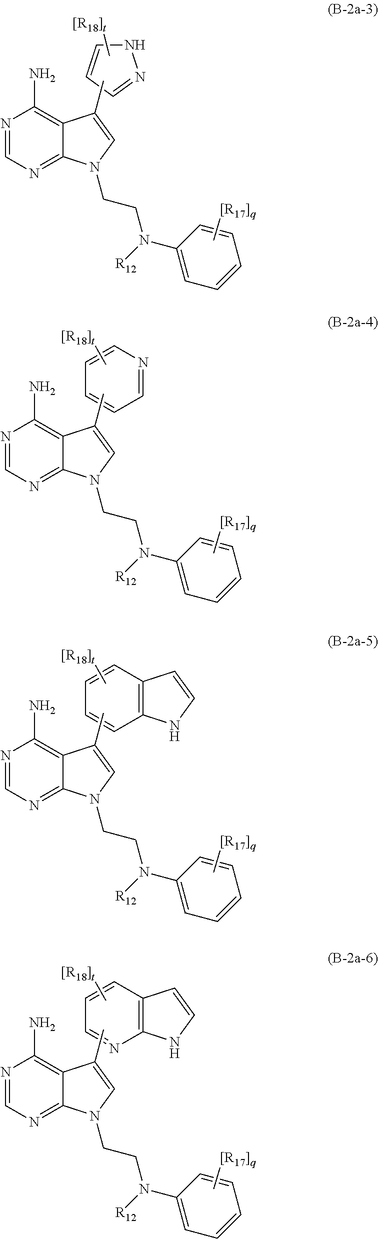 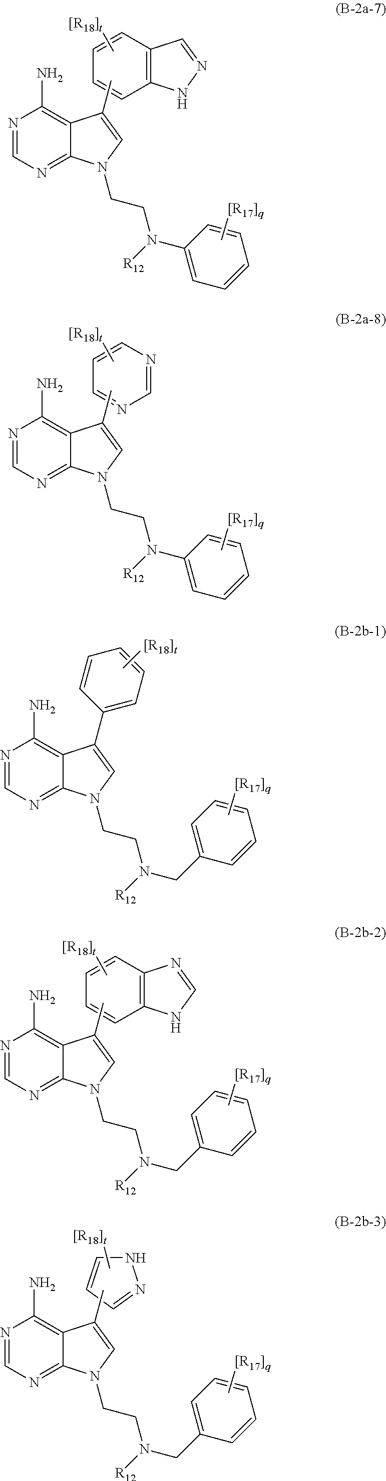 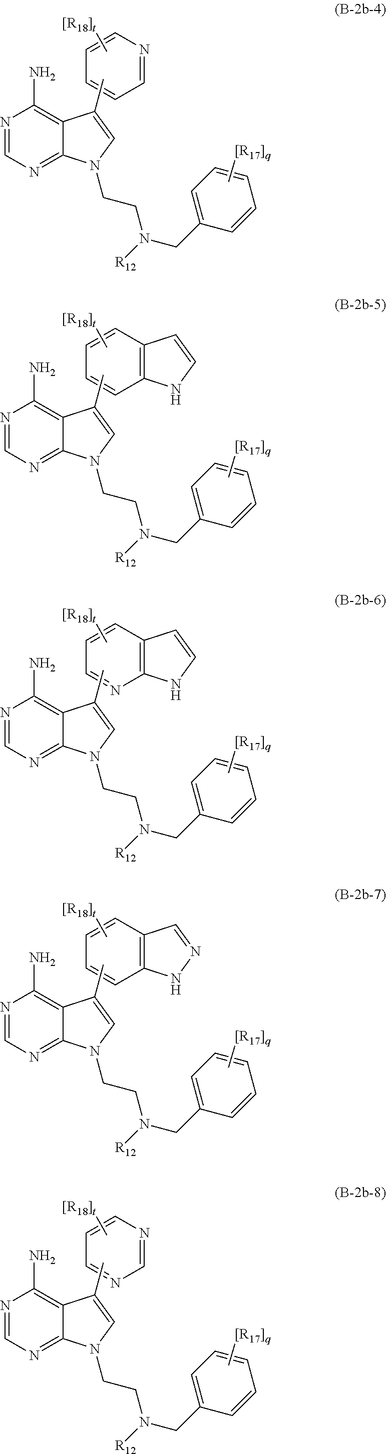 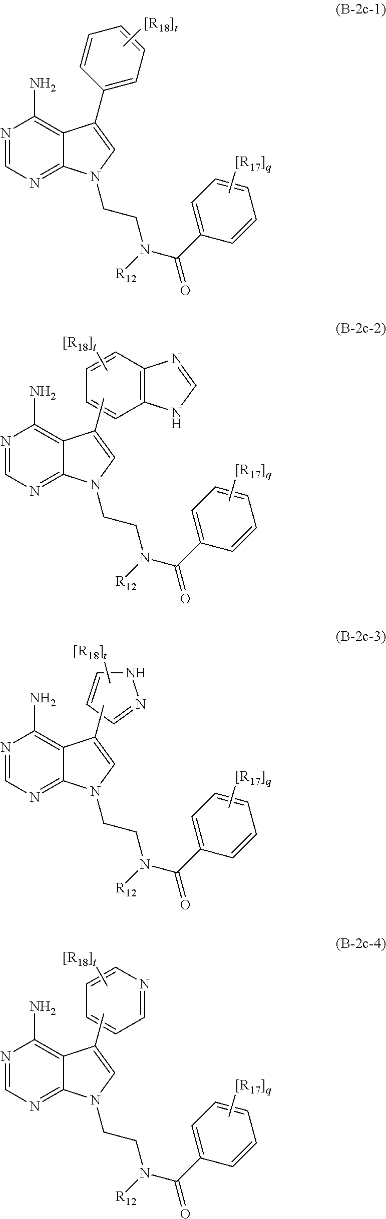 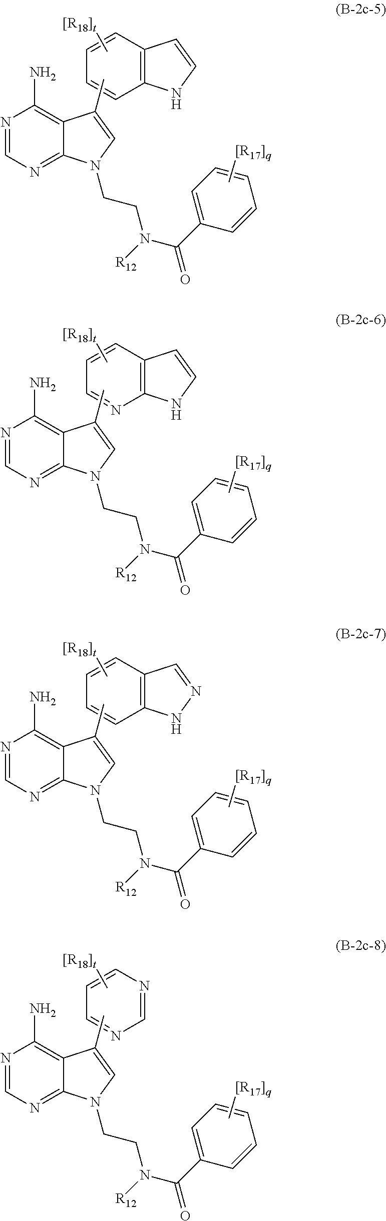 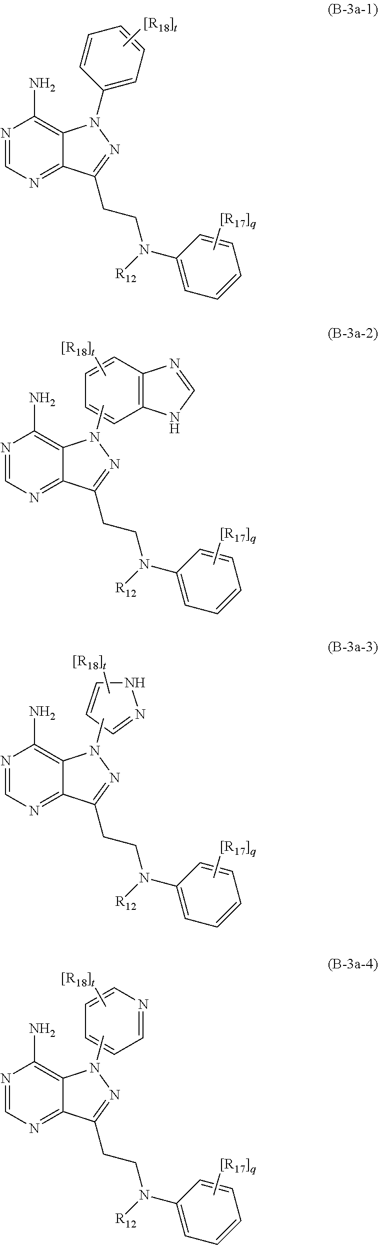 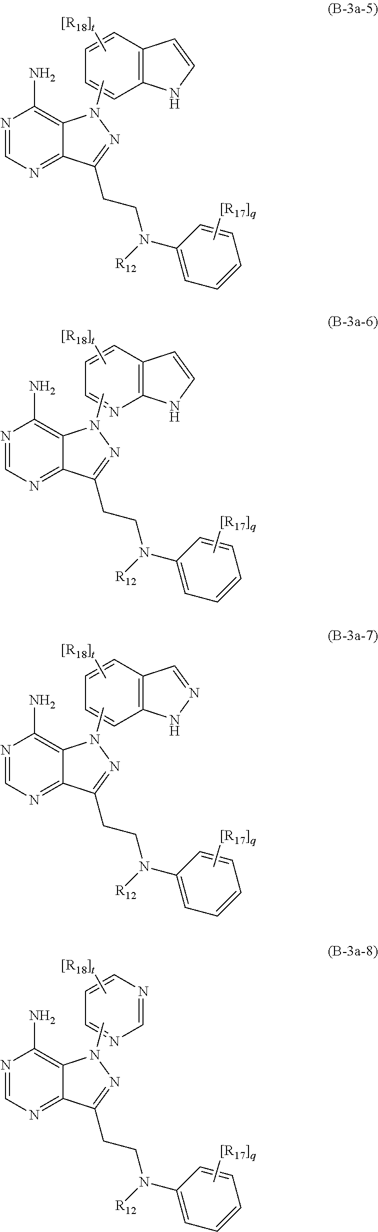 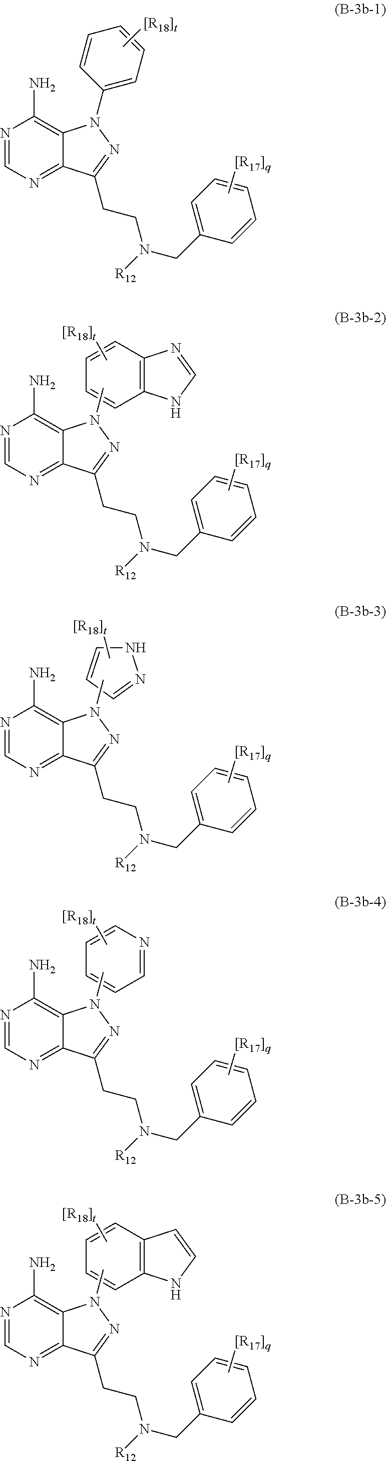 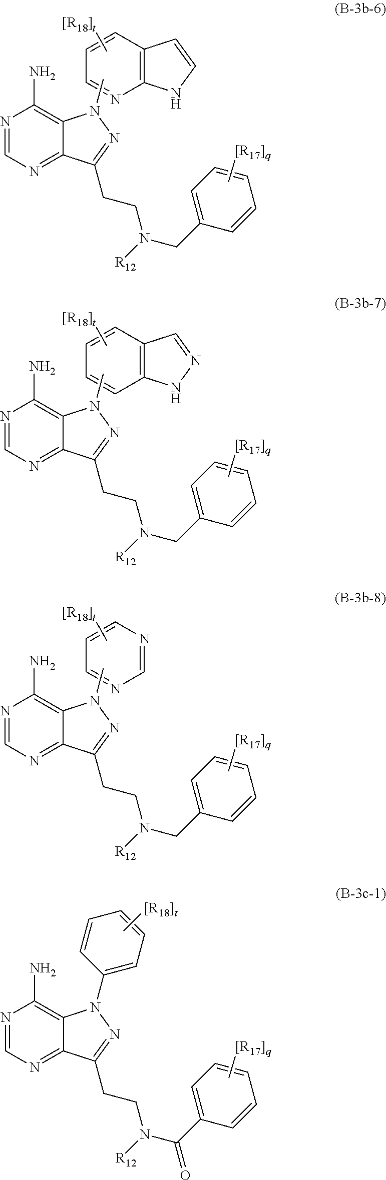 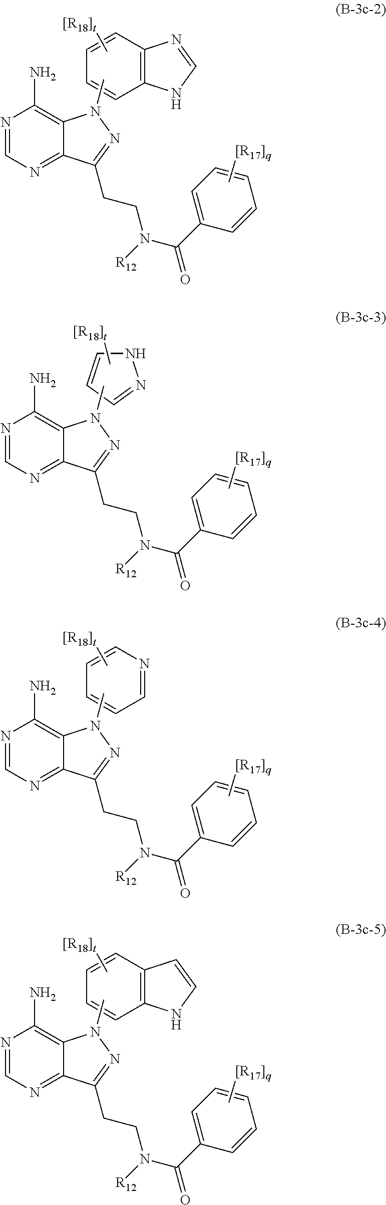 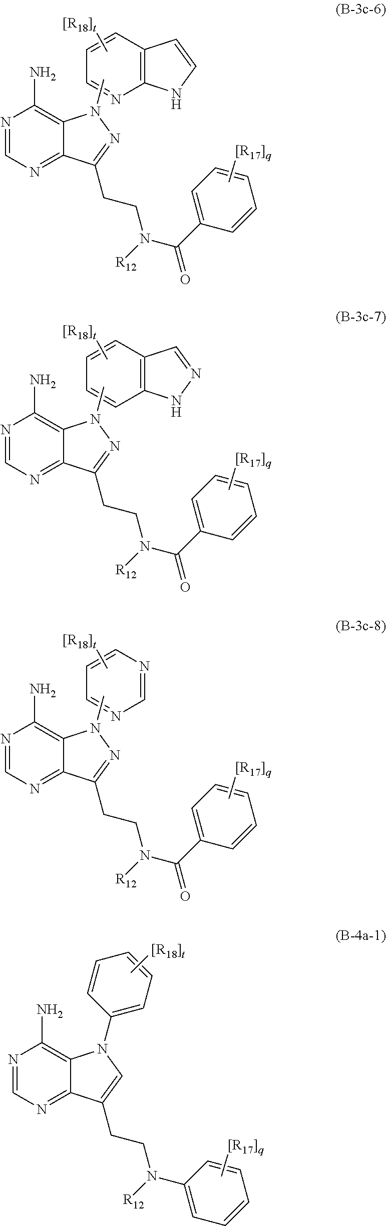 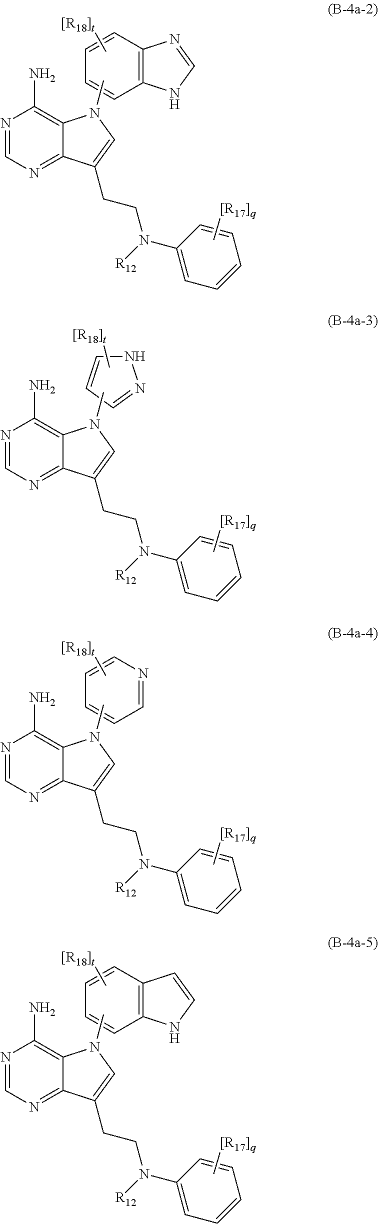 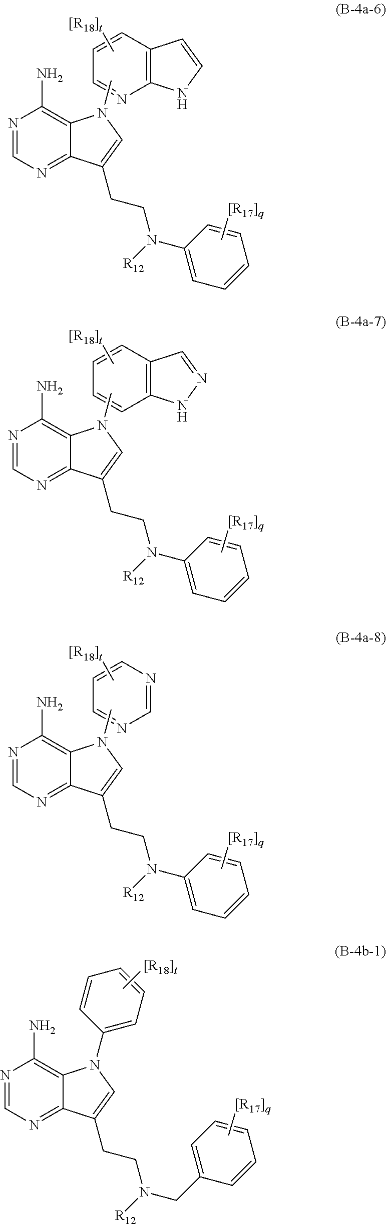 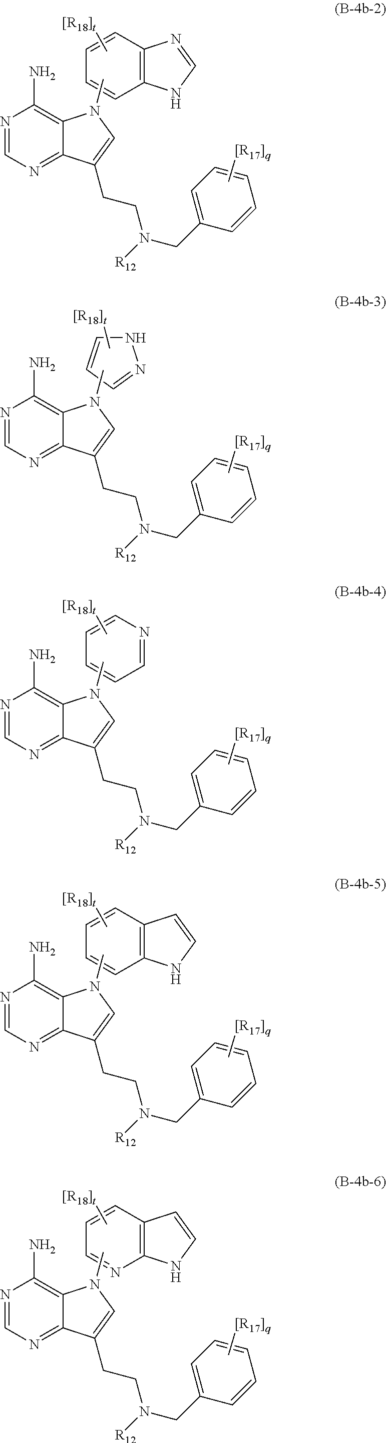 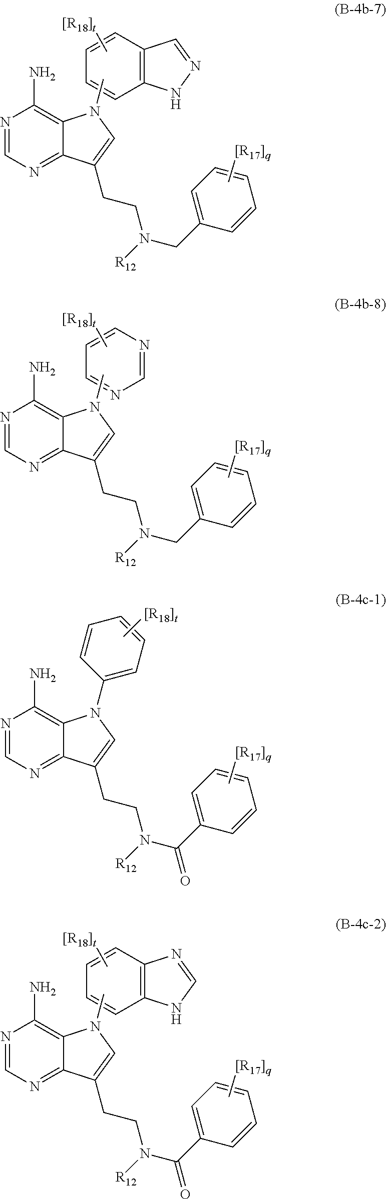 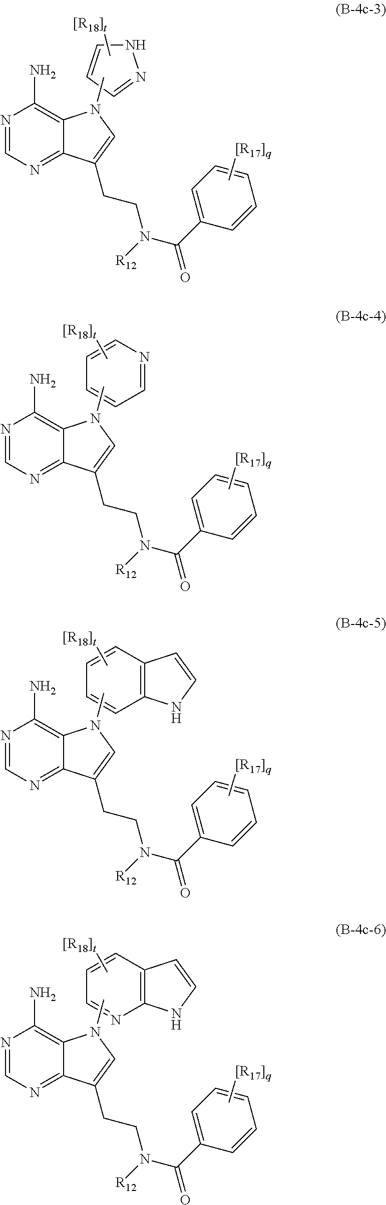 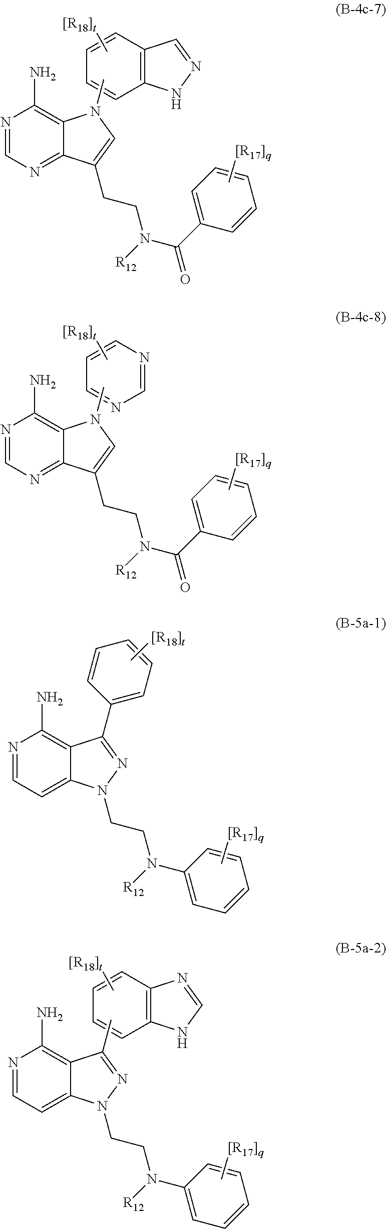 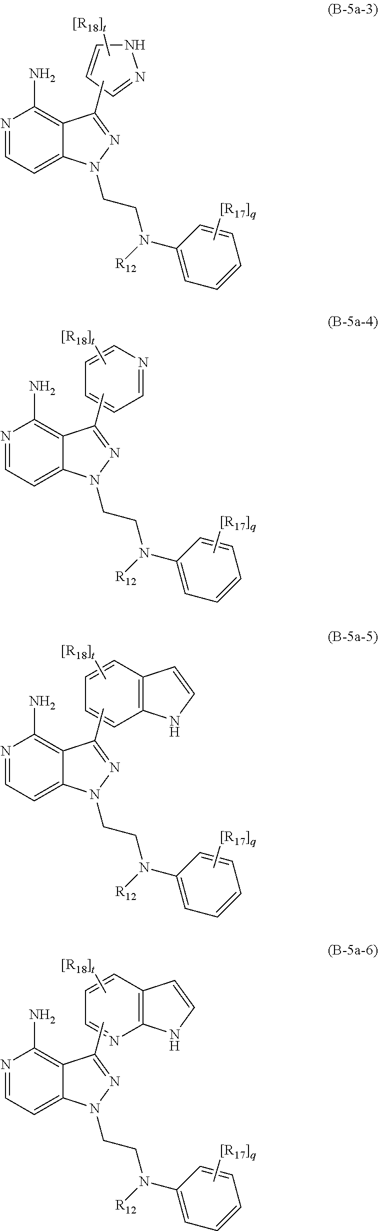 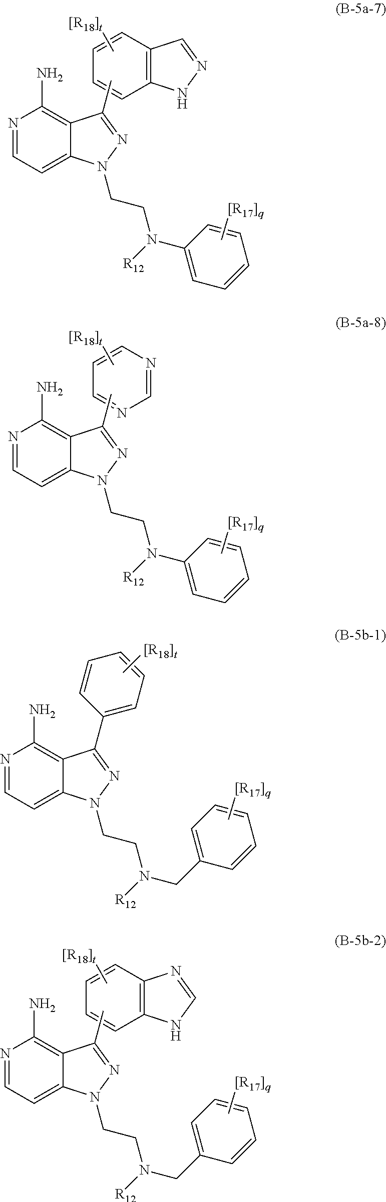 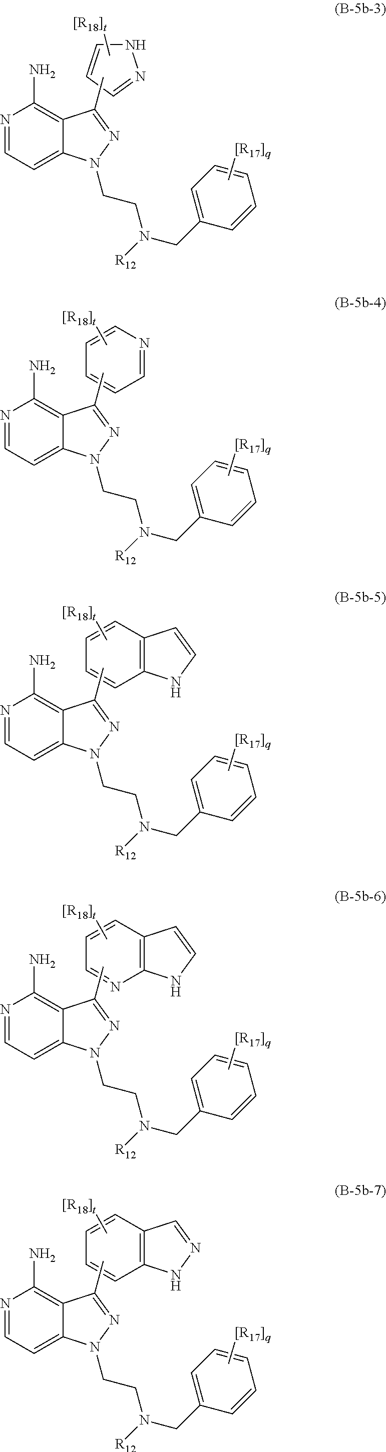 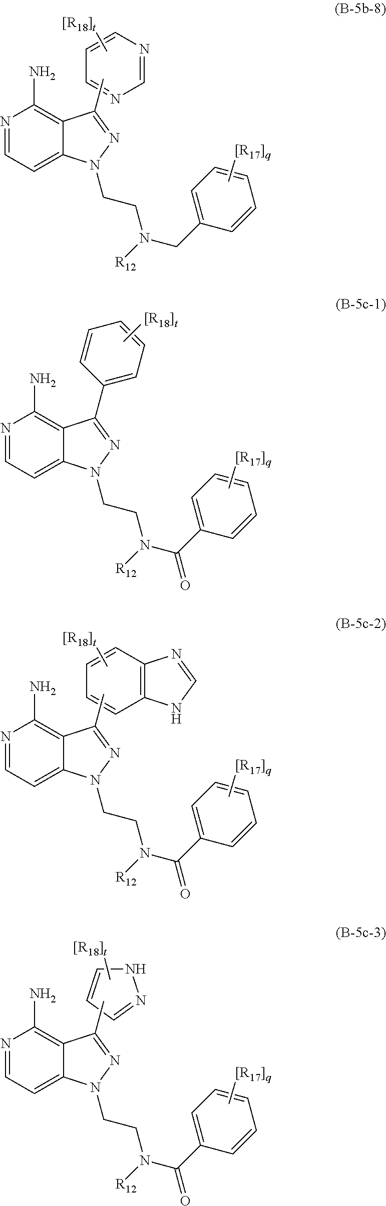 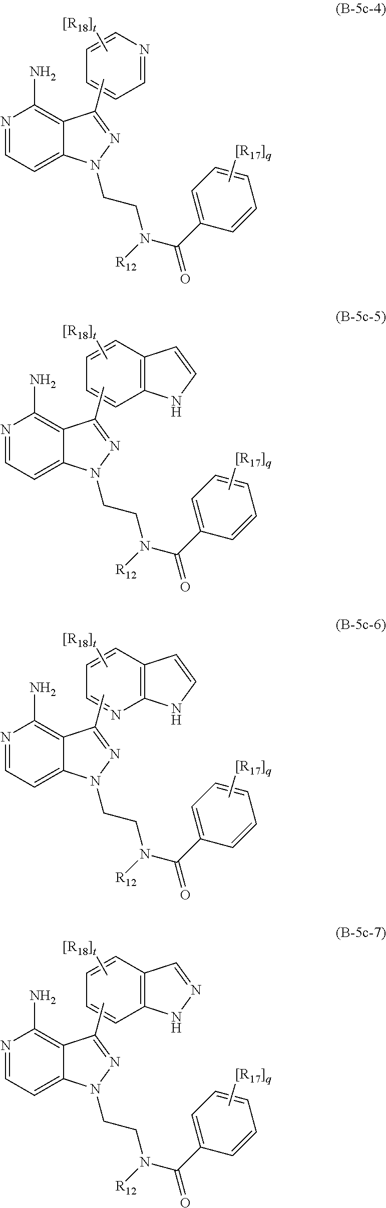 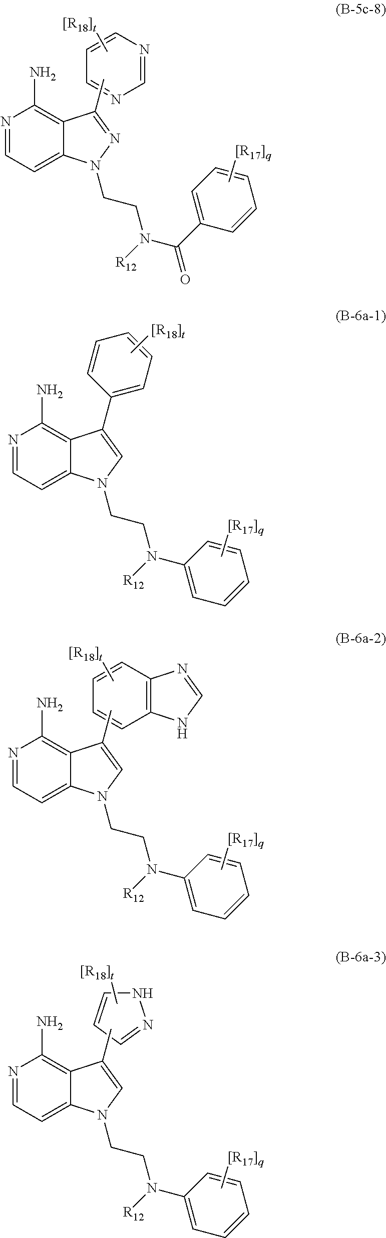 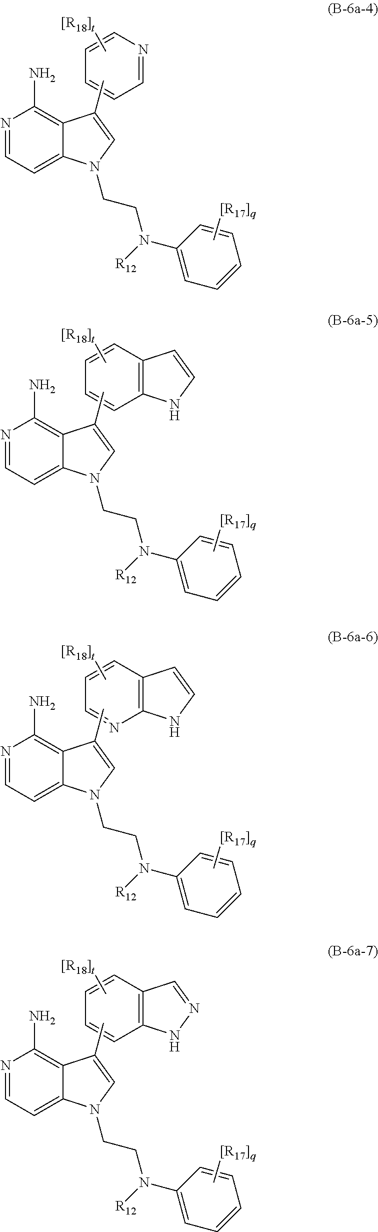 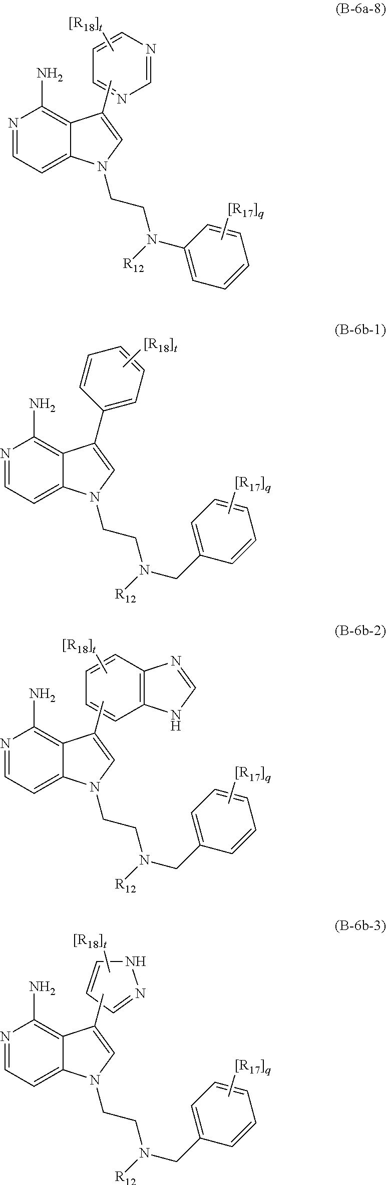 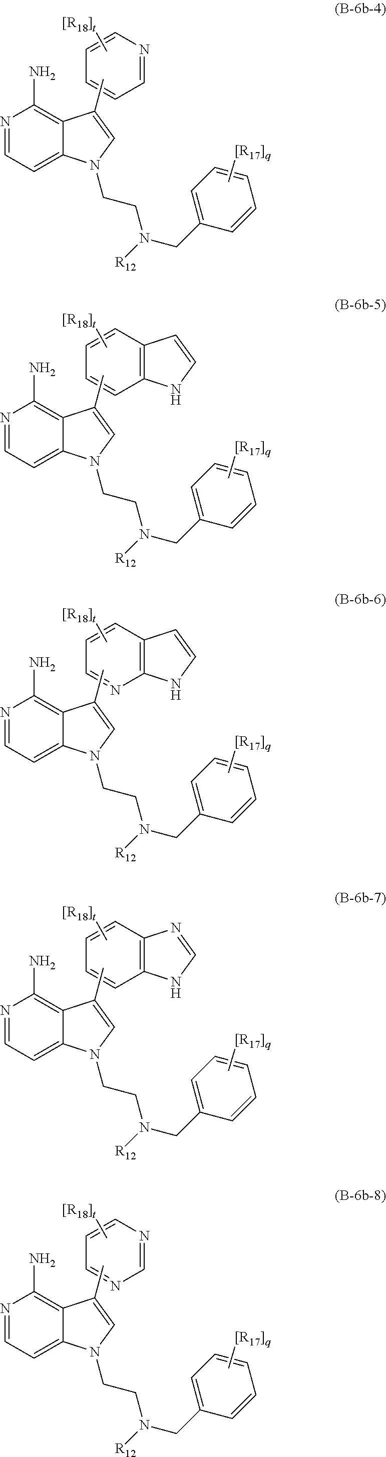 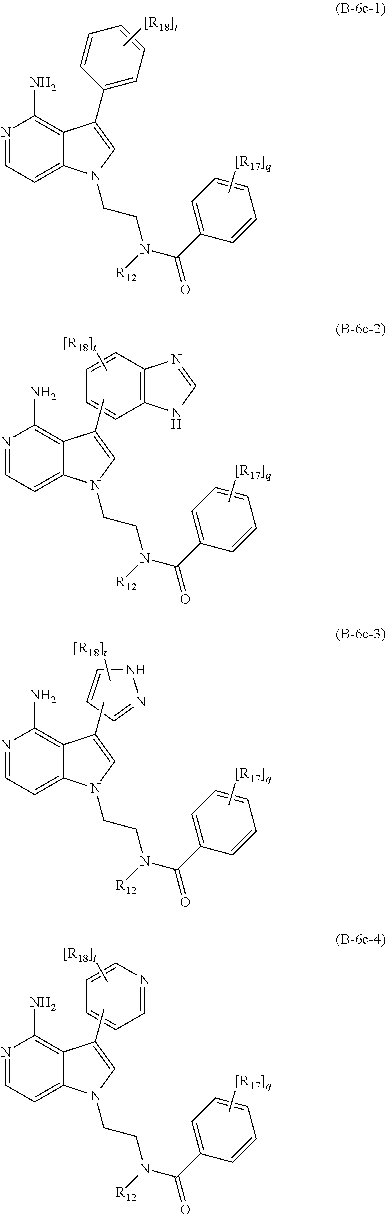 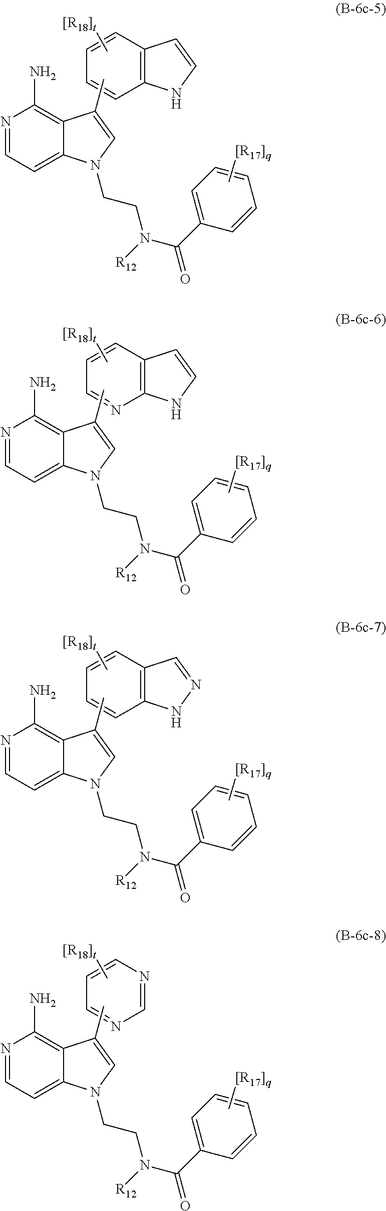 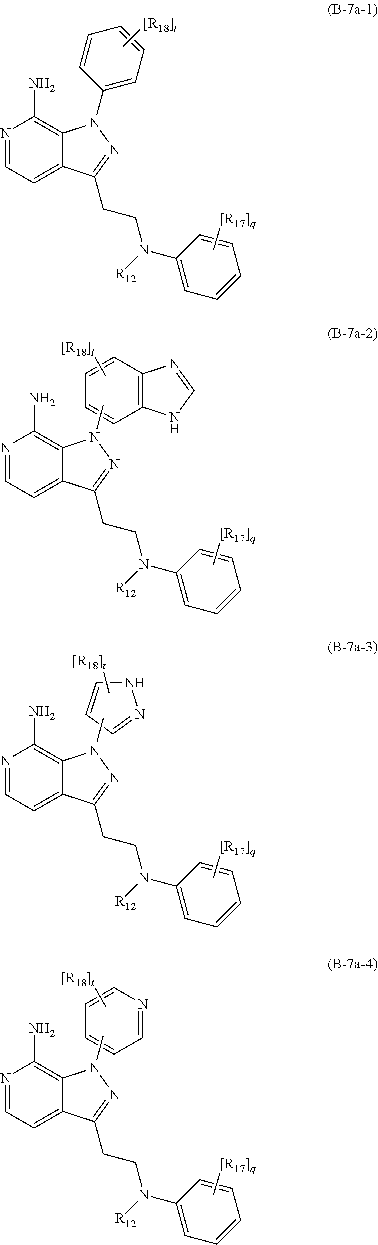 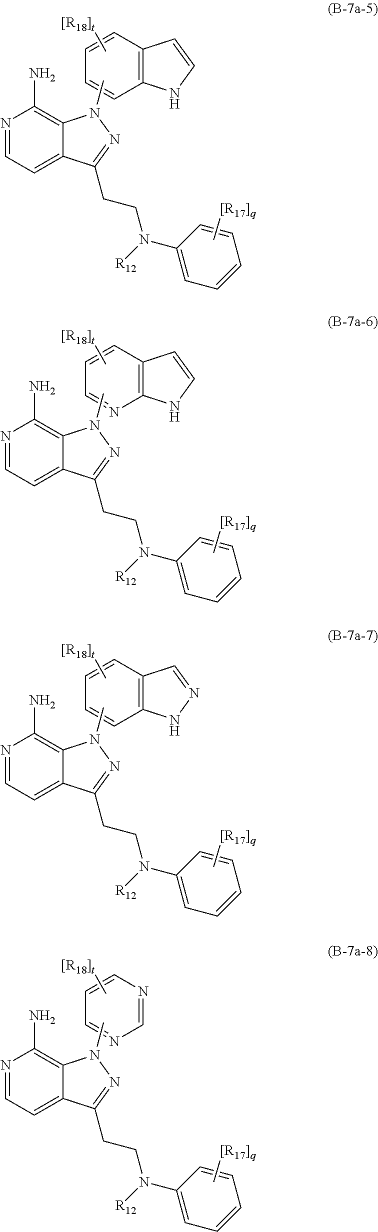 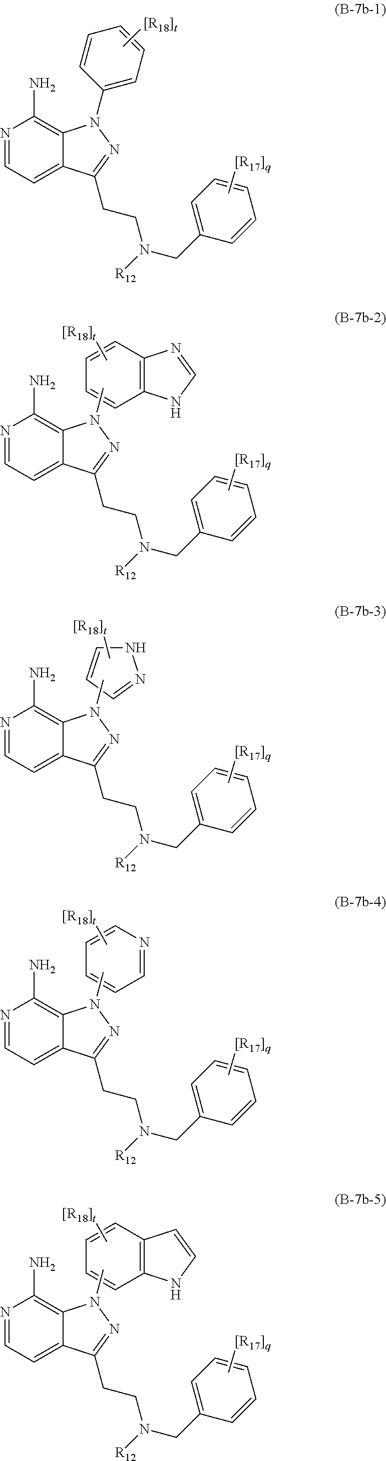 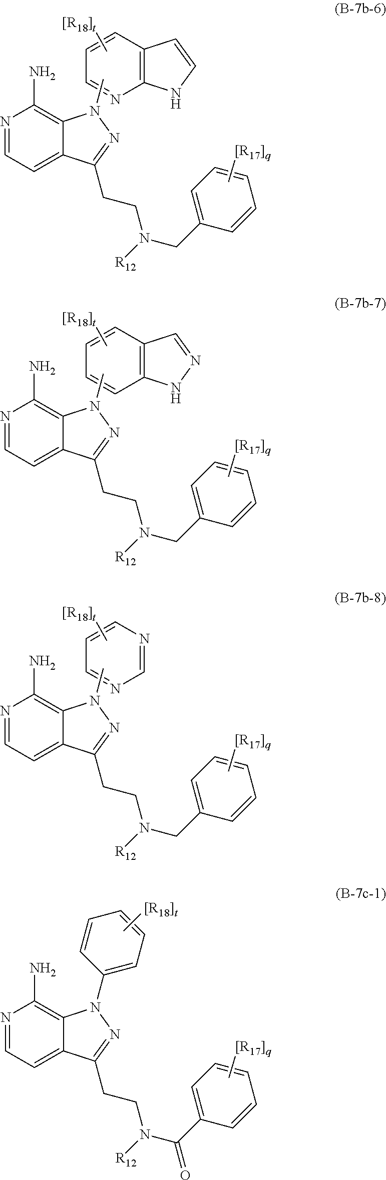 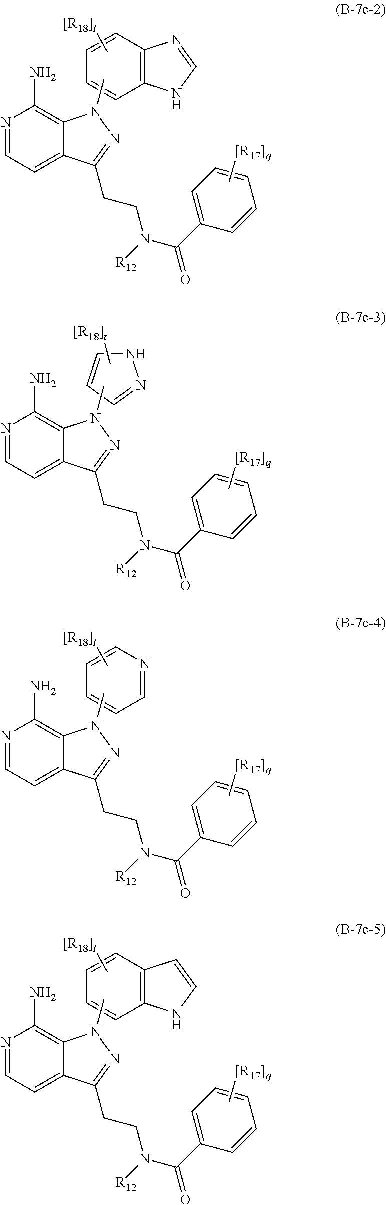 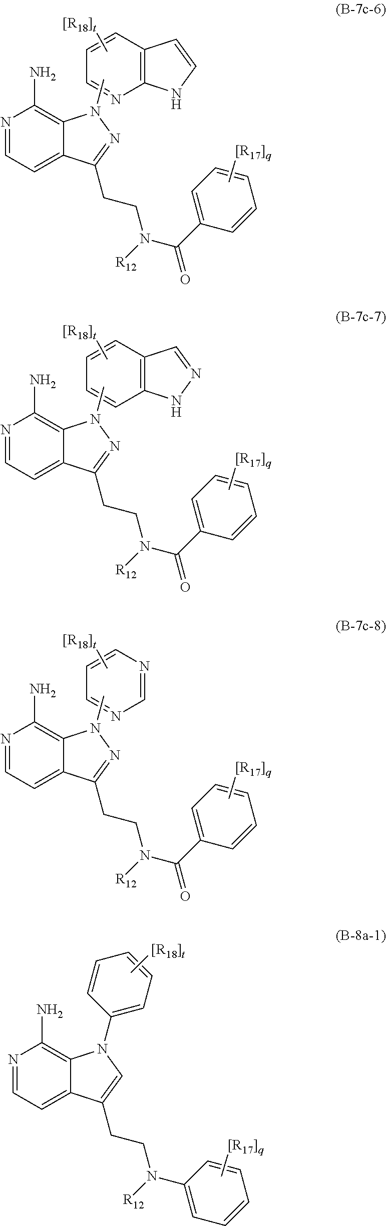 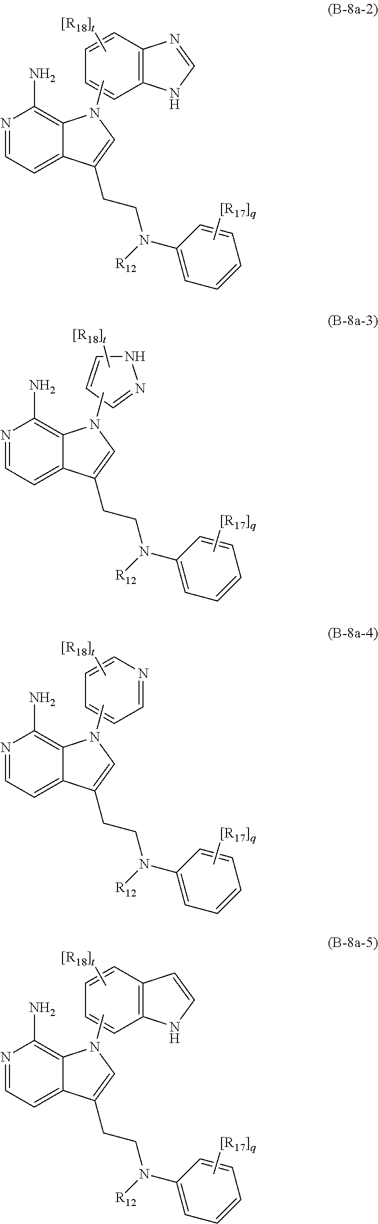 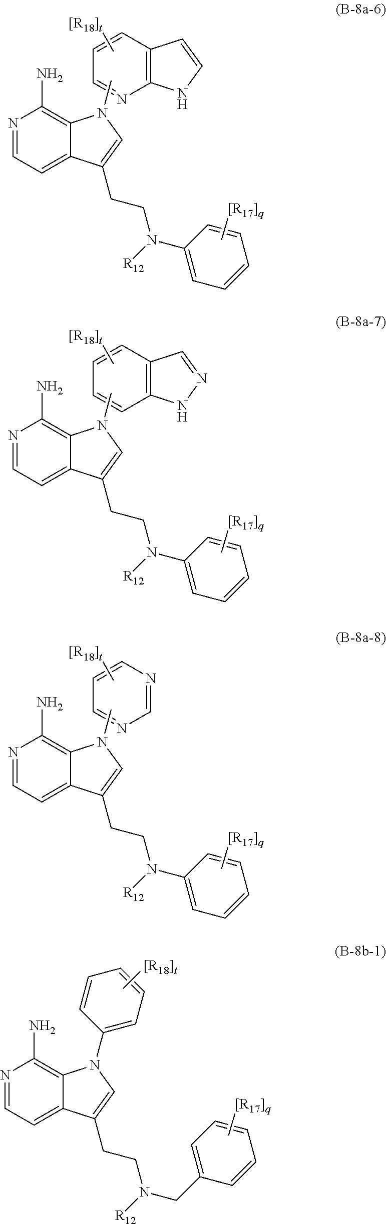 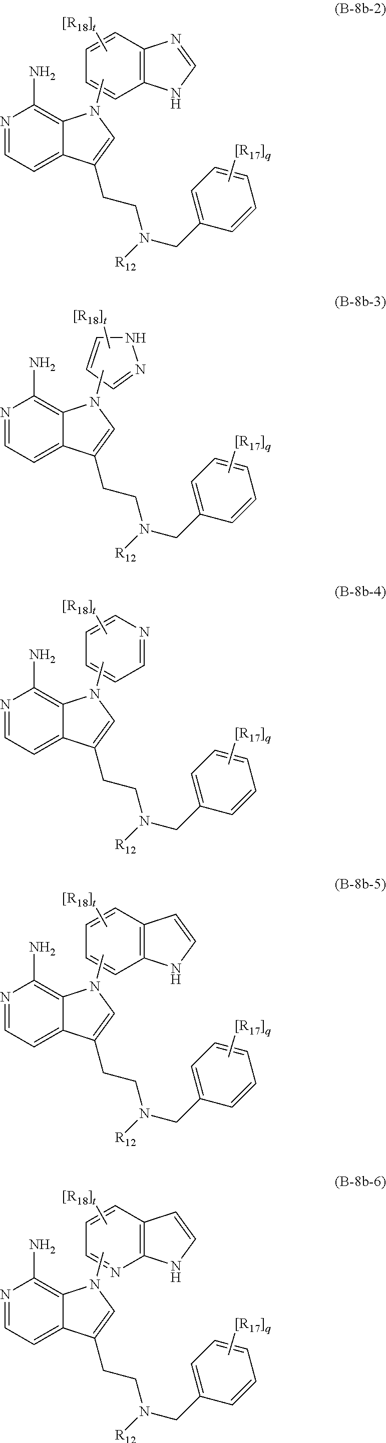 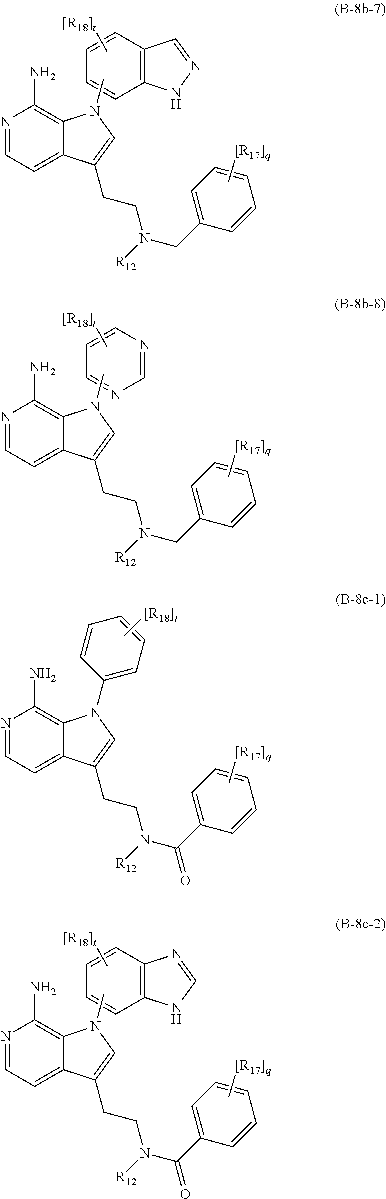 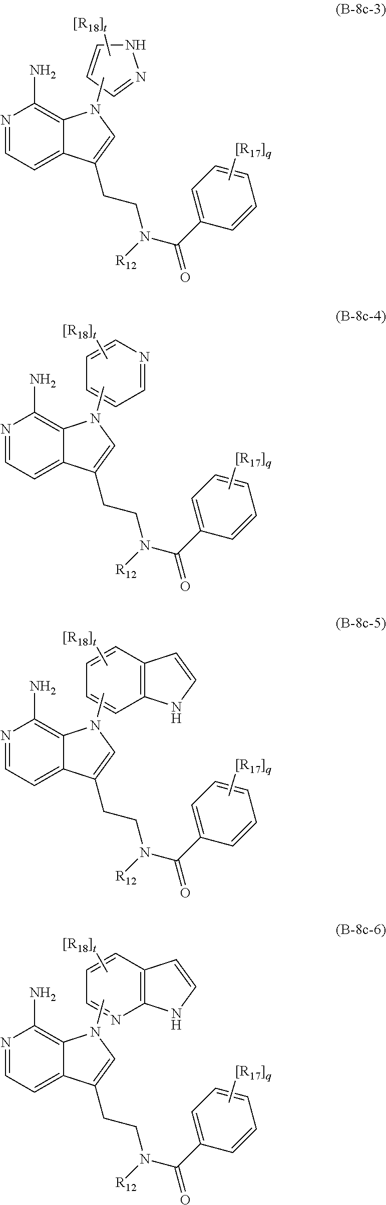 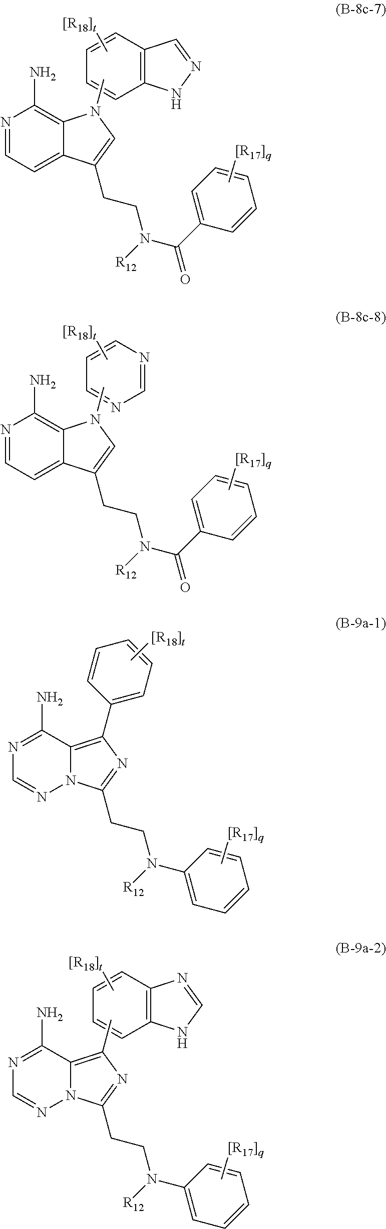 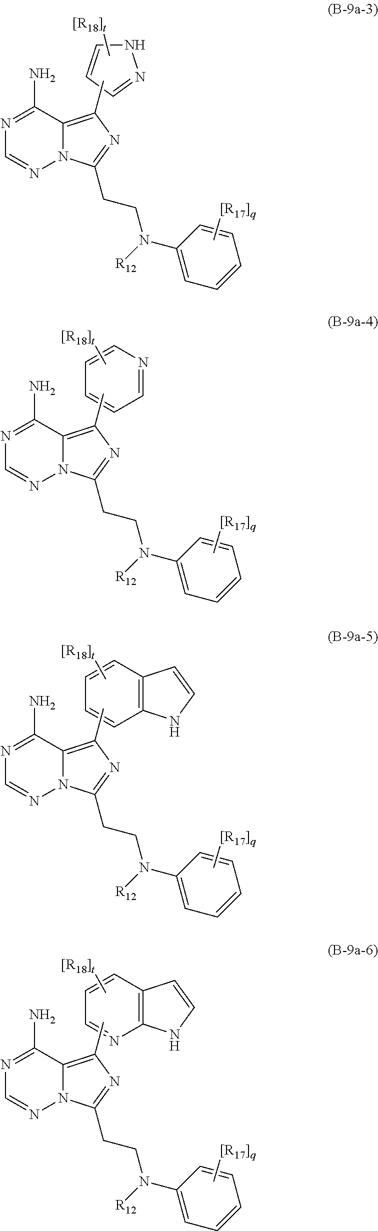 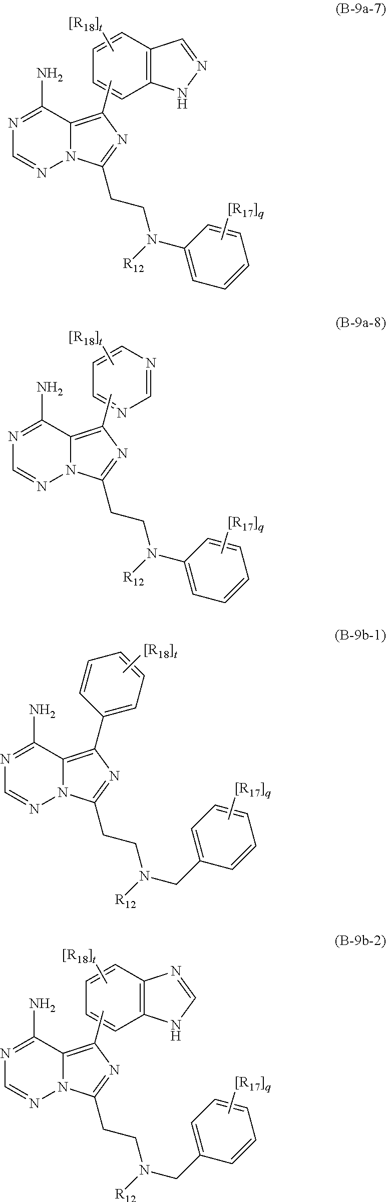 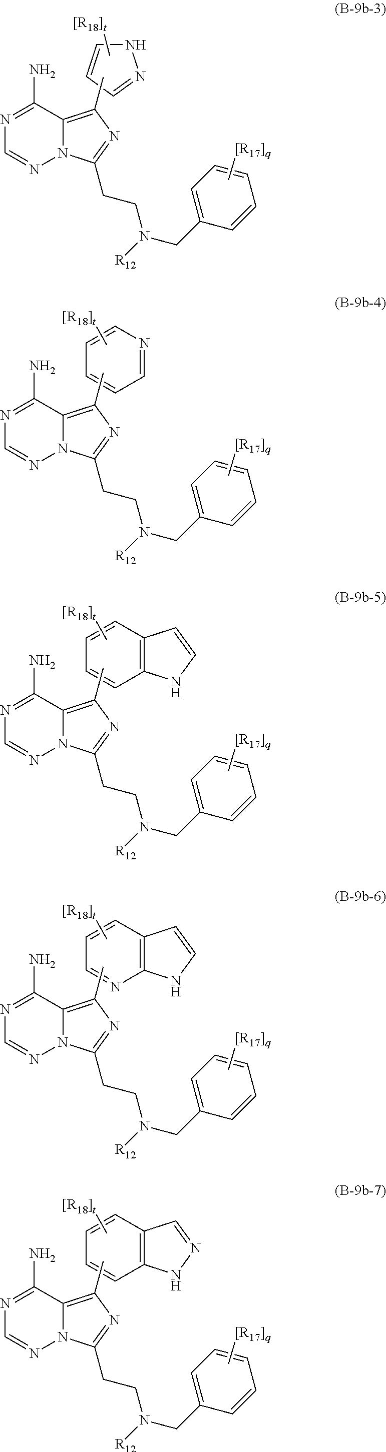 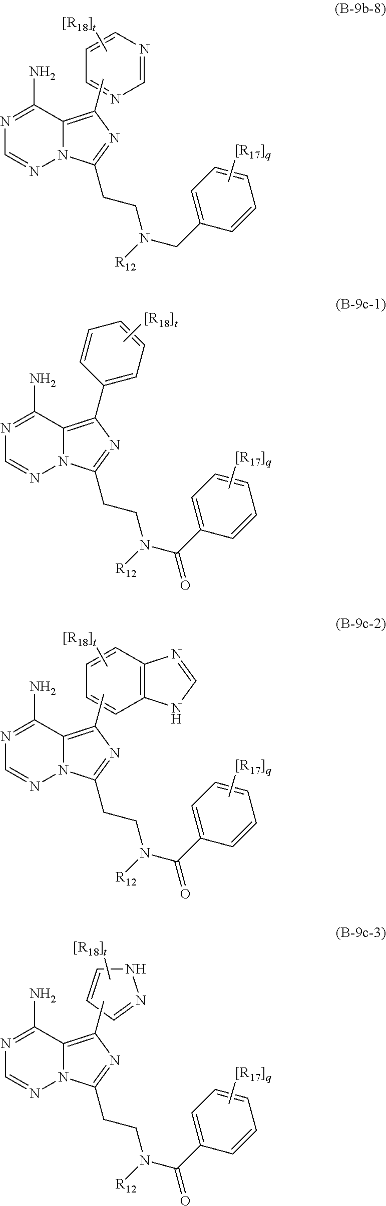 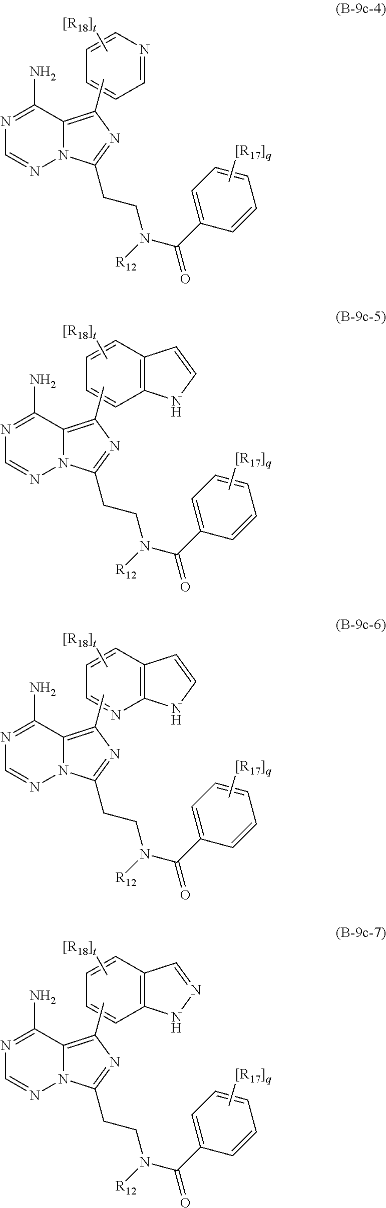 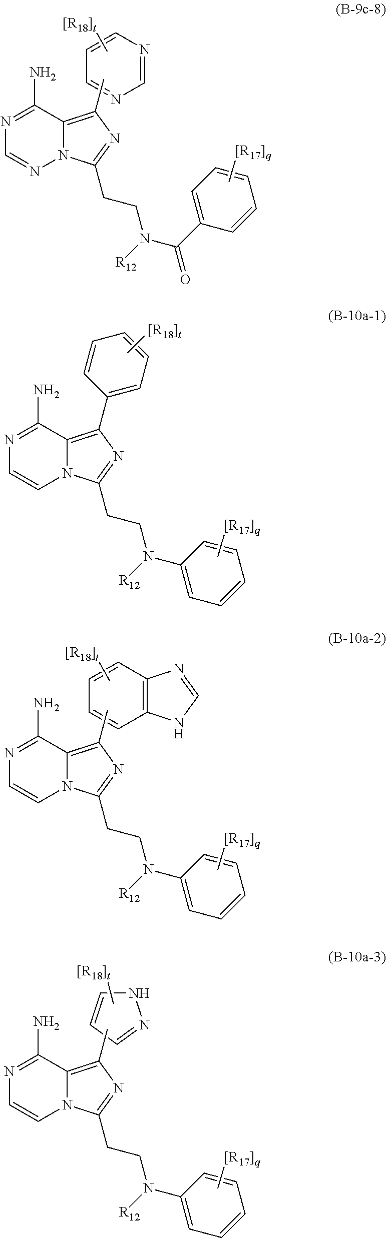 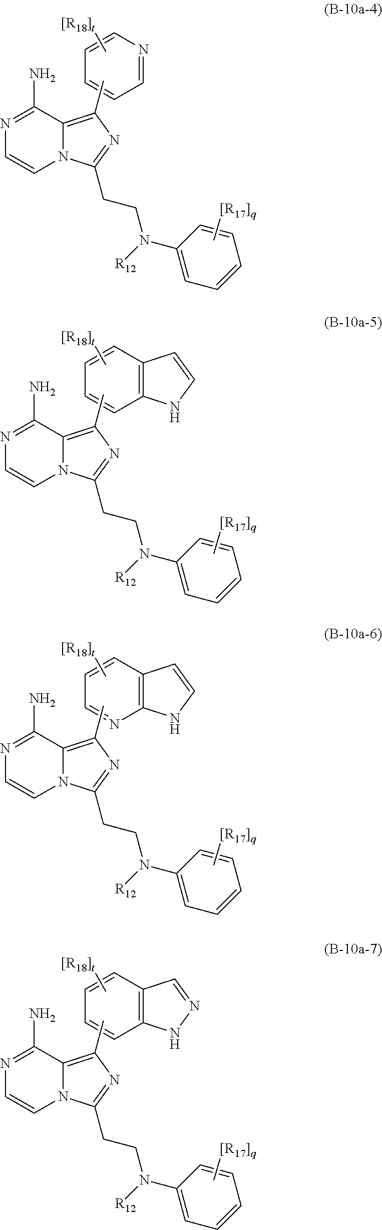 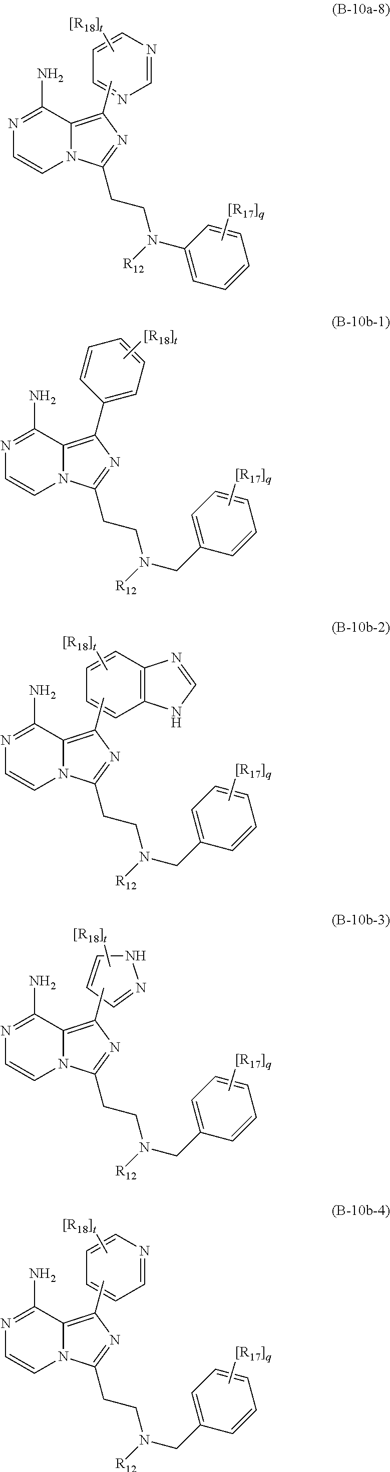 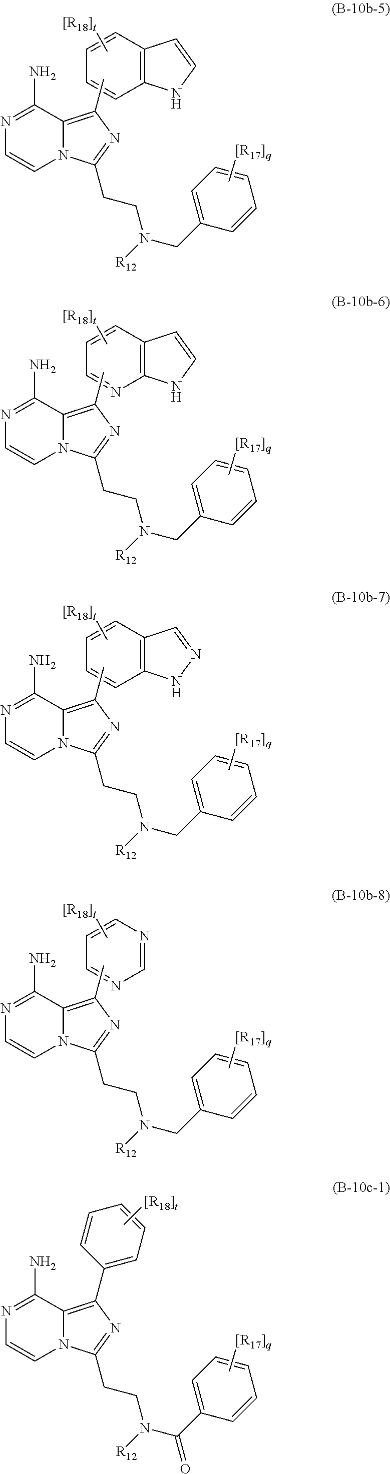 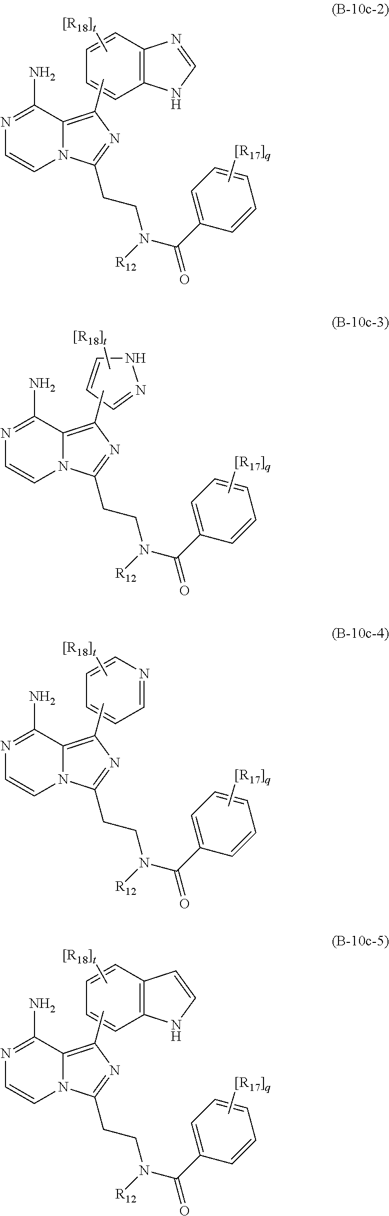 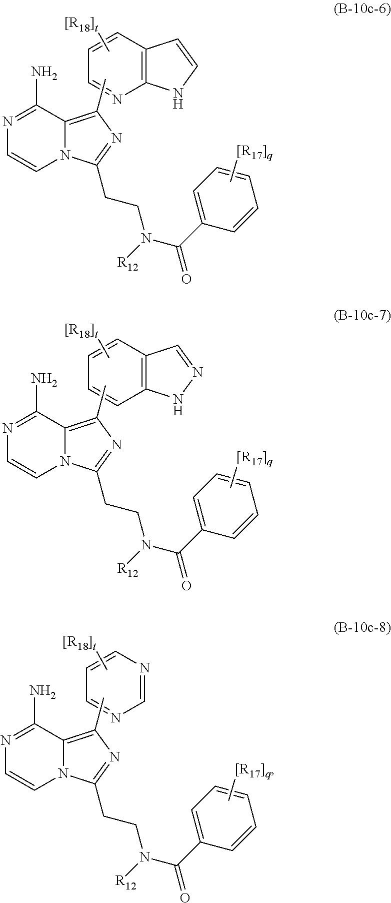
or a salt thereof;
wherein q, R₁₂ and R₁₇ are as described for formulae B-1a, B-1b, B-1c, B-2a, B-2b, B-2c, B-3a, B-3b, B-3c, B-4a, B-4b, B-4c, B-5a, B-5b, B-5c, B-6a, B-6b, B-6c, B-7a, B-7b, B-7c, B-8a, B-8b, B-8c, B-9a, B-9b, B-9c, B-10a, B-10b, and B-10c;
each R₁₈, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₃-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₁₂, or —NHR₁₂; and
t is 0 to 3.

28. The compound of claim 4, wherein the compound is of the structure C-1a, C-1b, C-1c, C-1d, C-1e, C-1f, C-1g, C-1 h, C-1i, C-1j, C-2a, C-2b, C-2c, C-2d, C-2e, C-2f, C-2g, C-2 h, C-2i, C-2j, C-3a, C-3b, C-3c, C-3d, C-3e, C-3f, C-3g, C-3 h, C-3i, or C-3j: 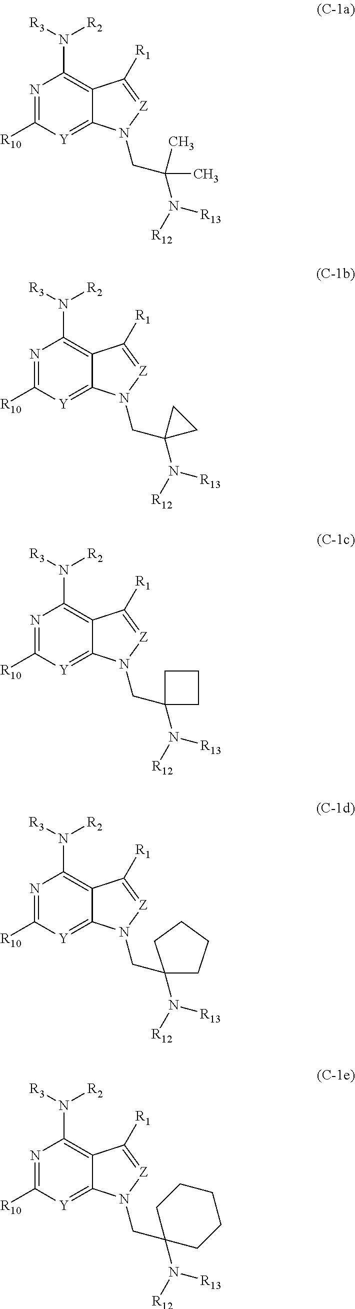 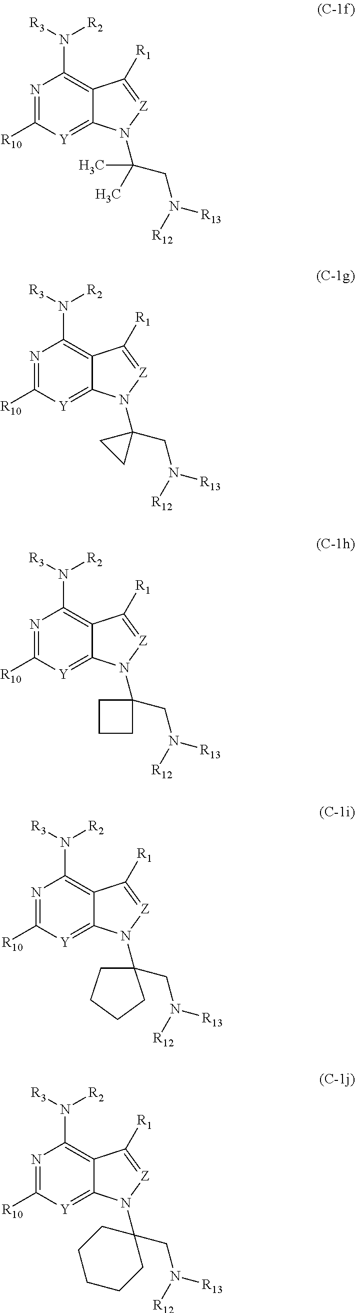 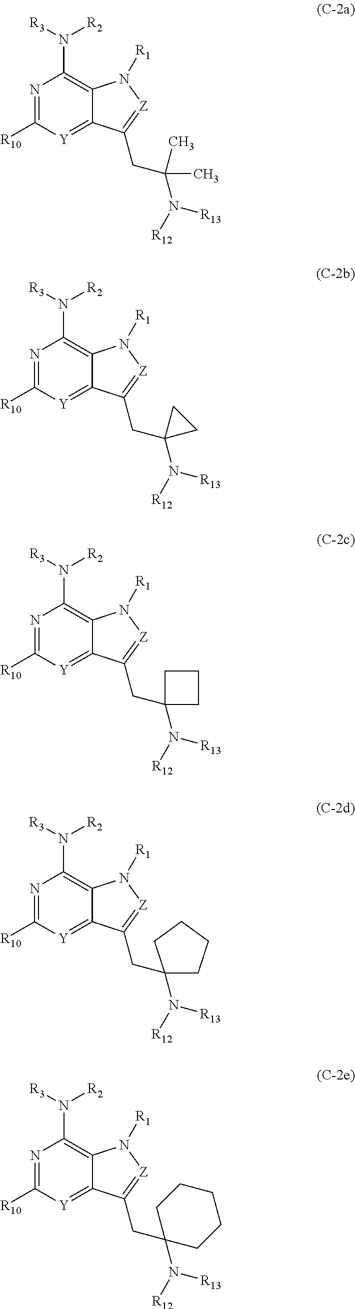 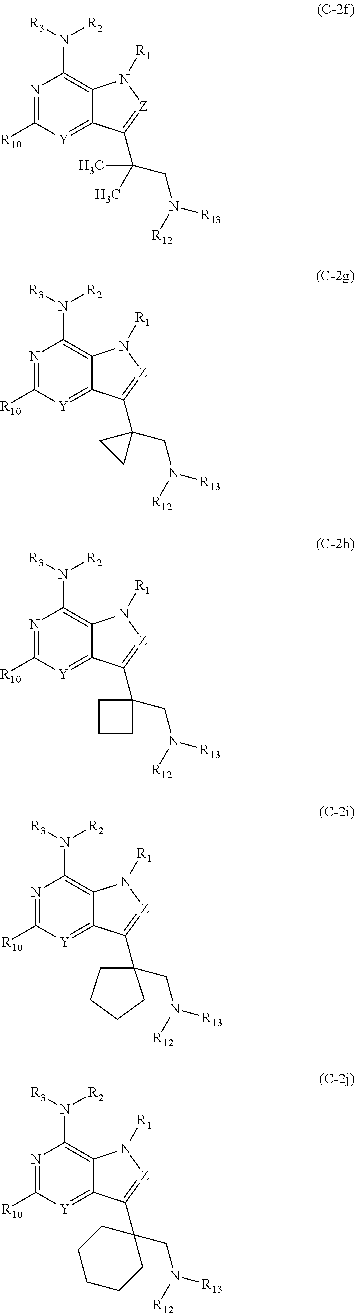 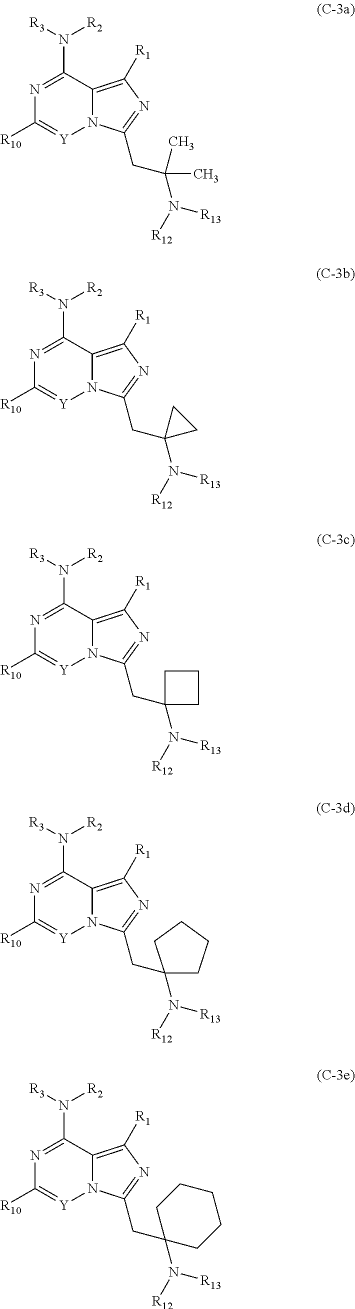 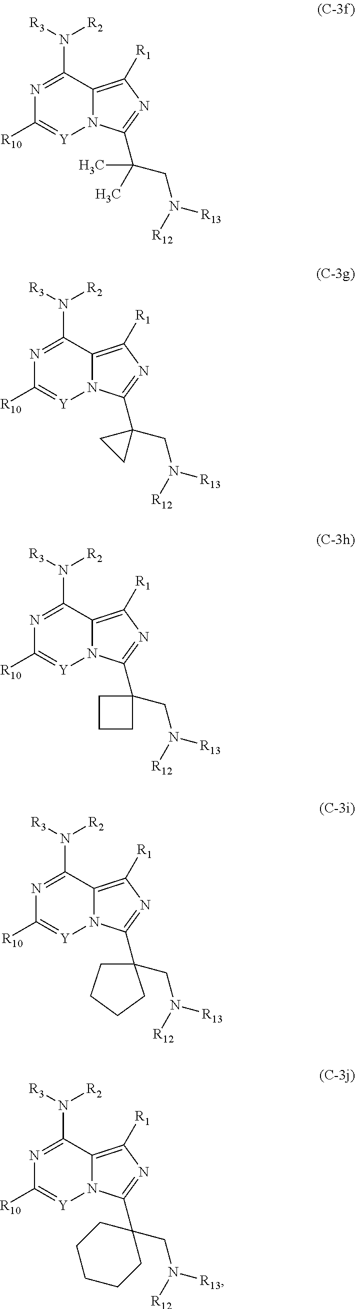
or a salt thereof;
wherein R₁, R₂, R₃, R₁₀, R₁₂, and R₁₃ are as described for formulae (IVa), (iVb), (Va), (Vb), (VIa), and (VIb).

29. The compound of claim 1, wherein the compound is of the structure (D-1), (D-2), or (D-3): 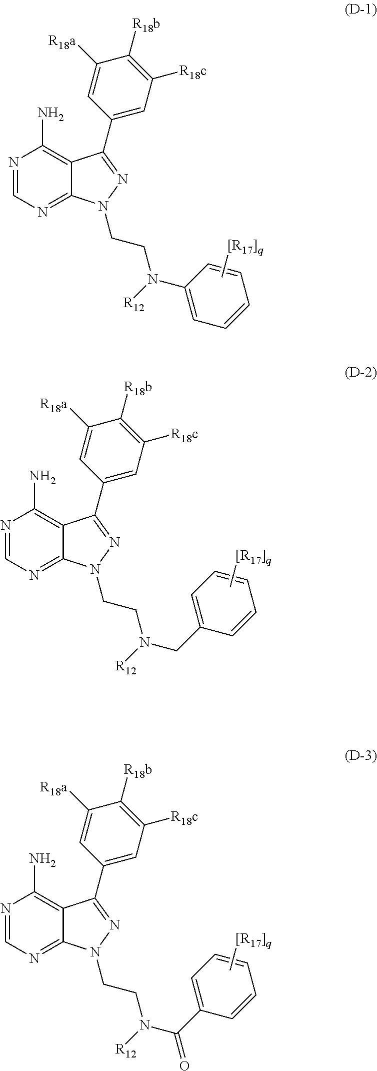
or a salt thereof, wherein R₁₂, R₁₇ and q are as described for formula (I); and each of R₁₈a, R₁₈b, and R₁₈c is independently selected from the group consisting of H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₁₂ and —NHR₁₂.

30. The compound of claim 1, wherein the compound is as described in Table 1 and Table 2, and pharmaceutically acceptable salts thereof.

31. A compound selected from the group consisting of:
3-(4-amino-1-(2-(phenylamino)ethyl)-1H-pyrazolo[3,4-d]pyrimidin-3-yl)-5-fluorophenol;
N-(2-(4-amino-3-(3-fluoro-5-hydroxyphenyl)-1H-pyrazolo[3,4-d]pyrimidin-1-yl)ethyl)-N-phenylacrylamide;
N-(2-(4-amino-3-(3-fluoro-5-hydroxyphenyl)-1H-pyrazolo[3,4-d]pyrimidin-1-yl)ethyl)-4-chloro-2-fluorobenzamide;
3-(4-amino-1-(2-(benzylamino)ethyl)-1H-pyrazolo[3,4-d]pyrimidin-3-yl)-5-fluorophenol;
N-(2-(4-amino-3-(3-fluoro-5-hydroxyphenyl)-1H-pyrazolo[3,4-d]pyrimidin-1-yl)ethyl)-N-benzylacrylamide;
N-(2-(4-amino-3-(3-fluoro-5-hydroxyphenyl)-1H-pyrazolo[3,4-d]pyrimidin-1-yl)ethyl)-N-benzylmethanesulfonamide;
N-(2-(4-amino-3-(3-fluoro-5-hydroxyphenyl)-1H-pyrazolo[3,4-d]pyrimidin-1-yl)ethyl)-N-benzylpropionamide;
N-(2-(4-amino-3-(3-fluoro-5-hydroxyphenyl)-1H-pyrazolo[3,4-d]pyrimidin-1-yl)ethyl)-N-benzylcyclopropanecarboxamide;
3-(4-amino-1-(2-(N-benzylacrylamido)ethyl)-1H-pyrazolo[3,4-d]pyrimidin-3-yl)-5-fluorophenyl acetate;
N-(2-(4-amino-3-(3-fluoro-5-hydroxyphenyl)-1H-pyrazolo[3,4-d]pyrimidin-1-yl)ethyl)-N-(4-fluorobenzyl)acrylamide;
N-(2-(4-amino-3-(3-fluoro-5-hydroxyphenyl)-1H-pyrazolo[3,4-d]pyrimidin-1-yl)ethyl)-N-(2-fluorobenzyl)acrylamide;
N-(2-(4-amino-3-(3-fluoro-5-hydroxyphenyl)-1H-pyrazolo[3,4-d]pyrimidin-1-yl)ethyl)-N-(4-chlorobenzyl)acrylamide;
N-(2-(4-amino-5-(4-chloro-3-hydroxyphenyl)-7H-pyrrolo[2,3-d]pyrimidin-7-yl)ethyl)-N-(4-fluorobenzyl)acrylamide;
N-(2-(4-amino-3-(4-chloro-3-hydroxyphenyl)-1H-pyrazolo[3,4-d]pyrimidin-1-yl)ethyl)-N-(4-fluorobenzyl)acrylamide;
N-(2-(4-amino-3-(4-chloro-3-hydroxyphenyl)-1H-pyrazolo[3,4-d]pyrimidin-1-yl)ethyl)-N-(4-chlorobenzyl)acrylamide;
N-(2-(4-amino-3-(3,4-difluoro-5-hydroxyphenyl)-1H-pyrazolo[3,4-d]pyrimidin-1-yl)ethyl)-N-(4-chlorobenzyl)acrylamide;
N-(2-(4-amino-3-(4-chloro-3-hydroxyphenyl)-1H-pyrazolo[3,4-d]pyrimidin-1-yl)ethyl)-N-(4-methoxybenzyl)acrylamide;
N-(2-(4-amino-3-(8-fluoro-3,4-dihydro-2H-benzo[b][1,4]oxazin-6-yl)-1H-pyrazolo[3,4-d]pyrimidin-1-yl)ethyl)-N-(4-chlorobenzyl)acrylamide;
N-(2-(4-amino-3-(4-chloro-3-hydroxyphenyl)-1H-pyrazolo[3,4-d]pyrimidin-1-yl)ethyl)-N-(thiophen-2-ylmethyl)acrylamide;
N-(2-(4-amino-3-(4-chloro-3-hydroxyphenyl)-1H-pyrazolo[3,4-d]pyrimidin-1-yl)ethyl)-N-(furan-2-ylmethyl)acrylamide;
5-(4-amino-1-(2-(pyrimidin-2-ylamino)ethyl)-1H-pyrazolo[3,4-d]pyrimidin-3-yl)-2,3-difluorophenol;
N-(2-(4-amino-3-(4-chloro-3-cyano-5-hydroxyphenyl)-1H-pyrazolo[3,4-d]pyrimidin-1-yl)ethyl)-N-benzylacrylamide;
5-(4-amino-1-(2-(pyridin-3-ylmethylamino)ethyl)-1H-pyrazolo[3,4-d]pyrimidin-3-yl)-2,3-difluorophenol; and
N-(2-(4-amino-3-(4-phenoxyphenyl)-1H-pyrazolo[3,4-d]pyrimidin-1-yl)ethyl)-N-benzylacrylamide; and
pharmaceutically acceptable salts thereof.

32. A compound selected from the group consisting of:
N-(2-(4-amino-3-(4-fluoro-3-hydroxyphenyl)-1H-pyrazolo[3,4-d]pyrimidin-1-yl)ethyl)-N-phenylacrylamide;
N-(2-(4-amino-3-(3-hydroxy-4-isopropoxyphenyl)-1H-pyrazolo[3,4-d]pyrimidin-1-yl)ethyl)-N-phenylacrylamide;
N-(2-(4-amino-3-(3-hydroxy-4-methoxyphenyl)-1H-pyrazolo[3,4-d]pyrimidin-1-yl)ethyl)-N-phenylacrylamide;
N-(2-(4-amino-3-(3-hydroxy-4-methylphenyl)-1H-pyrazolo[3,4-d]pyrimidin-1-yl)ethyl)-N-phenylacrylamide;
N-(2-(4-amino-3-(3,4-dihydroxyphenyl)-1H-pyrazolo[3,4-d]pyrimidin-1-yl)ethyl)-N-phenylacrylamide;
N-(2-(4-amino-3-(4-methoxy-3-(methylamino)phenyl)-1H-pyrazolo[3,4-d]pyrimidin-1-yl)ethyl)-N-phenylacrylamide;
N-(2-(4-amino-3-(4-fluoro-3-(methylamino)phenyl)-1H-pyrazolo[3,4-d]pyrimidin-1-yl)ethyl)-N-phenylacrylamide;
N-(2-(4-amino-3-(4-hydroxyphenyl)-1H-pyrazolo[3,4-d]pyrimidin-1-yl)ethyl)-N-phenylacrylamide;
N-(2-(4-amino-3-(4-hydroxy-3-methoxyphenyl)-1H-pyrazolo[3,4-d]pyrimidin-1-yl)ethyl)-N-phenylacrylamide;
N-(2-(4-amino-3-(4-hydroxy-3-(trifluoromethyl)phenyl)-1H-pyrazolo[3,4-d]pyrimidin-1-yl)ethyl)-N-phenylacrylamide;
N-(2-(4-amino-3-(3-cyano-4-hydroxyphenyl)-1H-pyrazolo[3,4-d]pyrimidin-1-yl)ethyl)-N-phenylacrylamide;
N-(2-(4-amino-3-(2-hydroxyphenylamino)-1H-pyrazolo[3,4-d]pyrimidin-1-yl)ethyl)-N-phenylacrylamide;
N-(2-(4-amino-3-(3-fluoro-5-methoxyphenyl)-1H-pyrazolo[3,4-d]pyrimidin-1-yl)ethyl)-N-phenylacrylamide;
N-(2-(4-amino-3-(4-bromo-3-hydroxyphenyl)-1H-pyrazolo[3,4-d]pyrimidin-1-yl)ethyl)-N-phenylacrylamide;
N-(2-(4-amino-3-(1H-indazol-5-yl)-1H-pyrazolo[3,4-d]pyrimidin-1-yl)ethyl)-N-phenylacrylamide;
N-(2-(4-amino-3-(1H-indol-6-yl)-1H-pyrazolo[3,4-d]pyrimidin-1-yl)ethyl)-N-phenylacrylamide;
N-(2-(4-amino-3-(1H-benzo[d]imidazol-6-yl)-1H-pyrazolo[3,4-d]pyrimidin-1-yl)ethyl)-N-phenylacrylamide;
N-(2-(4-amino-3-(1H-pyrazol-4-yl)-1H-pyrazolo[3,4-d]pyrimidin-1-yl)ethyl)-N-phenylacrylamide;
N-(2-(4-amino-3-(5-fluoro-6-methoxypyridin-3-yl)-1H-pyrazolo[3,4-d]pyrimidin-1-yl)ethyl)-N-phenylacrylamide;
4-(4-amino-1-(2-(N-phenylacrylamido)ethyl)-1H-pyrazolo[3,4-d]pyrimidin-3-yl)benzamide;
3-(4-amino-1-(2-(N-phenylacrylamido)ethyl)-1H-pyrazolo[3,4-d]pyrimidin-3-yl)benzamide;
N-(2-(4-amino-3-(3,4-dimethoxyphenyl)-1H-pyrazolo[3,4-d]pyrimidin-1-yl)ethyl)-N-phenylacrylamide;
N-(2-(4-amino-3-(4-ethoxy-3-methoxyphenyl)-1H-pyrazolo[3,4-d]pyrimidin-1-yl)ethyl)-N-phenylacrylamide;
N-(2-(4-amino-3-(4-chloro-3-hydroxyphenyl)-1H-pyrazolo[3,4-d]pyrimidin-1-yl)ethyl)-N-phenylacrylamide;
N-(2-(4-amino-3-(3-(hydroxymethyl)phenyl)-1H-pyrazolo[3,4-d]pyrimidin-1-yl)ethyl)-N-phenylacrylamide;
N-(2-(4-amino-3-(4-(hydroxymethyl)phenyl)-1H-pyrazolo[3,4-d]pyrimidin-1-yl)ethyl)-N-phenylacrylamide;
N-(2-(3-(3-(allyloxy)-5-fluorophenyl)-4-amino-1H-pyrazolo[3,4-d]pyrimidin-1-yl)ethyl)-N-phenylacrylamide;
N-(2-(3-(4-acrylamido-3-hydroxyphenyl)-4-amino-1H-pyrazolo[3,4-d]pyrimidin-1-yl)ethyl)-N-phenylacrylamide;
N-(2-(3-(3-acrylamido-5-hydroxyphenyl)-4-amino-1H-pyrazolo[3,4-d]pyrimidin-1-yl)ethyl)-N-phenylacrylamide;
N-(2-(4-amino-3-(1H-indol-1-yl)-1H-pyrazolo[3,4-d]pyrimidin-1-yl)ethyl)-N-phenylacrylamide;
N-(2-(4-amino-3-(5-fluoro-7-hydroxy-1H-indol-1-yl)-1H-pyrazolo[3,4-d]pyrimidin-1-yl)ethyl)-N-phenylacrylamide;
N-(2-(4-amino-3-(6-fluoro-5-hydroxy-1H-indol-1-yl)-1H-pyrazolo[3,4-d]pyrimidin-1-yl)ethyl)-N-phenylacrylamide;
N-(2-(4-amino-3-(4-amino-3-hydroxyphenyl)-1H-pyrazolo[3,4-d]pyrimidin-1-yl)ethyl)-N-phenylacrylamide;
N-(2-(4-amino-3-(3-hydroxy-4-(trifluoromethyl)phenyl)-1H-pyrazolo[3,4-d]pyrimidin-1-yl)ethyl)-N-phenylacrylamide;
N-(2-(3-(4-acryloyl-3-fluorophenyl)-4-amino-1H-pyrazolo[3,4-d]pyrimidin-1-yl)ethyl)-N-phenylacrylamide;
N-(2-(3-(3-acryloyl-5-fluorophenyl)-4-amino-1H-pyrazolo[3,4-d]pyrimidin-1-yl)ethyl)-N-phenylacrylamide;
N-(2-(4-amino-3-(4-fluoro-3-hydroxyphenyl)-1H-pyrazolo[3,4-d]pyrimidin-1-yl)ethyl)-N-benzylacrylamide;
N-(2-(4-amino-3-(3-hydroxy-4-isopropoxyphenyl)-1H-pyrazolo[3,4-d]pyrimidin-1-yl)ethyl)-N-benzylacrylamide;
N-(2-(4-amino-3-(3-hydroxy-4-methoxyphenyl)-1H-pyrazolo[3,4-d]pyrimidin-1-yl)ethyl)-N-benzylacrylamide;
N-(2-(4-amino-3-(3-hydroxy-4-methylphenyl)-1H-pyrazolo[3,4-d]pyrimidin-1-yl)ethyl)-N-benzylacrylamide;
N-(2-(4-amino-3-(3,4-dihydroxyphenyl)-1H-pyrazolo[3,4-d]pyrimidin-1-yl)ethyl)-N-benzylacrylamide;
N-(2-(4-amino-3-(4-methoxy-3-(methylamino)phenyl)-1H-pyrazolo[3,4-d]pyrimidin-1-yl)ethyl)-N-benzylacrylamide;
N-(2-(4-amino-3-(4-fluoro-3-(methylamino)phenyl)-1H-pyrazolo[3,4-d]pyrimidin-1-yl)ethyl)-N-benzylacrylamide;
N-(2-(4-amino-3-(4-hydroxyphenyl)-1H-pyrazolo[3,4-d]pyrimidin-1-yl)ethyl)-N-benzylacrylamide;
N-(2-(4-amino-3-(4-hydroxy-3-methoxyphenyl)-1H-pyrazolo[3,4-d]pyrimidin-1-yl)ethyl)-N-benzylacrylamide;
N-(2-(4-amino-3-(4-hydroxy-3-(trifluoromethyl)phenyl)-1H-pyrazolo[3,4-d]pyrimidin-1-yl)ethyl)-N-benzylacrylamide;
N-(2-(4-amino-3-(3-cyano-4-hydroxyphenyl)-1H-pyrazolo[3,4-d]pyrimidin-1-yl)ethyl)-N-benzylacrylamide;
N-(2-(4-amino-3-(2-hydroxyphenylamino)-1H-pyrazolo[3,4-d]pyrimidin-1-yl)ethyl)-N-benzylacrylamide;
N-(2-(4-amino-3-(3-fluoro-5-methoxyphenyl)-1H-pyrazolo[3,4-d]pyrimidin-1-yl)ethyl)-N-benzylacrylamide;
N-(2-(4-amino-3-(4-bromo-3-hydroxyphenyl)-1H-pyrazolo[3,4-d]pyrimidin-1-yl)ethyl)-N-benzylacrylamide;
N-(2-(4-amino-3-(1H-indazol-5-yl)-1H-pyrazolo[3,4-d]pyrimidin-1-yl)ethyl)-N-benzylacrylamide;
N-(2-(4-amino-3-(1H-indol-6-yl)-1H-pyrazolo[3,4-d]pyrimidin-1-yl)ethyl)-N-benzylacrylamide;
N-(2-(4-amino-3-(1H-benzo[d]imidazol-6-yl)-1H-pyrazolo[3,4-d]pyrimidin-1-yl)ethyl)-N-benzylbenzamide;
N-(2-(4-amino-3-(1H-pyrazol-4-yl)-1H-pyrazolo[3,4-d]pyrimidin-1-yl)ethyl)-N-benzylacrylamide;
N-(2-(4-amino-3-(5-fluoro-6-methoxypyridin-3-yl)-1H-pyrazolo[3,4-d]pyrimidin-1-yl)ethyl)-N-benzylacrylamide;
4-(4-amino-1-(2-(N-benzylacrylamido)ethyl)-1H-pyrazolo[3,4-d]pyrimidin-3-yl)benzamide;
3-(4-amino-1-(2-(N-benzylacrylamido)ethyl)-1H-pyrazolo[3,4-d]pyrimidin-3-yl)benzamide;
N-(2-(4-amino-3-(3,4-dimethoxyphenyl)-1H-pyrazolo[3,4-d]pyrimidin-1-yl)ethyl)-N-benzylacrylamide;
N-(2-(4-amino-3-(4-ethoxy-3-methoxyphenyl)-1H-pyrazolo[3,4-d]pyrimidin-1-yl)ethyl)-N-benzylacrylamide;
N-(2-(4-amino-3-(4-chloro-3-hydroxyphenyl)-1H-pyrazolo[3,4-d]pyrimidin-1-yl)ethyl)-N-benzylacrylamide;
N-(2-(4-amino-3-(3-(hydroxymethyl)phenyl)-1H-pyrazolo[3,4-d]pyrimidin-1-yl)ethyl)-N-benzylacrylamide;
N-(2-(4-amino-3-(4-(hydroxymethyl)phenyl)-1H-pyrazolo[3,4-d]pyrimidin-1-yl)ethyl)-N-benzylacrylamide;
N-(2-(3-(3-(allyloxy)-5-fluorophenyl)-4-amino-1H-pyrazolo[3,4-d]pyrimidin-1-yl)ethyl)-N-benzylacrylamide;
N-(2-(3-(4-acrylamido-3-hydroxyphenyl)-4-amino-1H-pyrazolo[3,4-d]pyrimidin-1-yl)ethyl)-N-benzylacrylamide;
N-(2-(3-(3-acrylamido-5-hydroxyphenyl)-4-amino-1H-pyrazolo[3,4-d]pyrimidin-1-yl)ethyl)-N-benzylacrylamide;
N-(2-(4-amino-3-(1H-indol-1-yl)-1H-pyrazolo[3,4-d]pyrimidin-1-yl)ethyl)-N-benzylacrylamide;
N-(2-(4-amino-3-(5-fluoro-7-hydroxy-1H-indol-1-yl)-1H-pyrazolo[3,4-d]pyrimidin-1-yl)ethyl)-N-benzylacryl amide;
N-(2-(4-amino-3-(6-fluoro-5-hydroxy-1H-indol-1-yl)-1H-pyrazolo[3,4-d]pyrimidin-1-yl)ethyl)-N-benzylacryl amide;
N-(2-(4-amino-3-(4-amino-3-hydroxyphenyl)-1H-pyrazolo[3,4-d]pyrimidin-1-yl)ethyl)-N-benzylacryl amide;
N-(2-(4-amino-3-(3-hydroxy-4-(trifluoromethyl)phenyl)-1H-pyrazolo [3,4-d]pyrimidin-1-yl)ethyl)-N-benzylacryl amide;
N-(2-(3-(4-acryloyl-3-fluorophenyl)-4-amino-1H-pyrazolo[3,4-d]pyrimidin-1-yl)ethyl)-N-benzylacryl amide;
N-(2-(3-(3-acryloyl-5-fluorophenyl)-4-amino-1H-pyrazolo[3,4-d]pyrimidin-1-yl)ethyl)-N-benzylacryl amide;
N-acryloyl-N-(2-(4-amino-3-(4-fluoro-3-hydroxyphenyl)-1H-pyrazolo [3,4-d]pyrimidin-1-yl)ethyl)benz amide;
N-acryloyl-N-(2-(4-amino-3-(3-hydroxy-4-isopropoxyphenyl)-1H-pyrazolo[3,4-d]pyrimidin-1-yl)ethyl)benz amide;
N-acryloyl-N-(2-(4-amino-3-(3-hydroxy-4-methoxyphenyl)-1H-pyrazolo [3,4-d]pyrimidin-1-yl)ethyl)benz amide;
N-acryloyl-N-(2-(4-amino-3-(3-hydroxy-4-methylphenyl)-1H-pyrazolo [3,4-d]pyrimidin-1-yl)ethyl)benz amide;
N-acryloyl-N-(2-(4-amino-3-(3,4-dihydroxyphenyl)-1H-pyrazolo[3,4-d]pyrimidin-1-yl)ethyl)benz amide;
N-acryloyl-N-(2-(4-amino-3-(4-methoxy-3-(methylamino)phenyl)-1H-pyrazolo [3,4-d]pyrimidin-1-yl)ethyl)benzamide;
N-acryloyl-N-(2-(4-amino-3-(4-fluoro-3-(methylamino)phenyl)-1H-pyrazolo [3,4-d]pyrimidin-1-yl)ethyl)benz amide;
N-acryloyl-N-(2-(4-amino-3-(4-hydroxyphenyl)-1H-pyrazolo[3,4-d]pyrimidin-1-yl)ethyl)benz amide;
N-acryloyl-N-(2-(4-amino-3-(4-hydroxy-3-methoxyphenyl)-1H-pyrazolo [3,4-d]pyrimidin-1-yl)ethyl)benz amide;
N-acryloyl-N-(2-(4-amino-3-(4-hydroxy-3-(trifluoromethyl)phenyl)-1H-pyrazolo[3,4-d]pyrimidin-1-yl)ethyl)benzamide;
N-acryloyl-N-(2-(4-amino-3-(3-cyano-4-hydroxyphenyl)-1H-pyrazolo[3,4-d]pyrimidin-1-yl)ethyl)benzamide;
N-acryloyl-N-(2-(4-amino-3-(2-hydroxyphenylamino)-1H-pyrazolo[3,4-d]pyrimidin-1-yl)ethyl)benzamide;
N-acryloyl-N-(2-(4-amino-3-(3-fluoro-5-methoxyphenyl)-1H-pyrazolo[3,4-d]pyrimidin-1-yl)ethyl)benzamide;
N-acryloyl-N-(2-(4-amino-3-(4-bromo-3-hydroxyphenyl)-1H-pyrazolo[3,4-d]pyrimidin-1-yl)ethyl)benzamide;
N-acryloyl-N-(2-(4-amino-3-(1H-indazol-5-yl)-1H-pyrazolo[3,4-d]pyrimidin-1-yl)ethyl)benzamide;
N-acryloyl-N-(2-(4-amino-3-(1H-indol-6-yl)-1H-pyrazolo[3,4-d]pyrimidin-1-yl)ethyl)benzamide;
N-(2-(4-amino-3-(1H-benzo[d]imidazol-6-yl)-1H-pyrazolo[3,4-d]pyrimidin-1-yl)ethyl)-N-benzoylbenzamide;
N-acryloyl-N-(2-(4-amino-3-(1H-pyrazol-4-yl)-1H-pyrazolo[3,4-d]pyrimidin-1-yl)ethyl)benzamide;
N-acryloyl-N-(2-(4-amino-3-(5-fluoro-6-methoxypyridin-3-yl)-1H-pyrazolo[3,4-d]pyrimidin-1-yl)ethyl)benzamide;
N-acryloyl-N-(2-(4-amino-3-(4-carbamoylphenyl)-1H-pyrazolo[3,4-d]pyrimidin-1-yl)ethyl)benzamide;
N-acryloyl-N-(2-(4-amino-3-(3-carbamoylphenyl)-1H-pyrazolo[3,4-d]pyrimidin-1-yl)ethyl)benzamide;
N-acryloyl-N-(2-(4-amino-3-(3,4-dimethoxyphenyl)-1H-pyrazolo[3,4-d]pyrimidin-1-yl)ethyl)benzamide;
N-acryloyl-N-(2-(4-amino-3-(4-ethoxy-3-methoxyphenyl)-1H-pyrazolo[3,4-d]pyrimidin-1-yl)ethyl)benzamide;
N-acryloyl-N-(2-(4-amino-3-(4-chloro-3-hydroxyphenyl)-1H-pyrazolo[3,4-d]pyrimidin-1-yl)ethyl)benzamide;
N-acryloyl-N-(2-(4-amino-3-(3-(hydroxymethyl)phenyl)-1H-pyrazolo[3,4-d]pyrimidin-1-yl)ethyl)benzamide;
N-acryloyl-N-(2-(4-amino-3-(4-(hydroxymethyl)phenyl)-1H-pyrazolo[3,4-d]pyrimidin-1-yl)ethyl)benzamide;
N-acryloyl-N-(2-(3-(3-(allyloxy)-5-fluorophenyl)-4-amino-1H-pyrazolo[3,4-d]pyrimidin-1-yl)ethyl)benzamide;
N-(2-(3-(4-acrylamido-3-hydroxyphenyl)-4-amino-1H-pyrazolo[3,4-d]pyrimidin-1-yl)ethyl)-N-acryloylbenzamide;
N-(2-(3-(3-acrylamido-5-hydroxyphenyl)-4-amino-1H-pyrazolo[3,4-d]pyrimidin-1-yl)ethyl)-N-acryloylbenzamide;
N-acryloyl-N-(2-(4-amino-3-(1H-indol-1-yl)-1H-pyrazolo[3,4-d]pyrimidin-1-yl)ethyl)benzamide;
N-acryloyl-N-(2-(4-amino-3-(5-fluoro-7-hydroxy-1H-indol-1-yl)-1H-pyrazolo[3,4-d]pyrimidin-1-yl)ethyl)benzamide;
N-acryloyl-N-(2-(4-amino-3-(6-fluoro-5-hydroxy-1H-indol-1-yl)-1H-pyrazolo[3,4-d]pyrimidin-1-yl)ethyl)benzamide;
N-acryloyl-N-(2-(4-amino-3-(4-amino-3-hydroxyphenyl)-1H-pyrazolo[3,4-d]pyrimidin-1-yl)ethyl)benzamide;
N-acryloyl-N-(2-(4-amino-3-(3-hydroxy-4-(trifluoromethyl)phenyl)-1H-pyrazolo[3,4-d]pyrimidin-1-yl)ethyl)benzamide;
N-acryloyl-N-(2-(3-(4-acryloyl-3-fluorophenyl)-4-amino-1H-pyrazolo[3,4-d]pyrimidin-1-yl)ethyl)benzamide;
N-acryloyl-N-(2-(3-(3-acryloyl-5-fluorophenyl)-4-amino-1H-pyrazolo[3,4-d]pyrimidin-1-yl)ethyl)benzamide;
N-(2-(4-amino-5-(4-fluoro-3-hydroxyphenyl)-7H-pyrrolo[2,3-d]pyrimidin-7-yl)ethyl)-N-phenylacrylamide;
N-(2-(4-amino-5-(3-hydroxy-4-isopropoxyphenyl)-7H-pyrrolo[2,3-d]pyrimidin-7-yl)ethyl)-N-phenylacrylamide;
N-(2-(4-amino-5-(3-hydroxy-4-methoxyphenyl)-7H-pyrrolo[2,3-d]pyrimidin-7-yl)ethyl)-N-phenylacrylamide;
N-(2-(4-amino-5-(3-hydroxy-4-methylphenyl)-7H-pyrrolo[2,3-d]pyrimidin-7-yl)ethyl)-N-phenylacrylamide;
N-(2-(4-amino-5-(3,4-dihydroxyphenyl)-7H-pyrrolo[2,3-d]pyrimidin-7-yl)ethyl)-N-phenylacrylamide;
N-(2-(4-amino-5-(4-methoxy-3-(methylamino)phenyl)-7H-pyrrolo[2,3-d]pyrimidin-7-yl)ethyl)-N-phenylacrylamide;
N-(2-(4-amino-5-(4-fluoro-3-(methylamino)phenyl)-7H-pyrrolo[2,3-d]pyrimidin-7-yl)ethyl)-N-phenylacrylamide;
N-(2-(4-amino-5-(4-hydroxyphenyl)-7H-pyrrolo[2,3-d]pyrimidin-7-yl)ethyl)-N-phenylacrylamide;
N-(2-(4-amino-5-(4-hydroxy-3-methoxyphenyl)-7H-pyrrolo[2,3-d]pyrimidin-7-yl)ethyl)-N-phenylacrylamide;
N-(2-(4-amino-5-(4-hydroxy-3-(trifluoromethyl)phenyl)-7H-pyrrolo[2,3-d]pyrimidin-7-yl)ethyl)-N-phenylacrylamide;
N-(2-(4-amino-5-(3-cyano-4-hydroxyphenyl)-7H-pyrrolo[2,3-d]pyrimidin-7-yl)ethyl)-N-phenylacrylamide;
N-(2-(4-amino-5-(2-hydroxyphenylamino)-7H-pyrrolo[2,3-d]pyrimidin-7-yl)ethyl)-N-phenylacrylamide;
N-(2-(4-amino-5-(3-fluoro-5-methoxyphenyl)-7H-pyrrolo[2,3-d]pyrimidin-7-yl)ethyl)-N-phenylacrylamide;
N-(2-(4-amino-5-(4-bromo-3-hydroxyphenyl)-7H-pyrrolo[2,3-d]pyrimidin-7-yl)ethyl)-N-phenylacrylamide;
N-(2-(4-amino-5-(1H-indazol-5-yl)-7H-pyrrolo[2,3-d]pyrimidin-7-yl)ethyl)-N-phenylacrylamide;
N-(2-(4-amino-5-(1H-indol-6-yl)-7H-pyrrolo[2,3-d]pyrimidin-7-yl)ethyl)-N-phenylacrylamide;
N-(2-(4-amino-5-(1H-benzo[d]imidazol-6-yl)-7H-pyrrolo[2,3-d]pyrimidin-7-yl)ethyl)-N-phenylacrylamide;
N-(2-(4-amino-5-(1H-pyrazol-4-yl)-7H-pyrrolo[2,3-d]pyrimidin-7-yl)ethyl)-N-phenylacrylamide;
N-(2-(4-amino-5-(5-fluoro-6-methoxypyridin-3-yl)-7H-pyrrolo[2,3-d]pyrimidin-7-yl)ethyl)-N-phenylacrylamide;
4-(4-amino-7-(2-(N-phenylacrylamido)ethyl)-7H-pyrrolo[2,3-d]pyrimidin-5-yl)benzamide;
3-(4-amino-7-(2-(N-phenylacrylamido)ethyl)-7H-pyrrolo[2,3-d]pyrimidin-5-yl)benzamide;
N-(2-(4-amino-5-(3,4-dimethoxyphenyl)-7H-pyrrolo[2,3-d]pyrimidin-7-yl)ethyl)-N-phenylacrylamide;
N-(2-(4-amino-5-(4-ethoxy-3-methoxyphenyl)-7H-pyrrolo[2,3-d]pyrimidin-7-yl)ethyl)-N-phenylacrylamide;
N-(2-(4-amino-5-(4-chloro-3-hydroxyphenyl)-7H-pyrrolo[2,3-d]pyrimidin-7-yl)ethyl)-N-phenylacrylamide;
N-(2-(4-amino-5-(3-(hydroxymethyl)phenyl)-7H-pyrrolo[2,3-d]pyrimidin-7-yl)ethyl)-N-phenylacrylamide;
N-(2-(4-amino-5-(4-(hydroxymethyl)phenyl)-7H-pyrrolo[2,3-d]pyrimidin-7-yl)ethyl)-N-phenylacrylamide;
N-(2-(5-(3-(allyloxy)-5-fluorophenyl)-4-amino-7H-pyrrolo[2,3-d]pyrimidin-7-yl)ethyl)-N-phenylacrylamide;
N-(2-(5-(4-acrylamido-3-hydroxyphenyl)-4-amino-7H-pyrrolo[2,3-d]pyrimidin-7-yl)ethyl)-N-phenylacrylamide;
N-(2-(5-(3-acrylamido-5-hydroxyphenyl)-4-amino-7H-pyrrolo[2,3-d]pyrimidin-7-yl)ethyl)-N-phenylacrylamide;
N-(2-(4-amino-5-(1H-indol-1-yl)-7H-pyrrolo[2,3-d]pyrimidin-7-yl)ethyl)-N-phenylacrylamide;
N-(2-(4-amino-5-(5-fluoro-7-hydroxy-1H-indol-1-yl)-7H-pyrrolo[2,3-d]pyrimidin-7-yl)ethyl)-N-phenylacrylamide;
N-(2-(4-amino-5-(6-fluoro-5-hydroxy-1H-indol-1-yl)-7H-pyrrolo[2,3-d]pyrimidin-7-yl)ethyl)-N-phenylacrylamide;
N-(2-(4-amino-5-(4-amino-3-hydroxyphenyl)-7H-pyrrolo[2,3-d]pyrimidin-7-yl)ethyl)-N-phenylacrylamide;
N-(2-(4-amino-5-(3-hydroxy-4-(trifluoromethyl)phenyl)-7H-pyrrolo[2,3-d]pyrimidin-7-yl)ethyl)-N-phenylacrylamide;
N-(2-(5-(4-acryloyl-3-fluorophenyl)-4-amino-7H-pyrrolo[2,3-d]pyrimidin-7-yl)ethyl)-N-phenylacrylamide;
N-(2-(5-(3-acryloyl-5-fluorophenyl)-4-amino-7H-pyrrolo[2,3-d]pyrimidin-7-yl)ethyl)-N-phenylacrylamide;
N-(2-(7-amino-1-(4-fluoro-3-hydroxyphenyl)-1H-pyrazolo[4,3-d]pyrimidin-3-yl)ethyl)-N-phenylacrylamide;
N-(2-(7-amino-1-(3-hydroxy-4-isopropoxyphenyl)-1H-pyrazolo[4,3-d]pyrimidin-3-yl)ethyl)-N-phenylacrylamide;
N-(2-(7-amino-1-(3-hydroxy-4-methoxyphenyl)-1H-pyrazolo[4,3-d]pyrimidin-3-yl)ethyl)-N-phenylacrylamide;
N-(2-(7-amino-1-(3-hydroxy-4-methylphenyl)-1H-pyrazolo[4,3-d]pyrimidin-3-yl)ethyl)-N-phenylacrylamide;
N-(2-(7-amino-1-(3,4-dihydroxyphenyl)-1H-pyrazolo[4,3-d]pyrimidin-3-yl)ethyl)-N-phenylacrylamide;
N-(2-(7-amino-1-(4-methoxy-3-(methylamino)phenyl)-1H-pyrazolo[4,3-d]pyrimidin-3-yl)ethyl)-N-phenylacrylamide;
N-(2-(7-amino-1-(4-fluoro-3-(methylamino)phenyl)-1H-pyrazolo[4,3-d]pyrimidin-3-yl)ethyl)-N-phenylacrylamide;
N-(2-(7-amino-1-(4-hydroxyphenyl)-1H-pyrazolo[4,3-d]pyrimidin-3-yl)ethyl)-N-phenylacrylamide;
N-(2-(7-amino-1-(4-hydroxy-3-methoxyphenyl)-1H-pyrazolo[4,3-d]pyrimidin-3-yl)ethyl)-N-phenylacrylamide;
N-(2-(7-amino-1-(4-hydroxy-3-(trifluoromethyl)phenyl)-1H-pyrazolo[4,3-d]pyrimidin-3-yl)ethyl)-N-phenylacrylamide;
N-(2-(7-amino-1-(3-cyano-4-hydroxyphenyl)-1H-pyrazolo[4,3-d]pyrimidin-3-yl)ethyl)-N-phenylacrylamide;
N-(2-(7-amino-1-(2-hydroxyphenylamino)-1H-pyrazolo[4,3-d]pyrimidin-3-yl)ethyl)-N-phenylacrylamide;
N-(2-(7-amino-1-(3-fluoro-5-methoxyphenyl)-1H-pyrazolo[4,3-d]pyrimidin-3-yl)ethyl)-N-phenylacrylamide;
N-(2-(7-amino-1-(4-bromo-3-hydroxyphenyl)-1H-pyrazolo[4,3-d]pyrimidin-3-yl)ethyl)-N-phenylacrylamide;
N-(2-(7-amino-1-(1H-indazol-5-yl)-1H-pyrazolo[4,3-d]pyrimidin-3-yl)ethyl)-N-phenylacrylamide;
N-(2-(7-amino-1-(1H-indol-6-yl)-1H-pyrazolo[4,3-d]pyrimidin-3-yl)ethyl)-N-phenylacrylamide;
N-(2-(7-amino-1-(1H-benzo[d]imidazol-6-yl)-1H-pyrazolo[4,3-d]pyrimidin-3-yl)ethyl)-N-phenylacrylamide;
N-(2-(7-amino-1-(1H-pyrazol-4-yl)-1H-pyrazolo[4,3-d]pyrimidin-3-yl)ethyl)-N-phenylacrylamide;
N-(2-(7-amino-1-(5-fluoro-6-methoxypyridin-3-yl)-1H-pyrazolo[4,3-d]pyrimidin-3-yl)ethyl)-N-phenylacrylamide;
4-(7-amino-3-(2-(N-phenylacrylamido)ethyl)-1H-pyrazolo[4,3-d]pyrimidin-1-yl)benzamide;
3-(7-amino-3-(2-(N-phenylacrylamido)ethyl)-1H-pyrazolo[4,3-d]pyrimidin-1-yl)benzamide;
N-(2-(7-amino-1-(3,4-dimethoxyphenyl)-1H-pyrazolo[4,3-d]pyrimidin-3-yl)ethyl)-N-phenylacrylamide;
N-(2-(7-amino-1-(4-ethoxy-3-methoxyphenyl)-1H-pyrazolo[4,3-d]pyrimidin-3-yl)ethyl)-N-phenylacrylamide;
N-(2-(7-amino-1-(4-chloro-3-hydroxyphenyl)-1H-pyrazolo[4,3-d]pyrimidin-3-yl)ethyl)-N-phenylacrylamide;
N-(2-(7-amino-1-(3-(hydroxymethyl)phenyl)-1H-pyrazolo[4,3-d]pyrimidin-3-yl)ethyl)-N-phenylacrylamide;
N-(2-(7-amino-1-(4-(hydroxymethyl)phenyl)-1H-pyrazolo[4,3-d]pyrimidin-3-yl)ethyl)-N-phenylacrylamide;
N-(2-(1-(3-(allyloxy)-5-fluorophenyl)-7-amino-1H-pyrazolo[4,3-d]pyrimidin-3-yl)ethyl)-N-phenylacrylamide;
N-(2-(1-(4-acrylamido-3-hydroxyphenyl)-7-amino-1H-pyrazolo[4,3-d]pyrimidin-3-yl)ethyl)-N-phenylacrylamide;
N-(2-(1-(3-acrylamido-5-hydroxyphenyl)-7-amino-1H-pyrazolo[4,3-d]pyrimidin-3-yl)ethyl)-N-phenylacrylamide;
N-(2-(7-amino-1-(1H-indol-1-yl)-1H-pyrazolo[4,3-d]pyrimidin-3-yl)ethyl)-N-phenylacrylamide;
N-(2-(7-amino-1-(5-fluoro-7-hydroxy-1H-indol-1-yl)-1H-pyrazolo[4,3-d]pyrimidin-3-yl)ethyl)-N-phenylacrylamide;
N-(2-(7-amino-1-(6-fluoro-5-hydroxy-1H-indol-1-yl)-1H-pyrazolo[4,3-d]pyrimidin-3-yl)ethyl)-N-phenylacrylamide;
N-(2-(7-amino-1-(4-amino-3-hydroxyphenyl)-1H-pyrazolo[4,3-d]pyrimidin-3-yl)ethyl)-N-phenylacrylamide;
N-(2-(7-amino-1-(3-hydroxy-4-(trifluoromethyl)phenyl)-1H-pyrazolo[4,3-d]pyrimidin-3-yl)ethyl)-N-phenylacrylamide;
N-(2-(1-(4-acryloyl-3-fluorophenyl)-7-amino-1H-pyrazolo[4,3-d]pyrimidin-3-yl)ethyl)-N-phenylacrylamide;
N-(2-(1-(3-acryloyl-5-fluorophenyl)-7-amino-1H-pyrazolo[4,3-d]pyrimidin-3-yl)ethyl)-N-phenylacrylamide;
N-(2-(4-amino-3-(4-fluoro-3-hydroxyphenyl)-1H-pyrazolo[3,4-d]pyrimidin-1-yl)ethyl)-2-cyano-N-phenylacrylamide;
N-(2-(4-amino-3-(3-hydroxy-4-isopropoxyphenyl)-1H-pyrazolo[3,4-d]pyrimidin-1-yl)ethyl)-2-cyano-N-phenylacrylamide;
N-(2-(4-amino-3-(3-hydroxy-4-methoxyphenyl)-1H-pyrazolo[3,4-d]pyrimidin-1-yl)ethyl)-2-cyano-N-phenylacrylamide;
N-(2-(4-amino-3-(3-hydroxy-4-methylphenyl)-1H-pyrazolo[3,4-d]pyrimidin-1-yl)ethyl)-2-cyano-N-phenylacrylamide;
N-(2-(4-amino-3-(3,4-dihydroxyphenyl)-1H-pyrazolo[3,4-d]pyrimidin-1-yl)ethyl)-2-cyano-N-phenylacrylamide;
N-(2-(4-amino-3-(4-methoxy-3-(methylamino)phenyl)-1H-pyrazolo[3,4-d]pyrimidin-1-yl)ethyl)-2-cyano-N-phenylacrylamide;
N-(2-(4-amino-3-(4-fluoro-3-(methylamino)phenyl)-1H-pyrazolo[3,4-d]pyrimidin-1-yl)ethyl)-2-cyano-N-phenylacrylamide;
N-(2-(4-amino-3-(4-hydroxyphenyl)-1H-pyrazolo[3,4-d]pyrimidin-1-yl)ethyl)-2-cyano-N-phenylacrylamide;
N-(2-(4-amino-3-(4-hydroxy-3-methoxyphenyl)-1H-pyrazolo[3,4-d]pyrimidin-1-yl)ethyl)-2-cyano-N-phenylacrylamide;
N-(2-(4-amino-3-(4-hydroxy-3-(trifluoromethyl)phenyl)-1H-pyrazolo[3,4-d]pyrimidin-1-yl)ethyl)-2-cyano-N-phenylacrylamide;
N-(2-(4-amino-3-(3-cyano-4-hydroxyphenyl)-1H-pyrazolo[3,4-d]pyrimidin-1-yl)ethyl)-2-cyano-N-phenylacrylamide;
N-(2-(4-amino-3-(2-hydroxyphenylamino)-1H-pyrazolo[3,4-d]pyrimidin-1-yl)ethyl)-2-cyano-N-phenylacrylamide;
N-(2-(4-amino-3-(3-fluoro-5-methoxyphenyl)-1H-pyrazolo[3,4-d]pyrimidin-1-yl)ethyl)-2-cyano-N-phenylacrylamide;
N-(2-(4-amino-3-(4-bromo-3-hydroxyphenyl)-1H-pyrazolo[3,4-d]pyrimidin-1-yl)ethyl)-2-cyano-N-phenylacrylamide;
N-(2-(4-amino-3-(1H-indazol-5-yl)-1H-pyrazolo[3,4-d]pyrimidin-1-yl)ethyl)-2-cyano-N-phenylacrylamide;
N-(2-(4-amino-3-(1H-indol-6-yl)-1H-pyrazolo[3,4-d]pyrimidin-1-yl)ethyl)-2-cyano-N-phenylacrylamide;
N-(2-(4-amino-3-(1H-benzo[d]imidazol-6-yl)-1H-pyrazolo[3,4-d]pyrimidin-1-yl)ethyl)-2-cyano-N-phenylacrylamide;
N-(2-(4-amino-3-(1H-pyrazol-4-yl)-1H-pyrazolo[3,4-d]pyrimidin-1-yl)ethyl)-2-cyano-N-phenylacrylamide;
N-(2-(4-amino-3-(5-fluoro-6-methoxypyridin-3-yl)-1H-pyrazolo[3,4-d]pyrimidin-1-yl)ethyl)-2-cyano-N-phenylacrylamide;
4-(4-amino-1-(2-(2-cyano-N-phenylacrylamido)ethyl)-1H-pyrazolo[3,4-d]pyrimidin-3-yl)benzamide;
3-(4-amino-1-(2-(2-cyano-N-phenylacrylamido)ethyl)-1H-pyrazolo[3,4-d]pyrimidin-3-yl)benzamide;
N-(2-(4-amino-3-(3,4-dimethoxyphenyl)-1H-pyrazolo[3,4-d]pyrimidin-1-yl)ethyl)-2-cyano-N-phenylacrylamide;
N-(2-(4-amino-3-(4-ethoxy-3-methoxyphenyl)-1H-pyrazolo[3,4-d]pyrimidin-1-yl)ethyl)-2-cyano-N-phenylacrylamide;
N-(2-(4-amino-3-(4-chloro-3-hydroxyphenyl)-1H-pyrazolo[3,4-d]pyrimidin-1-yl)ethyl)-2-cyano-N-phenylacrylamide;
N-(2-(4-amino-3-(3-(hydroxymethyl)phenyl)-1H-pyrazolo[3,4-d]pyrimidin-1-yl)ethyl)-2-cyano-N-phenylacrylamide;
N-(2-(4-amino-3-(4-(hydroxymethyl)phenyl)-1H-pyrazolo[3,4-d]pyrimidin-1-yl)ethyl)-2-cyano-N-phenylacrylamide;
N-(2-(3-(3-(allyloxy)-5-fluorophenyl)-4-amino-1H-pyrazolo[3,4-d]pyrimidin-1-yl)ethyl)-2-cyano-N-phenylacrylamide;
N-(2-(3-(4-acrylamido-3-hydroxyphenyl)-4-amino-1H-pyrazolo[3,4-d]pyrimidin-1-yl)ethyl)-2-cyano-N-phenylacrylamide;
N-(2-(3-(3-acrylamido-5-hydroxyphenyl)-4-amino-1H-pyrazolo[3,4-d]pyrimidin-1-yl)ethyl)-2-cyano-N-phenylacrylamide;
N-(2-(4-amino-3-(1H-indol-1-yl)-1H-pyrazolo[3,4-d]pyrimidin-1-yl)ethyl)-2-cyano-N-phenylacrylamide;
N-(2-(4-amino-3-(5-fluoro-7-hydroxy-1H-indol-1-yl)-1H-pyrazolo[3,4-d]pyrimidin-1-yl)ethyl)-2-cyano-N-phenylacrylamide;
N-(2-(4-amino-3-(6-fluoro-5-hydroxy-1H-indol-1-yl)-1H-pyrazolo[3,4-d]pyrimidin-1-yl)ethyl)-2-cyano-N-phenylacrylamide;
N-(2-(4-amino-3-(4-amino-3-hydroxyphenyl)-1H-pyrazolo[3,4-d]pyrimidin-1-yl)ethyl)-2-cyano-N-phenylacrylamide;
N-(2-(4-amino-3-(3-hydroxy-4-(trifluoromethyl)phenyl)-1H-pyrazolo[3,4-d]pyrimidin-1-yl)ethyl)-2-cyano-N-phenylacrylamide;
N-(2-(3-(4-acryloyl-3-fluorophenyl)-4-amino-1H-pyrazolo[3,4-d]pyrimidin-1-yl)ethyl)-2-cyano-N-phenylacrylamide;
N-(2-(3-(3-acryloyl-5-fluorophenyl)-4-amino-1H-pyrazolo[3,4-d]pyrimidin-1-yl)ethyl)-2-cyano-N-phenylacrylamide;
N-((1-(4-amino-3-(4-fluoro-3-hydroxyphenyl)-1H-pyrazolo[3,4-d]pyrimidin-1-yl)cyclopropyl)methyl)-N-phenylacrylamide;
N-((1-(4-amino-3-(3-hydroxy-4-isopropoxyphenyl)-1H-pyrazolo[3,4-d]pyrimidin-1-yl)cyclopropyl)methyl)-N-phenylacrylamide;
N-((1-(4-amino-3-(3-hydroxy-4-methoxyphenyl)-1H-pyrazolo[3,4-d]pyrimidin-1-yl)cyclopropyl)methyl)-N-phenylacrylamide;
N-((1-(4-amino-3-(3-hydroxy-4-methylphenyl)-1H-pyrazolo[3,4-d]pyrimidin-1-yl)cyclopropyl)methyl)-N-phenylacrylamide;
N-((1-(4-amino-3-(3,4-dihydroxyphenyl)-1H-pyrazolo[3,4-d]pyrimidin-1-yl)cyclopropyl)methyl)-N-phenylacrylamide;
N-((1-(4-amino-3-(4-methoxy-3-(methylamino)phenyl)-1H-pyrazolo[3,4-d]pyrimidin-1-yl)cyclopropyl)methyl)-N-phenylacrylamide;
N-((1-(4-amino-3-(4-fluoro-3-(methylamino)phenyl)-1H-pyrazolo[3,4-d]pyrimidin-1-yl)cyclopropyl)methyl)-N-phenylacrylamide;
N-((1-(4-amino-3-(4-hydroxyphenyl)-1H-pyrazolo[3,4-d]pyrimidin-1-yl)cyclopropyl)methyl)-N-phenylacrylamide;
N-((1-(4-amino-3-(4-hydroxy-3-methoxyphenyl)-1H-pyrazolo[3,4-d]pyrimidin-1-yl)cyclopropyl)methyl)-N-phenylacrylamide;
N-((1-(4-amino-3-(4-hydroxy-3-(trifluoromethyl)phenyl)-1H-pyrazolo[3,4-d]pyrimidin-1-yl)cyclopropyl)methyl)-N-phenylacrylamide;
N-((1-(4-amino-3-(3-cyano-4-hydroxyphenyl)-1H-pyrazolo[3,4-d]pyrimidin-1-yl)cyclopropyl)methyl)-N-phenylacrylamide;
N-((1-(4-amino-3-(2-hydroxyphenylamino)-1H-pyrazolo[3,4-d]pyrimidin-1-yl)cyclopropyl)methyl)-N-phenylacrylamide;
N-((1-(4-amino-3-(3-fluoro-5-methoxyphenyl)-1H-pyrazolo[3,4-d]pyrimidin-1-yl)cyclopropyl)methyl)-N-phenylacrylamide;
N-((1-(4-amino-3-(4-bromo-3-hydroxyphenyl)-1H-pyrazolo[3,4-d]pyrimidin-1-yl)cyclopropyl)methyl)-N-phenylacrylamide;
N-((1-(4-amino-3-(1H-indazol-5-yl)-1H-pyrazolo[3,4-d]pyrimidin-1-yl)cyclopropyl)methyl)-N-phenylacrylamide;
N-((1-(4-amino-3-(1H-indol-6-yl)-1H-pyrazolo[3,4-d]pyrimidin-1-yl)cyclopropyl)methyl)-N-phenylacrylamide;
N-((1-(4-amino-3-(1H-benzo[d]imidazol-6-yl)-1H-pyrazolo[3,4-d]pyrimidin-1-yl)cyclopropyl)methyl)-N-phenylacrylamide;
N-((1-(4-amino-3-(1H-pyrazol-4-yl)-1H-pyrazolo[3,4-d]pyrimidin-1-yl)cyclopropyl)methyl)-N-phenylacrylamide;
N-((1-(4-amino-3-(5-fluoro-6-methoxypyridin-3-yl)-1H-pyrazolo[3,4-d]pyrimidin-1-yl)cyclopropyl)methyl)-N-phenylacrylamide;
4-(4-amino-1-(1-((N-phenylacrylamido)methyl)cyclopropyl)-1H-pyrazolo[3,4-d]pyrimidin-3-yl)benzamide;
3-(4-amino-1-(1-((N-phenylacrylamido)methyl)cyclopropyl)-1H-pyrazolo [3,4-d]pyrimidin-3-yl)benzamide;
N-((1-(4-amino-3-(3,4-dimethoxyphenyl)-1H-pyrazolo[3,4-d]pyrimidin-1-yl)cyclopropyl)methyl)-N-phenylacrylamide;
N-((1-(4-amino-3-(4-ethoxy-3-methoxyphenyl)-1H-pyrazolo[3,4-d]pyrimidin-1-yl)cyclopropyl)methyl)-N-phenylacrylamide;
N-((1-(4-amino-3-(4-chloro-3-hydroxyphenyl)-1H-pyrazolo[3,4-d]pyrimidin-1-yl)cyclopropyl)methyl)-N-phenylacrylamide;
N-((1-(4-amino-3-(3-(hydroxymethyl)phenyl)-1H-pyrazolo[3,4-d]pyrimidin-1-yl)cyclopropyl)methyl)-N-phenylacrylamide;
N-((1-(4-amino-3-(4-(hydroxymethyl)phenyl)-1H-pyrazolo[3,4-d]pyrimidin-1-yl)cyclopropyl)methyl)-N-phenylacrylamide;
N-((1-(3-(3-(allyloxy)-5-fluorophenyl)-4-amino-1H-pyrazolo[3,4-d]pyrimidin-1-yl)cyclopropyl)methyl)-N-phenylacrylamide;
N-((1-(3-(4-acrylamido-3-hydroxyphenyl)-4-amino-1H-pyrazolo[3,4-d]pyrimidin-1-yl)cyclopropyl)methyl)-N-phenylacrylamide;
N-((1-(3-(3-acrylamido-5-hydroxyphenyl)-4-amino-1H-pyrazolo[3,4-d]pyrimidin-1-yl)cyclopropyl)methyl)-N-phenylacrylamide;
N-((1-(4-amino-3-(1H-indol-1-yl)-1H-pyrazolo[3,4-d]pyrimidin-1-yl)cyclopropyl)methyl)-N-phenylacrylamide;
N-((1-(4-amino-3-(5-fluoro-7-hydroxy-1H-indol-1-yl)-1H-pyrazolo[3,4-d]pyrimidin-1-yl)cyclopropyl)methyl)-N-phenylacrylamide;
N-((1-(4-amino-3-(6-fluoro-5-hydroxy-1H-indol-1-yl)-1H-pyrazolo[3,4-d]pyrimidin-1-yl)cyclopropyl)methyl)-N-phenylacrylamide;
N-((1-(4-amino-3-(4-amino-3-hydroxyphenyl)-1H-pyrazolo[3,4-d]pyrimidin-1-yl)cyclopropyl)methyl)-N-phenylacrylamide;
N-((1-(4-amino-3-(3-hydroxy-4-(trifluoromethyl)phenyl)-1H-pyrazolo[3,4-d]pyrimidin-1-yl)cyclopropyl)methyl)-N-phenylacrylamide;
N-((1-(3-(4-acryloyl-3-fluorophenyl)-4-amino-1H-pyrazolo[3,4-d]pyrimidin-1-yl)cyclopropyl)methyl)-N-phenylacrylamide;
N-((1-(3-(3-acryloyl-5-fluorophenyl)-4-amino-1H-pyrazolo[3,4-d]pyrimidin-1-yl)cyclopropyl)methyl)-N-phenylacrylamide;
N-(2-(4-amino-3-(4-fluoro-3-hydroxyphenyl)-1H-pyrazolo[3,4-d]pyrimidin-1-yl)ethyl)-N-phenylethenesulfonamide;
N-(2-(4-amino-3-(3-hydroxy-4-isopropoxyphenyl)-1H-pyrazolo[3,4-d]pyrimidin-1-yl)ethyl)-N-phenylethenesulfonamide;
N-(2-(4-amino-3-(3-hydroxy-4-methoxyphenyl)-1H-pyrazolo[3,4-d]pyrimidin-1-yl)ethyl)-N-phenylethenesulfonamide;
N-(2-(4-amino-3-(3-hydroxy-4-methylphenyl)-1H-pyrazolo[3,4-d]pyrimidin-1-yl)ethyl)-N-phenylethenesulfonamide;
N-(2-(4-amino-3-(3,4-dihydroxyphenyl)-1H-pyrazolo[3,4-d]pyrimidin-1-yl)ethyl)-N-phenylethenesulfonamide;
N-(2-(4-amino-3-(4-methoxy-3-(methylamino)phenyl)-1H-pyrazolo[3,4-d]pyrimidin-1-yl)ethyl)-N-phenylethenesulfonamide;
N-(2-(4-amino-3-(4-fluoro-3-(methylamino)phenyl)-1H-pyrazolo[3,4-d]pyrimidin-1-yl)ethyl)-N-phenylethenesulfonamide;
N-(2-(4-amino-3-(4-hydroxyphenyl)-1H-pyrazolo[3,4-d]pyrimidin-1-yl)ethyl)-N-phenylethenesulfonamide;
N-(2-(4-amino-3-(4-hydroxy-3-methoxyphenyl)-1H-pyrazolo[3,4-d]pyrimidin-1-yl)ethyl)-N-phenylethenesulfonamide;
N-(2-(4-amino-3-(4-hydroxy-3-(trifluoromethyl)phenyl)-1H-pyrazolo[3,4-d]pyrimidin-1-yl)ethyl)-N-phenylethenesulfonamide;
N-(2-(4-amino-3-(3-cyano-4-hydroxyphenyl)-1H-pyrazolo[3,4-d]pyrimidin-1-yl)ethyl)-N-phenylethenesulfonamide;
N-(2-(4-amino-3-(2-hydroxyphenylamino)-1H-pyrazolo[3,4-d]pyrimidin-1-yl)ethyl)-N-phenylethenesulfonamide;
N-(2-(4-amino-3-(3-fluoro-5-methoxyphenyl)-1H-pyrazolo[3,4-d]pyrimidin-1-yl)ethyl)-N-phenylethenesulfonamide;
N-(2-(4-amino-3-(4-bromo-3-hydroxyphenyl)-1H-pyrazolo[3,4-d]pyrimidin-1-yl)ethyl)-N-phenylethenesulfonamide;
N-(2-(4-amino-3-(1H-indazol-5-yl)-1H-pyrazolo[3,4-d]pyrimidin-1-yl)ethyl)-N-phenylethenesulfonamide;
N-(2-(4-amino-3-(1H-indol-6-yl)-1H-pyrazolo[3,4-d]pyrimidin-1-yl)ethyl)-N-phenylethenesulfonamide;
N-(2-(4-amino-3-(1H-benzo[d]imidazol-6-yl)-1H-pyrazolo[3,4-d]pyrimidin-1-yl)ethyl)-N-phenylethenesulfonamide;
N-(2-(4-amino-3-(1H-pyrazol-4-yl)-1H-pyrazolo[3,4-d]pyrimidin-1-yl)ethyl)-N-phenylethenesulfonamide;
N-(2-(4-amino-3-(5-fluoro-6-methoxypyridin-3-yl)-1H-pyrazolo[3,4-d]pyrimidin-1-yl)ethyl)-N-phenylethenesulfonamide;
4-(4-amino-1-(2-(N-phenylvinylsulfonamido)ethyl)-1H-pyrazolo[3,4-d]pyrimidin-3-yl)benzamide;
3-(4-amino-1-(2-(N-phenylvinylsulfonamido)ethyl)-1H-pyrazolo[3,4-d]pyrimidin-3-yl)benzamide;
N-(2-(4-amino-3-(3,4-dimethoxyphenyl)-1H-pyrazolo[3,4-d]pyrimidin-1-yl)ethyl)-N-phenylethenesulfonamide;
N-(2-(4-amino-3-(4-ethoxy-3-methoxyphenyl)-1H-pyrazolo[3,4-d]pyrimidin-1-yl)ethyl)-N-phenylethenesulfonamide;
N-(2-(4-amino-3-(4-chloro-3-hydroxyphenyl)-1H-pyrazolo[3,4-d]pyrimidin-1-yl)ethyl)-N-phenylethenesulfonamide;
N-(2-(4-amino-3-(3-(hydroxymethyl)phenyl)-1H-pyrazolo[3,4-d]pyrimidin-1-yl)ethyl)-N-phenylethenesulfonamide;
N-(2-(4-amino-3-(4-(hydroxymethyl)phenyl)-1H-pyrazolo[3,4-d]pyrimidin-1-yl)ethyl)-N-phenylethenesulfonamide;
N-(2-(3-(3-(allyloxy)-5-fluorophenyl)-4-amino-1H-pyrazolo[3,4-d]pyrimidin-1-yl)ethyl)-N-phenylethenesulfonamide;
N-(4-(4-amino-1-(2-(N-phenylvinylsulfonamido)ethyl)-1H-pyrazolo[3,4-d]pyrimidin-3-yl)-2-hydroxyphenyl)acrylamide;
N-(3-(4-amino-1-(2-(N-phenylvinylsulfonamido)ethyl)-1H-pyrazolo[3,4-d]pyrimidin-3-yl)-5-hydroxyphenyl)acrylamide;
N-(2-(4-amino-3-(1H-indol-1-yl)-1H-pyrazolo[3,4-d]pyrimidin-1-yl)ethyl)-N-phenylethenesulfonamide;
N-(2-(4-amino-3-(5-fluoro-7-hydroxy-1H-indol-1-yl)-1H-pyrazolo[3,4-d]pyrimidin-1-yl)ethyl)-N-phenylethenesulfonamide;
N-(2-(4-amino-3-(6-fluoro-5-hydroxy-1H-indol-1-yl)-1H-pyrazolo[3,4-d]pyrimidin-1-yl)ethyl)-N-phenylethenesulfonamide;
N-(2-(4-amino-3-(4-amino-3-hydroxyphenyl)-1H-pyrazolo[3,4-d]pyrimidin-1-yl)ethyl)-N-phenylethenesulfonamide;
N-(2-(4-amino-3-(3-hydroxy-4-(trifluoromethyl)phenyl)-1H-pyrazolo[3,4-d]pyrimidin-1-yl)ethyl)-N-phenylethenesulfonamide;
N-(2-(3-(4-acryloyl-3-fluorophenyl)-4-amino-1H-pyrazolo[3,4-d]pyrimidin-1-yl)ethyl)-N-phenylethenesulfonamide;
N-(2-(3-(3-acryloyl-5-fluorophenyl)-4-amino-1H-pyrazolo[3,4-d]pyrimidin-1-yl)ethyl)-N-phenylethenesulfonamide;
N-(2-(4-amino-3-(4-fluoro-3-hydroxyphenyl)-1H-pyrazolo[3,4-d]pyrimidin-1-yl)ethyl)-N-(pyridin-3-yl)acrylamide;
N-(2-(4-amino-3-(4-fluoro-3-hydroxyphenyl)-1H-pyrazolo[3,4-d]pyrimidin-1-yl)ethyl)-N-(1H-benzo[d]imidazol-5-yl)acrylamide;
N-(2-(4-amino-3-(4-fluoro-3-hydroxyphenyl)-1H-pyrazolo[3,4-d]pyrimidin-1-yl)ethyl)-2-cyano-N-(pyridin-3-yl)acrylamide;
2-(4-amino-3-(4-fluoro-3-hydroxyphenyl)-1H-pyrazolo[3,4-d]pyrimidin-1-yl)-N-(cyanomethyl)-N-phenylacetamide;
2-(4-amino-3-(4-fluoro-3-hydroxyphenyl)-1H-pyrazolo[3,4-d]pyrimidin-1-yl)-N-(2-cyanoethyl)-N-phenylacetamide;
N-(2-(4-amino-3-(4-fluoro-3-hydroxyphenyl)-1H-pyrazolo[3,4-d]pyrimidin-1-yl)ethyl)-N-(cyanomethyl)benzamide;
N-(2-(4-amino-3-(4-fluoro-3-hydroxyphenyl)-1H-pyrazolo[3,4-d]pyrimidin-1-yl)ethyl)-N-phenylcyclopropanecarboxamide;
N-(2-(4-amino-3-(4-fluoro-3-hydroxyphenyl)-1H-pyrazolo[3,4-d]pyrimidin-1-yl)ethyl)-2-(methylsulfonyl)-N-phenylacrylamide; and
N1-(2-(4-amino-3-(4-fluoro-3-hydroxyphenyl)-1H-pyrazolo[3,4-d]pyrimidin-1-yl)ethyl)-2-methylene-N1-phenylmalonamide; and pharmaceutically acceptable salts thereof.

33. A pharmaceutical composition comprising a compound of claim 1, and a pharmaceutically acceptable carrier.

34. A method for the treatment of cancer or for the treatment of an autoimmune disease to a patient comprising the administration of a therapeutically effective amount of a Btk inhibitor to a patient in need thereof, wherein the Btk inhibitor is a compound of the formula (I) or any one of a compound of the formula B-1 to B-10; B-1a, to B-10c; B-1a-1 to B-10c-8; C-1a to C-3j; or (D-1) to (D-3); or a compound of Table 1 or 2, or a pharmaceutically acceptable salt thereof.

35. The method of claim 34, wherein the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma.

36. The method of claim 34, wherein the autoimmune disease is selected from the group consisting of rheumatoid arthritis and systemic lupus erythematosis.

37. A method for the treatment of cancer or for the treatment of an autoimmune disease to a patient comprising the administration of a therapeutically effective amount of a PI3Kδ inhibitor to a patient in need thereof, wherein the PI3Kδ inhibitor is a compound of the formula (I) or any one of a compound of the formula B-1 to B-10; B-1a, to B-10c; B-1a-1 to B-10c-8; C-1a to C-3j; or (D-1) to (D-3); or a compound of Table 1 or 2, or a pharmaceutically acceptable salt thereof.

38. The method of claim 37, wherein the cancer is selected from the group consisting of chronic lymphocytic leukemia, mantle cell lymphoma, B cell non Hodgkin lymphoma, multiple myeloma and acute myeloid lymphoma.

39. The method of claim 36, wherein the autoimmune disease is selected from the group consisting of rheumatoid arthritis, allergic asthma and myocardial infarction.

40. A method for the treatment of cancer or for the treatment of an autoimmune disease to a patient comprising the administration of a therapeutically effective amount of a dual Btk and PI3Kδ inhibitor to a patient in need thereof, wherein the dual Btk and PI3Kδ inhibitor is a compound of the formula (I) wherein the
